SEQUENCE LISTING (1635 sequences)

SEQ ID NO: 1 - DNA, 10 bp, Artificial
  SAGE Tag
agggccgact

SEQ ID NO: 2 - DNA, 10 bp, Artificial
  SAGE Tag
actgccacag

SEQ ID NO: 3 - DNA, 10 bp, Artificial
  SAGE Tag
ggaagagggt

SEQ ID NO: 4 - DNA, 10 bp, Artificial
  SAGE Tag
gggaagtcac

SEQ ID NO: 5 - DNA, 10 bp, Artificial
  SAGE Tag
ggctgtgcca

SEQ ID NO: 6 - DNA, 10 bp, Artificial
  SAGE Tag
attttttaac

SEQ ID NO: 7 - DNA, 10 bp, Artificial
  SAGE Tag
ccacacaagc

SEQ ID NO: 8 - DNA, 10 bp, Artificial
  SAGE Tag
cataccaggc

SEQ ID NO: 9 - DNA, 10 bp, Artificial
  SAGE Tag
caagccctgc

SEQ ID NO: 10 - DNA, 10 bp, Artificial
  SAGE Tag
tctgaccacc

SEQ ID NO: 11 - DNA, 10 bp, Artificial
  SAGE Tag
ttgagggggt

SEQ ID NO: 12 - DNA, 10 bp, Artificial
  SAGE Tag
ttagtcttca

SEQ ID NO: 13 - DNA, 10 bp, Artificial
  SAGE Tag
cctgtaaagc

SEQ ID NO: 14 - DNA, 10 bp, Artificial
  SAGE Tag
aagaaccaag

SEQ ID NO: 15 - DNA, 10 bp, Artificial
  SAGE Tag
gacttggagg

SEQ ID NO: 16 - DNA, 10 bp, Artificial
  SAGE Tag
aacccttggg

SEQ ID NO: 17 - DNA, 10 bp, Artificial
  SAGE Tag
cctgctccct

SEQ ID NO: 18 - DNA, 10 bp, Artificial
  SAGE Tag
gcaaggttgc

SEQ ID NO: 19 - DNA, 10 bp, Artificial
  SAGE Tag
aagcgctctc

SEQ ID NO: 20 - DNA, 10 bp, Artificial
  SAGE Tag
tggaggtggg

SEQ ID NO: 21 - DNA, 10 bp, Artificial
  SAGE Tag
gcccgtgcca

SEQ ID NO: 22 - DNA, 10 bp, Artificial
  SAGE Tag
ttttacatct

SEQ ID NO: 23 - DNA, 10 bp, Artificial
  SAGE Tag
tctgtgctca

SEQ ID NO: 24 - DNA, 10 bp, Artificial
  SAGE Tag
gagtgaaaga

SEQ ID NO: 25 - DNA, 10 bp, Artificial
  SAGE Tag
tgttctttgc

SEQ ID NO: 26 - DNA, 10 bp, Artificial
  SAGE Tag
aagcccaggc

SEQ ID NO: 27 - DNA, 10 bp, Artificial
  SAGE Tag
gaaggatgtg

SEQ ID NO: 28 - DNA, 10 bp, Artificial
  SAGE Tag
tgtgtcaaag

SEQ ID NO: 29 - DNA, 10 bp, Artificial
  SAGE Tag
cacacagttt

SEQ ID NO: 30 - DNA, 10 bp, Artificial
  SAGE Tag
accagctccc

SEQ ID NO: 31 - DNA, 10 bp, Artificial
  SAGE Tag
ccctggctgt

SEQ ID NO: 32 - DNA, 10 bp, Artificial
  SAGE Tag
tttggagcat

SEQ ID NO: 33 - DNA, 10 bp, Artificial
  SAGE Tag
ggacggcatt

SEQ ID NO: 34 - DNA, 10 bp, Artificial
  SAGE Tag
tctgtttact

SEQ ID NO: 35 - DNA, 10 bp, Artificial
  SAGE Tag
ctcacacatt

SEQ ID NO: 36 - DNA, 10 bp, Artificial
  SAGE Tag
ggcgtgaacc

SEQ ID NO: 37 - DNA, 10 bp, Artificial
  SAGE Tag
cctcactttc

SEQ ID NO: 38 - DNA, 10 bp, Artificial
  SAGE Tag
ggactgggag

SEQ ID NO: 39 - DNA, 10 bp, Artificial
  SAGE Tag
aagccagttt

SEQ ID NO: 40 - DNA, 10 bp, Artificial
  SAGE Tag
ctcaaccccc

SEQ ID NO: 41 - DNA, 10 bp, Artificial
  SAGE Tag
ggagctggcc

SEQ ID NO: 42 - DNA, 10 bp, Artificial
  SAGE Tag
agccaccacg

SEQ ID NO: 43 - DNA, 10 bp, Artificial
  SAGE Tag
gtggctgctg

SEQ ID NO: 44 - DNA, 10 bp, Artificial
  SAGETag
tggcttaaat

SEQ ID NO: 45 - DNA, 10 bp, Artificial
  SAGE Tag
tacgctcctt

SEQ ID NO: 46 - DNA, 10 bp, Artificial
  SAGE Tag
ctctgtaatt

SEQ ID NO: 47 - DNA, 10 bp, Artificial
  SAGE Tag
tggagagtcg

SEQ ID NO: 48 - DNA, 10 bp, Artificial
  SAGE Tag
cccatccgca

SEQ ID NO: 49 - DNA, 10 bp, Artificial
  SAGE Tag
aattcccgtc

SEQ ID NO: 50 - DNA, 10 bp, Artificial
  SAGE Tag
gccactaccc

SEQ ID NO: 51 - DNA, 10 bp, Artificial
  SAGE Tag
ggccctgcag

SEQ ID NO: 52 - DNA, 10 bp, Artificial
  SAGE Tag
aggttctgcc

SEQ ID NO: 53 - DNA, 10 bp, Artificial
  SAGE Tag
tcaacggtgt

SEQ ID NO: 54 - DNA, 10 bp, Artificial
  SAGE Tag
ttctgaagca

SEQ ID NO: 55 - DNA, 10 bp, Artificial
  SAGE Tag
gtctccggga

SEQ ID NO: 56 - DNA, 10 bp, Artificial
  SAGE Tag
gaggcgatca

SEQ ID NO: 57 - DNA, 10 bp, Artificial
  SAGE Tag
tagaattttc

SEQ ID NO: 58 - DNA, 10 bp, Artificial
  SAGE Tag
atcccactgc

SEQ ID NO: 59 - DNA, 10 bp, Artificial
  SAGE Tag
tggttctata

SEQ ID NO: 60 - DNA, 10 bp, Artificial
  SAGE Tag
acaaaatatg

SEQ ID NO: 61 - DNA, 10 bp, Artificial
  SAGE Tag
tgagcaagcc

SEQ ID NO: 62 - DNA, 10 bp, Artificial
  SAGE Tag
ctttgatcag

SEQ ID NO: 63 - DNA, 10 bp, Artificial
  SAGE Tag
aggccctgct

SEQ ID NO: 64 - DNA, 10 bp, Artificial
  SAGE Tag
ctgtggttac

SEQ ID NO: 65 - DNA, 10 bp, Artificial
  SAGE Tag
ctggtgatgg

SEQ ID NO: 66 - DNA, 10 bp, Artificial
  SAGE Tag
tacccccgag

SEQ ID NO: 67 - DNA, 10 bp, Artificial
  SAGE Tag
gtataatttg

SEQ ID NO: 68 - DNA, 10 bp, Artificial
  SAGE Tag
cagccaggac

SEQ ID NO: 69 - DNA, 10 bp, Artificial
  SAGE Tag
tctttcaccc

SEQ ID NO: 70 - DNA, 10 bp, Artificial
  SAGE Tag
gtggggggga

SEQ ID NO: 71 - DNA, 10 bp, Artificial
  SAGE Tag
tgtagctgca

SEQ ID NO: 72 - DNA, 10 bp, Artificial
  SAGE Tag
gccagctgac

SEQ ID NO: 73 - DNA, 10 bp, Artificial
  SAGE Tag
ttttcccctg

SEQ ID NO: 74 - DNA, 10 bp, Artificial
  SAGE Tag
acccaatcct

SEQ ID NO: 75 - DNA, 10 bp, Artificial
  SAGE Tag
caaactgggg

SEQ ID NO: 76 - DNA, 10 bp, Artificial
  SAGE Tag
ggctggggtc

SEQ ID NO: 77 - DNA, 10 bp, Artificial
  SAGE Tag
gctctgtcac

SEQ ID NO: 78 - DNA, 10 bp, Artificial
  SAGE Tag
cctttcaaaa

SEQ ID NO: 79 - DNA, 10 bp, Artificial
  SAGE Tag
tgcaatatgg

SEQ ID NO: 80 - DNA, 10 bp, Artificial
  SAGE Tag
ccgtccggaa

SEQ ID NO: 81 - DNA, 10 bp, Artificial
  SAGE Tag
agcggagtct

SEQ ID NO: 82 - DNA, 10 bp, Artificial
  SAGE Tag
accagagagc

SEQ ID NO: 83 - DNA, 10 bp, Artificial
  SAGE Tag
ggggctgggg

SEQ ID NO: 84 - DNA, 10 bp, Artificial
  SAGE Tag
gacagcctta

SEQ ID NO: 85 - DNA, 10 bp, Artificial
  SAGE Tag
gcggaacgca

SEQ ID NO: 86 - DNA, 10 bp, Artificial
  SAGE Tag
tggcaaagcc

SEQ ID NO: 87 - DNA, 10 bp, Artificial
  SAGE Tag
ggaacaacag

SEQ ID NO: 88 - DNA, 10 bp, Artificial
  SAGE Tag
gtatttgtgg

SEQ ID NO: 89 - DNA, 10 bp, Artificial
  SAGE Tag
ctgagcagaa

SEQ ID NO: 90 - DNA, 10 bp, Artificial
  SAGE Tag
gcttgctgcc

SEQ ID NO: 91 - DNA, 10 bp, Artificial
  SAGE Tag
gggggttggt

SEQ ID NO: 92 - DNA, 10 bp, Artificial
  SAGE Tag
cttctggtgc

SEQ ID NO: 93 - DNA, 10 bp, Artificial
  SAGE Tag
ggatcctcgg

SEQ ID NO: 94 - DNA, 10 bp, Artificial
  SAGE Tag
ggtgaatctc

SEQ ID NO: 95 - DNA, 10 bp, Artificial
  SAGE Tag
ggctggggcc

SEQ ID NO: 96 - DNA, 10 bp, Artificial
  SAGE Tag
gcctttctaa

SEQ ID NO: 97 - DNA, 10 bp, Artificial
  SAGE Tag
aactgtcctt

SEQ ID NO: 98 - DNA, 10 bp, Artificial
  SAGE Tag
tttttgtttt

SEQ ID NO: 99 - DNA, 10 bp, Artificial
  SAGE Tag
tgcaccttgg

SEQ ID NO: 100 - DNA, 10 bp, Artificial
  SAGE Tag
ttcctccacg

SEQ ID NO: 101 - DNA, 10 bp, Artificial
  SAGE Tag
aggtgcagag

SEQ ID NO: 102 - DNA, 10 bp, Artificial
  SAGE Tag
cagcccaacc

SEQ ID NO: 103 - DNA, 10 bp, Artificial
  SAGE Tag
ggctcgggat

SEQ ID NO: 104 - DNA, 10 bp, Artificial
  SAGE Tag
tgtgaacaca

SEQ ID NO: 105 - DNA, 10 bp, Artificial
  SAGE Tag
acaaaataaa

SEQ ID NO: 106 - DNA, 10 bp, Artificial
  SAGE Tag
aaacactctt

SEQ ID NO: 107 - DNA, 10 bp, Artificial
  SAGE Tag
ttgaattgaa

SEQ ID NO: 108 - DNA, 10 bp, Artificial
  SAGE Tag
atgactgtac

SEQ ID NO: 109 - DNA, 10 bp, Artificial
  SAGE Tag
ctcccaggtc

SEQ ID NO: 110 - DNA, 10 bp, Artificial
  SAGE Tag
ctgttggcat

SEQ ID NO: 111 - DNA, 10 bp, Artificial
  SAGE Tag
aacgggccgg

SEQ ID NO: 112 - DNA, 10 bp, Artificial
  SAGE Tag
ccacgttcca

SEQ ID NO: 113 - DNA, 10 bp, Artificial
  SAGE Tag
atgcgcaagg

SEQ ID NO: 114 - DNA, 10 bp, Artificial
  SAGE Tag
gtgcttgtac

SEQ ID NO: 115 - DNA, 10 bp, Artificial
  SAGE Tag
aggaagagcc

SEQ ID NO: 116 - DNA, 10 bp, Artificial
  SAGE Tag
acctgctggt

SEQ ID NO: 117 - DNA, 10 bp, Artificial
  SAGE Tag
gtggtgggtg

SEQ ID NO: 118 - DNA, 10 bp, Artificial
  SAGE Tag
ccggacctgt

SEQ ID NO: 119 - DNA, 10 bp, Artificial
  SAGE Tag
gtgcctagga

SEQ ID NO: 120 - DNA, 10 bp, Artificial
  SAGE Tag
ctcacttctt

SEQ ID NO: 121 - DNA, 10 bp, Artificial
  SAGE Tag
ctaaggtggg

SEQ ID NO: 122 - DNA, 10 bp, Artificial
  SAGE Tag
gttgtgatgt

SEQ ID NO: 123 - DNA, 10 bp, Artificial
  SAGE Tag
ttctggcact

SEQ ID NO: 124 - DNA, 10 bp, Artificial
  SAGE Tag
gggcgggggc

SEQ ID NO: 125 - DNA, 10 bp, Artificial
  SAGE Tag
tactgtggat

SEQ ID NO: 126 - DNA, 10 bp, Artificial
  SAGE Tag
cgggatgcag

SEQ ID NO: 127 - DNA, 10 bp, Artificial
  SAGE Tag
cgcttttgta

SEQ ID NO: 128 - DNA, 10 bp, Artificial
  SAGE Tag
cttatgatca

SEQ ID NO: 129 - DNA, 10 bp, Artificial
  SAGE Tag
cagaagaggc

SEQ ID NO: 130 - DNA, 10 bp, Artificial
  SAGE Tag
gctaggtatt

SEQ ID NO: 131 - DNA, 10 bp, Artificial
  SAGE Tag
gtaaaaccct

SEQ ID NO: 132 - DNA, 10 bp, Artificial
  SAGE Tag
tggaaataaa

SEQ ID NO: 133 - DNA, 10 bp, Artificial
  SAGE Tag
gtgaagtctt

SEQ ID NO: 134 - DNA, 10 bp, Artificial
  SAGE Tag
gagtagagaa

SEQ ID NO: 135 - DNA, 10 bp, Artificial
  SAGE Tag
agggaggcag

SEQ ID NO: 136 - DNA, 10 bp, Artificial
  SAGE Tag
agcaacagtg

SEQ ID NO: 137 - DNA, 10 bp, Artificial
  SAGE Tag
gagaatctgc

SEQ ID NO: 138 - DNA, 10 bp, Artificial
  SAGE Tag
aaaaaccata

SEQ ID NO: 139 - DNA, 10 bp, Artificial
  SAGE Tag
gtctttctgg

SEQ ID NO: 140 - DNA, 10 bp, Artificial
  SAGE Tag
gctactatta

SEQ ID NO: 141 - DNA, 10 bp, Artificial
  SAGE Tag
cagctggggc

SEQ ID NO: 142 - DNA, 10 bp, Artificial
  SAGE Tag
ttctctcaac

SEQ ID NO: 143 - DNA, 10 bp, Artificial
  SAGE Tag
acggctccga

SEQ ID NO: 144 - DNA, 10 bp, Artificial
  SAGE Tag
tgggctctga

SEQ ID NO: 145 - DNA, 10 bp, Artificial
  SAGE Tag
ggcgtcctgg

SEQ ID NO: 146 - DNA, 10 bp, Artificial
  SAGE Tag
tatgtgccac

SEQ ID NO: 147 - DNA, 10 bp, Artificial
  SAGE Tag
tgtgacctct

SEQ ID NO: 148 - DNA, 10 bp, Artificial
  SAGE Tag
aagacaagtg

SEQ ID NO: 149 - DNA, 10 bp, Artificial
  SAGE Tag
agaatgtacg

SEQ ID NO: 150 - DNA, 10 bp, Artificial
  SAGE Tag
ggtgctccct

SEQ ID NO: 151 - DNA, 10 bp, Artificial
  SAGE Tag
gcgagcagcg

SEQ ID NO: 152 - DNA, 10 bp, Artificial
  SAGE Tag
cactgagcca

SEQ ID NO: 153 - DNA, 10 bp, Artificial
  SAGE Tag
gccaggggcc

SEQ ID NO: 154 - DNA, 10 bp, Artificial
  SAGE Tag
cagagtgact

SEQ ID NO: 155 - DNA, 10 bp, Artificial
  SAGE Tag
ctatattttt

SEQ ID NO: 156 - DNA, 10 bp, Artificial
  SAGE Tag
atgaaacccc

SEQ ID NO: 157 - DNA, 10 bp, Artificial
  SAGE Tag
atgtgaggga

SEQ ID NO: 158 - DNA, 10 bp, Artificial
  SAGE Tag
tagtaagtca

SEQ ID NO: 159 - DNA, 10 bp, Artificial
  SAGE Tag
cctgaatctg

SEQ ID NO: 160 - DNA, 10 bp, Artificial
  SAGE Tag
gtggtgtgtg

SEQ ID NO: 161 - DNA, 10 bp, Artificial
  SAGE Tag
cagaagtgtc

SEQ ID NO: 162 - DNA, 10 bp, Artificial
  SAGE Tag
gctctgtaag

SEQ ID NO: 163 - DNA, 10 bp, Artificial
  SAGE Tag
gtaaaagttc

SEQ ID NO: 164 - DNA, 10 bp, Artificial
  SAGE Tag
tctctagaat

SEQ ID NO: 165 - DNA, 10 bp, Artificial
  SAGE Tag
aactcccagt

SEQ ID NO: 166 - DNA, 10 bp, Artificial
  SAGE Tag
tgcaaaaaaa

SEQ ID NO: 167 - DNA, 10 bp, Artificial
  SAGE Tag
gttgtggcca

SEQ ID NO: 168 - DNA, 10 bp, Artificial
  SAGE Tag
tttaattgtg

SEQ ID NO: 169 - DNA, 10 bp, Artificial
  SAGETag
gaggagtggg

SEQ ID NO: 170 - DNA, 10 bp, Artificial
  SAGE Tag
aatagggtca

SEQ ID NO: 171 - DNA, 10 bp, Artificial
  SAGE Tag
ctgagctgta

SEQ ID NO: 172 - DNA, 10 bp, Artificial
  SAGE Tag
tgtgtggggc

SEQ ID NO: 173 - DNA, 10 bp, Artificial
  SAGE Tag
gtggcgggcg

SEQ ID NO: 174 - DNA, 10 bp, Artificial
  SAGE Tag
tgtgagcccc

SEQ ID NO: 175 - DNA, 10 bp, Artificial
  SAGE Tag
cgtgaacaat

SEQ ID NO: 176 - DNA, 10 bp, Artificial
  SAGE Tag
agcctcgggc

SEQ ID NO: 177 - DNA, 10 bp, Artificial
  SAGE Tag
ggctgcagtc

SEQ ID NO: 178 - DNA, 10 bp, Artificial
  SAGE Tag
ttctctctgt

SEQ ID NO: 179 - DNA, 10 bp, Artificial
  SAGETag
ctgccaaaaa

SEQ ID NO: 180 - DNA, 10 bp, Artificial
  SAGE Tag
ctctcaatat

SEQ ID NO: 181 - DNA, 10 bp, Artificial
  SAGE Tag
aaaataaaga

SEQ ID NO: 182 - DNA, 10 bp, Artificial
  SAGE Tag
accaaattaa

SEQ ID NO: 183 - DNA, 10 bp, Artificial
  SAGE Tag
aaataaaagc

SEQ ID NO: 184 - DNA, 10 bp, Artificial
  SAGE Tag
cccagctaat

SEQ ID NO: 185 - DNA, 10 bp, Artificial
  SAGE Tag
gacccactac

SEQ ID NO: 186 - DNA, 10 bp, Artificial
  SAGE Tag
ctgtttgttc

SEQ ID NO: 187 - DNA, 10 bp, Artificial
  SAGE Tag
ggaacggatg

SEQ ID NO: 188 - DNA, 10 bp, Artificial
  SAGE Tag
ctttcttccc

SEQ ID NO: 189 - DNA, 10 bp, Artificial
  SAGE Tag
ggctatgcca

SEQ ID NO: 190 - DNA, 10 bp, Artificial
  SAGE Tag
taaacttcaa

SEQ ID NO: 191 - DNA, 10 bp, Artificial
  SAGE Tag
ctgctgagcc

SEQ ID NO: 192 - DNA, 10 bp, Artificial
  SAGE Tag
aacaacagtg

SEQ ID NO: 193 - DNA, 10 bp, Artificial
  SAGE Tag
tagtcatctt

SEQ ID NO: 194 - DNA, 10 bp, Artificial
  SAGE Tag
gtttggctgc

SEQ ID NO: 195 - DNA, 10 bp, Artificial
  SAGE Tag
gactaaaaaa

SEQ ID NO: 196 - DNA, 10 bp, Artificial
  SAGE Tag
gtgcaaaatg

SEQ ID NO: 197 - DNA, 10 bp, Artificial
  SAGE Tag
tgttgggttc

SEQ ID NO: 198 - DNA, 10 bp, Artificial
  SAGE Tag
gtggaccctg

SEQ ID NO: 199 - DNA, 10 bp, Artificial
  SAGE Tag
gctatgctcc

SEQ ID NO: 200 - DNA, 10 bp, Artificial
  SAGE Tag
cagagttgta

SEQ ID NO: 201 - DNA, 10 bp, Artificial
  SAGE Tag
gagccgcctc

SEQ ID NO: 202 - DNA, 10 bp, Artificial
  SAGE Tag
accccagcaa

SEQ ID NO: 203 - DNA, 10 bp, Artificial
  SAGE Tag
gaggtgctct

SEQ ID NO: 204 - DNA, 10 bp, Artificial
  SAGE Tag
aggatggcgg

SEQ ID NO: 205 - DNA, 10 bp, Artificial
  SAGE Tag
atggtctacg

SEQ ID NO: 206 - DNA, 10 bp, Artificial
  SAGE Tag
gaatgcagtt

SEQ ID NO: 207 - DNA, 10 bp, Artificial
  SAGE Tag
gccaaacttg

SEQ ID NO: 208 - DNA, 10 bp, Artificial
  SAGE Tag
tggcaaagta

SEQ ID NO: 209 - DNA, 10 bp, Artificial
  SAGE Tag
taatgacaat

SEQ ID NO: 210 - DNA, 10 bp, Artificial
  SAGE Tag
aagctgttgt

SEQ ID NO: 211 - DNA, 10 bp, Artificial
  SAGE Tag
tgtggccagg

SEQ ID NO: 212 - DNA, 10 bp, Artificial
  SAGE Tag
taacattgag

SEQ ID NO: 213 - DNA, 10 bp, Artificial
  SAGE Tag
ggaagtcctg

SEQ ID NO: 214 - DNA, 10 bp, Artificial
  SAGE Tag
gcctcccgcc

SEQ ID NO: 215 - DNA, 10 bp, Artificial
  SAGE Tag
agaattgtgt

SEQ ID NO: 216 - DNA, 10 bp, Artificial
  SAGE Tag
tgacacccac

SEQ ID NO: 217 - DNA, 10 bp, Artificial
  SAGE Tag
ggttgagtgt

SEQ ID NO: 218 - DNA, 10 bp, Artificial
  SAGE Tag
ccacaggaga

SEQ ID NO: 219 - DNA, 10 bp, Artificial
  SAGE Tag
aaggagtttg

SEQ ID NO: 220 - DNA, 10 bp, Artificial
  SAGE Tag
gcggcagtta

SEQ ID NO: 221 - DNA, 10 bp, Artificial
  SAGE Tag
tgaatactac

SEQ ID NO: 222 - DNA, 10 bp, Artificial
  SAGE Tag
tgcattgttt

SEQ ID NO: 223 - DNA, 10 bp, Artificial
  SAGE Tag
gtgtatattg

SEQ ID NO: 224 - DNA, 10 bp, Artificial
  SAGE Tag
gatgctgatt

SEQ ID NO: 225 - DNA, 10 bp, Artificial
  SAGE Tag
ccttacttta

SEQ ID NO: 226 - DNA, 10 bp, Artificial
  SAGE Tag
ccacttcctc

SEQ ID NO: 227 - DNA, 10 bp, Artificial
  SAGE Tag
catccaaaac

SEQ ID NO: 228 - DNA, 10 bp, Artificial
  SAGE Tag
gtggtatggc

SEQ ID NO: 229 - DNA, 10 bp, Artificial
  SAGE Tag
gccaagatgc

SEQ ID NO: 230 - DNA, 10 bp, Artificial
  SAGE Tag
tcagtttgtc

SEQ ID NO: 231 - DNA, 10 bp, Artificial
  SAGE Tag
ttggggttcc

SEQ ID NO: 232 - DNA, 10 bp, Artificial
  SAGE Tag
gtatcttaat

SEQ ID NO: 233 - DNA, 10 bp, Artificial
  SAGE Tag
tggggaaact

SEQ ID NO: 234 - DNA, 10 bp, Artificial
  SAGE Tag
atgaataaaa

SEQ ID NO: 235 - DNA, 10 bp, Artificial
  SAGE Tag
gctaaaaaca

SEQ ID NO: 236 - DNA, 10 bp, Artificial
  SAGE Tag
aagttgcatc

SEQ ID NO: 237 - DNA, 10 bp, Artificial
  SAGE Tag
taaccaaaca

SEQ ID NO: 238 - DNA, 10 bp, Artificial
  SAGE Tag
ggcagcctgg

SEQ ID NO: 239 - DNA, 10 bp, Artificial
  SAGE Tag
gcacaagtaa

SEQ ID NO: 240 - DNA, 10 bp, Artificial
  SAGE Tag
gtgaacttac

SEQ ID NO: 241 - DNA, 10 bp, Artificial
  SAGE Tag
gcaaacaaaa

SEQ ID NO: 242 - DNA, 10 bp, Artificial
  SAGE Tag
acaaatcctt

SEQ ID NO: 243 - DNA, 10 bp, Artificial
  SAGE Tag
atgcaagaga

SEQ ID NO: 244 - DNA, 10 bp, Artificial
  SAGE Tag
gagtgcaggt

SEQ ID NO: 245 - DNA, 10 bp, Artificial
  SAGE Tag
tatcactttt

SEQ ID NO: 246 - DNA, 10 bp, Artificial
  SAGE Tag
ctactgtcta

SEQ ID NO: 247 - DNA, 10 bp, Artificial
  SAGE Tag
ccagtgtgca

SEQ ID NO: 248 - DNA, 10 bp, Artificial
  SAGE Tag
gtgctattct

SEQ ID NO: 249 - DNA, 10 bp, Artificial
  SAGE Tag
gagctggtga

SEQ ID NO: 250 - DNA, 10 bp, Artificial
  SAGE Tag
gtctgacccc

SEQ ID NO: 251 - DNA, 10 bp, Artificial
  SAGE Tag
gccacgttgt

SEQ ID NO: 252 - DNA, 10 bp, Artificial
  SAGE Tag
acctagccac

SEQ ID NO: 253 - DNA, 10 bp, Artificial
  SAGE Tag
gactgttgct

SEQ ID NO: 254 - DNA, 10 bp, Artificial
  SAGE Tag
attaaagtgc

SEQ ID NO: 255 - DNA, 10 bp, Artificial
  SAGE Tag
ttgcttcttt

SEQ ID NO: 256 - DNA, 10 bp, Artificial
  SAGE Tag
tacacagaac

SEQ ID NO: 257 - DNA, 10 bp, Artificial
  SAGE Tag
cccctgcact

SEQ ID NO: 258 - DNA, 10 bp, Artificial
  SAGE Tag
gtttttgctt

SEQ ID NO: 259 - DNA, 10 bp, Artificial
  SAGE Tag
ccagtgtctg

SEQ ID NO: 260 - DNA, 10 bp, Artificial
  SAGE Tag
tgaataaagt

SEQ ID NO: 261 - DNA, 10 bp, Artificial
  SAGE Tag
actacaaata

SEQ ID NO: 262 - DNA, 10 bp, Artificial
  SAGE Tag
agcaagcaag

SEQ ID NO: 263 - DNA, 10 bp, Artificial
  SAGE Tag
cagtagcctt

SEQ ID NO: 264 - DNA, 10 bp, Artificial
  SAGE Tag
cccttctatt

SEQ ID NO: 265 - DNA, 10 bp, Artificial
  SAGE Tag
ggaatccaat

SEQ ID NO: 266 - DNA, 10 bp, Artificial
  SAGE Tag
ttgtaaatgc

SEQ ID NO: 267 - DNA, 10 bp, Artificial
  SAGE Tag
ggcaacagag

SEQ ID NO: 268 - DNA, 10 bp, Artificial
  SAGE Tag
aagacagcgg

SEQ ID NO: 269 - DNA, 10 bp, Artificial
  SAGE Tag
tccttattaa

SEQ ID NO: 270 - DNA, 10 bp, Artificial
  SAGE Tag
agtgccctga

SEQ ID NO: 271 - DNA, 10 bp, Artificial
  SAGE Tag
gatagttgtg

SEQ ID NO: 272 - DNA, 10 bp, Artificial
  SAGE Tag
tttggaaatc

SEQ ID NO: 273 - DNA, 10 bp, Artificial
  SAGE Tag
ctgccctagt

SEQ ID NO: 274 - DNA, 10 bp, Artificial
  SAGE Tag
gatgtagtgg

SEQ ID NO: 275 - DNA, 10 bp, Artificial
  SAGE Tag
cctgtcatcc

SEQ ID NO: 276 - DNA, 10 bp, Artificial
  SAGE Tag
gccttgcctg

SEQ ID NO: 277 - DNA, 10 bp, Artificial
  SAGE Tag
gtagcttatg

SEQ ID NO: 278 - DNA, 10 bp, Artificial
  SAGE Tag
tgttattaaa

SEQ ID NO: 279 - DNA, 10 bp, Artificial
  SAGE Tag
gttctgggcg

SEQ ID NO: 280 - DNA, 10 bp, Artificial
  SAGE Tag
tccttgcttc

SEQ ID NO: 281 - DNA, 10 bp, Artificial
  SAGE Tag
ctgtgtaagc

SEQ ID NO: 282 - DNA, 10 bp, Artificial
  SAGE Tag
cagccgtgat

SEQ ID NO: 283 - DNA, 10 bp, Artificial
  SAGE Tag
tcttgaacag

SEQ ID NO: 284 - DNA, 10 bp, Artificial
  SAGE Tag
ttttaaactt

SEQ ID NO: 285 - DNA, 10 bp, Artificial
  SAGE Tag
ggattccagt

SEQ ID NO: 286 - DNA, 10 bp, Artificial
  SAGE Tag
agtgggtatt

SEQ ID NO: 287 - DNA, 10 bp, Artificial
  SAGE Tag
ttcacttatg

SEQ ID NO: 288 - DNA, 10 bp, Artificial
  SAGE Tag
gtgtcagatg

SEQ ID NO: 289 - DNA, 10 bp, Artificial
  SAGE Tag
cccccaatgc

SEQ ID NO: 290 - DNA, 10 bp, Artificial
  SAGE Tag
aaaacaaaac

SEQ ID NO: 291 - DNA, 10 bp, Artificial
  SAGE Tag
agaagcaaga

SEQ ID NO: 292 - DNA, 10 bp, Artificial
  SAGE Tag
agtaaaccat

SEQ ID NO: 293 - DNA, 10 bp, Artificial
  SAGE Tag
ctgacgggta

SEQ ID NO: 294 - DNA, 10 bp, Artificial
  SAGE Tag
atttaaaaaa

SEQ ID NO: 295 - DNA, 10 bp, Artificial
  SAGE Tag
tttatttaag

SEQ ID NO: 296 - DNA, 10 bp, Artificial
  SAGE Tag
gaagcagggc

SEQ ID NO: 297 - DNA, 10 bp, Artificial
  SAGE Tag
ccaactctca

SEQ ID NO: 298 - DNA, 10 bp, Artificial
  SAGE Tag
gatgtggaca

SEQ ID NO: 299 - DNA, 10 bp, Artificial
  SAGE Tag
atggtcagta

SEQ ID NO: 300 - DNA, 10 bp, Artificial
  SAGE Tag
tatgccctat

SEQ ID NO: 301 - DNA, 10 bp, Artificial
  SAGE Tag
caagtatacc

SEQ ID NO: 302 - DNA, 10 bp, Artificial
  SAGE Tag
tgagggatgg

SEQ ID NO: 303 - DNA, 10 bp, Artificial
  SAGE Tag
gcaccataat

SEQ ID NO: 304 - DNA, 10 bp, Artificial
  SAGE Tag
agttcaagac

SEQ ID NO: 305 - DNA, 10 bp, Artificial
  SAGE Tag
gagtttgtgt

SEQ ID NO: 306 - DNA, 10 bp, Artificial
  SAGE Tag
caaataaatg

SEQ ID NO: 307 - DNA, 10 bp, Artificial
  SAGE Tag
ctgcaaggac

SEQ ID NO: 308 - DNA, 10 bp, Artificial
  SAGE Tag
ctggccatcg

SEQ ID NO: 309 - DNA, 10 bp, Artificial
  SAGE Tag
gggccaggaa

SEQ ID NO: 310 - DNA, 10 bp, Artificial
  SAGE Tag
ctgggatcat

SEQ ID NO: 311 - DNA, 10 bp, Artificial
  SAGE Tag
gcaccttatt

SEQ ID NO: 312 - DNA, 10 bp, Artificial
  SAGE Tag
gaagcaataa

SEQ ID NO: 313 - DNA, 10 bp, Artificial
  SAGE Tag
ggaagagcac

SEQ ID NO: 314 - DNA, 10 bp, Artificial
  SAGE Tag
gtggcagtgg

SEQ ID NO: 315 - DNA, 10 bp, Artificial
  SAGE Tag
accctgtaga

SEQ ID NO: 316 - DNA, 10 bp, Artificial
  SAGE Tag
cctgaaaact

SEQ ID NO: 317 - DNA, 10 bp, Artificial
  SAGE Tag
ctgggcaaca

SEQ ID NO: 318 - DNA, 10 bp, Artificial
  SAGE Tag
gttcatcctt

SEQ ID NO: 319 - DNA, 10 bp, Artificial
  SAGE Tag
tcagtgtgtg

SEQ ID NO: 320 - DNA, 10 bp, Artificial
  SAGE Tag
ctgggcaaac

SEQ ID NO: 321 - DNA, 10 bp, Artificial
  SAGE Tag
aagagagctt

SEQ ID NO: 322 - DNA, 10 bp, Artificial
  SAGE Tag
tgtaaggcac

SEQ ID NO: 323 - DNA, 10 bp, Artificial
  SAGE Tag
ttagaaacag

SEQ ID NO: 324 - DNA, 10 bp, Artificial
  SAGE Tag
gccttccgtg

SEQ ID NO: 325 - DNA, 10 bp, Artificial
  SAGE Tag
tacaataaac

SEQ ID NO: 326 - DNA, 10 bp, Artificial
  SAGE Tag
caggaaccac

SEQ ID NO: 327 - DNA, 10 bp, Artificial
  SAGE Tag
aggacaatgc

SEQ ID NO: 328 - DNA, 10 bp, Artificial
  SAGE Tag
ctcacaaggg

SEQ ID NO: 329 - DNA, 10 bp, Artificial
  SAGE Tag
agaggtggtg

SEQ ID NO: 330 - DNA, 10 bp, Artificial
  SAGE Tag
ataaagctac

SEQ ID NO: 331 - DNA, 10 bp, Artificial
  SAGE Tag
gcctcttgaa

SEQ ID NO: 332 - DNA, 10 bp, Artificial
  SAGE Tag
ttaaaagcct

SEQ ID NO: 333 - DNA, 10 bp, Artificial
  SAGE Tag
ttctaacata

SEQ ID NO: 334 - DNA, 10 bp, Artificial
  SAGE Tag
gcaaaactcc

SEQ ID NO: 335 - DNA, 10 bp, Artificial
  SAGE Tag
tttctgttaa

SEQ ID NO: 336 - DNA, 10 bp, Artificial
  SAGE Tag
caagccaaaa

SEQ ID NO: 337 - DNA, 10 bp, Artificial
  SAGE Tag
tttaggtaaa

SEQ ID NO: 338 - DNA, 10 bp, Artificial
  SAGE Tag
accttgctgg

SEQ ID NO: 339 - DNA, 10 bp, Artificial
  SAGE Tag
ctcaagtaat

SEQ ID NO: 340 - DNA, 10 bp, Artificial
  SAGE Tag
cgccgagcac

SEQ ID NO: 341 - DNA, 10 bp, Artificial
  SAGE Tag
caggggaagg

SEQ ID NO: 342 - DNA, 10 bp, Artificial
  SAGE Tag
ctgaattcag

SEQ ID NO: 343 - DNA, 10 bp, Artificial
  SAGE Tag
ggagacagag

SEQ ID NO: 344 - DNA, 10 bp, Artificial
  SAGE Tag
gcactgaaaa

SEQ ID NO: 345 - DNA, 10 bp, Artificial
  SAGE Tag
gaaatgagca

SEQ ID NO: 346 - DNA, 10 bp, Artificial
  SAGE Tag
atgatgcctc

SEQ ID NO: 347 - DNA, 10 bp, Artificial
  SAGE Tag
taacctcagg

SEQ ID NO: 348 - DNA, 10 bp, Artificial
  SAGE Tag
tgcgtaccac

SEQ ID NO: 349 - DNA, 10 bp, Artificial
  SAGE Tag
tctgcatttg

SEQ ID NO: 350 - DNA, 10 bp, Artificial
  SAGE Tag
ttacagagct

SEQ ID NO: 351 - DNA, 10 bp, Artificial
  SAGE Tag
acactttttt

SEQ ID NO: 352 - DNA, 10 bp, Artificial
  SAGE Tag
gacagtgacg

SEQ ID NO: 353 - DNA, 10 bp, Artificial
  SAGE Tag
ttctattttg

SEQ ID NO: 354 - DNA, 10 bp, Artificial
  SAGE Tag
gtttaaatgg

SEQ ID NO: 355 - DNA, 10 bp, Artificial
  SAGE Tag
ctagacagta

SEQ ID NO: 356 - DNA, 10 bp, Artificial
  SAGE Tag
cacctaattt

SEQ ID NO: 357 - DNA, 10 bp, Artificial
  SAGE Tag
cttctctctc

SEQ ID NO: 358 - DNA, 10 bp, Artificial
  SAGE Tag
acacccagaa

SEQ ID NO: 359 - DNA, 10 bp, Artificial
  SAGE Tag
ccttctcact

SEQ ID NO: 360 - DNA, 10 bp, Artificial
  SAGE Tag
gggggggggg

SEQ ID NO: 361 - DNA, 10 bp, Artificial
  SAGE Tag
tataataaat

SEQ ID NO: 362 - DNA, 10 bp, Artificial
  SAGE Tag
cgcctctcca

SEQ ID NO: 363 - DNA, 10 bp, Artificial
  SAGE Tag
cggccacaga

SEQ ID NO: 364 - DNA, 10 bp, Artificial
  SAGE Tag
taaacttctg

SEQ ID NO: 365 - DNA, 10 bp, Artificial
  SAGE Tag
ataccactaa

SEQ ID NO: 366 - DNA, 10 bp, Artificial
  SAGE Tag
acaaaaataa

SEQ ID NO: 367 - DNA, 10 bp, Artificial
  SAGE Tag
cctataataa

SEQ ID NO: 368 - DNA, 10 bp, Artificial
  SAGE Tag
ggatgtagag

SEQ ID NO: 369 - DNA, 10 bp, Artificial
  SAGE Tag
cccctttgca

SEQ ID NO: 370 - DNA, 10 bp, Artificial
  SAGE Tag
cggggatcat

SEQ ID NO: 371 - DNA, 10 bp, Artificial
  SAGE Tag
agttgtcccg

SEQ ID NO: 372 - DNA, 10 bp, Artificial
  SAGE Tag
caacagctct

SEQ ID NO: 373 - DNA, 10 bp, Artificial
  SAGE Tag
ccacagtaga

SEQ ID NO: 374 - DNA, 10 bp, Artificial
  SAGE Tag
cagcttcacc

SEQ ID NO: 375 - DNA, 10 bp, Artificial
  SAGE Tag
cggtcttatg

SEQ ID NO: 376 - DNA, 10 bp, Artificial
  SAGE Tag
tgaaagtgtg

SEQ ID NO: 377 - DNA, 10 bp, Artificial
  SAGE Tag
gggtgcaaaa

SEQ ID NO: 378 - DNA, 10 bp, Artificial
  SAGE Tag
ttcttgaaca

SEQ ID NO: 379 - DNA, 10 bp, Artificial
  SAGE Tag
ccagtagaag

SEQ ID NO: 380 - DNA, 10 bp, Artificial
  SAGE Tag
gagactcctg

SEQ ID NO: 381 - DNA, 10 bp, Artificial
  SAGE Tag
gacgtgtggg

SEQ ID NO: 382 - DNA, 10 bp, Artificial
  SAGE Tag
aatgactgaa

SEQ ID NO: 383 - DNA, 10 bp, Artificial
  SAGE Tag
atttgagagt

SEQ ID NO: 384 - DNA, 10 bp, Artificial
  SAGE Tag
gctgttttgt

SEQ ID NO: 385 - DNA, 10 bp, Artificial
  SAGE Tag
aagtgattct

SEQ ID NO: 386 - DNA, 10 bp, Artificial
  SAGE Tag
aaatggctaa

SEQ ID NO: 387 - DNA, 10 bp, Artificial
  SAGE Tag
tctgaagtca

SEQ ID NO: 388 - DNA, 10 bp, Artificial
  SAGE Tag
tatttattga

SEQ ID NO: 389 - DNA, 10 bp, Artificial
  SAGE Tag
ctttatgtga

SEQ ID NO: 390 - DNA, 10 bp, Artificial
  SAGE Tag
aggatatcca

SEQ ID NO: 391 - DNA, 10 bp, Artificial
  SAGE Tag
gcatattaaa

SEQ ID NO: 392 - DNA, 10 bp, Artificial
  SAGE Tag
cagctccgct

SEQ ID NO: 393 - DNA, 10 bp, Artificial
  SAGE Tag
ggagccagct

SEQ ID NO: 394 - DNA, 10 bp, Artificial
  SAGE Tag
cggcaaaaaa

SEQ ID NO: 395 - DNA, 10 bp, Artificial
  SAGE Tag
tttgtttcca

SEQ ID NO: 396 - DNA, 10 bp, Artificial
  SAGE Tag
ttctgcactg

SEQ ID NO: 397 - DNA, 10 bp, Artificial
  SAGE Tag
ggtggatgtg

SEQ ID NO: 398 - DNA, 10 bp, Artificial
  SAGE Tag
tgtgaacaac

SEQ ID NO: 399 - DNA, 10 bp, Artificial
  SAGE Tag
taagcagatg

SEQ ID NO: 400 - DNA, 10 bp, Artificial
  SAGE Tag
aactcttgaa

SEQ ID NO: 401 - DNA, 10 bp, Artificial
  SAGE Tag
acttgatttg

SEQ ID NO: 402 - DNA, 10 bp, Artificial
  SAGE Tag
gtattgaagt

SEQ ID NO: 403 - DNA, 10 bp, Artificial
  SAGE Tag
aggtctaagg

SEQ ID NO: 404 - DNA, 10 bp, Artificial
  SAGE Tag
agagactctt

SEQ ID NO: 405 - DNA, 10 bp, Artificial
  SAGE Tag
atatttcatt

SEQ ID NO: 406 - DNA, 10 bp, Artificial
  SAGE Tag
caggaggaaa

SEQ ID NO: 407 - DNA, 10 bp, Artificial
  SAGE Tag
tttgaccttt

SEQ ID NO: 408 - DNA, 10 bp, Artificial
  SAGE Tag
ttgaatttat

SEQ ID NO: 409 - DNA, 10 bp, Artificial
  SAGE Tag
ttctctttca

SEQ ID NO: 410 - DNA, 10 bp, Artificial
  SAGE Tag
cagctcttag

SEQ ID NO: 411 - DNA, 10 bp, Artificial
  SAGE Tag
tgtgggaacc

SEQ ID NO: 412 - DNA, 10 bp, Artificial
  SAGE Tag
aaatcctcaa

SEQ ID NO: 413 - DNA, 10 bp, Artificial
  SAGE Tag
gtagaaaaga

SEQ ID NO: 414 - DNA, 10 bp, Artificial
  SAGE Tag
cagaccctgc

SEQ ID NO: 415 - DNA, 10 bp, Artificial
  SAGE Tag
cttggcaccc

SEQ ID NO: 416 - DNA, 10 bp, Artificial
  SAGE Tag
gtacgcattc

SEQ ID NO: 417 - DNA, 10 bp, Artificial
  SAGE Tag
tgaacacccg

SEQ ID NO: 418 - DNA, 10 bp, Artificial
  SAGE Tag
cagtctggga

SEQ ID NO: 419 - DNA, 10 bp, Artificial
  SAGE Tag
ctgctgataa

SEQ ID NO: 420 - DNA, 10 bp, Artificial
  SAGE Tag
taatggatgt

SEQ ID NO: 421 - DNA, 10 bp, Artificial
  SAGE Tag
tcaatggaca

SEQ ID NO: 422 - DNA, 10 bp, Artificial
  SAGE Tag
tcacgccaat

SEQ ID NO: 423 - DNA, 10 bp, Artificial
  SAGE Tag
cacaaacaca

SEQ ID NO: 424 - DNA, 10 bp, Artificial
  SAGE Tag
tggagtgtac

SEQ ID NO: 425 - DNA, 10 bp, Artificial
  SAGE Tag
actgctcatt

SEQ ID NO: 426 - DNA, 10 bp, Artificial
  SAGE Tag
tcctgtgctg

SEQ ID NO: 427 - DNA, 10 bp, Artificial
  SAGE Tag
gcaaaaacag

SEQ ID NO: 428 - DNA, 10 bp, Artificial
  SAGE Tag
agttgtactt

SEQ ID NO: 429 - DNA, 10 bp, Artificial
  SAGE Tag
gcaacagaaa

SEQ ID NO: 430 - DNA, 10 bp, Artificial
  SAGE Tag
aggctaccgg

SEQ ID NO: 431 - DNA, 10 bp, Artificial
  SAGE Tag
caggcaactt

SEQ ID NO: 432 - DNA, 10 bp, Artificial
  SAGE Tag
ctgggaagca

SEQ ID NO: 433 - DNA, 10 bp, Artificial
  SAGE Tag
gagtatggag

SEQ ID NO: 434 - DNA, 10 bp, Artificial
  SAGE Tag
caaatatctt

SEQ ID NO: 435 - DNA, 10 bp, Artificial
  SAGE Tag
atcacaaaaa

SEQ ID NO: 436 - DNA, 10 bp, Artificial
  SAGE Tag
gccacagttg

SEQ ID NO: 437 - DNA, 10 bp, Artificial
  SAGE Tag
ccacaccctg

SEQ ID NO: 438 - DNA, 10 bp, Artificial
  SAGE Tag
taagactata

SEQ ID NO: 439 - DNA, 10 bp, Artificial
  SAGE Tag
gcccagccag

SEQ ID NO: 440 - DNA, 10 bp, Artificial
  SAGE Tag
cagttcttga

SEQ ID NO: 441 - DNA, 10 bp, Artificial
  SAGE Tag
gagaagactg

SEQ ID NO: 442 - DNA, 10 bp, Artificial
  SAGE Tag
gaagggaccg

SEQ ID NO: 443 - DNA, 10 bp, Artificial
  SAGE Tag
tcagaaactg

SEQ ID NO: 444 - DNA, 10 bp, Artificial
  SAGE Tag
ccaacaacta

SEQ ID NO: 445 - DNA, 10 bp, Artificial
  SAGE Tag
gctgctgcct

SEQ ID NO: 446 - DNA, 10 bp, Artificial
  SAGE Tag
gtaaaaaaag

SEQ ID NO: 447 - DNA, 10 bp, Artificial
  SAGE Tag
ggcatttttg

SEQ ID NO: 448 - DNA, 10 bp, Artificial
  SAGE Tag
agttcaggag

SEQ ID NO: 449 - DNA, 10 bp, Artificial
  SAGE Tag
agacgctttc

SEQ ID NO: 450 - DNA, 10 bp, Artificial
  SAGE Tag
ttgaagcacc

SEQ ID NO: 451 - DNA, 10 bp, Artificial
  SAGE Tag
ctcagaaaaa

SEQ ID NO: 452 - DNA, 10 bp, Artificial
  SAGE Tag
cagtgaaaaa

SEQ ID NO: 453 - DNA, 10 bp, Artificial
  SAGE Tag
gagcttacat

SEQ ID NO: 454 - DNA, 10 bp, Artificial
  SAGE Tag
acgctcatcg

SEQ ID NO: 455 - DNA, 10 bp, Artificial
  SAGE Tag
ttttcttcat

SEQ ID NO: 456 - DNA, 10 bp, Artificial
  SAGE Tag
tactgcctct

SEQ ID NO: 457 - DNA, 10 bp, Artificial
  SAGE Tag
cggcaggaga

SEQ ID NO: 458 - DNA, 10 bp, Artificial
  SAGE Tag
aacattgaca

SEQ ID NO: 459 - DNA, 10 bp, Artificial
  SAGE Tag
ggaagtgcca

SEQ ID NO: 460 - DNA, 10 bp, Artificial
  SAGE Tag
aggccacctc

SEQ ID NO: 461 - DNA, 10 bp, Artificial
  SAGE Tag
aactttctgg

SEQ ID NO: 462 - DNA, 10 bp, Artificial
  SAGE Tag
tggaagctag

SEQ ID NO: 463 - DNA, 10 bp, Artificial
  SAGE Tag
gatagaggga

SEQ ID NO: 464 - DNA, 10 bp, Artificial
  SAGE Tag
gttcctttgg

SEQ ID NO: 465 - DNA, 10 bp, Artificial
  SAGE Tag
tgactggctt

SEQ ID NO: 466 - DNA, 10 bp, Artificial
  SAGE Tag
aacagtcaaa

SEQ ID NO: 467 - DNA, 10 bp, Artificial
  SAGE Tag
cagttgctgg

SEQ ID NO: 468 - DNA, 10 bp, Artificial
  SAGE Tag
cgccgtcgca

SEQ ID NO: 469 - DNA, 10 bp, Artificial
  SAGE Tag
ctagtgaaac

SEQ ID NO: 470 - DNA, 10 bp, Artificial
  SAGE Tag
gtgatgtctg

SEQ ID NO: 471 - DNA, 10 bp, Artificial
  SAGE Tag
gcaggccaag

SEQ ID NO: 472 - DNA, 10 bp, Artificial
  SAGE Tag
ttcttttcat

SEQ ID NO: 473 - DNA, 10 bp, Artificial
  SAGE Tag
atcctctgcg

SEQ ID NO: 474 - DNA, 10 bp, Artificial
  SAGE Tag
gccttttact

SEQ ID NO: 475 - DNA, 10 bp, Artificial
  SAGE Tag
gctgggaacc

SEQ ID NO: 476 - DNA, 10 bp, Artificial
  SAGE Tag
gccagcaaat

SEQ ID NO: 477 - DNA, 10 bp, Artificial
  SAGE Tag
cagcccctct

SEQ ID NO: 478 - DNA, 10 bp, Artificial
  SAGE Tag
taggtgtatg

SEQ ID NO: 479 - DNA, 10 bp, Artificial
  SAGE Tag
tcactgctct

SEQ ID NO: 480 - DNA, 10 bp, Artificial
  SAGE Tag
ggcatttatt

SEQ ID NO: 481 - DNA, 10 bp, Artificial
  SAGE Tag
aatccatatg

SEQ ID NO: 482 - DNA, 10 bp, Artificial
  SAGE Tag
ccagagtctc

SEQ ID NO: 483 - DNA, 10 bp, Artificial
  SAGE Tag
ttacttatac

SEQ ID NO: 484 - DNA, 10 bp, Artificial
  SAGE Tag
atcttggctc

SEQ ID NO: 485 - DNA, 10 bp, Artificial
  SAGE Tag
agctcagcgt

SEQ ID NO: 486 - DNA, 10 bp, Artificial
  SAGE Tag
ggcccgctgc

SEQ ID NO: 487 - DNA, 10 bp, Artificial
  SAGE Tag
cctagtccaa

SEQ ID NO: 488 - DNA, 10 bp, Artificial
  SAGE Tag
gggctaactc

SEQ ID NO: 489 - DNA, 10 bp, Artificial
  SAGE Tag
gcactacaga

SEQ ID NO: 490 - DNA, 10 bp, Artificial
  SAGE Tag
cacaaggtaa

SEQ ID NO: 491 - DNA, 10 bp, Artificial
  SAGE Tag
gatgtggctg

SEQ ID NO: 492 - DNA, 10 bp, Artificial
  SAGE Tag
catcctgtgc

SEQ ID NO: 493 - DNA, 10 bp, Artificial
  SAGE Tag
ggtgggacca

SEQ ID NO: 494 - DNA, 10 bp, Artificial
  SAGE Tag
gtgggaccat

SEQ ID NO: 495 - DNA, 10 bp, Artificial
  SAGE Tag
gcaacacccc

SEQ ID NO: 496 - DNA, 10 bp, Artificial
  SAGE Tag
ggaaggaggg

SEQ ID NO: 497 - DNA, 10 bp, Artificial
  SAGE Tag
tcattcttaa

SEQ ID NO: 498 - DNA, 10 bp, Artificial
  SAGE Tag
gcaaagattg

SEQ ID NO: 499 - DNA, 10 bp, Artificial
  SAGE Tag
caccccctcg

SEQ ID NO: 500 - DNA, 10 bp, Artificial
  SAGE Tag
caagaaggcc

SEQ ID NO: 501 - DNA, 10 bp, Artificial
  SAGE Tag
tacagaaagg

SEQ ID NO: 502 - DNA, 10 bp, Artificial
  SAGE Tag
aaacaaggca

SEQ ID NO: 503 - DNA, 10 bp, Artificial
  SAGE Tag
agcagcgggg

SEQ ID NO: 504 - DNA, 10 bp, Artificial
  SAGE Tag
gcagacattg

SEQ ID NO: 505 - DNA, 10 bp, Artificial
  SAGE Tag
cagtcccaca

SEQ ID NO: 506 - DNA, 10 bp, Artificial
  SAGE Tag
tcagctgtac

SEQ ID NO: 507 - DNA, 10 bp, Artificial
  SAGE Tag
taggtatagg

SEQ ID NO: 508 - DNA, 10 bp, Artificial
  SAGE Tag
aaactggaca

SEQ ID NO: 509 - DNA, 10 bp, Artificial
  SAGE Tag
aactttgtgg

SEQ ID NO: 510 - DNA, 10 bp, Artificial
  SAGE Tag
tgtctgatgc

SEQ ID NO: 511 - DNA, 10 bp, Artificial
  SAGE Tag
cccagttaaa

SEQ ID NO: 512 - DNA, 10 bp, Artificial
  SAGE Tag
taaccatcac

SEQ ID NO: 513 - DNA, 10 bp, Artificial
  SAGE Tag
gaacagatag

SEQ ID NO: 514 - DNA, 10 bp, Artificial
  SAGE Tag
gcaaattgtt

SEQ ID NO: 515 - DNA, 10 bp, Artificial
  SAGE Tag
cagatgtgga

SEQ ID NO: 516 - DNA, 10 bp, Artificial
  SAGE Tag
ctacacagta

SEQ ID NO: 517 - DNA, 10 bp, Artificial
  SAGE Tag
ctcattcagc

SEQ ID NO: 518 - DNA, 10 bp, Artificial
  SAGE Tag
tgcagatggt

SEQ ID NO: 519 - DNA, 10 bp, Artificial
  SAGE Tag
gcctgatttt

SEQ ID NO: 520 - DNA, 10 bp, Artificial
  SAGE Tag
tgtgctaata

SEQ ID NO: 521 - DNA, 10 bp, Artificial
  SAGE Tag
cctgaaattt

SEQ ID NO: 522 - DNA, 10 bp, Artificial
  SAGE Tag
ccttacctac

SEQ ID NO: 523 - DNA, 10 bp, Artificial
  SAGE Tag
tggtgacatt

SEQ ID NO: 524 - DNA, 10 bp, Artificial
  SAGE Tag
cgtttaatgt

SEQ ID NO: 525 - DNA, 10 bp, Artificial
  SAGE Tag
gcgagacccc

SEQ ID NO: 526 - DNA, 10 bp, Artificial
  SAGE Tag
ccatacagac

SEQ ID NO: 527 - DNA, 10 bp, Artificial
  SAGE Tag
gctgggcggc

SEQ ID NO: 528 - DNA, 10 bp, Artificial
  SAGE Tag
gaagaacaga

SEQ ID NO: 529 - DNA, 10 bp, Artificial
  SAGE Tag
ctcatacacc

SEQ ID NO: 530 - DNA, 10 bp, Artificial
  SAGE Tag
gacaagatgc

SEQ ID NO: 531 - DNA, 10 bp, Artificial
  SAGE Tag
tccaaaaaaa

SEQ ID NO: 532 - DNA, 10 bp, Artificial
  SAGE Tag
gtggcacgtg

SEQ ID NO: 533 - DNA, 10 bp, Artificial
  SAGE Tag
tgggctgggg

SEQ ID NO: 534 - DNA, 10 bp, Artificial
  SAGE Tag
tcgccgcgac

SEQ ID NO: 535 - DNA, 10 bp, Artificial
  SAGE Tag
tcttctgcca

SEQ ID NO: 536 - DNA, 10 bp, Artificial
  SAGE Tag
tactgtagtc

SEQ ID NO: 537 - DNA, 10 bp, Artificial
  SAGE Tag
tggtgacagt

SEQ ID NO: 538 - DNA, 10 bp, Artificial
  SAGE Tag
aacccaggag

SEQ ID NO: 539 - DNA, 10 bp, Artificial
  SAGE Tag
tgcaataaga

SEQ ID NO: 540 - DNA, 10 bp, Artificial
  SAGE Tag
cacagtcaaa

SEQ ID NO: 541 - DNA, 10 bp, Artificial
  SAGE Tag
cctgcacact

SEQ ID NO: 542 - DNA, 10 bp, Artificial
  SAGE Tag
cttgcaaacc

SEQ ID NO: 543 - DNA, 10 bp, Artificial
  SAGE Tag
cggcagagct

SEQ ID NO: 544 - DNA, 10 bp, Artificial
  SAGE Tag
taagtggaat

SEQ ID NO: 545 - DNA, 10 bp, Artificial
  SAGE Tag
ttaaagattt

SEQ ID NO: 546 - DNA, 10 bp, Artificial
  SAGE Tag
gcggcagcgg

SEQ ID NO: 547 - DNA, 10 bp, Artificial
  SAGE Tag
aaagcaaacc

SEQ ID NO: 548 - DNA, 10 bp, Artificial
  SAGE Tag
tgacttttct

SEQ ID NO: 549 - DNA, 10 bp, Artificial
  SAGE Tag
atgtagagtg

SEQ ID NO: 550 - DNA, 10 bp, Artificial
  SAGE Tag
atttcacatt

SEQ ID NO: 551 - DNA, 10 bp, Artificial
  SAGE Tag
ggctgagaat

SEQ ID NO: 552 - DNA, 10 bp, Artificial
  SAGE Tag
caaggaagca

SEQ ID NO: 553 - DNA, 10 bp, Artificial
  SAGE Tag
cctgtggtcc

SEQ ID NO: 554 - DNA, 10 bp, Artificial
  SAGE Tag
cctacagata

SEQ ID NO: 555 - DNA, 10 bp, Artificial
  SAGE Tag
ggggaaaaaa

SEQ ID NO: 556 - DNA, 10 bp, Artificial
  SAGE Tag
acgtaattag

SEQ ID NO: 557 - DNA, 10 bp, Artificial
  SAGE Tag′
cccggctctt

SEQ ID NO: 558 - DNA, 10 bp, Artificial
  SAGE Tag
cctggataaa

SEQ ID NO: 559 - DNA, 10 bp, Artificial
  SAGE Tag
taacctgcta

SEQ ID NO: 560 - DNA, 10 bp, Artificial
  SAGE Tag
tgatgttcca

SEQ ID NO: 561 - DNA, 10 bp, Artificial
  SAGE Tag
ctgcctggca

SEQ ID NO: 562 - DNA, 10 bp, Artificial
  SAGE Tag
caggtcaaga

SEQ ID NO: 563 - DNA, 10 bp, Artificial
  SAGE Tag
acttgccatt

SEQ ID NO: 564 - DNA, 10 bp, Artificial
  SAGE Tag
ggagagacag

SEQ ID NO: 565 - DNA, 10 bp, Artificial
  SAGE Tag
gctttcattg

SEQ ID NO: 566 - DNA, 10 bp, Artificial
  SAGE Tag
attgtgccac

SEQ ID NO: 567 - DNA, 10 bp, Artificial
  SAGE Tag
cggcttttct

SEQ ID NO: 568 - DNA, 10 bp, Artificial
  SAGE Tag
gatctcgcaa

SEQ ID NO: 569 - DNA, 10 bp, Artificial
  SAGE Tag
tgtggcgtat

SEQ ID NO: 570 - DNA, 10 bp, Artificial
  SAGE Tag
cgctgttttt

SEQ ID NO: 571 - DNA, 10 bp, Artificial
  SAGE Tag
tgccctcagg

SEQ ID NO: 572 - DNA, 10 bp, Artificial
  SAGE Tag
atgaaaagaa

SEQ ID NO: 573 - DNA, 10 bp, Artificial
  SAGE Tag
gtaacaagct

SEQ ID NO: 574 - DNA, 10 bp, Artificial
  SAGE Tag
ctcccaagct

SEQ ID NO: 575 - DNA, 10 bp, Artificial
  SAGE Tag
aaagagaaaa

SEQ ID NO: 576 - DNA, 10 bp, Artificial
  SAGE Tag
tgcagaccca

SEQ ID NO: 577 - DNA, 10 bp, Artificial
  SAGE Tag
caggagcccc

SEQ ID NO: 578 - DNA, 10 bp, Artificial
  SAGE Tag
gagaatatcc

SEQ ID NO: 579 - DNA, 10 bp, Artificial
  SAGE Tag
gctgcaaagg

SEQ ID NO: 580 - DNA, 10 bp, Artificial
  SAGE Tag
tttactggta

SEQ ID NO: 581 - DNA, 10 bp, Artificial
  SAGE Tag
gcggtaaaaa

SEQ ID NO: 582 - DNA, 10 bp, Artificial
  SAGE Tag
tactcggttg

SEQ ID NO: 583 - DNA, 10 bp, Artificial
  SAGE Tag
gcttttattc

SEQ ID NO: 584 - DNA, 10 bp, Artificial
  SAGE Tag
gaaagagctg

SEQ ID NO: 585 - DNA, 10 bp, Artificial
  SAGE Tag
aagcaaaagg

SEQ ID NO: 586 - DNA, 10 bp, Artificial
  SAGE Tag
tgtcaaaaaa

SEQ ID NO: 587 - DNA, 10 bp, Artificial
  SAGE Tag
tcaaatgcaa

SEQ ID NO: 588 - DNA, 10 bp, Artificial
  SAGE Tag
aactctggac

SEQ ID NO: 589 - DNA, 10 bp, Artificial
  SAGE Tag
attccacaaa

SEQ ID NO: 590 - DNA, 10 bp, Artificial
  SAGE Tag
tgaacacgtt

SEQ ID NO: 591 - DNA, 10 bp, Artificial
  SAGE Tag
ggctgggggg

SEQ ID NO: 592 - DNA, 10 bp, Artificial
  SAGE Tag
aataaatgca

SEQ ID NO: 593 - DNA, 10 bp, Artificial
  SAGE Tag
tattccccac

SEQ ID NO: 594 - DNA, 10 bp, Artificial
  SAGE Tag
tctccaacaa

SEQ ID NO: 595 - DNA, 10 bp, Artificial
  SAGE Tag
tgacctattt

SEQ ID NO: 596 - DNA, 10 bp, Artificial
  SAGE Tag
ttggtgaggg

SEQ ID NO: 597 - DNA, 10 bp, Artificial
  SAGE Tag
acattctgca

SEQ ID NO: 598 - DNA, 10 bp, Artificial
  SAGE Tag
gcagtctagc

SEQ ID NO: 599 - DNA, 10 bp, Artificial
  SAGE Tag
tgtaagaaaa

SEQ ID NO: 600 - DNA, 10 bp, Artificial
  SAGE Tag
ggtaaaatta

SEQ ID NO: 601 - DNA, 10 bp, Artificial
  SAGE Tag
accgcttgtt

SEQ ID NO: 602 - DNA, 10 bp, Artificial
  SAGE Tag
gttaaatatg

SEQ ID NO: 603 - DNA, 10 bp, Artificial
  SAGE Tag
ggtggaaaaa

SEQ ID NO: 604 - DNA, 10 bp, Artificial
  SAGE Tag
aaaatgtatt

SEQ ID NO: 605 - DNA, 10 bp, Artificial
  SAGE Tag
ctaaagtact

SEQ ID NO: 606 - DNA, 10 bp, Artificial
  SAGE Tag
ttggcctctg

SEQ ID NO: 607 - DNA, 10 bp, Artificial
  SAGE Tag
ttcattaaaa

SEQ ID NO: 608 - DNA, 10 bp, Artificial
  SAGE Tag
caggactaag

SEQ ID NO: 609 - DNA, 10 bp, Artificial
  SAGE Tag
aacagctgga

SEQ ID NO: 610 - DNA, 10 bp, Artificial
  SAGE Tag
gaaacaaaat

SEQ ID NO: 611 - DNA, 10 bp, Artificial
  SAGE Tag
catcattaat

SEQ ID NO: 612 - DNA, 10 bp, Artificial
  SAGE Tag
taagatggca

SEQ ID NO: 613 - DNA, 10 bp, Artificial
  SAGE Tag
ggatgggtgt

SEQ ID NO: 614 - DNA, 10 bp, Artificial
  SAGE Tag
gtgaaacgcc

SEQ ID NO: 615 - DNA, 10 bp, Artificial
  SAGE Tag
caaccaacta

SEQ ID NO: 616 - DNA, 10 bp, Artificial
  SAGE Tag
tattaaatag

SEQ ID NO: 617 - DNA, 10 bp, Artificial
  SAGE Tag
caaactgaag

SEQ ID NO: 618 - DNA, 10 bp, Artificial
  SAGE Tag
ctgtactagg

SEQ ID NO: 619 - DNA, 10 bp, Artificial
  SAGE Tag
accattgtgt

SEQ ID NO: 620 - DNA, 10 bp, Artificial
  SAGE Tag
acacttcttg

SEQ ID NO: 621 - DNA, 10 bp, Artificial
  SAGE Tag
gttgaaactc

SEQ ID NO: 622 - DNA, 10 bp, Artificial
  SAGE Tag
tcatttcctg

SEQ ID NO: 623 - DNA, 10 bp, Artificial
  SAGE Tag
ggccttcctt

SEQ ID NO: 624 - DNA, 10 bp, Artificial
  SAGE Tag
attaaaaaaa

SEQ ID NO: 625 - DNA, 10 bp, Artificial
  SAGE Tag
gtttttactt

SEQ ID NO: 626 - DNA, 10 bp, Artificial
  SAGE Tag
attttagaat

SEQ ID NO: 627 - DNA, 10 bp, Artificial
  SAGE Tag
ccaaaagtaa

SEQ ID NO: 628 - DNA, 10 bp, Artificial
  SAGE Tag
taggatgggg

SEQ ID NO: 629 - DNA, 10 bp, Artificial
  SAGE Tag
tgcatatcat

SEQ ID NO: 630 - DNA, 10 bp, Artificial
  SAGE Tag
tgttaatgtt

SEQ ID NO: 631 - DNA, 10 bp, Artificial
  SAGE Tag
tacataatta

SEQ ID NO: 632 - DNA, 10 bp, Artificial
  SAGE Tag
tacttgtgtg

SEQ ID NO: 633 - DNA, 10 bp, Artificial
  SAGE Tag
aaagtgcatc

SEQ ID NO: 634 - DNA, 10 bp, Artificial
  SAGE Tag
tttggaatgt

SEQ ID NO: 635 - DNA, 10 bp, Artificial
  SAGE Tag
cagagtgctg

SEQ ID NO: 636 - DNA, 10 bp, Artificial
  SAGE Tag
cagccataaa

SEQ ID NO: 637 - DNA, 10 bp, Artificial
  SAGE Tag
tattttacgt

SEQ ID NO: 638 - DNA, 10 bp, Artificial
  SAGE Tag
tttcttaaag

SEQ ID NO: 639 - DNA, 10 bp, Artificial
  SAGE Tag
catttgtaaa

SEQ ID NO: 640 - DNA, 10 bp, Artificial
  SAGE Tag
tctactgtta

SEQ ID NO: 641 - DNA, 10 bp, Artificial
  SAGE Tag
cttattcctt

SEQ ID NO: 642 - DNA, 10 bp, Artificial
  SAGE Tag
tcttttcaaa

SEQ ID NO: 643 - DNA, 10 bp, Artificial
  SAGE Tag
taaacattgt

SEQ ID NO: 644 - DNA, 10 bp, Artificial
  SAGE Tag
tatatacatt

SEQ ID NO: 645 - DNA, 10 bp, Artificial
  SAGE Tag
gaagcaaaaa

SEQ ID NO: 646 - DNA, 10 bp, Artificial
  SAGE Tag
gtctcagtca

SEQ ID NO: 647 - DNA, 10 bp, Artificial
  SAGE Tag
ctcttatttc

SEQ ID NO: 648 - DNA, 10 bp, Artificial
  SAGE Tag
cagtatgtcc

SEQ ID NO: 649 - DNA, 10 bp, Artificial
  SAGE Tag
tatccatacc

SEQ ID NO: 650 - DNA, 10 bp, Artificial
  SAGE Tag
cactgtgtgt

SEQ ID NO: 651 - DNA, 10 bp, Artificial
  SAGE Tag
ggggctgtgg

SEQ ID NO: 652 - DNA, 10 bp, Artificial
  SAGE Tag
tggatgtact

SEQ ID NO: 653 - DNA, 10 bp, Artificial
  SAGE Tag
gggcccctgg

SEQ ID NO: 654 - DNA, 10 bp, Artificial
  SAGE Tag
tatcctggct

SEQ ID NO: 655 - DNA, 10 bp, Artificial
  SAGE Tag
gtggtgtctg

SEQ ID NO: 656 - DNA, 10 bp, Artificial
  SAGE Tag
atagatgggg

SEQ ID NO: 657 - DNA, 10 bp, Artificial
  SAGE Tag
gccccttgca

SEQ ID NO: 658 - DNA, 10 bp, Artificial
  SAGE Tag
agaaggatgc

SEQ ID NO: 659 - DNA, 10 bp, Artificial
  SAGE Tag
aatctggttg

SEQ ID NO: 660 - DNA, 10 bp, Artificial
  SAGE Tag
agtttaagca

SEQ ID NO: 661 - DNA, 10 bp, Artificial
  SAGE Tag
tcatcatctg

SEQ ID NO: 662 - DNA, 10 bp, Artificial
  SAGE Tag
gtccctgggt

SEQ ID NO: 663 - DNA, 10 bp, Artificial
  SAGE Tag
atgtgtttca

SEQ ID NO: 664 - DNA, 10 bp, Artificial
  SAGE Tag
taccaaggat

SEQ ID NO: 665 - DNA, 10 bp, Artificial
  SAGE Tag
attttaacaa

SEQ ID NO: 666 - DNA, 10 bp, Artificial
  SAGE Tag
aaaaaataaa

SEQ ID NO: 667 - DNA, 10 bp, Artificial
  SAGE Tag
gtaaatttaa

SEQ ID NO: 668 - DNA, 10 bp, Artificial
  SAGE Tag\
ggctgaattt

SEQ ID NO: 669 - DNA, 10 bp, Artificial
  SAGE Tag
tttctgaaaa

SEQ ID NO: 670 - DNA, 10 bp, Artificial
  SAGE Tag
cttcctatac

SEQ ID NO: 671 - DNA, 10 bp, Artificial
  SAGE Tag
ctcagctgcc

SEQ ID NO: 672 - DNA, 10 bp, Artificial
  SAGE Tag
gggggcaagc

SEQ ID NO: 673 - DNA, 10 bp, Artificial
  SAGE Tag
tcttaatggg

SEQ ID NO: 674 - DNA, 10 bp, Artificial
  SAGE Tag
gttgtaaata

SEQ ID NO: 675 - DNA, 10 bp, Artificial
  SAGE Tag
aaatgccctc

SEQ ID NO: 676 - DNA, 10 bp, Artificial
  SAGE Tag
ctatgaagat

SEQ ID NO: 677 - DNA, 10 bp, Artificial
  SAGE Tag
tttacaaacc

SEQ ID NO: 678 - DNA, 10 bp, Artificial
  SAGE Tag
taggacttgg

SEQ ID NO: 679 - DNA, 10 bp, Artificial
  SAGE Tag
agctcagtga

SEQ ID NO: 680 - DNA, 10 bp, Artificial
  SAGE Tag
cgtgtcgata

SEQ ID NO: 681 - DNA, 10 bp, Artificial
  SAGE Tag
atcaagaatc

SEQ ID NO: 682 - DNA, 10 bp, Artificial
  SAGE Tag
ttagcactgt

SEQ ID NO: 683 - DNA, 10 bp, Artificial
  SAGE Tag
cttggtgctg

SEQ ID NO: 684 - DNA, 10 bp, Artificial
  SAGE Tag
tcaacacagt

SEQ ID NO: 685 - DNA, 10 bp, Artificial
  SAGE Tag
tctggacttt

SEQ ID NO: 686 - DNA, 10 bp, Artificial
  SAGE Tag
agccaattcc

SEQ ID NO: 687 - DNA, 10 bp, Artificial
  SAGE Tag
gaatgaggac

SEQ ID NO: 688 - DNA, 10 bp, Artificial
  SAGE Tag
gcaaagaaaa

SEQ ID NO: 689 - DNA, 10 bp, Artificial
  SAGE Tag
tactaaaaaa

SEQ ID NO: 690 - DNA, 10 bp, Artificial
  SAGE Tag
cagatttcca

SEQ ID NO: 691 - DNA, 10 bp, Artificial
  SAGE Tag
ttacactgga

SEQ ID NO: 692 - DNA, 10 bp, Artificial
  SAGE Tag
ccaagaatgt

SEQ ID NO: 693 - DNA, 10 bp, Artificial
  SAGE Tag
cttgctctcc

SEQ ID NO: 694 - DNA, 10 bp, Artificial
  SAGE Tag
taagttcctt

SEQ ID NO: 695 - DNA, 10 bp, Artificial
  SAGE Tag
cagtggggtt

SEQ ID NO: 696 - DNA, 10 bp, Artificial
  SAGE Tag
tgggagcttt

SEQ ID NO: 697 - DNA, 10 bp, Artificial
  SAGE Tag
tgatcaaaat

SEQ ID NO: 698 - DNA, 10 bp, Artificial
  SAGE Tag
ggcaacaaaa

SEQ ID NO: 699 - DNA, 10 bp, Artificial
  SAGE Tag
ttaaaacaaa

SEQ ID NO: 700 - DNA, 10 bp, Artificial
  SAGE Tag
aaggtcctag

SEQ ID NO: 701 - DNA, 10 bp, Artificial
  SAGE Tag
tgttagattt

SEQ ID NO: 702 - DNA, 10 bp, Artificial
  SAGE Tag
gcgtcgggga

SEQ ID NO: 703 - DNA, 10 bp, Artificial
  SAGE Tag
acctccccag

SEQ ID NO: 704 - DNA, 10 bp, Artificial
  SAGE Tag
tgctacgatc

SEQ ID NO: 705 - DNA, 10 bp, Artificial
  SAGE Tag
tgtaccttgt

SEQ ID NO: 706 - DNA, 10 bp, Artificial
  SAGE Tag
gagagcttaa

SEQ ID NO: 707 - DNA, 10 bp, Artificial
  SAGE Tag
tataaacctt

SEQ ID NO: 708 - DNA, 10 bp, Artificial
  SAGE Tag
ctttttggaa

SEQ ID NO: 709 - DNA, 10 bp, Artificial
  SAGE Tag
gagatgtctg

SEQ ID NO: 710 - DNA, 10 bp, Artificial
  SAGE Tag
gtaagattag

SEQ ID NO: 711 - DNA, 10 bp, Artificial
  SAGE Tag
ctcgttaaga

SEQ ID NO: 712 - DNA, 10 bp, Artificial
  SAGE Tag
ctgaaaattc

SEQ ID NO: 713 - DNA, 10 bp, Artificial
  SAGE Tag
ctactgtacc

SEQ ID NO: 714 - DNA, 10 bp, Artificial
  SAGE Tag
ctagcagagc

SEQ ID NO: 715 - DNA, 10 bp, Artificial
  SAGE Tag
gtcttaccta

SEQ ID NO: 716 - DNA, 10 bp, Artificial
  SAGE Tag
ggtgttgccg

SEQ ID NO: 717 - DNA, 10 bp, Artificial
  SAGE Tag
acataagatc

SEQ ID NO: 718 - DNA, 10 bp, Artificial
  SAGE Tag
tgaccaccta

SEQ ID NO: 719 - DNA, 10 bp, Artificial
  SAGE Tag
aaacattagc

SEQ ID NO: 720 - DNA, 10 bp, Artificial
  SAGE Tag
gccaaagtgt

SEQ ID NO: 721 - DNA, 10 bp, Artificial
  SAGE Tag
actttaacag

SEQ ID NO: 722 - DNA, 10 bp, Artificial
  SAGE Tag
tttatcccaa

SEQ ID NO: 723 - DNA, 10 bp, Artificial
  SAGE Tag
gatttttcat

SEQ ID NO: 724 - DNA, 10 bp, Artificial
  SAGE Tag
ggatgtgttt

SEQ ID NO: 725 - DNA, 10 bp, Artificial
  SAGE Tag
ttgatcgaag

SEQ ID NO: 726 - DNA, 10 bp, Artificial
  SAGE Tag
atcatttgtt

SEQ ID NO: 727 - DNA, 10 bp, Artificial
  SAGE Tag
ttttaaaaat

SEQ ID NO: 728 - DNA, 10 bp, Artificial
  SAGE Tag
atcttagctt

SEQ ID NO: 729 - DNA, 10 bp, Artificial
  SAGE Tag
ccaaatgatg

SEQ ID NO: 730 - DNA, 10 bp, Artificial
  SAGE Tag
ctttttaaat

SEQ ID NO: 731 - DNA, 10 bp, Artificial
  SAGE Tag
aggctttatg

SEQ ID NO: 732 - DNA, 10 bp, Artificial
  SAGE Tag
ctagaacatc

SEQ ID NO: 733 - DNA, 10 bp, Artificial
  SAGE Tag
gagctcttgt

SEQ ID NO: 734 - DNA, 10 bp, Artificial
  SAGE Tag
gaggcttaat

SEQ ID NO: 735 - DNA, 10 bp, Artificial
  SAGE Tag
tgagttgggt

SEQ ID NO: 736 - DNA, 10 bp, Artificial
  SAGE Tag
agtgaggata

SEQ ID NO: 737 - DNA, 10 bp, Artificial
  SAGE Tag
aacttaaaaa

SEQ ID NO: 738 - DNA, 10 bp, Artificial
  SAGE Tag
agtaaaaaaa

SEQ ID NO: 739 - DNA, 10 bp, Artificial
  SAGE Tag
aacccggaag

SEQ ID NO: 740 - DNA, 10 bp, Artificial
  SAGE Tag
aatttaaaaa

SEQ ID NO: 741 - DNA, 10 bp, Artificial
  SAGE Tag
caaattacaa

SEQ ID NO: 742 - DNA, 10 bp, Artificial
  SAGE Tag
ggatctgcaa

SEQ ID NO: 743 - DNA, 10 bp, Artificial
  SAGE Tag
gtggtggacg

SEQ ID NO: 744 - DNA, 10 bp, Artificial
  SAGE Tag
gcacagagcc

SEQ ID NO: 745 - DNA, 10 bp, Artificial
  SAGE Tag
tatcaaaaca

SEQ ID NO: 746 - DNA, 10 bp, Artificial
  SAGE Tag
gccaagtttg

SEQ ID NO: 747 - DNA, 10 bp, Artificial
  SAGE Tag
aaggagatta

SEQ ID NO: 748 - DNA, 10 bp, Artificial
  SAGE Tag
cagcgcaagt

SEQ ID NO: 749 - DNA, 10 bp, Artificial
  SAGE Tag
aattcccttt

SEQ ID NO: 750 - DNA, 10 bp, Artificial
  SAGE Tag
tatttttggc

SEQ ID NO: 751 - DNA, 10 bp, Artificial
  SAGE Tag
atgataaaac

SEQ ID NO: 752 - DNA, 10 bp, Artificial
  SAGE Tag
ttacgatgaa

SEQ ID NO: 753 - DNA, 10 bp, Artificial
  SAGE Tag
ggagatggag

SEQ ID NO: 754 - DNA, 10 bp, Artificial
  SAGE Tag
caaaaaaaat

SEQ ID NO: 755 - DNA, 10 bp, Artificial
  SAGE Tag
gcctggcacc

SEQ ID NO: 756 - DNA, 10 bp, Artificial
  SAGE Tag
ccctatgttt

SEQ ID NO: 757 - DNA, 10 bp, Artificial
  SAGE Tag
ggacaagata

SEQ ID NO: 758 - DNA, 10 bp, Artificial
  SAGE Tag
tgactgctgc

SEQ ID NO: 759 - DNA, 10 bp, Artificial
  SAGE Tag
tccagtatga

SEQ ID NO: 760 - DNA, 10 bp, Artificial
  SAGE Tag
actccaagaa

SEQ ID NO: 761 - DNA, 10 bp, Artificial
  SAGE Tag
gatacgtaag

SEQ ID NO: 762 - DNA, 10 bp, Artificial
  SAGE Tag
atgtggcaca

SEQ ID NO: 763 - DNA, 10 bp, Artificial
  SAGE Tag
cagttactta

SEQ ID NO: 764 - DNA, 10 bp, Artificial
  SAGE Tag
gtgtccctgt

SEQ ID NO: 765 - DNA, 10 bp, Artificial
  SAGE Tag
tctgcacctc

SEQ ID NO: 766 - DNA, 10 bp, Artificial
  SAGE Tag
ccaatttaca

SEQ ID NO: 767 - DNA, 10 bp, Artificial
  SAGE Tag
agtgaccttc

SEQ ID NO: 768 - DNA, 10 bp, Artificial
  SAGE Tag
attttgtgca

SEQ ID NO: 769 - DNA, 10 bp, Artificial
  SAGE Tag
caaaccttta

SEQ ID NO: 770 - DNA, 10 bp, Artificial
  SAGE Tag
gcatacctgc

SEQ ID NO: 771 - DNA, 10 bp, Artificial
  SAGE Tag
ttgttatatt

SEQ ID NO: 772 - DNA, 10 bp, Artificial
  SAGE Tag
gagtcaggag

SEQ ID NO: 773 - DNA, 10 bp, Artificial
  SAGE Tag
agagccctag

SEQ ID NO: 774 - DNA, 10 bp, Artificial
  SAGE Tag
accaaagccc

SEQ ID NO: 775 - DNA, 10 bp, Artificial
  SAGE Tag
tatgtatgtt

SEQ ID NO: 776 - DNA, 10 bp, Artificial
  SAGE Tag
gaaaagagat

SEQ ID NO: 777 - DNA, 10 bp, Artificial
  SAGE Tag
ttagttaagc

SEQ ID NO: 778 - DNA, 10 bp, Artificial
  SAGE Tag
gaaactagga

SEQ ID NO: 779 - DNA, 10 bp, Artificial
  SAGE Tag
tgtgccacta

SEQ ID NO: 780 - DNA, 10 bp, Artificial
  SAGE Tag
ttgcaatgca

SEQ ID NO: 781 - DNA, 10 bp, Artificial
  SAGE Tag
atcagtgtga

SEQ ID NO: 782 - DNA, 10 bp, Artificial
  SAGE Tag
ccttatattt

SEQ ID NO: 783 - DNA, 10 bp, Artificial
  SAGE Tag
taaattcacc

SEQ ID NO: 784 - DNA, 10 bp, Artificial
  SAGE Tag
ttgttctttg

SEQ ID NO: 785 - DNA, 10 bp, Artificial
  SAGE Tag
ttgaaaaaaa

SEQ ID NO: 786 - DNA, 10 bp, Artificial
  SAGE Tag
atcatccaat

SEQ ID NO: 787 - DNA, 10 bp, Artificial
  SAGE Tag
caggctactg

SEQ ID NO: 788 - DNA, 10 bp, Artificial
  SAGE Tag
tatttttgtt

SEQ ID NO: 789 - DNA, 10 bp, Artificial
  SAGE Tag
tcagcacctg

SEQ ID NO: 790 - DNA, 10 bp, Artificial
  SAGE Tag
gagtcagcat

SEQ ID NO: 791 - DNA, 10 bp, Artificial
  SAGE Tag
tctgttgagt

SEQ ID NO: 792 - DNA, 10 bp, Artificial
  SAGE Tag
tctgttattg

SEQ ID NO: 793 - DNA, 10 bp, Artificial
  SAGE Tag
gaagagctgg

SEQ ID NO: 794 - DNA, 10 bp, Artificial
  SAGE Tag
aagagttttg

SEQ ID NO: 795 - DNA, 10 bp, Artificial
  SAGE Tag
gtttcaggta

SEQ ID NO: 796 - DNA, 10 bp, Artificial
  SAGE Tag
cctataatcc

SEQ ID NO: 797 - DNA, 10 bp, Artificial
  SAGE Tag
ttcattgtag

SEQ ID NO: 798 - DNA, 10 bp, Artificial
  SAGE Tag
tcttaatgaa

SEQ ID NO: 799 - DNA, 10 bp, Artificial
  SAGE Tag
cctaaaggag

SEQ ID NO: 800 - DNA, 10 bp, Artificial
  SAGE Tag
catttttccc

SEQ ID NO: 801 - DNA, 10 bp, Artificial
  SAGE Tag
ttagataagc

SEQ ID NO: 802 - DNA, 10 bp, Artificial
  SAGE Tag
caagttagtg

SEQ ID NO: 803 - DNA, 10 bp, Artificial
  SAGE Tag
actggagttt

SEQ ID NO: 804 - DNA, 10 bp, Artificial
  SAGE Tag
aggaatgctt

SEQ ID NO: 805 - DNA, 10 bp, Artificial
  SAGE Tag
gaaatttaaa

SEQ ID NO: 806 - DNA, 10 bp, Artificial
  SAGE Tag
gggggggtct

SEQ ID NO: 807 - DNA, 10 bp, Artificial
  SAGE Tag
ggcagttaac

SEQ ID NO: 808 - DNA, 10 bp, Artificial
  SAGE Tag
ccactgcgct

SEQ ID NO: 809 - DNA, 10 bp, Artificial
  SAGE Tag
acagtgggga

SEQ ID NO: 810 - DNA, 10 bp, Artificial
  SAGE Tag
gtccggagtc

SEQ ID NO: 811 - DNA, 10 bp, Artificial
  SAGE Tag
atagaggcaa

SEQ ID NO: 812 - DNA, 10 bp, Artificial
  SAGE Tag
gattccactg

SEQ ID NO: 813 - DNA, 10 bp, Artificial
  SAGE Tag
tactagtcct

SEQ ID NO: 814 - DNA, 10 bp, Artificial
  SAGE Tag
tgcctgtgaa

SEQ ID NO: 815 - DNA, 10 bp, Artificial
  SAGE Tag
gtcaacagta

SEQ ID NO: 816 - DNA, 10 bp, Artificial
  SAGE Tag
caattccttc

SEQ ID NO: 817 - DNA, 10 bp, Artificial
  SAGE Tag
tgttgatttt

SEQ ID NO: 818 - DNA, 10 bp, Artificial
  SAGE Tag
cccttagcaa

SEQ ID NO: 819 - DNA, 10 bp, Artificial
  SAGE Tag
gggaaccgga

SEQ ID NO: 820 - DNA, 10 bp, Artificial
  SAGE Tag
gtgttgactg

SEQ ID NO: 821 - DNA, 10 bp, Artificial
  SAGE Tag
gtgatatcca

SEQ ID NO: 822 - DNA, 10 bp, Artificial
  SAGE Tag
cctagtaaaa

SEQ ID NO: 823 - DNA, 10 bp, Artificial
  SAGE Tag
atgagctatg

SEQ ID NO: 824 - DNA, 10 bp, Artificial
  SAGE Tag
taatgaacta

SEQ ID NO: 825 - DNA, 10 bp, Artificial
  SAGE Tag
tccaatcttg

SEQ ID NO: 826 - DNA, 10 bp, Artificial
  SAGE Tag
atgtttgcca

SEQ ID NO: 827 - DNA, 10 bp, Artificial
  SAGE Tag
attcaaaaaa

SEQ ID NO: 828 - DNA, 10 bp, Artificial
  SAGE Tag
gcgcctcaac

SEQ ID NO: 829 - DNA, 10 bp, Artificial
  SAGE Tag
tatcctgtgt

SEQ ID NO: 830 - DNA, 10 bp, Artificial
  SAGE Tag
tcttctgctt

SEQ ID NO: 831 - DNA, 10 bp, Artificial
  SAGE Tag
ttccccaccc

SEQ ID NO: 832 - DNA, 10 bp, Artificial
  SAGE Tag
taatttgaaa

SEQ ID NO: 833 - DNA, 10 bp, Artificial
  SAGE Tag
ataaaaaaaa

SEQ ID NO: 834 - DNA, 10 bp, Artificial
  SAGE Tag
tagctgtctt

SEQ ID NO: 835 - DNA, 10 bp, Artificial
  SAGE Tag
tgcacacaca

SEQ ID NO: 836 - DNA, 10 bp, Artificial
  SAGE Tag
ggttctgtag

SEQ ID NO: 837 - DNA, 10 bp, Artificial
  SAGE Tag
tattagacaa

SEQ ID NO: 838 - DNA, 10 bp, Artificial
  SAGE Tag
ttgtacaatt

SEQ ID NO: 839 - DNA, 10 bp, Artificial
  SAGE Tag
cttagtacat

SEQ ID NO: 840 - DNA, 10 bp, Artificial
  SAGE Tag
acatttcatc

SEQ ID NO: 841 - DNA, 10 bp, Artificial
  SAGE Tag
gatcctatta

SEQ ID NO: 842 - DNA, 10 bp, Artificial
  SAGE Tag
aatatttcaa

SEQ ID NO: 843 - DNA, 10 bp, Artificial
  SAGE Tag
gtaggggcct

SEQ ID NO: 844 - DNA, 10 bp, Artificial
  SAGE Tag
gtggtgccgc

SEQ ID NO: 845 - DNA, 10 bp, Artificial
  SAGE Tag
aacaactggc

SEQ ID NO: 846 - DNA, 10 bp, Artificial
  SAGE Tag
tcagaacagt

SEQ ID NO: 847 - DNA, 10 bp, Artificial
  SAGE Tag
aatatgggtg

SEQ ID NO: 848 - DNA, 10 bp, Artificial
  SAGE Tag
ttgtatcaga

SEQ ID NO: 849 - DNA, 10 bp, Artificial
  SAGE Test
gtgctttcct

SEQ ID NO: 850 - DNA, 10 bp, Artificial
  SAGE Tag
aaaatgactt

SEQ ID NO: 851 - DNA, 10 bp, Artificial
  SAGE Tag
gtataaacga

SEQ ID NO: 852 - DNA, 10 bp, Artificial
  SAGE Tag
acaaacaaaa

SEQ ID NO: 853 - DNA, 10 bp, Artificial
  SAGE Tag
tactggttta

SEQ ID NO: 854 - DNA, 10 bp, Artificial
  SAGE Tag
gtgaatgaca

SEQ ID NO: 855 - DNA, 10 bp, Artificial
  SAGE Tag
ggtgaaaccc

SEQ ID NO: 856 - DNA, 10 bp, Artificial
  SAGE Tag
tcctgctggc

SEQ ID NO: 857 - DNA, 10 bp, Artificial
  SAGE Tag
tgctaactgc

SEQ ID NO: 858 - DNA, 10 bp, Artificial
  SAGE Tag
tctgcctcgt

SEQ ID NO: 859 - DNA, 10 bp, Artificial
  SAGE Tag
ggtgagggag

SEQ ID NO: 860 - DNA, 10 bp, Artificial
  SAGE Tag
tgccacattt

SEQ ID NO: 861 - DNA, 10 bp, Artificial
  SAGE Tag
caagagcgag

SEQ ID NO: 862 - DNA, 10 bp, Artificial
  SAGE Tag\
agaatttgca

SEQ ID NO: 863 - DNA, 10 bp, Artificial
  SAGE Tag
gatctgattg

SEQ ID NO: 864 - DNA, 10 bp, Artificial
  SAGE Tag
gtgaccaggt

SEQ ID NO: 865 - DNA, 10 bp, Artificial
  SAGE Tag
tcatctacaa

SEQ ID NO: 866 - DNA, 10 bp, Artificial
  SAGE Tag
ttaagtggaa

SEQ ID NO: 867 - DNA, 10 bp, Artificial
  SAGE Tag
ctgcaggagt

SEQ ID NO: 868 - DNA, 10 bp, Artificial
  SAGE Tag
tttgcttctg

SEQ ID NO: 869 - DNA, 10 bp, Artificial
  SAGE Tag
aagcacttct

SEQ ID NO: 870 - DNA, 10 bp, Artificial
  SAGE Tag
atggacaggc

SEQ ID NO: 871 - DNA, 10 bp, Artificial
  SAGE Tag
ttcttgtaag

SEQ ID NO: 872 - DNA, 10 bp, Artificial
  SAGE Tag
aaaatgacat

SEQ ID NO: 873 - DNA, 10 bp, Artificial
  SAGETag
ccaagatttg

SEQ ID NO: 874 - DNA, 10 bp, Artificial
  SAGE Tag
agttagcagt

SEQ ID NO: 875 - DNA, 10 bp, Artificial
  SAGE Tag
gtttcagcac

SEQ ID NO: 876 - DNA, 10 bp, Artificial
  SAGE Tag
tggagaagag

SEQ ID NO: 877 - DNA, 10 bp, Artificial
  SAGE Tag
tatgctgtaa

SEQ ID NO: 878 - DNA, 10 bp, Artificial
  SAGE Tag
caagtttacc

SEQ ID NO: 879 - DNA, 10 bp, Artificial
  SAGE Tag
taacttaagc

SEQ ID NO: 880 - DNA, 10 bp, Artificial
  SAGE Tag
tatcgttgcc

SEQ ID NO: 881 - DNA, 10 bp, Artificial
  SAGE Tag
atcacgccac

SEQ ID NO: 882 - DNA, 10 bp, Artificial
  SAGE Tag
tcataagcaa

SEQ ID NO: 883 - DNA, 10 bp, Artificial
  SAGE Tag
caagacgggg

SEQ ID NO: 884 - DNA, 10 bp, Artificial
  SAGE Tag
ctcccggcga

SEQ ID NO: 885 - DNA, 10 bp, Artificial
  SAGE Tag
agaacagaaa

SEQ ID NO: 886 - DNA, 10 bp, Artificial
  SAGE Tag
tcccagccca

SEQ ID NO: 887 - DNA, 10 bp, Artificial
  SAGE Tag
ggagaggcca

SEQ ID NO: 888 - DNA, 10 bp, Artificial
  SAGE Tag
ttccctggga

SEQ ID NO: 889 - DNA, 10 bp, Artificial
  SAGE Tag
agcacagagg

SEQ ID NO: 890 - DNA, 10 bp, Artificial
  SAGE Tag
ttcacaacaa

SEQ ID NO: 891 - DNA, 10 bp, Artificial
  SAGE Tag
ggtcagggaa

SEQ ID NO: 892 - DNA, 10 bp, Artificial
  SAGE Tag
ccactgtgct

SEQ ID NO: 893 - DNA, 10 bp, Artificial
  SAGE Tag
ggttgcaggg

SEQ ID NO: 894 - DNA, 10 bp, Artificial
  SAGE Tag
ttcccaaggc

SEQ ID NO: 895 - DNA, 10 bp, Artificial
  SAGE Tag
gcatttagtt

SEQ ID NO: 896 - DNA, 10 bp, Artificial
  SAGE Tag
gtaattattg

SEQ ID NO: 897 - DNA, 10 bp, Artificial
  SAGE Tag
tgatgtggaa

SEQ ID NO: 898 - DNA, 10 bp, Artificial
  SAGE Tag
tttttgaagg

SEQ ID NO: 899 - DNA, 10 bp, Artificial
  SAGE Tag
ttcttgccag

SEQ ID NO: 900 - DNA, 10 bp, Artificial
  SAGE Tag
ctcatatttt

SEQ ID NO: 901 - DNA, 10 bp, Artificial
  SAGE Tag
ttggtcttcc

SEQ ID NO: 902 - DNA, 10 bp, Artificial
  SAGE Tag
taaaggcttt

SEQ ID NO: 903 - DNA, 10 bp, Artificial
  SAGE Tag
ccaagctgcc

SEQ ID NO: 904 - DNA, 10 bp, Artificial
  SAGE Tag
cagtcccagc

SEQ ID NO: 905 - DNA, 10 bp, Artificial
  SAGE Tag
agcactgtac

SEQ ID NO: 906 - DNA, 10 bp, Artificial
  SAGE Tag
ctatgtaaat

SEQ ID NO: 907 - DNA, 10 bp, Artificial
  SAGE Tag
ttggaatcca

SEQ ID NO: 908 - DNA, 10 bp, Artificial
  SAGE Tag
ggagcatctg

SEQ ID NO: 909 - DNA, 10 bp, Artificial
  SAGE Tag
ataggtagag

SEQ ID NO: 910 - DNA, 10 bp, Artificial
  SAGE Tag
ccattttaaa

SEQ ID NO: 911 - DNA, 10 bp, Artificial
  SAGE Tag
ctggtgtgtt

SEQ ID NO: 912 - DNA, 10 bp, Artificial
  SAGE Tag
ttttgtacca

SEQ ID NO: 913 - DNA, 10 bp, Artificial
  SAGE Tag
gcattgtgac

SEQ ID NO: 914 - DNA, 10 bp, Artificial
  SAGE Tag
cctctatgtt

SEQ ID NO: 915 - DNA, 10 bp, Artificial
  SAGE Tag
gtaatttaaa

SEQ ID NO: 916 - DNA, 10 bp, Artificial
  SAGE Tag
tttgcttcag

SEQ ID NO: 917 - DNA, 10 bp, Artificial
  SAGE Tag
atatggggtg

SEQ ID NO: 918 - DNA, 10 bp, Artificial
  SAGE Tag
aaatcctaga

SEQ ID NO: 919 - DNA, 10 bp, Artificial
  SAGE Tag
tataaaagtg

SEQ ID NO: 920 - DNA, 10 bp, Artificial
  SAGE Tag
aggctgggag

SEQ ID NO: 921 - DNA, 10 bp, Artificial
  SAGE Tag
ctcttcacgg

SEQ ID NO: 922 - DNA, 10 bp, Artificial
  SAGE Tag
acattggtta

SEQ ID NO: 923 - DNA, 10 bp, Artificial
  SAGE Tag
accgcacaaa

SEQ ID NO: 924 - DNA, 10 bp, Artificial
  SAGE Tag
atcaagtgga

SEQ ID NO: 925 - DNA, 10 bp, Artificial
  SAGE Tag
ggagctcttg

SEQ ID NO: 926 - DNA, 10 bp, Artificial
  SAGE Tag
tattttaaaa

SEQ ID NO: 927 - DNA, 10 bp, Artificial
  SAGE Tag
aagaaaaaaa

SEQ ID NO: 928 - DNA, 10 bp, Artificial
  SAGE Tag
ggtactttta

SEQ ID NO: 929 - DNA, 10 bp, Artificial
  SAGE Tag
tactgatgaa

SEQ ID NO: 930 - DNA, 10 bp, Artificial
  SAGE Tag
tagccccagc

SEQ ID NO: 931 - DNA, 10 bp, Artificial
  SAGE Tag
tgcaaataaa

SEQ ID NO: 932 - DNA, 10 bp, Artificial
  SAGE Tag
cagtagacag

SEQ ID NO: 933 - DNA, 10 bp, Artificial
  SAGE Tag
taaatacagt

SEQ ID NO: 934 - DNA, 10 bp, Artificial
  SAGE Tag
tagagtgtaa

SEQ ID NO: 935 - DNA, 10 bp, Artificial
  SAGE Tag
aagcctgtag

SEQ ID NO: 936 - DNA, 10 bp, Artificial
  SAGE Tag
cagtctcaga

SEQ ID NO: 937 - DNA, 10 bp, Artificial
  SAGE Tag
tttcattaat

SEQ ID NO: 938 - DNA, 10 bp, Artificial
  SAGE Tag
ttaatcgaag

SEQ ID NO: 939 - DNA, 10 bp, Artificial
  SAGE Tag
taagaaagaa

SEQ ID NO: 940 - DNA, 10 bp, Artificial
  SAGE Tag
ctgccccacc

SEQ ID NO: 941 - DNA, 10 bp, Artificial
  SAGE Tag
tattatttca

SEQ ID NO: 942 - DNA, 10 bp, Artificial
  SAGE Tag
ggatttggct

SEQ ID NO: 943 - DNA, 10 bp, Artificial
  SAGE Tag
acctcactta

SEQ ID NO: 944 - DNA, 10 bp, Artificial
  SAGE Tag
ctgtgacatt

SEQ ID NO: 945 - DNA, 10 bp, Artificial
  SAGE Tag
ctgctgcaag

SEQ ID NO: 946 - DNA, 10 bp, Artificial
  SAGE Tag
cataaaatag

SEQ ID NO: 947 - DNA, 10 bp, Artificial
  SAGE Tag
gatgcaatat

SEQ ID NO: 948 - DNA, 10 bp, Artificial
  SAGE Tag
gtgcccggca

SEQ ID NO: 949 - DNA, 10 bp, Artificial
  SAGE Tag
tctgtaggct

SEQ ID NO: 950 - DNA, 10 bp, Artificial
  SAGE Tag
agggaattaa

SEQ ID NO: 951 - DNA, 10 bp, Artificial
  SAGE Tag
ggccacgtag

SEQ ID NO: 952 - DNA, 10 bp, Artificial
  SAGE Tag
gtaaaatcct

SEQ ID NO: 953 - DNA, 10 bp, Artificial
  SAGE Tag
cccgcaagca

SEQ ID NO: 954 - DNA, 10 bp, Artificial
  SAGE Tag
tcgtgtttca

SEQ ID NO: 955 - DNA, 10 bp, Artificial
  SAGE Tag
gagtggagag

SEQ ID NO: 956 - DNA, 10 bp, Artificial
  SAGE Tag
ggtgacagtg

SEQ ID NO: 957 - DNA, 10 bp, Artificial
  SAGE Tag
actggtggtc

SEQ ID NO: 958 - DNA, 10 bp, Artificial
  SAGE Tag
cgcggtgctt

SEQ ID NO: 959 - DNA, 10 bp, Artificial
  SAGE Tag
gctgacactg

SEQ ID NO: 960 - DNA, 10 bp, Artificial
  SAGE Tag
gatgtttgaa

SEQ ID NO: 961 - DNA, 10 bp, Artificial
  SAGE Tag
tcaaataaag

SEQ ID NO: 962 - DNA, 10 bp, Artificial
  SAGE Tag
tttctgtttt

SEQ ID NO: 963 - DNA, 10 bp, Artificial
  SAGE Tag
atctctatcc

SEQ ID NO: 964 - DNA, 10 bp, Artificial
  SAGE Tag
ttagcccatc

SEQ ID NO: 965 - DNA, 10 bp, Artificial
  SAGE Tag
gcaaaagcaa

SEQ ID NO: 966 - DNA, 10 bp, Artificial
  SAGE Tag
tcagtggggg

SEQ ID NO: 967 - DNA, 10 bp, Artificial
  SAGE Tag
agaaggcctt

SEQ ID NO: 968 - DNA, 10 bp, Artificial
  SAGE Tag
cttcgacgaa

SEQ ID NO: 969 - DNA, 10 bp, Artificial
  SAGE Tag
agaacaaaaa

SEQ ID NO: 970 - DNA, 10 bp, Artificial
  SAGE Tag
attgaaaaaa

SEQ ID NO: 971 - DNA, 10 bp, Artificial
  SAGE Tag
gacttaaggt

SEQ ID NO: 972 - DNA, 10 bp, Artificial
  SAGE Tag
ttgcaaaggg

SEQ ID NO: 973 - DNA, 10 bp, Artificial
  SAGE Tag
gacaaggaca

SEQ ID NO: 974 - DNA, 10 bp, Artificial
  SAGE Tag
tgctgttgga

SEQ ID NO: 975 - DNA, 10 bp, Artificial
  SAGE Tag
gttcttctgt

SEQ ID NO: 976 - DNA, 10 bp, Artificial
  SAGE Tag
tgaagaggca

SEQ ID NO: 977 - DNA, 10 bp, Artificial
  SAGE Tag
caacagcaaa

SEQ ID NO: 978 - DNA, 10 bp, Artificial
  SAGE Tag
aagccatcgt

SEQ ID NO: 979 - DNA, 10 bp, Artificial
  SAGE Tag
aaagaagact

SEQ ID NO: 980 - DNA, 10 bp, Artificial
  SAGE Tag
attttaaggg

SEQ ID NO: 981 - DNA, 10 bp, Artificial
  SAGE Tag
gtagggcaaa

SEQ ID NO: 982 - DNA, 10 bp, Artificial
  SAGE Tag
tgggtgtgta

SEQ ID NO: 983 - DNA, 10 bp, Artificial
  SAGE Tag
tggacagaaa

SEQ ID NO: 984 - DNA, 10 bp, Artificial
  SAGE Tag
tctcaattct

SEQ ID NO: 985 - DNA, 10 bp, Artificial
  SAGE Tag
tatgaatgct

SEQ ID NO: 986 - DNA, 10 bp, Artificial
  SAGE Tag
ttcctggtag

SEQ ID NO: 987 - DNA, 10 bp, Artificial
  SAGE Tag
gaggacccaa

SEQ ID NO: 988 - DNA, 10 bp, Artificial
  SAGE Tag
tcctgaaata

SEQ ID NO: 989 - DNA, 10 bp, Artificial
  SAGE Tag
cagccctccc

SEQ ID NO: 990 - DNA, 10 bp, Artificial
  SAGE Tag
taataaatgc

SEQ ID NO: 991 - DNA, 10 bp, Artificial
  SAGE Tag
tacagaggga

SEQ ID NO: 992 - DNA, 10 bp, Artificial
  SAGE Tag
cctttggcta

SEQ ID NO: 993 - DNA, 10 bp, Artificial
  SAGE Tag
ccgtgactct

SEQ ID NO: 994 - DNA, 10 bp, Artificial
  SAGE Tag
ttggcagtat

SEQ ID NO: 995 - DNA, 10 bp, Artificial
  SAGE Tag
tgttagaaaa

SEQ ID NO: 996 - DNA, 10 bp, Artificial
  SAGE Tag
gaggaagaag

SEQ ID NO: 997 - DNA, 10 bp, Artificial
  SAGE Tag
ttcgggtgtg

SEQ ID NO: 998 - DNA, 10 bp, Artificial
  SAGE Tag
gatgccctcc

SEQ ID NO: 999 - DNA, 10 bp, Artificial
  SAGE Tag
gtgtgtttgt

SEQ ID NO: 1000 - DNA, 10 bp, Artificial
  SAGE Tag
ctgcagggcc

SEQ ID NO: 1001 - DNA, 10 bp, Artificial
  SAGE Tag
gcacttgcat

SEQ ID NO: 1002 - DNA, 10 bp, Artificial
  SAGE Tag
cactcgtgtg

SEQ ID NO: 1003 - DNA, 10 bp, Artificial
  SAGE Tag
aaagcagttt

SEQ ID NO: 1004 - DNA, 10 bp, Artificial
  SAGE Tag
tctggggacg

SEQ ID NO: 1005 - DNA, 10 bp, Artificial
  SAGE Tag
ggtactcgat

SEQ ID NO: 1006 - DNA, 10 bp, Artificial
  SAGE Tag
tctgcaaagg

SEQ ID NO: 1007 - DNA, 10 bp, Artificial
  SAGE Tag
tcctgtaaag

SEQ ID NO: 1008 - DNA, 10 bp, Artificial
  SAGE Tag
ctgcacttac

SEQ ID NO: 1009 - DNA, 10 bp, Artificial
  SAGE Tag
caccattcag

SEQ ID NO: 1010 - DNA, 10 bp, Artificial
  SAGE Tag
ctggtgagtg

SEQ ID NO: 1011 - DNA, 10 bp, Artificial
  SAGE Tag
cttaaggatt

SEQ ID NO: 1012 - DNA, 10 bp, Artificial
  SAGE Tag
tatgtttcag

SEQ ID NO: 1013 - DNA, 10 bp, Artificial
  SAGE Tag
ctgccgagct

SEQ ID NO: 1014 - DNA, 10 bp, Artificial
  SAGE Tag
aatatgtggg

SEQ ID NO: 1015 - DNA, 10 bp, Artificial
  SAGE Tag
aacagaagca

SEQ ID NO: 1016 - DNA, 10 bp, Artificial
  SAGE Tag
gaagatgtgg

SEQ ID NO: 1017 - DNA, 10 bp, Artificial
  SAGE Tag
tctcccttca

SEQ ID NO: 1018 - DNA, 10 bp, Artificial
  SAGE Tag
taagatttca

SEQ ID NO: 1019 - DNA, 10 bp, Artificial
  SAGE Tag
gaattcctcg

SEQ ID NO: 1020 - DNA, 10 bp, Artificial
  SAGE Tag
tgcagatatt

SEQ ID NO: 1021 - DNA, 10 bp, Artificial
  SAGE Tag
ctgtgcccag

SEQ ID NO: 1022 - DNA, 10 bp, Artificial
  SAGE Tag
gtggtttgct

SEQ ID NO: 1023 - DNA, 10 bp, Artificial
  SAGE Tag
taccttcctt

SEQ ID NO: 1024 - DNA, 10 bp, Artificial
  SAGE Tag
gaatgtaagt

SEQ ID NO: 1025 - DNA, 10 bp, Artificial
  SAGE Tag
gggctcacct

SEQ ID NO: 1026 - DNA, 10 bp, Artificial
  SAGETag
ataataaagc

SEQ ID NO: 1027 - DNA, 10 bp, Artificial
  SAGE Tag
accagcatag

SEQ ID NO: 1028 - DNA, 10 bp, Artificial
  SAGE Tag
aaagtcattg

SEQ ID NO: 1029 - DNA, 10 bp, Artificial
  SAGE Tag
gttccagccg

SEQ ID NO: 1030 - DNA, 10 bp, Artificial
  SAGE Tag
ttaggcaagt

SEQ ID NO: 1031 - DNA, 10 bp, Artificial
  SAGE Tag
ccgagatgaa

SEQ ID NO: 1032 - DNA, 10 bp, Artificial
  SAGE Tag
taaataattt

SEQ ID NO: 1033 - DNA, 10 bp, Artificial
  SAGE Tag
ggtttacctg

SEQ ID NO: 1034 - DNA, 10 bp, Artificial
  SAGE Tag
tgactgttat

SEQ ID NO: 1035 - DNA, 10 bp, Artificial
  SAGE Tag
gctcacctgt

SEQ ID NO: 1036 - DNA, 10 bp, Artificial
  SAGE Tag
aggctggatg

SEQ ID NO: 1037 - DNA, 10 bp, Artificial
  SAGE Tag
tataatcttt

SEQ ID NO: 1038 - DNA, 10 bp, Artificial
  SAGE Tag
aggaatgtta

SEQ ID NO: 1039 - DNA, 10 bp, Artificial
  SAGE Tag
cttaagaaaa

SEQ ID NO: 1040 - DNA, 10 bp, Artificial
  SAGE Tag
tgactggaaa

SEQ ID NO: 1041 - DNA, 10 bp, Artificial
  SAGE Tag
caggggtgac

SEQ ID NO: 1042 - DNA, 10 bp, Artificial
  SAGE Tag
aagaggtttg

SEQ ID NO: 1043 - DNA, 10 bp, Artificial
  SAGE Tag
aagctgagtg

SEQ ID NO: 1044 - DNA, 10 bp, Artificial
  SAGE Tag
ttggcattgt

SEQ ID NO: 1045 - DNA, 10 bp, Artificial
  SAGE Tag
caagacagaa

SEQ ID NO: 1046 - DNA, 10 bp, Artificial
  SAGE Tag
tactggagta

SEQ ID NO: 1047 - DNA, 10 bp, Artificial
  SAGE Tag
gtgagaccct

SEQ ID NO: 1048 - DNA, 10 bp, Artificial
  SAGE Tag
ttaccctgaa

SEQ ID NO: 1049 - DNA, 10 bp, Artificial
  SAGE Tag
gaacatagcc

SEQ ID NO: 1050 - DNA, 10 bp, Artificial
  SAGE Tag
tgtcatcaca

SEQ ID NO: 1051 - DNA, 10 bp, Artificial
  SAGE Tag
tgaagcagta

SEQ ID NO: 1052 - DNA, 10 bp, Artificial
  SAGE Tag
aaccaaaaaa

SEQ ID NO: 1053 - DNA, 10 bp, Artificial
  SAGE Tag
taccacacta

SEQ ID NO: 1054 - DNA, 10 bp, Artificial
  SAGE Tag
gcgagaccct

SEQ ID NO: 1055 - DNA, 10 bp, Artificial
  SAGE Tag
aatcgctaat

SEQ ID NO: 1056 - DNA, 10 bp, Artificial
  SAGE Tag
cctggccaga

SEQ ID NO: 1057 - DNA, 10 bp, Artificial
  SAGE Tag
taaaattgct

SEQ ID NO: 1058 - DNA, 10 bp, Artificial
  SAGE Tag
tgaacttaca

SEQ ID NO: 1059 - DNA, 10 bp, Artificial
  SAGE Tag
cagaggaact

SEQ ID NO: 1060 - DNA, 10 bp, Artificial
  SAGE Tag
cactgaactc

SEQ ID NO: 1061 - DNA, 10 bp, Artificial
  SAGE Tag
tacaaaacca

SEQ ID NO: 1062 - DNA, 10 bp, Artificial
  SAGE Tag
taccagcaca

SEQ ID NO: 1063 - DNA, 10 bp, Artificial
  SAGE Tag
taaagctgtt

SEQ ID NO: 1064 - DNA, 10 bp, Artificial
  SAGE Tag
tgtactactt

SEQ ID NO: 1065 - DNA, 10 bp, Artificial
  SAGE Tag
tgctgcctgt

SEQ ID NO: 1066 - DNA, 10 bp, Artificial
  SAGE Tag
tttgctgtag

SEQ ID NO: 1067 - DNA, 10 bp, Artificial
  SAGE Tag
ggagtgtgct

SEQ ID NO: 1068 - DNA, 10 bp, Artificial
  SAGE Tag
tcacagctgt

SEQ ID NO: 1069 - DNA, 10 bp, Artificial
  SAGE Tag
ctaacttcgt

SEQ ID NO: 1070 - DNA, 10 bp, Artificial
  SAGE Tag
tttgttgctt

SEQ ID NO: 1071 - DNA, 10 bp, Artificial
  SAGE Tag
ctgacagttc

SEQ ID NO: 1072 - DNA, 10 bp, Artificial
  SAGE Tag
tcactactgg

SEQ ID NO: 1073 - DNA, 10 bp, Artificial
  SAGE Tag
tggacccaac

SEQ ID NO: 1074 - DNA, 10 bp, Artificial
  SAGE Tag
attatttttc

SEQ ID NO: 1075 - DNA, 10 bp, Artificial
  SAGE Tag
gaaaagcctt

SEQ ID NO: 1076 - DNA, 10 bp, Artificial
  SAGE Tag
tgccatctgt

SEQ ID NO: 1077 - DNA, 10 bp, Artificial
  SAGE Tag
ttgatgtaca

SEQ ID NO: 1078 - DNA, 10 bp, Artificial
  SAGE Tag
tagtttgtgg

SEQ ID NO: 1079 - DNA, 10 bp, Artificial
  SAGE Tag
gtgctcttga

SEQ ID NO: 1080 - DNA, 10 bp, Artificial
  SAGE Tag
cctcctctga

SEQ ID NO: 1081 - DNA, 10 bp, Artificial
  SAGE Tag
tgaaggatgc

SEQ ID NO: 1082 - DNA, 10 bp, Artificial
  SAGE Tag
acagcgtctg

SEQ ID NO: 1083 - DNA, 10 bp, Artificial
  SAGE Tag
gatgaccccg

SEQ ID NO: 1084 - DNA, 10 bp, Artificial
  SAGE Tag
gattggggat

SEQ ID NO: 1085 - DNA, 10 bp, Artificial
  SAGE Tag
tatctgtcta

SEQ ID NO: 1086 - DNA, 10 bp, Artificial
  SAGE Tag
ttaaacctca

SEQ ID NO: 1087 - DNA, 10 bp, Artificial
  SAGE Tag
ccagtacaat

SEQ ID NO: 1088 - DNA, 10 bp, Artificial
  SAGE Tag
gcatatgagc

SEQ ID NO: 1089 - DNA, 10 bp, Artificial
  SAGE Tag
taaaccggaa

SEQ ID NO: 1090 - DNA, 10 bp, Artificial
  SAGE Tag
ggcattttaa

SEQ ID NO: 1091 - DNA, 10 bp, Artificial
  SAGE Tag
taaataaata

SEQ ID NO: 1092 - DNA, 10 bp, Artificial
  SAGE Tag
ttttaaaata

SEQ ID NO: 1093 - DNA, 10 bp, Artificial
  SAGE Tag
cttgcagatg

SEQ ID NO: 1094 - DNA, 10 bp, Artificial
  SAGE Tag
acactgcagt

SEQ ID NO: 1095 - DNA, 10 bp, Artificial
  SAGE Tag
cacctgtagt

SEQ ID NO: 1096 - DNA, 10 bp, Artificial
  SAGE Tag
tttctgtgta

SEQ ID NO: 1097 - DNA, 10 bp, Artificial
  SAGE Tag
ccactgccct

SEQ ID NO: 1098 - DNA, 10 bp, Artificial
  SAGE Tag
tcatcatatt

SEQ ID NO: 1099 - DNA, 10 bp, Artificial
  SAGE Tag
cacatctctg

SEQ ID NO: 1100 - DNA, 10 bp, Artificial
  SAGE Tag
tgggcccctt

SEQ ID NO: 1101 - DNA, 10 bp, Artificial
  SAGE Tag
tcatctttgt

SEQ ID NO: 1102 - DNA, 10 bp, Artificial
  SAGE Tag
cagataacat

SEQ ID NO: 1103 - DNA, 10 bp, Artificial
  SAGE Tag
tttatttggc

SEQ ID NO: 1104 - DNA, 10 bp, Artificial
  SAGE Tag\
aggagggtgg

SEQ ID NO: 1105 - DNA, 10 bp, Artificial
  SAGE Tag
tggaaaaaaa

SEQ ID NO: 1106 - DNA, 10 bp, Artificial
  SAGE Tag
aggctgccca

SEQ ID NO: 1107 - DNA, 10 bp, Artificial
  SAGE Tag
gagaaccacc

SEQ ID NO: 1108 - DNA, 10 bp, Artificial
  SAGE Tag
tacttgggag

SEQ ID NO: 1109 - DNA, 10 bp, Artificial
  SAGE Tag
atgtatgggg

SEQ ID NO: 1110 - DNA, 10 bp, Artificial
  SAGE Tag
aatacacaga

SEQ ID NO: 1111 - DNA, 10 bp, Artificial
  SAGE Tag
ttccctcgtg

SEQ ID NO: 1112 - DNA, 10 bp, Artificial
  SAGE Tag
tttacctgcc

SEQ ID NO: 1113 - DNA, 10 bp, Artificial
  SAGE Tag
tcagagtaat

SEQ ID NO: 1114 - DNA, 10 bp, Artificial
  SAGE Tag
ggttatttag

SEQ ID NO: 1115 - DNA, 10 bp, Artificial
  SAGE Tag
agaagcagac

SEQ ID NO: 1116 - DNA, 10 bp, Artificial
  SAGE Tag
atgtacaggt

SEQ ID NO: 1117 - DNA, 10 bp, Artificial
  SAGE Tag
cagctcagct

SEQ ID NO: 1118 - DNA, 10 bp, Artificial
  SAGE Tag
agaaagatgt

SEQ ID NO: 1119 - DNA, 10 bp, Artificial
  SAGE Tag
aatgagtttg

SEQ ID NO: 1120 - DNA, 10 bp, Artificial
  SAGE Tag
gggagcctca

SEQ ID NO: 1121 - DNA, 10 bp, Artificial
  SAGE Tag
taccccacct

SEQ ID NO: 1122 - DNA, 10 bp, Artificial
  SAGE Tag
tgctgtgcat

SEQ ID NO: 1123 - DNA, 10 bp, Artificial
  SAGE Tag
gtgagacccc

SEQ ID NO: 1124 - DNA, 10 bp, Artificial
  SAGE Tag
atctcaaaga

SEQ ID NO: 1125 - DNA, 10 bp, Artificial
  SAGE Tag
tctgcttgtc

SEQ ID NO: 1126 - DNA, 10 bp, Artificial
  SAGE Tag
taagaaaagg

SEQ ID NO: 1127 - DNA, 10 bp, Artificial
  SAGE Tag
tactggccgc

SEQ ID NO: 1128 - DNA, 10 bp, Artificial
  SAGE Tag
gtagacacct

SEQ ID NO: 1129 - DNA, 10 bp, Artificial
  SAGE Tag
gcaggtggtt

SEQ ID NO: 1130 - DNA, 10 bp, Artificial
  SAGE Tag
tagggctctc

SEQ ID NO: 1131 - DNA, 10 bp, Artificial
  SAGE Tag
gactgagctt

SEQ ID NO: 1132 - DNA, 10 bp, Artificial
  SAGE Tag
gttgacttac

SEQ ID NO: 1133 - DNA, 10 bp, Artificial
  SAGE Tag
gtgagcccat

SEQ ID NO: 1134 - DNA, 10 bp, Artificial
  SAGE Tag
tgtacctgta

SEQ ID NO: 1135 - DNA, 10 bp, Artificial
  SAGE Tag
atgctaaaaa

SEQ ID NO: 1136 - DNA, 10 bp, Artificial
  SAGE Tag
cctgtgtgca

SEQ ID NO: 1137 - DNA, 10 bp, Artificial
  SAGE Tag
gaaggagata

SEQ ID NO: 1138 - DNA, 10 bp, Artificial
  SAGE Tag
caagaggcaa

SEQ ID NO: 1139 - DNA, 10 bp, Artificial
  SAGE Tag
attgtttatg

SEQ ID NO: 1140 - DNA, 10 bp, Artificial
  SAGE Tag
tgttttcgcc

SEQ ID NO: 1141 - DNA, 10 bp, Artificial
  SAGE Tag
caagttcttt

SEQ ID NO: 1142 - DNA, 10 bp, Artificial
  SAGE Tag
caccaaaaaa

SEQ ID NO: 1143 - DNA, 10 bp, Artificial
  SAGE Tag
attgatcttg

SEQ ID NO: 1144 - DNA, 10 bp, Artificial
  SAGE Tag
ttctgtgaat

SEQ ID NO: 1145 - DNA, 10 bp, Artificial
  SAGE Tag
tgtataaaaa

SEQ ID NO: 1146 - DNA, 10 bp, Artificial
  SAGE Tag
cgttcctgcg

SEQ ID NO: 1147 - DNA, 10 bp, Artificial
  SAGE Tag
gcttaccttt

SEQ ID NO: 1148 - DNA, 10 bp, Artificial
  SAGE Tag
ttgcctgtat

SEQ ID NO: 1149 - DNA, 10 bp, Artificial
  SAGE Tag
gccttccaat

SEQ ID NO: 1150 - DNA, 10 bp, Artificial
  SAGE Tag
caagtggcaa

SEQ ID NO: 1151 - DNA, 10 bp, Artificial
  SAGE Tag
aaaggaaagt

SEQ ID NO: 1152 - DNA, 10 bp, Artificial
  SAGE Tag
actgaggaaa

SEQ ID NO: 1153 - DNA, 10 bp, Artificial
  SAGE Tag
ttaaccctct

SEQ ID NO: 1154 - DNA, 10 bp, Artificial
  SAGE Tag
agctcaggag

SEQ ID NO: 1155 - DNA, 10 bp, Artificial
  SAGE Tag
accacccatt

SEQ ID NO: 1156 - DNA, 10 bp, Artificial
  SAGE Tag
ttccttttta

SEQ ID NO: 1157 - DNA, 10 bp, Artificial
  SAGE Tag
cctgtaatct

SEQ ID NO: 1158 - DNA, 10 bp, Artificial
  SAGE Tag
tttgcacttg

SEQ ID NO: 1159 - DNA, 10 bp, Artificial
  SAGE Tag
ttactgggtt

SEQ ID NO: 1160 - DNA, 10 bp, Artificial
  SAGE Tag
gtttctgcaa

SEQ ID NO: 1161 - DNA, 10 bp, Artificial
  SAGE Tag
tataatgtgc

SEQ ID NO: 1162 - DNA, 10 bp, Artificial
  SAGE Tag
ttttcaagaa

SEQ ID NO: 1163 - DNA, 10 bp, Artificial
  SAGE Tag
tacaaaaaaa

SEQ ID NO: 1164 - DNA, 10 bp, Artificial
  SAGE Tag
caaataaaat

SEQ ID NO: 1165 - DNA, 10 bp, Artificial
  SAGE Tag
ttcaggaggg

SEQ ID NO: 1166 - DNA, 10 bp, Artificial
  SAGE Tag
aacaagccaa

SEQ ID NO: 1167 - DNA, 10 bp, Artificial
  SAGE Tag
atgtttgccc

SEQ ID NO: 1168 - DNA, 10 bp, Artificial
  SAGE Tag
aggaaagcca

SEQ ID NO: 1169 - DNA, 10 bp, Artificial
  SAGE Tag
aagaacgtag

SEQ ID NO: 1170 - DNA, 10 bp, Artificial
  SAGE Tag
ttggtaagac

SEQ ID NO: 1171 - DNA, 10 bp, Artificial
  SAGE Tag
cagatttctg

SEQ ID NO: 1172 - DNA, 10 bp, Artificial
  SAGE Tag
tctgcaggtc

SEQ ID NO: 1173 - DNA, 10 bp, Artificial
  SAGE Tag
gctgggacag

SEQ ID NO: 1174 - DNA, 10 bp, Artificial
  SAGE Tag
tgtctggatg

SEQ ID NO: 1175 - DNA, 10 bp, Artificial
  SAGE Tag
tcctagtagg

SEQ ID NO: 1176 - DNA, 10 bp, Artificial
  SAGE Tag
gtgaagcccc

SEQ ID NO: 1177 - DNA, 10 bp, Artificial
  SAGE Tag
gtttgttggg

SEQ ID NO: 1178 - DNA, 10 bp, Artificial
  SAGE Tag
ttctgaagac

SEQ ID NO: 1179 - DNA, 10 bp, Artificial
  SAGE Tag
tattctcaat

SEQ ID NO: 1180 - DNA, 10 bp, Artificial
  SAGE Tag
gagcagctgg

SEQ ID NO: 1181 - DNA, 10 bp, Artificial
  SAGE Tag
gaactgcctc

SEQ ID NO: 1182 - DNA, 10 bp, Artificial
  SAGE Tag
ggcagacaat

SEQ ID NO: 1183 - DNA, 10 bp, Artificial
  SAGE Tag
ttttccctgt

SEQ ID NO: 1184 - DNA, 10 bp, Artificial
  SAGE Tag
ccaaaattag

SEQ ID NO: 1185 - DNA, 10 bp, Artificial
  SAGE Tag
ttaaaataca

SEQ ID NO: 1186 - DNA, 10 bp, Artificial
  SAGE Tag
gggctggggg

SEQ ID NO: 1187 - DNA, 10 bp, Artificial
  SAGE Tag
cagctatttc

SEQ ID NO: 1188 - DNA, 10 bp, Artificial
  SAGE Tag
caggacagtt

SEQ ID NO: 1189 - DNA, 10 bp, Artificial
  SAGE Tag
ctgctaggaa

SEQ ID NO: 1190 - DNA, 10 bp, Artificial
  SAGE Tag
ccggggagca

SEQ ID NO: 1191 - DNA, 10 bp, Artificial
  SAGE Tag
tcttgtaact

SEQ ID NO: 1192 - DNA, 10 bp, Artificial
  SAGE Tag
tctgttgttc

SEQ ID NO: 1193 - DNA, 10 bp, Artificial
  SAGE Tag
tgtgaataaa

SEQ ID NO: 1194 - DNA, 10 bp, Artificial
  SAGE Tag
tggcaagatg

SEQ ID NO: 1195 - DNA, 10 bp, Artificial
  SAGE Tag
tctaaagaat

SEQ ID NO: 1196 - DNA, 10 bp, Artificial
  SAGE Tag
ggcgttagag

SEQ ID NO: 1197 - DNA, 10 bp, Artificial
  SAGE Tag
taccccagaa

SEQ ID NO: 1198 - DNA, 10 bp, Artificial
  SAGE Tag
tgtacatttt

SEQ ID NO: 1199 - DNA, 10 bp, Artificial
  SAGE Tag
cagctccaaa

SEQ ID NO: 1200 - DNA, 10 bp, Artificial
  SAGE Tag
ggaaactgat

SEQ ID NO: 1201 - DNA, 10 bp, Artificial
  SAGE Tag
cttctgtttt

SEQ ID NO: 1202 - DNA, 10 bp, Artificial
  SAGE Tag
tgtcaggaac

SEQ ID NO: 1203 - DNA, 10 bp, Artificial
  SAGE Tag
aaagggggca

SEQ ID NO: 1204 - DNA, 10 bp, Artificial
  SAGE Tag
ttcaacgagg

SEQ ID NO: 1205 - DNA, 10 bp, Artificial
  SAGE Tag
taaggatttt

SEQ ID NO: 1206 - DNA, 10 bp, Artificial
  SAGE Tag
taaaagacaa

SEQ ID NO: 1207 - DNA, 10 bp, Artificial
  SAGE Tag
ggctctgtca

SEQ ID NO: 1208 - DNA, 10 bp, Artificial
  SAGE Tag
tgaacccgtt

SEQ ID NO: 1209 - DNA, 10 bp, Artificial
  SAGE Tag
gctgcatccc

SEQ ID NO: 1210 - DNA, 10 bp, Artificial
  SAGE Tag
tactggaagt

SEQ ID NO: 1211 - DNA, 10 bp, Artificial
  SAGE Tag
gttataagat

SEQ ID NO: 1212 - DNA, 10 bp, Artificial
  SAGE Tag
gtacacatcc

SEQ ID NO: 1213 - DNA, 10 bp, Artificial
  SAGE Tag
tgaaatactg

SEQ ID NO: 1214 - DNA, 10 bp, Artificial
  SAGE Tag
ggctttgatt

SEQ ID NO: 1215 - DNA, 10 bp, Artificial
  SAGE Tag
tagcttcttc

SEQ ID NO: 1216 - DNA, 10 bp, Artificial
  SAGE Tag
ctaaaaggag

SEQ ID NO: 1217 - DNA, 10 bp, Artificial
  SAGE Tag
gaggctcaat

SEQ ID NO: 1218 - DNA, 10 bp, Artificial
  SAGE Tag
tatgcgtttg

SEQ ID NO: 1219 - DNA, 10 bp, Artificial
  SAGE Tag
atgtactaaa

SEQ ID NO: 1220 - DNA, 10 bp, Artificial
  SAGE Tag
caaatgctgt

SEQ ID NO: 1221 - DNA, 10 bp, Artificial
  SAGE Tag
tcagaagttt

SEQ ID NO: 1222 - DNA, 10 bp, Artificial
  SAGE Tag
gggggtgggt

SEQ ID NO: 1223 - DNA, 10 bp, Artificial
  SAGE Tag
tgtaagtctg

SEQ ID NO: 1224 - DNA, 10 bp, Artificial
  SAGE Tag
ctgtcatttg

SEQ ID NO: 1225 - DNA, 10 bp, Artificial
  SAGE Tag
ctcagcaatg

SEQ ID NO: 1226 - DNA, 10 bp, Artificial
  SAGE Tag
acaaactgtg

SEQ ID NO: 1227 - DNA, 10 bp, Artificial
  SAGE Tag
taataaagca

SEQ ID NO: 1228 - DNA, 10 bp, Artificial
  SAGE Tag
gtagacccca

SEQ ID NO: 1229 - DNA, 10 bp, Artificial
  SAGE Tag
tgcctgtaat

SEQ ID NO: 1230 - DNA, 10 bp, Artificial
  SAGE Tag
tgcaataagc

SEQ ID NO: 1231 - DNA, 10 bp, Artificial
  SAGE Tag
gtgtatcttt

SEQ ID NO: 1232 - DNA, 10 bp, Artificial
  SAGE Tag
tgccaaaaaa

SEQ ID NO: 1233 - DNA, 10 bp, Artificial
  SAGE Tag
aggacttctg

SEQ ID NO: 1234 - DNA, 10 bp, Artificial
  SAGE Tag
actggggaat

SEQ ID NO: 1235 - DNA, 10 bp, Artificial
  SAGE Tag
cagtttgtac

SEQ ID NO: 1236 - DNA, 10 bp, Artificial
  SAGE Tag
gcagctaatt

SEQ ID NO: 1237 - DNA, 10 bp, Artificial
  SAGE Tag
ccccagtcgg

SEQ ID NO: 1238 - DNA, 10 bp, Artificial
  SAGE Tag
tcaacagcgt

SEQ ID NO: 1239 - DNA, 10 bp, Artificial
  SAGE Tag
tagctgagac

SEQ ID NO: 1240 - DNA, 10 bp, Artificial
  SAGE Tag
gattttgtag

SEQ ID NO: 1241 - DNA, 10 bp, Artificial
  SAGE Tag
taccctaaaa

SEQ ID NO: 1242 - DNA, 10 bp, Artificial
  SAGE Tag
taccagtgta

SEQ ID NO: 1243 - DNA, 10 bp, Artificial
  SAGE Tag
taaccatttt

SEQ ID NO: 1244 - DNA, 10 bp, Artificial
  SAGE Tag
ttgctatgaa

SEQ ID NO: 1245 - DNA, 10 bp, Artificial
  SAGE Tag
acacttggag

SEQ ID NO: 1246 - DNA, 10 bp, Artificial
  SAGE Tag
attgcttttg

SEQ ID NO: 1247 - DNA, 10 bp, Artificial
  SAGE Tag
agctctgctg

SEQ ID NO: 1248 - DNA, 10 bp, Artificial
  SAGE Tag
taactaacaa

SEQ ID NO: 1249 - DNA, 10 bp, Artificial
  SAGE Tag
tactgtatgt

SEQ ID NO: 1250 - DNA, 10 bp, Artificial
  SAGE Tag
gttcataggt

SEQ ID NO: 1251 - DNA, 10 bp, Artificial
  SAGE Tag
tggtccacgg

SEQ ID NO: 1252 - DNA, 10 bp, Artificial
  SAGE Tag
tatgattatt

SEQ ID NO: 1253 - DNA, 10 bp, Artificial
  SAGE Tag
cctcggagat

SEQ ID NO: 1254 - DNA, 10 bp, Artificial
  SAGE Tag
taggtagctc

SEQ ID NO: 1255 - DNA, 10 bp, Artificial
  SAGE Tag
ttcttctcgt

SEQ ID NO: 1256 - DNA, 10 bp, Artificial
  SAGE Tag
atgaacacgg

SEQ ID NO: 1257 - DNA, 10 bp, Artificial
  SAGE Tag
gcttagaagt

SEQ ID NO: 1258 - DNA, 10 bp, Artificial
  SAGE Tag
gaatggatgc

SEQ ID NO: 1259 - DNA, 10 bp, Artificial
  SAGE Tag
cttgattcct

SEQ ID NO: 1260 - DNA, 10 bp, Artificial
  SAGE Tag
tgtttaatgt

SEQ ID NO: 1261 - DNA, 10 bp, Artificial
  SAGE Tag
catttgaaag

SEQ ID NO: 1262 - DNA, 10 bp, Artificial
  SAGE Tag
cccctcaaaa

SEQ ID NO: 1263 - DNA, 10 bp, Artificial
  SAGE Tag
ttctgtgtat

SEQ ID NO: 1264 - DNA, 10 bp, Artificial
  SAGE Tag
tagccaaaaa

SEQ ID NO: 1265 - DNA, 10 bp, Artificial
  SAGE Tag
gaggatcact

SEQ ID NO: 1266 - DNA, 10 bp, Artificial
  SAGE Tag
aagtgtgttt

SEQ ID NO: 1267 - DNA, 10 bp, Artificial
  SAGE Tag
ttgtttaatt

SEQ ID NO: 1268 - DNA, 10 bp, Artificial
  SAGE Tag
actggtatac

SEQ ID NO: 1269 - DNA, 10 bp, Artificial
  SAGE Tag
gccgcttcta

SEQ ID NO: 1270 - DNA, 10 bp, Artificial
  SAGE Tag
tgtgatcaca

SEQ ID NO: 1271 - DNA, 10 bp, Artificial
  SAGE Tag
gcccccaatt

SEQ ID NO: 1272 - DNA, 10 bp, Artificial
  SAGE Tag
tgaagagact

SEQ ID NO: 1273 - DNA, 10 bp, Artificial
  SAGE Tag
tgggttaata

SEQ ID NO: 1274 - DNA, 10 bp, Artificial
  SAGE Tag
taagcccttt

SEQ ID NO: 1275 - DNA, 10 bp, Artificial
  SAGE Tag
tgccccgggc

SEQ ID NO: 1276 - DNA, 10 bp, Artificial
  SAGE Tag
cacattaggt

SEQ ID NO: 1277 - DNA, 10 bp, Artificial
  SAGE Tag
tttctcagtg

SEQ ID NO: 1278 - DNA, 10 bp, Artificial
  SAGE Tag
gaacaccact

SEQ ID NO: 1279 - DNA, 10 bp, Artificial
  SAGE Tag
tatggtctgg

SEQ ID NO: 1280 - DNA, 10 bp, Artificial
  SAGE Tag
aatgtccagt

SEQ ID NO: 1281 - DNA, 10 bp, Artificial
  SAGE Tag
taacaaagga

SEQ ID NO: 1282 - DNA, 10 bp, Artificial
  SAGE Tag
tacatttcaa

SEQ ID NO: 1283 - DNA, 10 bp, Artificial
  SAGE Tag
gtttcaggag

SEQ ID NO: 1284 - DNA, 10 bp, Artificial
  SAGE Tag
caaaggccct

SEQ ID NO: 1285 - DNA, 10 bp, Artificial
  SAGE Tag
ggacagggca

SEQ ID NO: 1286 - DNA, 10 bp, Artificial
  SAGE Tag
gagaactccc

SEQ ID NO: 1287 - DNA, 10 bp, Artificial
  SAGE Tag
gcacttcaaa

SEQ ID NO: 1288 - DNA, 10 bp, Artificial
  SAGE Tag
accctgggca

SEQ ID NO: 1289 - DNA, 10 bp, Artificial
  SAGE Tag
tttattccct

SEQ ID NO: 1290 - DNA, 10 bp, Artificial
  SAGE Tag
taactgtcaa

SEQ ID NO: 1291 - DNA, 10 bp, Artificial
  SAGE Tag
agactatatt

SEQ ID NO: 1292 - DNA, 10 bp, Artificial
  SAGE Tag
ttatgctttc

SEQ ID NO: 1293 - DNA, 10 bp, Artificial
  SAGE Tag
ggacttagaa

SEQ ID NO: 1294 - DNA, 10 bp, Artificial
  SAGE Tag
ttttgtaatt

SEQ ID NO: 1295 - DNA, 10 bp, Artificial
  SAGE Tag
ttttgtgcat

SEQ ID NO: 1296 - DNA, 10 bp, Artificial
  SAGE Tag
tcaaggcccc

SEQ ID NO: 1297 - DNA, 10 bp, Artificial
  SAGE Tag
ggaccgaggg

SEQ ID NO: 1298 - DNA, 10 bp, Artificial
  SAGE Tag
cttgtgtgta

SEQ ID NO: 1299 - DNA, 10 bp, Artificial
  SAGE Tag
cccattcact

SEQ ID NO: 1300 - DNA, 10 bp, Artificial
  SAGE Tag
tacacccgct

SEQ ID NO: 1301 - DNA, 10 bp, Artificial
  SAGE Tag
catccttggg

SEQ ID NO: 1302 - DNA, 10 bp, Artificial
  SAGE Tag
tgggccaggc

SEQ ID NO: 1303 - DNA, 10 bp, Artificial
  SAGE Tag
gctggatttt

SEQ ID NO: 1304 - DNA, 10 bp, Artificial
  SAGE Tag
gccaagcccc

SEQ ID NO: 1305 - DNA, 10 bp, Artificial
  SAGE Tag
ctgaaacccc

SEQ ID NO: 1306 - DNA, 10 bp, Artificial
  SAGE Tag
gcatccctga

SEQ ID NO: 1307 - DNA, 10 bp, Artificial
  SAGE Tag
caagtttcca

SEQ ID NO: 1308 - DNA, 10 bp, Artificial
  SAGE Tag
tctgcttttg

SEQ ID NO: 1309 - DNA, 10 bp, Artificial
  SAGE Tag
agctgacagg

SEQ ID NO: 1310 - DNA, 10 bp, Artificial
  SAGE Tag
tccatagatt

SEQ ID NO: 1311 - DNA, 10 bp, Artificial
  SAGE Tag
ctccttaaga

SEQ ID NO: 1312 - DNA, 10 bp, Artificial
  SAGE Tag
ggacagctca

SEQ ID NO: 1313 - DNA, 10 bp, Artificial
  SAGE Tag
caaataaact

SEQ ID NO: 1314 - DNA, 10 bp, Artificial
  SAGE Tag
ctcctggccc

SEQ ID NO: 1315 - DNA, 10 bp, Artificial
  SAGE Tag
gtttggggct

SEQ ID NO: 1316 - DNA, 10 bp, Artificial
  SAGE Tag
gccttaacaa

SEQ ID NO: 1317 - DNA, 10 bp, Artificial
  SAGE Tag
ctgtacagac

SEQ ID NO: 1318 - DNA, 10 bp, Artificial
  SAGE Tag
aagaaagttc

SEQ ID NO: 1319 - DNA, 10 bp, Artificial
  SAGE Tag
gctggatgcg

SEQ ID NO: 1320 - DNA, 10 bp, Artificial
  SAGE Tag
tggaaagagc

SEQ ID NO: 1321 - DNA, 10 bp, Artificial
  SAGE Tag
gcaagcccca

SEQ ID NO: 1322 - DNA, 10 bp, Artificial
  SAGE Tag
tggaactgta

SEQ ID NO: 1323 - DNA, 10 bp, Artificial
  SAGE Tag
tgaatgtcaa

SEQ ID NO: 1324 - DNA, 10 bp, Artificial
  SAGE Tag
cctttgtaag

SEQ ID NO: 1325 - DNA, 10 bp, Artificial
  SAGE Tag
atggcggcga

SEQ ID NO: 1326 - DNA, 10 bp, Artificial
  SAGE Tag
tggacaagct

SEQ ID NO: 1327 - DNA, 10 bp, Artificial
  SAGE Tag
agctgtattc

SEQ ID NO: 1328 - DNA, 10 bp, Artificial
  SAGE Tag
atgtgaagag

SEQ ID NO: 1329 - DNA, 10 bp, Artificial
  SAGE Tag
ccgaggcttg

SEQ ID NO: 1330 - DNA, 10 bp, Artificial
  SAGE Tag
catacagaaa

SEQ ID NO: 1331 - DNA, 10 bp, Artificial
  SAGE Tag
cagattgctg

SEQ ID NO: 1332 - DNA, 10 bp, Artificial
  SAGE Tag
tagataatgg

SEQ ID NO: 1333 - DNA, 10 bp, Artificial
  SAGE Tag
tcaatcaaga

SEQ ID NO: 1334 - DNA, 10 bp, Artificial
  SAGE Tag
atgtagtagt

SEQ ID NO: 1335 - DNA, 10 bp, Artificial
  SAGE Tag
tctgaaaacc

SEQ ID NO: 1336 - DNA, 10 bp, Artificial
  SAGE Tag
ttacaacatt

SEQ ID NO: 1337 - DNA, 10 bp, Artificial
  SAGE Tag
gattggcggc

SEQ ID NO: 1338 - DNA, 10 bp, Artificial
  SAGE Tag
ccacgtgtcc

SEQ ID NO: 1339 - DNA, 10 bp, Artificial
  SAGE Tag
tgccttactt

SEQ ID NO: 1340 - DNA, 10 bp, Artificial
  SAGE Tag
gcagcagagg

SEQ ID NO: 1341 - DNA, 10 bp, Artificial
  SAGE Tag
cgtatgcctc

SEQ ID NO: 1342 - DNA, 10 bp, Artificial
  SAGE Tag
gacactacac

SEQ ID NO: 1343 - DNA, 10 bp, Artificial
  SAGE Tag
caacactgtg

SEQ ID NO: 1344 - DNA, 10 bp, Artificial
  SAGE Tag
ggactttcct

SEQ ID NO: 1345 - DNA, 10 bp, Artificial
  SAGE Tag
tcaacttctg

SEQ ID NO: 1346 - DNA, 10 bp, Artificial
  SAGE Tag
acccgcctga

SEQ ID NO: 1347 - DNA, 10 bp, Artificial
  SAGE Tag
gtggtggcag

SEQ ID NO: 1348 - DNA, 10 bp, Artificial
  SAGE Tag
ggaggaattc

SEQ ID NO: 1349 - DNA, 10 bp, Artificial
  SAGE Tag
gagcaagggg

SEQ ID NO: 1350 - DNA, 10 bp, Artificial
  SAGE Tag
tgggaacata

SEQ ID NO: 1351 - DNA, 10 bp, Artificial
  SAGE Tag
ctcgcgctgg

SEQ ID NO: 1352 - DNA, 10 bp, Artificial
  SAGE Tag
gtaagtgtac

SEQ ID NO: 1353 - DNA, 10 bp, Artificial
  SAGE Tag
aacagcaaaa

SEQ ID NO: 1354 - DNA, 10 bp, Artificial
  SAGE Tag
taacagttgt

SEQ ID NO: 1355 - DNA, 10 bp, Artificial
  SAGE Tag
cccccgcgga

SEQ ID NO: 1356 - DNA, 10 bp, Artificial
  SAGE Tag
gctggtctga

SEQ ID NO: 1357 - DNA, 10 bp, Artificial
  SAGE Tag
agacattgta

SEQ ID NO: 1358 - DNA, 10 bp, Artificial
  SAGE Tag
agcctgctca

SEQ ID NO: 1359 - DNA, 10 bp, Artificial
  SAGE Tag
tcagcaaggg

SEQ ID NO: 1360 - DNA, 10 bp, Artificial
  SAGE Tag
atggggcagg

SEQ ID NO: 1361 - DNA, 10 bp, Artificial
  SAGE Tag
gaggtctcct

SEQ ID NO: 1362 - DNA, 10 bp, Artificial
  SAGE Tag
aaggaggtgg

SEQ ID NO: 1363 - DNA, 10 bp, Artificial
  SAGE Tag
tataacttgt

SEQ ID NO: 1364 - DNA, 10 bp, Artificial
  SAGE Tag
tgtggcgggt

SEQ ID NO: 1365 - DNA, 10 bp, Artificial
  SAGE Tag
aagaaagggg

SEQ ID NO: 1366 - DNA, 10 bp, Artificial
  SAGE Tag
agggggttcg

SEQ ID NO: 1367 - DNA, 10 bp, Artificial
  SAGE Tag
gttattgagg

SEQ ID NO: 1368 - DNA, 10 bp, Artificial
  SAGE Tag
tgcagcgcct

SEQ ID NO: 1369 - DNA, 10 bp, Artificial
  SAGE Tag
gctttgcttt

SEQ ID NO: 1370 - DNA, 10 bp, Artificial
  SAGE Tag
ggaacagggg

SEQ ID NO: 1371 - DNA, 10 bp, Artificial
  SAGE Tag
actgccccaa

SEQ ID NO: 1372 - DNA, 10 bp, Artificial
  SAGE Tag
ggccaagtga

SEQ ID NO: 1373 - DNA, 10 bp, Artificial
  SAGE Tag
gtcttaaagt

SEQ ID NO: 1374 - DNA, 10 bp, Artificial
  SAGE Tag
aaataagtca

SEQ ID NO: 1375 - DNA, 10 bp, Artificial
  SAGE Tag
gcagaaatga

SEQ ID NO: 1376 - DNA, 10 bp, Artificial
  SAGE Tag
aagggggcaa

SEQ ID NO: 1377 - DNA, 10 bp, Artificial
  SAGE Tag
cgggggtggg

SEQ ID NO: 1378 - DNA, 10 bp, Artificial
  SAGE Tag
tccttggacc

SEQ ID NO: 1379 - DNA, 10 bp, Artificial
  SAGE Tag
ttcgctgagg

SEQ ID NO: 1380 - DNA, 10 bp, Artificial
  SAGE Tag
gccttggtaa

SEQ ID NO: 1381 - DNA, 10 bp, Artificial
  SAGE Tag
ccgccgaagt

SEQ ID NO: 1382 - DNA, 10 bp, Artificial
  SAGE Tag
gtctcctaat

SEQ ID NO: 1383 - DNA, 10 bp, Artificial
  SAGE Tag
ccacaatcct

SEQ ID NO: 1384 - DNA, 10 bp, Artificial
  SAGE Tag
tgattgccct

SEQ ID NO: 1385 - DNA, 10 bp, Artificial
  SAGE Tag
cacccctgat

SEQ ID NO: 1386 - DNA, 10 bp, Artificial
  SAGE Tag
ggttattttg

SEQ ID NO: 1387 - DNA, 10 bp, Artificial
  SAGE Tag
attagtgttg

SEQ ID NO: 1388 - DNA, 10 bp, Artificial
  SAGE Tag
agaggttgat

SEQ ID NO: 1389 - DNA, 10 bp, Artificial
  SAGE Tag
gaacaccgtc

SEQ ID NO: 1390 - DNA, 10 bp, Artificial
  SAGE Tag
aatgacacaa

SEQ ID NO: 1391 - DNA, 10 bp, Artificial
  SAGE Tag
agcttgcagg

SEQ ID NO: 1392 - DNA, 10 bp, Artificial
  SAGE Tag
tgatggtgat

SEQ ID NO: 1393 - DNA, 10 bp, Artificial
  SAGE Tag
ctcaacagat

SEQ ID NO: 1394 - DNA, 10 bp, Artificial
  SAGE Tag
aacagtgtgc

SEQ ID NO: 1395 - DNA, 10 bp, Artificial
  SAGE Tag
tgctgaggaa

SEQ ID NO: 1396 - DNA, 10 bp, Artificial
  SAGE Tag
cgagtgagag

SEQ ID NO: 1397 - DNA, 10 bp, Artificial
  SAGE Tag
cccaaagaca

SEQ ID NO: 1398 - DNA, 10 bp, Artificial
  SAGE Tag
tgcacaatat

SEQ ID NO: 1399 - DNA, 10 bp, Artificial
  SAGE Tag
gaaactgaag

SEQ ID NO: 1400 - DNA, 10 bp, Artificial
  SAGE Tag
gacaaagaga

SEQ ID NO: 1401 - DNA, 10 bp, Artificial
  SAGE Tag
cagcctgtcg

SEQ ID NO: 1402 - DNA, 10 bp, Artificial
  SAGE Tag
aacaaggtga

SEQ ID NO: 1403 - DNA, 10 bp, Artificial
  SAGE Tag
tgagatcttg

SEQ ID NO: 1404 - DNA, 10 bp, Artificial
  SAGE Tag
tgcgttgaga

SEQ ID NO: 1405 - DNA, 10 bp, Artificial
  SAGE Tag
tagacccctt

SEQ ID NO: 1406 - DNA, 10 bp, Artificial
  SAGE Tag
cagtggaggg

SEQ ID NO: 1407 - DNA, 10 bp, Artificial
  SAGE Tag
ggactggggt

SEQ ID NO: 1408 - DNA, 10 bp, Artificial
  SAGE Tag
tcggagctgt

SEQ ID NO: 1409 - DNA, 10 bp, Artificial
  SAGE Tag
gtggaaaagt

SEQ ID NO: 1410 - DNA, 10 bp, Artificial
  SAGE Tag
ttggatcatt

SEQ ID NO: 1411 - DNA, 10 bp, Artificial
  SAGE Tag
ctcatatgtt

SEQ ID NO: 1412 - DNA, 10 bp, Artificial
  SAGE Tag
accccaggca

SEQ ID NO: 1413 - DNA, 10 bp, Artificial
  SAGE Tag
aaagcgtaaa

SEQ ID NO: 1414 - DNA, 10 bp, Artificial
  SAGE Tag
gtggtgcacc

SEQ ID NO: 1415 - DNA, 10 bp, Artificial
  SAGE Tag
acagaatggc

SEQ ID NO: 1416 - DNA, 10 bp, Artificial
  SAGE Tag
cgtctgtaag

SEQ ID NO: 1417 - DNA, 10 bp, Artificial
  SAGE Tag
gcatttcagg

SEQ ID NO: 1418 - DNA, 10 bp, Artificial
  SAGE Tag
gcggtcattg

SEQ ID NO: 1419 - DNA, 10 bp, Artificial
  SAGE Tag
gttgcccaga

SEQ ID NO: 1420 - DNA, 10 bp, Artificial
  SAGE Tag
ttgaagggcc

SEQ ID NO: 1421 - DNA, 10 bp, Artificial
  SAGE Tag
tagactgctg

SEQ ID NO: 1422 - DNA, 10 bp, Artificial
  SAGE Tag
atgcttgctt

SEQ ID NO: 1423 - DNA, 10 bp, Artificial
  SAGE Tag
cttgacatac

SEQ ID NO: 1424 - DNA, 10 bp, Artificial
  SAGE Tag
gctagtgatg

SEQ ID NO: 1425 - DNA, 10 bp, Artificial
  SAGE Tag
aataaatgga

SEQ ID NO: 1426 - DNA, 10 bp, Artificial
  SAGE Tag
tgcacgacta

SEQ ID NO: 1427 - DNA, 10 bp, Artificial
  SAGE Tag
ttggaacaat

SEQ ID NO: 1428 - DNA, 10 bp, Artificial
  SAGE Tag
atgtgtgttg

SEQ ID NO: 1429 - DNA, 10 bp, Artificial
  SAGE Tag
tgctgcattg

SEQ ID NO: 1430 - DNA, 10 bp, Artificial
  SAGE Tag
tttggtgttt

SEQ ID NO: 1431 - DNA, 10 bp, Artificial
  SAGE Tag
cctcccaagg

SEQ ID NO: 1432 - DNA, 10 bp, Artificial
  SAGE Tag
tgttactgga

SEQ ID NO: 1433 - DNA, 10 bp, Artificial
  SAGE Tag
gtggcctttg

SEQ ID NO: 1434 - DNA, 10 bp, Artificial
  SAGE Tag
tgatgactgt

SEQ ID NO: 1435 - DNA, 10 bp, Artificial
  SAGE Tag
gcggctgcgc

SEQ ID NO: 1436 - DNA, 10 bp, Artificial
  SAGE Tag
gcaggatcgg

SEQ ID NO: 1437 - DNA, 10 bp, Artificial
  SAGE Tag
gcaggaaata

SEQ ID NO: 1438 - DNA, 10 bp, Artificial
  SAGE Tag
acctgaaacc

SEQ ID NO: 1439 - DNA, 10 bp, Artificial
  SAGE Tag
cccgaggcag

SEQ ID NO: 1440 - DNA, 10 bp, Artificial
  SAGE Tag
ggggctggca

SEQ ID NO: 1441 - DNA, 10 bp, Artificial
  SAGE Tag
tcctttggat

SEQ ID NO: 1442 - DNA, 10 bp, Artificial
  SAGE Tag
aagatcctca

SEQ ID NO: 1443 - DNA, 10 bp, Artificial
  SAGE Tag
gtgttcccat

SEQ ID NO: 1444 - DNA, 10 bp, Artificial
  SAGE Tag
gcacgtgagc

SEQ ID NO: 1445 - DNA, 10 bp, Artificial
  SAGE Tag
aaatgcagta

SEQ ID NO: 1446 - DNA, 10 bp, Artificial
  SAGE Tag
taagcattaa

SEQ ID NO: 1447 - DNA, 10 bp, Artificial
  SAGE Tag
gcgagactca

SEQ ID NO: 1448 - DNA, 10 bp, Artificial
  SAGE Tag
gtgtgggaga

SEQ ID NO: 1449 - DNA, 10 bp, Artificial
  SAGE Tag
agggctccca

SEQ ID NO: 1450 - DNA, 10 bp, Artificial
  SAGE Tag
tggtcaaggt

SEQ ID NO: 1451 - DNA, 10 bp, Artificial
  SAGE Tag
ccagccaggt

SEQ ID NO: 1452 - DNA, 10 bp, Artificial
  SAGE Tag
atcatcgctg

SEQ ID NO: 1453 - DNA, 10 bp, Artificial
  SAGE Tag
atacagattg

SEQ ID NO: 1454 - DNA, 10 bp, Artificial
  SAGE Tag
tgtgtgtgtg

SEQ ID NO: 1455 - DNA, 10 bp, Artificial
  SAGE Tag
gaggccaaga

SEQ ID NO: 1456 - DNA, 10 bp, Artificial
  SAGE Tag
atggcaggcg

SEQ ID NO: 1457 - DNA, 10 bp, Artificial
  SAGE Tag
tcctgggcct

SEQ ID NO: 1458 - DNA, 10 bp, Artificial
  SAGE Tag
gctatttcct

SEQ ID NO: 1459 - DNA, 10 bp, Artificial
  SAGE Tag
gtctgccctc

SEQ ID NO: 1460 - DNA, 10 bp, Artificial
  SAGE Tag
gcctgagctg

SEQ ID NO: 1461 - DNA, 10 bp, Artificial
  SAGE Tag
tggggacagg

SEQ ID NO: 1462 - DNA, 10 bp, Artificial
  SAGE Tag
ttggccaggg

SEQ ID NO: 1463 - DNA, 10 bp, Artificial
  SAGE Tag
cagttgccag

SEQ ID NO: 1464 - DNA, 10 bp, Artificial
  SAGE Tag
ggcctggcta

SEQ ID NO: 1465 - DNA, 10 bp, Artificial
  SAGE Tag
gttgtatgta

SEQ ID NO: 1466 - DNA, 10 bp, Artificial
  SAGE Tag
cccacctcaa

SEQ ID NO: 1467 - DNA, 10 bp, Artificial
  SAGE Tag
attgcaccac

SEQ ID NO: 1468 - DNA, 10 bp, Artificial
  SAGE Tag
gcacacgttt

SEQ ID NO: 1469 - DNA, 10 bp, Artificial
  SAGE Tag
cgtaaaaagg

SEQ ID NO: 1470 - DNA, 10 bp, Artificial
  SAGE Tag
atggtggggg

SEQ ID NO: 1471 - DNA, 10 bp, Artificial
  SAGE Tag
ccttgagtac

SEQ ID NO: 1472 - DNA, 10 bp, Artificial
  SAGE Tag
tacttgtatt

SEQ ID NO: 1473 - DNA, 10 bp, Artificial
  SAGE Tag
gctgccatcc

SEQ ID NO: 1474 - DNA, 10 bp, Artificial
  SAGE Tag
gagcaaagga

SEQ ID NO: 1475 - DNA, 10 bp, Artificial
  SAGE Tag
acatcaaagt

SEQ ID NO: 1476 - DNA, 10 bp, Artificial
  SAGE Tag
ccatccaggc

SEQ ID NO: 1477 - DNA, 10 bp, Artificial
  SAGE Tag
gtgagtgtgt

SEQ ID NO: 1478 - DNA, 10 bp, Artificial
  SAGE Tag
tttgtgtgtg

SEQ ID NO: 1479 - DNA, 10 bp, Artificial
  SAGE Tag
tggctgggtg

SEQ ID NO: 1480 - DNA, 10 bp, Artificial
  SAGE Tag
tgtggaaacc

SEQ ID NO: 1481 - DNA, 10 bp, Artificial
  SAGE Tag
aagattggtg

SEQ ID NO: 1482 - DNA, 10 bp, Artificial
  SAGE Tag
ggcatcaggg

SEQ ID NO: 1483 - DNA, 10 bp, Artificial
  SAGE Tag
tgcatctggt

SEQ ID NO: 1484 - DNA, 10 bp, Artificial
  SAGE Tag
aggagcgggg

SEQ ID NO: 1485 - DNA, 10 bp, Artificial
  SAGE Tag
aactatgcca

SEQ ID NO: 1486 - DNA, 10 bp, Artificial
  SAGE Tag
tgcagaagta

SEQ ID NO: 1487 - DNA, 10 bp, Artificial
  SAGE Tag
aaaatgaaga

SEQ ID NO: 1488 - DNA, 10 bp, Artificial
  SAGE Tag
tgtgtaccta

SEQ ID NO: 1489 - DNA, 10 bp, Artificial
  SAGE Tag
atcctgctgg

SEQ ID NO: 1490 - DNA, 10 bp, Artificial
  SAGE Tag
tgctgcctca

SEQ ID NO: 1491 - DNA, 10 bp, Artificial
  SAGE Tag
ctggacattg

SEQ ID NO: 1492 - DNA, 10 bp, Artificial
  SAGE Tag
gagggaggtt

SEQ ID NO: 1493 - DNA, 10 bp, Artificial
  SAGE Tag
ggcacctggc

SEQ ID NO: 1494 - DNA, 10 bp, Artificial
  SAGE Tag
tgctaagaat

SEQ ID NO: 1495 - DNA, 10 bp, Artificial
  SAGE Tag
agtagcgaac

SEQ ID NO: 1496 - DNA, 10 bp, Artificial
  SAGE Tag
catagagcca

SEQ ID NO: 1497 - DNA, 10 bp, Artificial
  SAGE Tag
aagtttgtgg

SEQ ID NO: 1498 - DNA, 10 bp, Artificial
  SAGE Tag
ggtgcccagt

SEQ ID NO: 1499 - DNA, 10 bp, Artificial
  SAGE Tag
gtccggagat

SEQ ID NO: 1500 - DNA, 10 bp, Artificial
  SAGE Tag
tgctttgctt

SEQ ID NO: 1501 - DNA, 10 bp, Artificial
  SAGE Tag
tctgttctgg

SEQ ID NO: 1502 - DNA, 10 bp, Artificial
  SAGE Tag
tttactttgg

SEQ ID NO: 1503 - DNA, 10 bp, Artificial
  SAGE Tag
cccctctgag

SEQ ID NO: 1504 - DNA, 10 bp, Artificial
  SAGE Tag
aaggacattc

SEQ ID NO: 1505 - DNA, 10 bp, Artificial
  SAGE Tag
gacttgatat

SEQ ID NO: 1506 - DNA, 10 bp, Artificial
  SAGE Tag
gcagctgcct

SEQ ID NO: 1507 - DNA, 10 bp, Artificial
  SAGE Tag
tttgagggaa

SEQ ID NO: 1508 - DNA, 10 bp, Artificial
  SAGE Tag
tggagatgtg

SEQ ID NO: 1509 - DNA, 10 bp, Artificial
  SAGE Tag
aatccaaagg

SEQ ID NO: 1510 - DNA, 10 bp, Artificial
  SAGE Tag
gtcacccaaa

SEQ ID NO: 1511 - DNA, 10 bp, Artificial
  SAGE Tag
ttaattgata

SEQ ID NO: 1512 - DNA, 10 bp, Artificial
  SAGE Tag
aaaagatact

SEQ ID NO: 1513 - DNA, 10 bp, Artificial
  SAGE Tag
gcaaatctga

SEQ ID NO: 1514 - DNA, 10 bp, Artificial
  SAGE Tag
tattattaaa

SEQ ID NO: 1515 - DNA, 10 bp, Artificial
  SAGE Tag
ccctgccttg

SEQ ID NO: 1516 - DNA, 10 bp, Artificial
  SAGE Tag
atccggaccc

SEQ ID NO: 1517 - DNA, 10 bp, Artificial
  SAGE Tag
gcgggggtgt

SEQ ID NO: 1518 - DNA, 10 bp, Artificial
  SAGE Tag
tggctgcata

SEQ ID NO: 1519 - DNA, 10 bp, Artificial
  SAGE Tag
tgcccagggg

SEQ ID NO: 1520 - DNA, 10 bp, Artificial
  SAGE Tag
ggagagaaaa

SEQ ID NO: 1521 - DNA, 10 bp, Artificial
  SAGE Tag
ttacttggcc

SEQ ID NO: 1522 - DNA, 10 bp, Artificial
  SAGE Tag
aactcttcac

SEQ ID NO: 1523 - DNA, 10 bp, Artificial
  SAGE Tag
gcaccattcc

SEQ ID NO: 1524 - DNA, 10 bp, Artificial
  SAGE Tag
agaacacagt

SEQ ID NO: 1525 - DNA, 10 bp, Artificial
  SAGE Tag
ttccccagaa

SEQ ID NO: 1526 - DNA, 10 bp, Artificial
  SAGE Tag
gatcaatgga

SEQ ID NO: 1527 - DNA, 10 bp, Artificial
  SAGE Tag
caaatgagga

SEQ ID NO: 1528 - DNA, 10 bp, Artificial
  SAGE Tag
agaagactgg

SEQ ID NO: 1529 - DNA, 10 bp, Artificial
  SAGE Tag
agtctgtcca

SEQ ID NO: 1530 - DNA, 10 bp, Artificial
  SAGE Tag
gactcaggga

SEQ ID NO: 1531 - DNA, 10 bp, Artificial
  SAGE Tag
ctgagcacaa

SEQ ID NO: 1532 - DNA, 10 bp, Artificial
  SAGE Tag
tcactgatgg

SEQ ID NO: 1533 - DNA, 10 bp, Artificial
  SAGE Tag
tacattctgt

SEQ ID NO: 1534 - DNA, 10 bp, Artificial
  SAGE Tag
ttactaaatg

SEQ ID NO: 1535 - DNA, 10 bp, Artificial
  SAGE Tag
tcagagatga

SEQ ID NO: 1536 - DNA, 10 bp, Artificial
  SAGE Tag
atgaaaggtt

SEQ ID NO: 1537 - DNA, 10 bp, Artificial
  SAGE Tag
gtgtcttgta

SEQ ID NO: 1538 - DNA, 10 bp, Artificial
  SAGE Tag
taacagccag

SEQ ID NO: 1539 - DNA, 10 bp, Artificial
  SAGE Tag
tgttcccttt

SEQ ID NO: 1540 - DNA, 10 bp, Artificial
  SAGE Tag
gctgggaggg

SEQ ID NO: 1541 - DNA, 10 bp, Artificial
  SAGE Tag
aacttgatac

SEQ ID NO: 1542 - DNA, 10 bp, Artificial
  SAGE Tag
gaaaaatagt

SEQ ID NO: 1543 - DNA, 10 bp, Artificial
  SAGE Tag
tttgaaatga

SEQ ID NO: 1544 - DNA, 10 bp, Artificial
  SAGE Tag
gcagctgacg

SEQ ID NO: 1545 - DNA, 10 bp, Artificial
  SAGE Tag
cactgccttg

SEQ ID NO: 1546 - DNA, 10 bp, Artificial
  SAGE Tag
gggggctcct

SEQ ID NO: 1547 - DNA, 10 bp, Artificial
  SAGE Tag
taccaccaat

SEQ ID NO: 1548 - DNA, 10 bp, Artificial
  SAGE Tag
ttctcatagg

SEQ ID NO: 1549 - DNA, 10 bp, Artificial
  SAGE Tag
gtggtgatgt

SEQ ID NO: 1550 - DNA, 10 bp, Artificial
  SAGE Tag
taccatcgat

SEQ ID NO: 1551 - DNA, 10 bp, Artificial
  SAGE Tag
ctgcagaata

SEQ ID NO: 1552 - DNA, 10 bp, Artificial
  SAGE Tag
ccactgtaag

SEQ ID NO: 1553 - DNA, 10 bp, Artificial
  SAGE Tag
gacttctgag

SEQ ID NO: 1554 - DNA, 10 bp, Artificial
  SAGE Tag
caaaaggctc

SEQ ID NO: 1555 - DNA, 10 bp, Artificial
  SAGE Tag
gcgtgagtgc

SEQ ID NO: 1556 - DNA, 10 bp, Artificial
  SAGE Tag
cgtgggtggg

SEQ ID NO: 1557 - DNA, 10 bp, Artificial
  SAGE Tag
gattgaacct

SEQ ID NO: 1558 - DNA, 10 bp, Artificial
  SAGE Tag
acctggggag

SEQ ID NO: 1559 - DNA, 10 bp, Artificial
  SAGE Tag
gacttggcgg

SEQ ID NO: 1560 - DNA, 10 bp, Artificial
  SAGE Tag
ggagacttcc

SEQ ID NO: 1561 - DNA, 10 bp, Artificial
  SAGE Tag
gtgacagaat

SEQ ID NO: 1562 - DNA, 10 bp, Artificial
  SAGE Tag
agtatgtatg

SEQ ID NO: 1563 - DNA, 10 bp, Artificial
  SAGE Tag
aggagcaaag

SEQ ID NO: 1564 - DNA, 10 bp, Artificial
  SAGE Tag
tctttacttg

SEQ ID NO: 1565 - DNA, 10 bp, Artificial
  SAGE Tag
caatggagct

SEQ ID NO: 1566 - DNA, 10 bp, Artificial
  SAGE Tag
taacccaaca

SEQ ID NO: 1567 - DNA, 10 bp, Artificial
  SAGE Tag
tcagtttgga

SEQ ID NO: 1568 - DNA, 10 bp, Artificial
  SAGE Tag
ttgttgaagc

SEQ ID NO: 1569 - DNA, 10 bp, Artificial
  SAGE Tag
aataaagcaa

SEQ ID NO: 1570 - DNA, 10 bp, Artificial
  SAGE Tag
ctaataaatg

SEQ ID NO: 1571 - DNA, 10 bp, Artificial
  SAGE Tag
atgtccaatt

SEQ ID NO: 1572 - DNA, 10 bp, Artificial
  SAGE Tag
agcgagagag

SEQ ID NO: 1573 - DNA, 10 bp, Artificial
  SAGE Tag
acagcctgca

SEQ ID NO: 1574 - DNA, 10 bp, Artificial
  SAGE Tag
agaacctgca

SEQ ID NO: 1575 - DNA, 10 bp, Artificial
  SAGE Tag
cctgtgacag

SEQ ID NO: 1576 - DNA, 10 bp, Artificial
  SAGE Tag
atcttgttac

SEQ ID NO: 1577 - DNA, 10 bp, Artificial
  SAGE Tag
aggccaacag

SEQ ID NO: 1578 - DNA, 10 bp, Artificial
  SAGE Tag
ttgtttgttt

SEQ ID NO: 1579 - DNA, 10 bp, Artificial
  SAGE Tag
tgtaggtatt

SEQ ID NO: 1580 - DNA, 10 bp, Artificial
  SAGE Tag
ttccgcgtgc

SEQ ID NO: 1581 - DNA, 10 bp, Artificial
  SAGE Tag
aaaaaaaaac

SEQ ID NO: 1582 - DNA, 10 bp, Artificial
  SAGE Tag
caccaccacg

SEQ ID NO: 1583 - DNA, 10 bp, Artificial
  SAGE Tag
tgaggacaca

SEQ ID NO: 1584 - DNA, 10 bp, Artificial
  SAGE Tag
tggcttgctc

SEQ ID NO: 1585 - DNA, 10 bp, Artificial
  SAGE Tag
aacatcaaac

SEQ ID NO: 1586 - DNA, 10 bp, Artificial
  SAGE Tag
tacctgtgcc

SEQ ID NO: 1587 - DNA, 10 bp, Artificial
  SAGE Tag
aagaactaaa

SEQ ID NO: 1588 - DNA, 10 bp, Artificial
  SAGE Tag
tggcgtggcc

SEQ ID NO: 1589 - DNA, 10 bp, Artificial
  SAGE Tag
taacttgtga

SEQ ID NO: 1590 - DNA, 10 bp, Artificial
  SAGE Tag
atcagcaagt

SEQ ID NO: 1591 - DNA, 10 bp, Artificial
  SAGE Tag
gagctgttgg

SEQ ID NO: 1592 - DNA, 10 bp, Artificial
  SAGE Tag
taatattttt

SEQ ID NO: 1593 - DNA, 10 bp, Artificial
  SAGE Tag
ctggggagtg

SEQ ID NO: 1594 - DNA, 10 bp, Artificial
  SAGE Tag
aaccagaatg

SEQ ID NO: 1595 - DNA, 10 bp, Artificial
  SAGE Tag
gccttaaaaa

SEQ ID NO: 1596 - DNA, 10 bp, Artificial
  SAGE Tag
agccaccaca

SEQ ID NO: 1597 - DNA, 10 bp, Artificial
  SAGE Tag
gaaccctggg

SEQ ID NO: 1598 - DNA, 10 bp, Artificial
  SAGE Tag
aggtgcctcg

SEQ ID NO: 1599 - DNA, 10 bp, Artificial
  SAGE Tag
aatctgcgcc

SEQ ID NO: 1600 - DNA, 10 bp, Artificial
  SAGE Tag
aggtcaaaaa

SEQ ID NO: 1601 - DNA, 10 bp, Artificial
  SAGE Tag
gttcaaagac

SEQ ID NO: 1602 - DNA, 10 bp, Artificial
  SAGE Tag
aaggtgcctc

SEQ ID NO: 1603 - DNA, 10 bp, Artificial
  SAGE Tag
atgggcactg

SEQ ID NO: 1604 - DNA, 10 bp, Artificial
  SAGE Tag
cagctgtagt

SEQ ID NO: 1605 - DNA, 10 bp, Artificial
  SAGE Tag
gggtttgaac

SEQ ID NO: 1606 - DNA, 10 bp, Artificial
  SAGE Tag
tatttattcc

SEQ ID NO: 1607 - DNA, 10 bp, Artificial
  SAGE Tag
agggtgaaac

SEQ ID NO: 1608 - DNA, 10 bp, Artificial
  SAGE Tag
tggaacagga

SEQ ID NO: 1609 - DNA, 10 bp, Artificial
  SAGE Tag
tgatcttttc

SEQ ID NO: 1610 - DNA, 10 bp, Artificial
  SAGE Tag
gacactgaaa

SEQ ID NO: 1611 - DNA, 10 bp, Artificial
  SAGE Tag
attccaagaa

SEQ ID NO: 1612 - DNA, 10 bp, Artificial
  SAGE Tag
cctcagccag

SEQ ID NO: 1613 - DNA, 10 bp, Artificial
  SAGE Tag
gctaatagta

SEQ ID NO: 1614 - DNA, 10 bp, Artificial
  SAGE Tag
taaataaagg

SEQ ID NO: 1615 - DNA, 10 bp, Artificial
  SAGE Tag
gctcattaaa

SEQ ID NO: 1616 - DNA, 10 bp, Artificial
  SAGE Tag
ttttatagct

SEQ ID NO: 1617 - DNA, 10 bp, Artificial
  SAGE Tag
taaaacgtga

SEQ ID NO: 1618 - DNA, 10 bp, Artificial
  SAGE Tag
aaggtaacta

SEQ ID NO: 1619 - DNA, 10 bp, Artificial
  SAGE Tag
aaagattgga

SEQ ID NO: 1620 - DNA, 10 bp, Artificial
  SAGE Tag
cctgtacccc

SEQ ID NO: 1621 - DNA, 10 bp, Artificial
  SAGE Tag
tagacttatt

SEQ ID NO: 1622 - DNA, 10 bp, Artificial
  SAGE Tag
ggggtctggg

SEQ ID NO: 1623 - DNA, 10 bp, Artificial
  SAGE Tag
agctggagtc

SEQ ID NO: 1624 - DNA, 10 bp, Artificial
  SAGE Tag
cgaccgtggc

SEQ ID NO: 1625 - DNA, 10 bp, Artificial
  SAGE Tag
aaggaccttt

SEQ ID NO: 1626 - DNA, 10 bp, Artificial
  SAGE Tag
tctgcctggg

SEQ ID NO: 1627 - DNA, 10 bp, Artificial
  SAGE Tag
tgtttggggg

SEQ ID NO: 1628 - DNA, 10 bp, Artificial
  SAGE Tag
tttgaggatt

SEQ ID NO: 1629 - DNA, 10 bp, Artificial
  SAGE Tag
agttgaaatt

SEQ ID NO: 1630 - DNA, 10 bp, Artificial
  SAGE Tag
ttccagacct

SEQ ID NO: 1631 - DNA, 10 bp, Artificial
  SAGE Tag
cacaccattg

SEQ ID NO: 1632 - DNA, 10 bp, Artificial
  SAGE Tag
gaaaggtctg

SEQ ID NO: 1633 - DNA, 10 bp, Artificial
  SAGE Tag
gttttcattc

SEQ ID NO: 1634 - DNA, 10 bp, Artificial
  SAGE Tag
atgggcttga

SEQ ID NO: 1635 - DNA, 10 bp, Artificial
  SAGE Tag
gtgcctaggg

(19) United States
(12) Patent Application Publication
(10) Pub. No.: US 2007/0224596 A1
(43) Pub. Date: Sep. 27, 2007

(54) Compositions and methods for the modifying hypoxia induced gene regulation

(76) Inventors: Mariana Nacht, Belmont, MA (US); Yide Alan Jiang, Newton, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/247,103

(22) Filed: Jan. 20, 2006

(30) Foreign Application Priority Data
Apr. 9, 2004 (WO) PCT/US04/11087

(51) Int. Cl. C12Q 1/68; G06F 19/00

(52) U.S. Cl. 435/6; 702/20

(57) ABSTRACT

This invention provides compositions and methods to identify candidate agents capable of altering the biological activity of a polypeptide encoded by a polynucleotide involved in hypoxia-related tumorigenesis. In one aspect, the biological activity is the induction of hypoxia-related gene enolase 2 or a biological equivalent thereof. In another aspect, the biological activity is the induction of a hypoxia-related gene, inducible in the absence of the von Hippel-Lindau tumor suppressor (VHL). In yet a further aspect, the biological activity is differential expression in a neoplastic cell under hypoxia. In an alternative aspect, the biological activity is induction of a gene that is inducible in the absence of VHL, but not hypoxia.

### CROSS-REFERENCE TO RELATED APPLICATIONS

This application claims priority under 35 U.S.C. § 119(e) to provisional application Ser. No. 60/461,712, filed Apr. 9, 2003, the contents of which are hereby incorporated by reference into the present disclosure.

### TECHNICAL FIELD

This invention relates to isolated polynucleotides shown to be up or down regulated in a pathological cell as compared to a counterpart normal, healthy cell.

### BACKGROUND OF THE INVENTION

It is known that many, but not all genes present in a cell are expressed at any given time. Fundamental questions of biology require knowledge of which genes are transcribed and the relative abundance of transcripts in different cells. Typically, when and to what degree a given gene is expressed has been analyzed one gene at a time.

Thus, information regarding the identity of all expressed genes in a cell and the level of expression of these genes would facilitate the study of many cellular processes such as activation, differentiation, aging, viral transformation, morphogenesis, and mitosis. A comparison of the expressed genes of a particular cell or the same cell from various individuals or species, under the same or different environmental stimuli, provides valuable insight into the molecular biology of the cell. SAGE (Serial Analysis of Gene Expression or “SAGE”) disclosed in Velculescu, et al. (1995) Science 270:484-487 and U.S. Pat. No. 5,695,937) is a technique that provides such information. Using SAGE, the expressed genes in normal renal proximal tubule cells were compared to renal carcinoma cells that do, and do not express the tumor suppressor gene known as the von Hippel-Lindau tumor suppressor (“VHL”).

It is widely accepted that most solid tumors larger than several mm³ have undergone an angiogenic switch, a crucial step for tumor growth and metastasis. Thus, blocking angiogenesis could be a strategy to arrest tumor growth (Folkman (1971) New Eng. J. Med. 285(21):1182-1186). Various signals from both genetic mutations and environmental factors are involved in this switch (Carmeliet and Jam (2000) Nature 407:249-257), with one of the most potent environmental signals being hypoxia (low oxygen tension). Although new blood vessel formation is one of the consequences of hypoxia and results in the alleviation of the hypoxic environment, some regions of large solid tumors continue to be under hypoxia. Adaptation to hypoxia is of fundamental importance in developmental, physiological, and pathophysiological processes (Bunn and Poyton (1996) Physiol. Rev. 76(3):839-885; Guillemin and Krasnow (1997) Cell 89(1):9-12). At the molecular level, this adaptation to hypoxia depends in part on appropriate expression of many physiologically relevant genes that are regulated transcriptionally, posttranscriptionally or posttranslationally. The gene expression changes in response to hypoxia are important to many fundamental biological processes such as apoptosis, cell cycle control, stress adaptation, anaerobic metabolism, tissue remodeling and angiogenesis. Genes whose expression change in response to hypoxia include: erythropoietin (Epo) which plays a crucial role in regulating the oxygen-carrying capacity of the blood; vascular endothelial growth factor (VEGF) which is a potent angiogenic growth factor that induces new blood vessel formation in both physiological and pathophysiological processes; and genes involved in the long-term adaptation of energy metabolism in an environment of decreased oxygen tension such as glucose transporter 1 (GLUT 1) and phosphoglycerate kinase 1 (PGK1) (Bunn and Poyton (1996) supra; Semenza (1999) Ann. Rev. Cell Dev. Biol. 15:551-578). Enhanced glucose metabolism and angiogenesis are hallmarks of tumor growth and involve up-regulation of genes that are normally induced by hypoxia.

Hypoxia-inducible factor (HIF) is a critical transcriptional regulator which modulates many hypoxia-associated genes, including GLUT I and VEGF (Semenza (2000) J. Appl. Physiol. 88(4):1474-1480; and Semenza (2000) Genes Dev. 14(16):1983-1991). HIF is a heterodimer comprised of HIF-α and ARNT, two basic helix-loop-helix proteins in the PAS family (Semenza (1999) supra). ARNT mRNA and protein levels are not significantly effected by ambient oxygen tension. There are at least two mammalian HIF-α isoforms, HIF-1α and HIF-2α, that appear to be predominantly regulated by protein stability, although there is some evidence that HIF mRNA levels may also be effected by oxygen (Wenger (2000) J. Exp. Biol. 203(Pt. 8):1253-1263). HIF-α proteins are only minimally present under normoxia due to their rapid degradation through the ubiquitin-proteasome pathway (Wenger (2000) supra). In contrast, under low oxygen tension, HIF-α. proteins are stabilized (Semenza (2000) Genes Dev. 14(16):1983-1991).

Inactivation of the VHL tumor suppressor gene results in the development of von Hippel-Lindau disease, a hereditary cancer syndrome distinguished by highly angiogenic tumors of the kidney, retina, pancreas and central nervous system (Clifford and Maher (2001) Adv. Cancer Res. (82):85-105; Kondo and Kaelin (2001) Exp. Cell Res. 264(1):117-125). Several lines of evidence have emerged to suggest that pVHL, the VHL gene product, is a multifunctional protein. The most well characterized function of pVHL is its role as a component of the ubiquitin E3 ligase complex that targets HIF-α for ubiquitin-dependent proteasome degradation (Cockman et al. (2000) J. Biol. Chem. 275(33):25733-25741). Recent studies demonstrated that the VHL-dependent proteolytic degradation of both HIF-1α and HIF-2α occurs through enzymatic hydroxylation of specific prolyl residues within the HIF-α ODDD (oxygen dependent degradation domain) (Ivan et al. (2001) Science 292(5516):464-468).

In hypoxic cells, HIF-α degradation is suppressed leading to enhanced transcription of target genes, including pro-angiogenic genes. In renal cell carcinoma (RCC), a common manifestation of VHL disease, HIF-α overexpression is observed throughout the tumor. Two of the known transcriptional targets of HIF-a, GLUT 1 and VEGF (Wiesener et al. (2001) Cancer Res. 61(13):5215-5222), are also overexpressed in RCC tumors and likely contribute to the activation of the angiogenic switch found in tumorigenesis. The renal cell carcinoma cell line 786-O does not express detectable HIF-1α, although expression of HIF-2α is constitutively high in these cells (Maxwell et al. (1999) Nature 399(6733):271-275).

Thus, there is a need in the art to elucidate the molecular mechanisms of tumor cell behavior, including hypoxia signaling pathways, that are now believed to have a profound impact on the diagnosis, prognosis, and treatment of tumors (Livingston and Shivdasani (2001) JAMA 285(5):588-593). This invention satisfies this need and provides related advantages as well.

### DESCRIPTION OF THE INVENTION

The von Hippel-Lindau tumor suppressor, pVHL, is a key player in one of the best-characterized hypoxia signaling pathways, the VHL-HIF pathway. Serial Analysis of Gene Expression (SAGE) supra, was used to investigate hypoxia-regulated gene expression in renal carcinoma cells (786-0) with and without VHL. The gene expression profiles of the cancer cells were compared to SAGE profiles from normal renal proximal tubule cells grown under both normoxia and hypoxia.

The genes identified in Table 2 infra, were shown to be differentially expressed in cancer cells as compared to normal renal cells when grown under normoxia and hypoxia. Most of these have not been previously connected to differential expression under these conditions.

Based on these findings, this invention provides screening methods to identify candidate agents capable of altering the biological activity of a polypeptide encoded by a polynucleotide involved in hypoxia-related tumorigenesis by contacting a test agent with a target cell expressing the polynucleotide, and monitoring activity of the expressed polypeptide product, wherein the test agent which modifies the activity of the polypeptide is a candidate agent. In one aspect, the biological activity is the induction of hypoxia-related gene enolase 2 (GenBank No. Y00691M27) or a biological equivalent thereof.

In another aspect, the biological activity is the induction of a hypoxia-related gene, inducible in the absence of VHL, wherein the gene is selected from the group comprising at least one of integrin alpha E (GenBank No. L25851), endothelin 2 (GenBank No. X55177), stress-induced endoplastic reticulum protein 1 (GenBank No. AB022427), phosphoglutamase 1 (GenBank No. M83088), cell-division cycle 25B (AL10980) and biological equivalents thereof.

In a further aspect, the biological activity is differential expression in a neoplastic cell under hypoxia, as compared to a normal counterpart cell and in a neoplastic cell that has lost VHL and the gene is selected from the group comprising at least one of enolase 2 (GenBank No. Y00691 M27) glia maturation factor B (GenBank No. AB 001106) and biological equivalents thereof.

In yet a further aspect, the biological activity is induction of a gene, induced in the absence of VHL but not hypoxia, wherein the gene is selected from the group comprising at least one of metalloprotease 1 (GenBank No. AK001183), insulin-like growth factor binding protein 3 (GenBank No. Hs. 77326), and biological equivalents thereof. In one aspect, the target cell of the assay is a renal carcinoma cell.

In another aspect, the screen further requires contacting a normal, healthy counterpart cell (e.g., renal cell such as a renal proximal tubule cell) to the test cell and monitoring the activity of the polypeptide.

The screen also is useful to determine if a drug or therapy is effective for an individual in need of such treatment by obtaining the target cell from the individual, wherein the target cell is suspected of being involved in the pathology or tissue to be treated. It also is useful to monitor the efficacy of a drug or therapy by performing the screen prior to treatment and/or at different time points during treatment, and comparing the results obtained at different time points.

In one aspect the target cell is present in a subject and therefore in vivo. Subjects, for example, rats, mice, simians or the like, containing a target cell can be used as animal models for the discovery of new potential therapeutics and/or to personalize treatment to the individual subject (as noted above) or to monitor treatment.

The screen can be practiced in silico by comparing the transciptome of a test or target cell to a database containing one or more sequences identified in Table 2. A computer-readable medium having stored one or more gene sequences or biological equivalents thereto is further provided by this invention. Several therapeutic benefits are achievable by the modulation, e.g., enhancement or suppression of expression, of the genes of this invention. For example, by inhibiting the biological activity of enolase 2 (GenBank No. Y00691M27) or a biological equivalent thereof, one can negatively affect cell metabolism and inhibit cell growth and/or promote apoptosis (cell death). Alternatively, by enhancing the expression of this gene or its equivalent, one can enhance growth in adverse oxygen environments such as ischemic heart disease.

Also provided are methods and compositions to inhibit a hypoxia-related gene, inducible in the absence of VHL, wherein the gene is selected from the group comprising at least one of integrin alpha E (GenBank No. L25851), endothelin 2 (GenBank No. X55177), stress-induced endoplastic reticulum protein 1 (GenBank No. AB022427), phosphoglutamase 1 (GenBank No. M83088), cell-division cycle 25B (AL10980), and biological equivalents thereof. By use of these compositions and methods, one can specifically target mutant neoplastic cells and prevent cell growth and/or promote apoptosis (cell death). Detection of such genes can be diagnostic for neoplastic cells that have lost VHL expression and thus can dictate treatment options for such cancers. Alternatively, by enhancing the expression of one of these genes or its equivalent, one can enhance cell growth or prevent apoptosis.

In a further aspect, inhibition of a gene selected from the group comprising at least one of enolase 2 (GenBank No. Y00691 M27), glia maturation factor B (GenBank No. AB001106) and biological equivalents thereof, provides a method to negatively affect cell metabolism to inhibit cell growth and/or promote apoptosis (cell death). Alternatively, by enhancing the expression of at least one of these genes or its equivalent, one can enhance growth in adverse oxygen environments such as ischemic heart disease.

In yet a further aspect, inhibition of a gene, induced in the absence of VHL but not hypoxia, wherein the gene is selected from the group comprising at least one of metalloprotease 1 (GenBank No. AK001183), insulin-like growth factor binding protein 3 (GenBank No. Hs. 77326), and biological equivalents thereof, provides a method to distinguish mutant neoplastic cells from cells grown in low oxygen. Inhibition of such a gene can negatively affect cell growth specifically in neoplastic cells that have lost VHL but not in cells growing under hypoxia or in normal cells. Alternatively, by enhancing the expression of one of these genes or its equivalent, one can enhance cell growth in populations of cells that have lost VHL gene function.

Agents and compositions known to inhibit gene function include but are not limited to agents and compositions that degrade mRNA (ribozymes), anti-sense nucleotides, small interfering RNAs, antibodies that block action of polynucleotides and small molecule drug inhibitors. For the purpose of illustration only, specific examples include, but are not limited to, Herceptin™ (Genentech, Inc.), Gleevac™ (Novartis) and thalidomide.

Agents and compositions known to enhance gene function include but are not limited to agents and compositions that enhance gene expression (e.g., enhancers) or that block or inhibit negative gene regulators such as activator proteins and proteins or nucleotides that bind to negative regulatory elements on DNA. For the purpose of illustration only, specific examples include, but are not limited to, AP-2, and the trichothecene mycotoxin vomitoxin (VT).

Compositions useful in the screens and methods described herein are further provided by this invention.

### BRIEF DESCRIPTION OF THE FIGURES

FIG. 1 shows genes that are negatively regulated by VHL in 786-0 cells. The black circle on the left denotes genes that are up-regulated ∃4-fold in 786-0 VHL^{!}Nor. cells compared to 786-0 VHL⁺Nor. cells; the white circle on the right indicates the genes that are up-regulated ∃4-fold in 786-0 VHL+Hyp. cells compared to 786-0 VHL⁺Nor. cells. The gray intersection of the two circles denotes the genes that are up-regulated in both comparisons. The number of genes included in each set is indicated. Letters in each section refer to representative genes from each set which can be found in the corresponding section of Tables 2 and 3.

FIG. 2 shows the effects of VHL on gene expression in 786-0 cells. The black circle on the left denotes the genes that are up-regulated ∃4-fold in 786-0 VHL^{!}Nor. cells compared to Renal Proximal Tubule (RPTEC) Nor. cells; the white circle on the right indicates the genes that are up-regulated ∃4-fold in 786-0 VHL⁺Nor. cells. Because two independent SAGE libraries were made from the RPTECs under each growth condition, the tag values were averaged before comparing. The gray intersection of the two circles denotes the genes that are up-regulated in both comparisons. The number of genes induced in each set is indicated. Letters in each section refer to representative genes from each set which can be found in the corresponding sections of Table 2 and 4.

FIG. 3 shows an alternative hypoxia-responsive pathway in 786-0 cells expressing VHL. The black circle on the left denotes the genes that are up-regulated ∃4-fold in 786-0 VHL⁺Hyp. cells compared to 786-0 VHL⁺Nor. cells; the white circle on the right indicates the genes that are up-regulated ∃4-fold in RPTEC Hyp. cells compared to RPTEC Nor. cells. SAGE tag values for the two independent libraries of RPTECs grown under each condition were averaged before comparing. The gray intersection of the two circles denotes the genes that are up-regulated in both comparisons. The number of genes induced in each set is indicated. Letters in each section refer to representative genes from each set which can be found in the corresponding sections of Table 2 and 5.

FIG. 4 schematically shows hypoxia-responsive genes in 786-0 cells lacking VHL. The black circle on the left denotes the genes that are up-regulated ∃4-fold in 786-0 VHL^{!}Hyp. cells compared to ∃4-fold in 786-0 VHL^{!}Nor. cells; the white circle on the right indicates the genes that are up-regulated ∃4-fold in 786-0 VHL⁺Hyp. compared to 786-0 VHL⁺Nor. cells. The gray intersection of the two circles denotes the genes that are up-regulated in both comparisons. The number of genes included in each set is indicated. Letters in each section refer to representative genes from each set which can be found in the corresponding sections of Table 2 and 6.

FIG. 5 graphically shows that real-time quantitative RT-PCR expression confirms SAGE expression. Quantitative RT-PCR was performed on three genes observed to be induced by SAGE. RNA from an independently generated set of 786-0-VHL⁺ cells (WT8) and 786-0 VHL cells (pRC3) was used to confirm induced expression of CCNβ1, TGFI, and GLUT1 in the RCC cells compared to RPTECs.

### BRIEF DESCRIPTION OF THE TABLES

Table 1 shows SAGE libraries of cancer cells (TRCC 768-0) and normal cells.

Table 2, subsections (a) through (i), identifies several SAGE tags that were found to be differentially expressed in renal cells grown under deficient conditions.

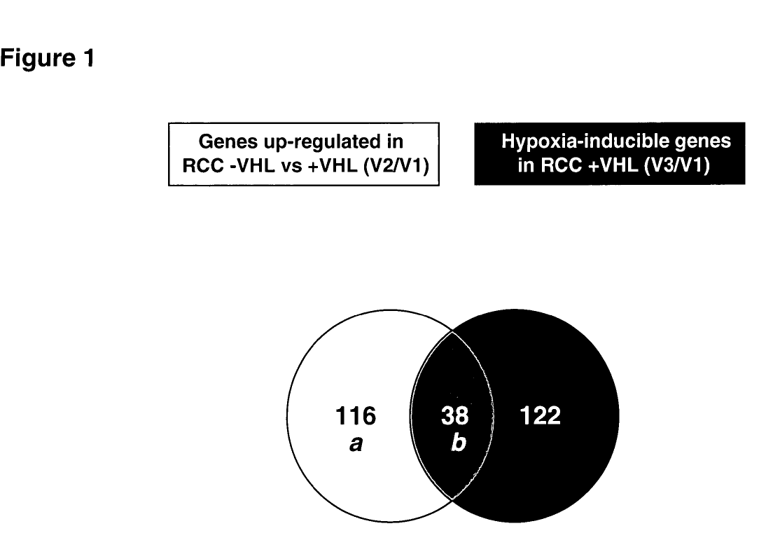

Table 3A identifies 116 genes represented in FIG. 1(A) (109 genes; 7 tags with no matches are not shown). SAGE data for genes up-regulated ∃4-fold in 786-0 VHL^{!}Nor. (V2) when compared to 786-0 VHL⁺Nor. (V1). Also shown, for comparison, is the SAGE data for each gene in the 786-0 VHL⁺Hyp. (V3) with respect to 786-0 VHL⁺Nor. (V1). SAGE tag abundances were normalized before calculating ratios, as shown. The table is presented in descending order of V2/V1.

Table 3B identifies 38 genes represented in FIG. 1(B) (36 genes; 2 tags with no matches are not shown). SAGE data for genes up-regulated >4-fold in 786-0 VHL^{!}Nor. (V2) compared to 786-0 VHL⁺Nor. (V1), and induced by hypoxia in 786-0 VHL⁺Hyp. (V3) compared to 786-0 VHL⁺Nor. (V1). SAGE tag abundances were normalized before calculating ratios, as shown. The table is presented in descending order of V2/V1.

Table 3C identifies 122 genes represented in FIG. 1 (105 genes; 17 tags with no matches are not shown). SAGE data for genes up-regulated ∃4-fold by hypoxia in 786-0 VHL⁺Hype. (V3) when compared to 786-0 VHL⁺Nor. (V1). Also shown, for comparison, is the SAGE data for each gene in the 786-0 VHL^{!}Nor. (V2) with respect to 786-0 VHL⁺Nor. (V1). SAGE tag abundances were normalized before calculating ratios, as shown. The table is presented in descending order of V3/V1.

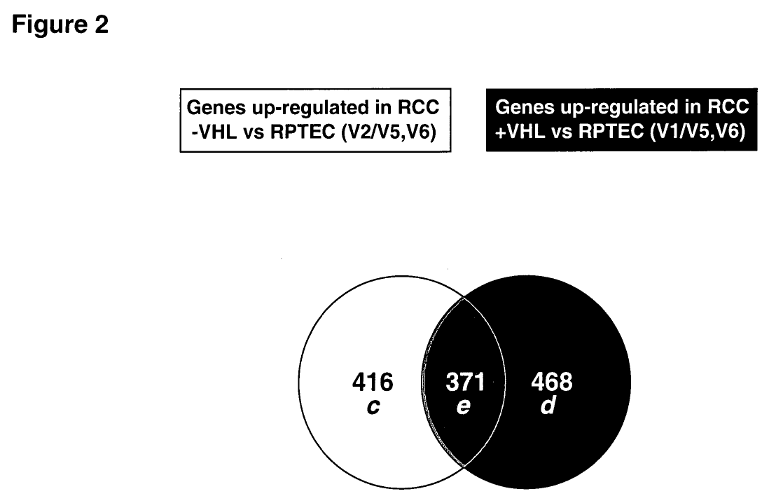

Table 4A identifies 416 genes represented in FIG. 2(C) (369 genes; 47 tags with no matches are not shown). SAGE data for genes up-regulated ≧4-fold in 786-0 VHL^{!}Nor. (V2) when compared to the average of two independent cultures of Renal Proximal Tubule cells (RPTECs) grown under nomoxia (V5, V6). Also shown, for comparison, is the SAGE data for each gene in the 786-0 VHL⁺Nor. (V1) with respect to its average expression in the two independent cultures of Renal Proximal Tubule cells (RPTECs) grown under nomoxia (V5, V6). SAGE tag abundances were normalized before calculating ratios, as shown. The table is presented in descending order of V2/V5, V6.

Table 4B also identifies 468 genes represented in FIG. 2(D) (421 genes; 47 tags with no matches are not shown). SAGE data for genes up-regulated ≧4-fold in 786-0 VHL⁺Nor. (V1) when compared to the average of two independent cultures of Renal Proximal Tubule cells (RPTECs) grown under nomoxia (V5, V6). Also shown, for comparison, is the SAGE data for each gene in the 786-0 VHL^{!}Nor. (V2) with respect to its average expression in the two independent cultures of Renal Proximal Tubule cells (RPTECs) grown under nomoxia (V5, V6). SAGE tag abundances were normalized before calculating ratios, as shown. The table is presented in descending order of V1/V5, V6.

Table 4C further identifies 371 genes represented in FIG. 2(E) (351 genes; 20 tags with no matches are not shown). SAGE data for genes up-regulated ≧4-fold in 786-0 cells, regardless of VHL status (VHL⁺, V1 and VHL^{!}, V2) with respect to the average of two independent cultures of Renal Proximal Tubule cells (RPTECs) grown under nomoxia (V5, V6). The table is presented in descending order of V2/V5, V6.

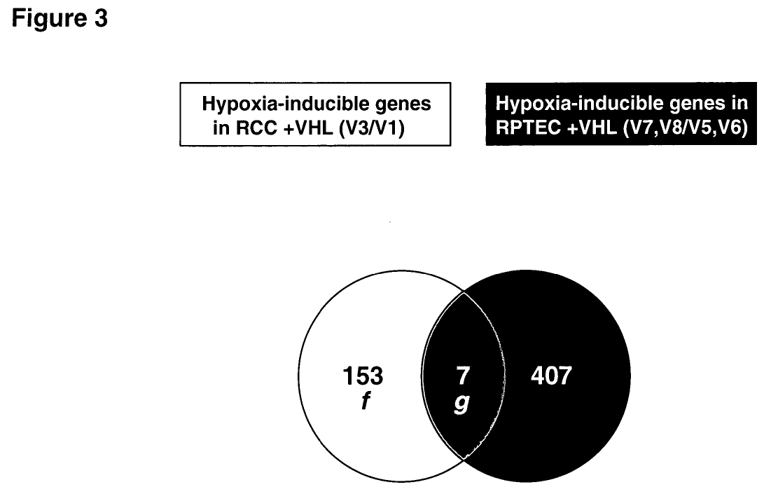

Table 5A identifies 153 genes represented in FIG. 3(F) (135 genes; 18 tags with no matches are not shown). SAGE data for genes up-regulated ≧4-fold by hypoxia in 786-0 VHL⁺Hyp. (V3) when compared to 786-0 VHL⁺Nor. (V1). Also shown, for comparison, is the SAGE data for each gene as a ratio of its average expression in the two independent culture of RPTECs grown under hypoxia (V7, V8) with respect to their average expression in the two independent cultures of RPTECs grown under nomoxia (V5, V6). SAGE tag abundances were normalized before calculating ratios, as shown. The table is presented in descending order of V3/V1.

Table 5B also identifies 7 genes represented in FIG. 3(G) (6 genes; 1 novel tag are not shown). SAGE data for genes up-regulated ∃4-fold by hypoxia in both 786-0 VHL⁺ cells and RPTECs. Normalized SAGE data from 786-0 VHL⁺Hyp. (V3) was compared to normalized data from 786-0 VHL⁺Nor. (V1). The average tag abundance for the two independent cultures of RPTECs grown under hypoxia (V7, V8) and nomoxia (V5, V6) was used. The table is presented in descending order of V3/V1.

Table 5C further identifies 407 genes represented in FIG. 3 (347 genes; 60 tags with no matches are not shown). SAGE data for genes up-regulated ≧4-fold by hypoxia in RPTECs. The average tag abundance for the two independent cultures of RPTECs grown under hypoxia (V7, V8) and nomoxia (V5, V6) was used. Also shown, for comparison, is the SAGE data for each gene in 786-0 VHL⁺Hyp. (3) compared to 786-0 VHL^{!}Nor. (V1). SAGE tag abundance were normalized before calculating ratios, as shown. The table is presented in descending order of V7, V8/V5, V6.

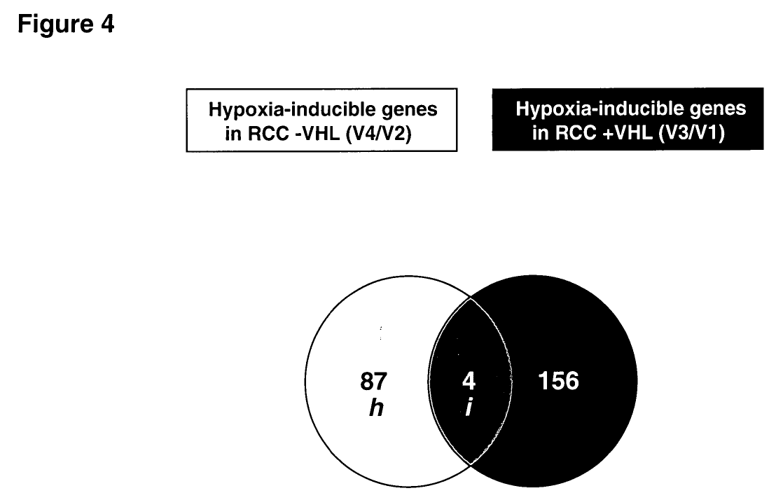

Table 6A identifies 87 genes represented in FIG. 4(H) (83 genes; 4 tags with no matches are not shown). SAGE data for genes up-regulated ≧4-fold by hypoxia in 786-0 VHL^{!}Hyp. (V4) when compared to those in 786-0 VHL^{!}Nor. (V2). Also shown, for comparison, is the SAGE data for each gene in 786-0 VHL⁺Hyp. (V3) compared to 786-0 VHL⁺Nor. (V1). SAGE tag abundances were normalized before calculating ratios, as shown. The table is presented in descending order of V4/V2.

Table 6B also identifies 4 genes represented in FIG. 4(I). SAGE data for genes up-regulated ≧4-fold by hypoxia in 786-0 cells, regardless of VHL status (VHL⁺, V3 and VHL^{!}, V4) with respect to 786-0 cells grown under nomoxia (VHL⁺, V1 and VHL^{!}, V2) SAGE tag abundances were normalized before calculating ratio, as shown. The table is presented in descending order of V4/V2.

Table 6C further identifies 156 genes represented in FIG. 4 (137 genes; 19 tags with no matches are not shown). SAGE data for genes up-regulated ≧4-fold by hypoxia in 786-0 VHL⁺Hyp. (V3) when compared to 786-0 VHL⁺Nor. (V1). Also shown, for comparison, is the SAGE data for each gene 786-0 VHL^{!}Hyp. (V4) when compared to 786-0 VHL^{!}Nor. (V2). SAGE tag abundances were normalized before calculating ratios, as shown. The table is presented in descending order of V3/V1.

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

The practice of the present invention will employ, unless otherwise indicated, conventional techniques of immunology, molecular biology, organic chemistry, microbiology, cell biology and recombinant DNA. These methods are described in the following publications. See, e.g., Sambrook, et al. MOLECULAR CLONING: A LABORATORY MANUAL, 2^{nd} edition (1989); CURRENT PROTOCOLS IN MOLECULAR BIOLOGY (F. M. Ausubel, et al. eds, (1987)); the series METHODS IN ENZYMOLOGY (Academic Press, Inc.); “PCR: A PRACTICAL APPROACH “(M. MacPherson, et al., IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane, Eds. (1988)); and ANIMAL CELL CULTURE (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

The term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The terms “polynucleotide” and “nucleic acid molecule” are used interchangeable to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

A “gene” refers to a polynucleotide containing at least one open reading frame that is capable of encoding a particular polypeptide or protein after being transcribed and translated.

A biologically equivalent polynucleotide is a polynucleotide that hybridizes under moderate or stringent conditions to a sequence identified in Table 2 or its complement. “Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed using traditional hybridization techniques under different stringency. In general, a low stringency hybridization reaction is carried out at about 40° C. in 10×SSC or a solution of equivalent ionic strength/temperature. A moderate stringency hybridization is typically performed at 10 about 50° C. in 6×SSC, and a high stringency hybridization reaction is generally performed at about 60° C. in 1×SSC. Alternatively, TMAC hybridization technology can be used for hybridization reactions probed with pooled oligonucleotides such as the SAGE tags. The advantage of using TMAC hybridization is that the reaction condition is not dependent on the G+C content of the oligonucleotide, and the melting temperature is determined only by the length of the Qligomers to be used.

When hybridization occurs in an anti-parallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules. A polynucleotide that is 100% complementary to a second polynucleotide are understood to be “complements” of each other.

Equivalent polynucleotides also include polynucleotides that are greater than 75%, or 80%, or more than 90%, or more than 95% homologous to the sequence shown in Table 2 and as further isolated and identified using sequence homology searches. Sequence homology is determined using a sequence alignment program run under default parameters and correcting for ambiguities in the sequence data, changes in nucleotide sequence that do not alter the amino acid sequence because of degeneracy of the genetic code, conservative amino acid substitutions and corresponding changes in nucleotide sequence, and variations in the lengths of the aligned sequences due to splicing variants or small deletions or insertions between sequences that do not affect function.

A variety of sequence alignment software programs are available in the art. Non-limiting examples of these programs are BLAST family programs including BLASTN, BLASTP, BLASTX, TBLASTN, and TBLASTX (BLAST is available from the worldwide web at ncbi.nlm.nih.gov/BLAST/), FastA, Compare, DotPlot, BestFit, GAP, FrameAlign, ClustalW, and Pileup. These programs are obtained commercially available in a comprehensive package of sequence analysis software such as GCG Inc.'s Wisconsin Package. Other similar analysis and alignment programs can be purchased from various providers such as DNA Star's MegAlign, or the alignment programs in GeneJockey. Alternatively, sequence analysis and alignment programs can be accessed through the world wide web at sites such as the CMS Molecular Biology Resource at sdsc.edu/ResTools/cmshp.html. Any sequence database that contains DNA or protein sequences corresponding to a gene or a segment thereof can be used for sequence analysis. Commonly employed databases include but are not limited to GenBank, EMBL, DDBJ, PDB, SWISS-PROT, EST, STS, GSS, and HTGS.

Parameters for determining the extent of homology set forth by one or more of the aforementioned alignment programs are known. They include but are not limited to p value, percent sequence identity and the percent sequence similarity. P value is the probability that the alignment is produced by chance. For a single alignment, the p value can be calculated according to Karlin et al. (1990) PNAS 87:2246. For multiple alignments, the p value can be calculated using a heuristic approach such as the one programmed in BLAST. Percent sequence identify is defined by the ratio of the number of nucleotide or amino acid matches between the query sequence and the known sequence when the two are optimally aligned. The percent sequence similarity is calculated in the same way as percent identity except one scores amino acids that are different but similar as positive when calculating the percent similarity. Thus, conservative changes that occur frequently without altering function, such as a change from one basic amino acid to another or a change from one hydrophobic amino acid to another are scored as if they were identical.

A “gene product” refers to the amino acid (e.g., peptide or polypeptide) generated when a gene is transcribed and translated.

As used herein, the term “modulate” means to alter or modify a process or biological function.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “cDNAs” refers to complementary DNA, that is mRNA molecules present in a cell or,organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors.”

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

The term “genetically modified” means containing and/or expressing a foreign gene or nucleic acid sequence which in turn, modifies the genotype or phenotype of the cell or its progeny. “Foreign nucleic acid” includes, but is not limited to promoters, enhancers and gene activators. For example, a genetically modified cell includes a cell that contains a polynucleotide that may be in its native environment, but not expressed and expression has been turned on or alternatively, the level of expression has been enhanced or lowered by the upstream insertion of a gene activator.

“Differentially expressed” as applied to a gene, refers to the differential production of the mRNA transcribed from the gene or the protein product encoded by the gene. A differentially expressed gene may be overexpressed or underexpressed as compared to the expression level of a normal or control cell. The term “differentially expressed” also refers to nucleotide sequences in a cell or tissue which are expressed where silent in a control cell or not expressed where expressed in a control cell.

A “composition” is intended to mean a combination of active agent and another compound or composition, inert (for example, a detectable agent or label or a pharmaceutically acceptable carrier) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed., Mack Publ. Co., Easton, Pa. (1975).

An “effective amount” is an amount sufficient to effect beneficial or desired results. An effective amount can be administered in one or more administrations, applications or dosages.

A “gene delivery vehicle” is defined as any molecule that can carry inserted polynucleotides into a host cell. Examples of gene delivery vehicles are liposomes, cationic liposomes, viruses, such as baculovirus, adenovirus, adeno-associated virus, and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like. In aspects where gene transfer is mediated by a retroviral vector, a vector construct refers to the polynucleotide comprising the retroviral genome or part thereof, and the inserted polynucleotide. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism. Retroviruses carry their genetic information in the form of RNA; however, once the virus infects a cell, the RNA is reverse-transcribed into the DNA form that integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a polynucleotide to be inserted. Adenoviruses (Ads) are a relatively well characterized, homogenous group of viruses, including over 50 serotypes. (see, e.g., WO 95/27071). Ads are easy to grow and do not require integration into the host cell genome. Recombinant Ad-derived vectors, particularly those that reduce the potential for recombination and generation of wild-type virus, have also been constructed. See, WO 95/00655; WO 95/11984. Wild-type AAV has high infectivity and specificity integrating into the host cells genome (Hermonat and Muzyczka (1984) PNAS 81:6466-6470; Lebkowski et al. (1988) Mol. Cell. Biol. 8:3988-3996).

Vectors that contain both a promoter and a cloning site into which a polynucleotide can be operatively linked are known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

Polynucleotides are inserted into vector genomes using methods known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; stabilizing elements 3′ to the inserted polynucleotide, and T7 and SP6 RNA promoters for in vitro transcription of sense and antisense RNA. Other means are known and available in the art.

“Host cell” is intended to include any individual cell or cell culture which can be or have been recipients for vectors or the incorporation of exogenous polynucleotides, polypeptides and/or proteins. It also is intended to include progeny of a single cell, and the progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, plant cells, insect cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

As used herein, “expression” refers to the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgamo sequence and the start codon AUG (Sambrook et al. (1989) supra). Similarly, an eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods known in the art.

A “subject” is a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

The terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition. Tumor cells often express antigens that are tumor specific. The term “tumor associated antigen” or “TAA” refers to an antigen thaf is associated with or specific to a tumor.

As used herein, “solid phase support” is not limited to a specific type of support. Rather a large number of supports are available and are known to one of ordinary skill in the art. Solid phase supports include, but are not limited to, silica gels, resins, derivatized plastic films, glass beads, cotton, plastic beads, alumina gels, arrays and chips. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel™, Rapp Polymere, Tubingen, Germany) or polydimethylacrylamide resin (obtained from Milligen/Biosearch, California). In one embodiment for peptide synthesis, solid phase support can refer to polydimethylacrylamide resin.

A “transgenic animal” refers to a genetically engineered animal or offspring of genetically engineered animals. The transgenic animal can contain genetic material from at least one unrelated organism (such as from a bacteria, virus, plant, or other animal) or can contain a mutation which interferes with expression of a gene product.

Polynucleotides

This invention provides methods for modulating cellular function by delivering to a cell or tissue a gene identified in Table 2, or its biological equivalent. In a further aspect, agents that modulate the expression of a gene or its equivalent of Table 2 can be identified by insertion of the one or more genes into a cell that will express the gene. In this way, target cells are generated for use in the in vitro screens, noted above. Alternatively, when the gene is administered to a subject under conditions that allow for expression of the gene, such as an animal, the animal also is useful in the screens identified above. Alternatively, deficient expression of a gene can be restored by administration of a gene or its equivalent of Table 2 under conditions that allow expression of the gene. This gene therapy is useful to treat or ameliorate the symptoms associated with various disorders, e.g., enzyme deficiencies or replacement of tumor suppressor genes in cancers.

A number of alternative approaches can be employed to administer or deliver the polynucleotide. The gene can be delivered to cells deficient in the gene's specific activity using a gene delivery vehicle, wherein the polynucleotide is operatively linked to an appropriate promoter element. Methods for administering an effective amount of a gene delivery vehicle to a cell have been developed and are known to those skilled in the art and described herein. Alternatively, the polypeptide encoded by the polynucleotide of interest can be delivered in an effective amount to a cell that is deficient in the polypeptide's function.

SAGE analysis revealed that certain genes are up-regulated in tumor cells. Thus, these genes are useful as markers for neoplastic cells and targets for visualizing and diagnosing these cells. Methods for detecting gene expression in a cell are known in the art and include techniques such as in hybridization to DNA microarrays, in situ hybridization, PCR, RNase protection assays and Northern blot analysis. Such methods are useful to detect and quantitate expression of the gene in a cell. Alternatively expression of the encoded polypeptide can be detected by various methods. In particular it is useful to prepare polyclonal or monoclonal antibodies that are specifically reactive with the target polypeptide. Such antibodies are useful for visualizing cells that express the polypeptide using techniques such as immunohistology, ELISA, and Western blotting. These techniques can be used to determine expression level and diagnose tumors in a subject.

As noted above, the genes identified on Table 2 and their equivalents were selected for further study as they were found to be differentially expressed in cancer cells. Genes that are up-regulated or down-regulated encode proteins involved in several distinct biochemical pathways. These include, but are not limited to, cell growth, attachment to extracellular matrix components, inter-cellular signaling, intra-cellular signaling and metabolism.

The genes identified in Table 2 and their equivalents are useful candidates for developing therapeutic agents for a variety of disease conditions related to tumorigenesis and angiogenesis. These include, but are not limited to, well-vascularized tumors and hyperplasia.

The agents and compositions of the present invention can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally, transdermally or by inhalation therapy, and may take the form of tablets, lozenges, granules, capsules, pills, ampoules, suppositories or aerosol form. They may also take the form of gene therapy, suspensions, solutions and emulsions of the ingredient in aqueous or nonaqueous diluents, syrups, granulates or powders. In addition to an agent of the present invention, the pharmaceutical compositions can also contain other pharmaceutically active compounds or a plurality of compounds of the invention.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

The polynucleotides of the invention can comprise additional sequences, such as coding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable to provide embodiments of this invention.

The polynucleotides of the invention can be introduced by any suitable gene delivery method or vector. They also can be expressed in a suitable host cell for generating a cell-based therapy. These methods are known in the art.

This invention also provides genetically modified cells that produce enhanced expression of the genes of Table 2 or their equivalents. The genetically modified cells can be produced by insertion of upstream regulatory sequences such as promoters or gene activators (see, U.S. Pat. No. 5,733,761).

The polynucleotides and polypeptides can be conjugated to a detectable marker, e.g. an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In one aspect, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates can be employed to provide a means visible to the human eye or spectrophotometrically, to identify specific hybridization with complementary nucleic acid-containing samples. Thus, this invention further provides a method for detecting a single-stranded polynucleotide identified encoding are of more gene of Table 2 or its equivalent, or its complement, by contacting target single-stranded polynucleotides with a labeled, single-stranded polynucleotide (a probe) which is a portion of the polynucleotides identified in Table 2 (or the corresponding complement) under conditions permitting hybridization (preferably moderately stringent hybridization conditions) of complementary single-stranded polynucleotides, or more preferably, under highly stringent hybridization conditions. Hybridized polynucleotide pairs are separated from un-hybridized, single-stranded polynucleotides. The hybridized polynucleotide pairs are detected using methods known to those of skill in the art and set forth, for example, in Sambrook et al. (1989) supra.

The polynucleotides and sequences embodied in this invention can be obtained using chemical synthesis, recombinant cloning methods, PCR, or any combination thereof. Methods of chemical polynucleotide synthesis are known in the art and need not be described in detail herein. One of skill in the art can use the sequence data provided herein to obtain a desired polynucleotide by employing a DNA synthesizer or ordering from a commercial service.

Suitable cell or tissue samples used for this invention encompass body fluid, solid tissue samples, tissue cultures or cells derived therefrom and the progeny thereof, and sections or smears prepared from any of these sources, or any other samples that may contain neoplastic tumor tissue.

The polynucleotides of this invention can be isolated or replicated using PCR. The PCR technology is the subject matter of U.S. Pat. Nos. 4,683,195; 4,800,159; 4,754,065; and 4,683,202 and described in PCR: THE POLYMERASE CHAIN REACTION (Mullis et al. eds, Birkhauser Press, Boston (1994)) or MacPherson, et al. (1991) and (1995) supra, and references cited therein. Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (prokaryotic or eukaryotic) for replication and amplification. The DNA so amplified can be isolated from the cell by methods known to those of skill in the art. A process for obtaining polynucleotides by this method is further provided herein as well as the polynucleotides so obtained.

RNA can be obtained by first inserting a DNA polynucleotide into a suitable host cell. The DNA can be delivered by any appropriate method, e.g., by the use of an appropriate gene delivery vehicle (e.g., liposome, plasmid or vector) or by electroporation. When the cell replicates and the DNA is transcribed into RNA; the RNA can then be isolated using methods known to those of skill in the art, for example, as set forth in Sambrook et al. (1989) supra. For instance, m-RNA can be isolated using various lytic enzymes or chemical solutions according to the procedures set forth in Sambrook et al. (1989) supra, or extracted by nucleic-acid-binding resins following the accompanying instructions provided by manufactures.

Polynucleotides exhibiting sequence complementarity or homology to a polynucleotide identified in Table 2 find utility as hybridization probes. Since the full coding sequence of the transcript is known, any portion of this sequence or homologous sequences, can be used in the methods of this invention.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting the aforementioned mRNA is at least about 80% identical to the homologous region. More preferably, the probe is 85% identical to the corresponding gene sequence after alignment of the homologous region; even more preferably, it exhibits 90% identity.

These probes can be used in radioassays (e.g. Southern and Northern blot analysis) to detect, prognose, diagnose or monitor various cells or tissues containing these cells. The probes also can be attached to a solid support or an array such as a chip for use in high throughput screening assays for the detection of expression of the gene corresponding a polynucleotide of this invention. Accordingly, this invention also provides a probe comprising or corresponding to a polynucleotide identified in Table 2 or its equivalent, or its complement, or a fragment thereof, attached to a solid support for use in high throughput screens.

The total size of fragment, as well as the size of the complementary stretches, will depend on the intended use or application of the particular nucleic acid segment. Smaller fragments will generally find use in hybridization embodiments, wherein the length of the complementary region may be varied, such as between at least 5 to 10 to about 100 nucleotides, or even full length according to the complementary sequences one wishes to detect.

Nucleotide probes having complementary sequences over stretches greater than 5 to 10 nucleotides in length are generally preferred, so as to increase stability and selectivity of the hybrid, and thereby improving the specificity of particular hybrid molecules obtained.

More preferably, one can design polynucleotides having gene-complementary stretches of 10 or more or more than 50 nucleotides in length, or even longer where desired. Such fragments may be readily prepared by, for example, directly synthesizing the fragment by chemical means, by application of nucleic acid reproduction technology, such as the PCR technology with two priming oligonucleotides as described in U.S. Pat. No. 4,603,102 or by introducing selected sequences into recombinant vectors for recombinant production. In one aspect, a probe is about 50-75 or more alternatively, 50-100, nucleotides in length.

The polynucleotides of the present invention can serve as primers for the detection of genes or gene transcripts that are expressed in neoplastic cells described herein. In this context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. For illustraton purposes only, a primer is the same length as that identified for probes, above.

One method to amplify polynucleotides is PCR. However, PCR conditions used for each reaction are empirically determined. A number of parameters influence the success of a reaction. Among them are annealing temperature and time, extension time, Mg²⁺ concentration, pH, and the relative concentration of primers, templates, and deoxyribonucleotides. After amplification, the resulting DNA fragments can be detected by any appropriate method known in the art, e.g., by agarose gel electrophoresis followed by visualization with ethidium bromide staining and ultraviolet illumination.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are known in the art and commercially available. For general methodology and cloning strategies, see GENE EXPRESSION TECHNOLOGY (Goeddel ed., Academic Press, Inc. (1991)) and references cited therein and VECTORS: ESSENTIAL DATA SERIES (Gacesa and Ramji, eds., John Wiley & Sons, N.Y. (1994)) which contains maps, functional properties, commercial suppliers and a reference to GenEMBL accession numbers for various suitable vectors.

In one embodiment, polynucleotides derived from the polynucleotides of the invention encode polypeptides or proteins having diagnostic and therapeutic utilities as described herein as well as probes to identify transcripts of the protein that may or may not be present. These nucleic acid fragments can by prepared, for example, by restriction enzyme digestion of larger polynucleotides and then labeled with a detectable marker. Alternatively, random fragments can be generated using nick translation of the molecule. For methodology for the preparation and labeling of such fragments, see Sambrook, et al. (1989) supra.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors and liposomes. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using known methods. See Sambrook, et al. (1989) supra. In addition to the use of viral vector for insertion of exogenous nucleic acid into cells, the nucleic acid can be inserted into the host cell by methods known in the art such as transformation for bacterial cells; transfection using calcium phosphate precipitation for mammalian cells; or DEAE-dextran; electroporation; or microinjection. See, Sambrook et al. (1989) supra, for methodology. Thus, this invention also provides a host cell, e.g. a mammalian cell, an animal cell (rat or mouse), a human cell, or a prokaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller A. D. et al. (1989) BioTechniques 7:980-990). The methodology of using replication-incompetent retroviruses for retroviral-mediated gene transfer of gene markers has been established. (Bordignon (1989) PNAS USA 86:8912-8952; Culver, K. (1991) PNAS USA 88:3155; and Rill, D. R. (1991) Blood 79(10):2694-2700). Clinical investigations have shown that there are few or no adverse effects associated with the viral vectors. (Anderson (1992) Science 256:808-813).

This invention also provides “knock-out” animals, in which the gene of interest selected from Table 2, is deleted or mutated sufficiently to disrupt its function. The resulting transgenic animals can be used to further analyze the function of the gene or as an assay system for therapeutic agents and methods. These “knock-out” animals are made by taking advantage of the phenomena of homologous recombination using methods known in the art. Briefly, targeting DNA vectors contain (1) two blocks of DNA sequences that are homologous to separate regions of the target site; (2) a DNA sequence that codes for resistance to the compound G418 (Neo^{r}) between the two blocks of homologous DNA (i. e. positive selection marker) and (3) DNA sequences coding for herpes simplex virus thymidine kinases (HSV-tk1 and HSV-tk2) outside of the homologous blocks (i.e. negative selection marker). When this vector is introduced into the embryonic stem cell, homologous recombination inserts the Neor gene into the target genome, disrupting function of that gene.

Proteins

This invention provides proteins or polypeptides expressed from the polynucleotides or their equivalents, which is intended to include wild-type and recombinantly produced polypeptides and proteins from prokaryotic and eukaryotic host cells, as well as muteins, analogs and fragments thereof. In some embodiments, the term also includes antibodies and anti-idiotypic antibodies. Such polypeptides can be isolated or produced using the methods identified below.

It is understood that functional equivalents or variants of the wild-type polypeptide or protein also are within the scope of this invention, for example, those having conservative amino acid substitutions of the sequences identified in Table 2. Other analogs include fusion proteins comprising a protein or polypeptide, identified in Table 2, or a fragment of that protein or polypeptide.

The proteins and polypeptides of this invention are obtainable by a number of processes known to those of skill in the art, which include purification, chemical synthesis and recombinant methods. Full length proteins can be purified from a neoplastic cell or a tumor biopsy as identified above. Sources for purifying the protein can also be from an individual, such as a patient with ischemic heart disease. Proteins can be purified by methods such as immunoprecipitation with antibody, and standard techniques such as gel filtration, ion-exchange, reversed-phase, and affinity chromatography using a fusion protein as shown herein. For such methodology, see for example Deutscher et al. (1999) GUIDE TO protein PURIFICATION: METHODS IN ENZYMOLOGY (Vol. 182, Academic Press). Accordingly, this invention also provides the processes for obtaining these proteins and polypeptides as well as the products obtainable and obtained by these processes.

The proteins and polypeptides also can be obtained by chemical synthesis using a commercially available automated peptide synthesizer such as those manufactured by Perkin/ Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al. (1989) supra, using the host cell and vector systems described above.

Also provided by this application are the polypeptides and proteins described herein conjugated to a detectable agent for use in the diagnostic methods. For example, detectably labeled proteins and polypeptides can be bound to a column and used for the detection and purification of antibodies. They also are useful as immunogens for the production of antibodies as described below. The proteins and fragments of this invention are useful in an in vitro assay system to screen for agents or drugs, which modulate cellular processes.

The proteins of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides.

This invention also provides a pharmaceutical composition comprising any of a protein, analog, mutein, or polypeptide fragment of this invention, alone or in combination with each other or other agents, and an acceptable carrier. These compositions are useful for various diagnostic and therapeutic methods as described herein.

Antibodies

Also provided by this invention is an antibody capable of specifically forming a complex with the proteins or polypeptides as described above. The term “antibody” includes polyclonal antibodies and monoclonal antibodies. The antibodies include, but are not limited to mouse, rat, and rabbit or human antibodies, and varients thereof.

Laboratory methods for producing polyclonal antibodies and monoclonal antibodies, as well as deducing their corresponding nucleic acid sequences, are known in the art, see Harlow and Lane (1988) supra, and Sambrook, et al. (1989) supra. The monoclonal antibodies of this invention can be biologically produced by introducing protein or a fragment thereof into an animal, e.g., a mouse or a rabbit. The antibody producing cells in the animal are isolated and fused with myeloma cells or heteromyeloma cells to produce hybrid cells or hybridomas. Accordingly, the hybridoma cells producing the monoclonal antibodies of this invention also are provided.

Thus, using the protein or fragment thereof, and known methods, one of skill in the art can produce and screen the hybridoma cells and antibodies of this invention for antibodies having the ability to bind the proteins or polypeptides.

If a monoclonal antibody being tested binds with the protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) PNAS 82:8653 or Spira et al. (1984) J. Immunol. Meth. 74.307.

This invention also provides biological active fragments of the polyclonal and monoclonal antibodies described above. These “antibody fragments” retain some ability to selectively bind with its antigen or immunogen. Such antibody fragments can include, but are not limited to: Fab; Fab′; F(ab′)₂, Fv; and SCA. A specific example of “a biologically active antibody fragment” is a CDR region of the antibody. Methods of making these fragments are known in the art, see for example, Harlow and Lane (1988) supra.

The antibodies of this invention also can be modified to create chimeric antibodies and humanized antibodies (Oi et al. (1986) BioTechniques 4(3):214). Chimeric antibodies are those in which the various domains of the antibodies' heavy and light chains are coded for by DNA from more than one species.

The isolation of other hybridomas secreting monoclonal antibodies with the specificity of the monoclonal antibodies of the invention can be accomplished by one of ordinary skill in the art by producing anti-idiotypic antibodies (Herlyn et al. (1986) Science 232:100). An anti-idiotypic antibody is an antibody that recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies that mimic an epitope. For example, an anti-idiotypic monoclonal antibody made to a first monoclonal antibody will have a binding domain in the hypervariable region that is the mirror image of the epitope bound by the first monoclonal antibody. Thus, in this instance, the anti-idiotypic monoclonal antibody could be used for immunization for production of these antibodies.

As used in this invention, the term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

The antibodies of this invention can be linked to a detectable agent or label. There are many different labels and methods of labeling known to those of ordinary skill in the art.

The antibody-label complex is useful to detect the protein or fragments in a sample, using standard immunochemical techniques such as immunohistochemistry as described by Harlow and Lane (1988) supra. Competitive and non-competitive immunoassays in either a direct or indirect format are examples of such assays, e.g., enzyme linked immunoassay (ELISA) radioimmunoassay (RIA) and the sandwich (immunometric) assay. Those of skill in the art will know, or can readily discern, other immunoassay formats without undue experimentation.

The coupling of antibodies to low molecular weight haptens can increase the sensitivity of the assay. The haptens can then be specifically detected by means of a second reaction. For example, it is common to use haptens such as biotin, which reacts avidin, or dinitropherryl, pyridoxal, and fluorescein, which can react with specific anti-hapten antibodies. See Harlow and Lane (1988) supra.

The monoclonal antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

Compositions containing the antibodies, fragments thereof or cell lines which produce the antibodies, are encompassed by this invention. When these compositions are to be used pharmaceutically, they are combined with a pharmaceutically acceptable carrier.

Functional Analysis with Antibodies

Antibodies of this invention can be used to purify the polypeptides of this invention and to identify biological equivalent polynucleotides. They also can be used to identify agents that modify the function of the polynucleotides of this invention. These antibodies include polyclonal antisera, monoclonal antibodies, and various reagents derived from these preparations that are familiar to those practiced in the art and described above. Antibodies can be used in immuno-histochemistry to determine the physical location of the proteins encoded by the identified genes in subjects.

Antibodies that neutralize the activities of proteins encoded by identified genes can also be used in vivo and in vitro to demonstrate function by adding such neutralizing antibodies into in vivo and in vitro test systems. They also are useful as pharmaceutical agents to modulate the activity of polypeptides of the invention.

Various antibody preparations can also be used in analytical methods such as ELISA assays or Western blots to demonstrate the expression of proteins encoded by the identified genes by test cells in vitro or in vivo. Fragments of such proteins generated by protease degradation during metabolism can also be identified by using appropriate polyclonal antisera with samples derived from experimental samples.

Screening Assays

The present invention provides methods for screening various agents that modulate the expression of a polynucleotide of the invention or the function of a protein product encoded by the polynucleotide of interest in a neoplastic cell. For the purposes of this invention, an “agent” is intended to include, but not be limited to a biological or chemical compound such as a simple or complex organic or inorganic molecule, a peptide, a protein (e.g. antibody), a polynucleotide (e.g. anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

One preferred embodiment is a method for screening small molecules capable of interacting with the protein or polynucleotide of the invention. For the purpose of this invention, “small molecules” are molecules having low molecular weights (MW) that are, in one embodiment, capable of binding to a protein of interest thereby altering the function of the protein. Preferably, the MW of a small molecule is no more than 1,000. Methods for screening small molecules capable of altering protein function are known in the art. For example, a miniaturized arrayed assay for detecting small molecule-protein interactions in cells is discussed by You et al. (1997) Chem. Biol. 4:961-968.

To practice the screening method in vitro, suitable cell cultures or tissue cultures containing this type of neoplastic cell are first provided. The cell can be a cultured cell or a genetically modified cell in which a transcript from a gene of Table 2 or its biological equivalent or its complement is expressed. Alternatively, the cells can be from a tissue biopsy. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, suitable cells can be cultured in microtiter plates and several agents can be assayed at the same time by noting genotypic changes, phenotypic changes or cell death.

When the agent is a composition other than a DNA or RNA, such as a small molecule as described above, the agent can be directly added to the cell culture or added to culture medium for addition. As is apparent to those skilled in the art, an “effective” a mount must be added which can be empirically determined. When agent is a polynucleotide, it can be directly added by use of a gene gun or electroporation. Alternatively, it can be inserted into the cell using a gene delivery vehicle or other method as described above.

Kits containing the agents and instructions necessary to perform the screen in vitro method as described herein also are claimed.

The assays also can be performed in a subject. When the subject is an animal such as a rat, mouse or simian, the method provides a convenient animal model system that can be used prior to clinical testing of an agent. In this system , a candidate agent is a potential drug if transcript expression is altered, i.e., upregulated et al. (such as restoring tumor suppressor function), downregulated or eliminated as with drug resistant genes or oncogenes, or if symptoms associated or correlated to the presence of cells containing transcript expression are ameliorated, each as compared to untreated, animal having the pathological cells. It also can be useful to have a separate negative control group of cells or animals that are healthy and not treated, which provides a basis for comparison. After administration of the agent to subject, suitable cells or tissue samples are collected and assayed for altered gene expression or protein function.

Identification, Analysis, and Manipulation of Genetic Polymorphisms with SNP Technology

The polynucleotides of Table 2 are useful to search for and identify single nucleotide polymorphism (SNP's), which are mutant variants of the gene in the human population. Identification of such polymorphisms is useful to define human diseases to which mutations in the gene contribute and to perfect therapies for disease processes in which the protein encoded by these genes. Mutant variants of the gene identified in this manner can then be employed in the development, screening, and analysis of pharmaceutical agents to treat these diseases. Methods to detect such SNP's can be formatted to create diagnostic tests. Furthermore, various mutations in the gene which effect the response of different individuals to therapeutic agents can be identified and then diagnosed through analysis of SNP's, to guide the prescription of appropriate treatments. Also, SNP's identified in the gene can provide useful sequence markers for genetic tests to analyze other genes and mutations in the region of the genome where the gene is located. Thus it is useful to incorporate these SNP's into polymorphism databases.

Skilled practitioners of the art are familiar with an array of methods for identifying and analyzing SNP's. High throughput DNA sequencing procedures such as sequencing by hybridization (Drmanac et al. (1993) Science 260:1649-1652), mini-sequencing primer extension (Syvanen (1999) Hum. Mutut. 13(1):1-10), or other sequencing methods can be used to detect SNP's in defined regions of the gene. Alternatively, other methods such as hybridization to oligonucleotides on DNA microarrays (Lipshutz et al. (1999) Nat. Genet. 21(1 Suppl.):20-24); analysis of single strand conformational polymorphisms in DNA or RNA molecules by various analytical methods (Nataraj (1999) Electrophoresis 20(6):1177-85); PCR-based mutational analyses such as PCR with primers spanning the polymorphic sequence; or protection of SNP-containing oligonucleotides from nuclease protection such as by use of the bacterial mutS protein, can be employed. Many sophisticated high-throughput technologies based on methods such as automated capillary electrophoresis (Larsen et al. (1999) Hum. Mut. 13(4):318-327), time-of-flight mass spectroscopy (Li et al. (1999) supra), high density micro-arrays (Sapolsky et al. (1999) Genet. Anal. 14(5-6):187-92), semiconductor microchips (Gilles et al. (1999) Nat. Biotechnol. 17(4):365-70), and others have been demonstrated that can be employed with the oncogenic osteomalacia-related gene to perform the uses described above.

Genomics Applications

This invention also provides a process for preparing a database for the analysis of a cell's expressed genes by storing in a digital storage medium information related to the sequences of the invention. Using this method, a data processing system for standardized representation of the expressed genes of a cell is compiled. The database contains at least one polynucleotide, its biological equivalent or complement, of this invention. In an alternative embodiment, the database contains any combination of polynucleotides of this invention, alone or in combination with other polynucleotides. The data processing system is useful to characterize the genotype or phenotype of a cell and to analyze gene expression between two cells by first selecting a cell and then identifying and sequencing the expressed sequences (transcriptome) of the cell. This information also is stored in a computer-readable storage medium as the transcriptome. The transcriptome is then compared with at least one sequence(s) of transcription fragments from a polynucleotide of this invention. The compared sequences are then analyzed. Uniquely expressed sequences and sequences differentially expressed between the polynucleotides of this invention and the selected cell can be identified by this method.

In other words, this invention provides a computer based method for screening the homology of an unknown DNA or mRNA sequence against one or more of the polynucleotides of this invention by first providing the complete set of expressed genes, i.e., the transcriptome, in computer readable form and homology screening the DNA or mRNA of the unknown sequence against the polynucleotides of this invention or database containing same and determining whether the DNA sequence of the unknown contains similarities to any portion of the transcriptome listed in the computer readable form. In one aspect, the transcriptome of the test cell is compared to a gene identified in Table 2.

Thus, the information provided herein also provides a means to compare the relative abundance of gene transcripts in different biological specimens by use of high-throughput sequence-specific analysis of individual RNAs or their corresponding cDNAs using a modification of the systems described in WO 95/2068, WO 96/23078 and U.S. Pat. No. 5,618,672.

Non-Human Transgenic Animals

In another aspect, the novel polynucleotide sequences associated with a pathological state of a cell can be used to generate transgenic animal models. In recent years, geneticists have succeeded in creating transgenic animals, for example mice, by manipulating the genes of developing embryos and introducing foreign genes into these embryos. Once these genes have integrated into the genome of the recipient embryo, the resulting embryos or adult animals can be analyzed to determine the function of the gene. The mutant animals are produced to understand the function of known genes in vivo and to create animal models of human diseases. (Joyner et al. (1992) in POST-IMPLANTATION DEVELOPMENT IN THE MOUSE (Chadwick and Marsh, eds., John Wiley & Sons, United Kingdom) pp: 277-297; Dorin et al. (1992) Nature 359:211-215).

The following examples are intended to illustrate, but not limit the invention.

### EXPERIMENTAL

The total of eight independent SAGE libraries were generated from eight different normal and tumor samples, as shown in Table 1. The samples varied in wild type VHL gene expression and exposure to hypoxia. Two samples were derived from the RCC 786-0 cell line, which is defective in the VHL gene, engineered to express wild type VHL through infection with a retroviral vector carrying the gene. These cells were grown under normoxic (786-0 VHL⁺Nor.) and hypoxic (786-0 VHL⁺Hyp.) conditions. Two samples were generated from the RCC 786-0 cells infected with an empty retroviral vector, also grown under normoxic (786-0 VHL⁻Nor.) and hypoxic (786-0 VHL⁻Hyp.) conditions. Two samples were from independent primary cultures of normal renal proximal tubule cells (RPTECs), which are believed to give rise to renal cell carcinoma, grown under normoxic conditions (RPTECs VHL⁺Nor.). Finally, two samples were from the same independent RPTECs grown under hypoxic conditions (RPTECs VHL⁺ hyp.). In total, over 380,000 SAGE tags were sequenced and more than 61,000 unique tags were identified. Gene expression profiles of the cells grown under different conditions were compared to find potential regulators or targets of VHL and/or hypoxia-mediated signaling. For this analysis, normalized SAGE tag abundance was used to compare data from two or more libraries, and genes were considered induced or repressed if their expression levels changed by at least 4-fold.

Effects of VHL on gene expression in 786-0 cells. As a multifunctional tumor suppressor, pVHL plays an important role in many aspects of cell biology. Genes exclusively up-regulated in 786-0 cells lacking VHL include genes that encode growth factors and their receptors, proteins involved in cell-cell and cell-extracellular matrix interactions, and proteins that control cellular metabolism (Table 2) were assayed. Many of the genes in this group were shown to be hypoxia-inducible genes (Table 2c), including GLUT 1, insulin-like growth factor binding protein 3 (IGFBP3) and VEGF, all of which are regulated by HIF-1α (Semenza (1999) Am. Rev. Cell Dev. Biol. 15:551-578).

It is worth noting that a SAGE tag corresponding to VEGF was induced 4-fold in the 786-0 VHL⁻ cells compared to RPTEC, and was also induced in the RPTECs grown under hypoxia (Table 2c). This SAGE tag is located in the 3′ untranslated region of multiple isoforms of VEGF (Claffey et al. (1998) Mol. Biol. Cell 9:469-481) and therefore does not identify which isoform(s) is predominantly expressed in these cells. Interestingly, VEGF¹⁸⁹ was previously observed by SAGE at induced levels in glioblastoma cells grown under hypoxia (Lal et al. (2001) J. Natl. Cancer Inst. 93(17):1337-1343) but was not induced at least 4-fold in any of our samples exposed to hypoxia.

Hypoxia-overexpressed gene 3 (HOG3) was also expressed more highly in the 786-0 VHL⁻Nor. cells than in the RPTEC Nor. cells (Table 2c). HOG3 is an HIF-1-dependent hypoxia inducible gene originally identified in human glioblastoma cells (Lal et al. (2001) supra.). HIG2 (Hypoxia inducible gene 2), which was previously identified as a hypoxia inducible gene in cultured human cervical epithelial cells and in cervical tumor xenografts deprived of oxygen (Denko, N. et al. (2000) Clin. Cancer Res. 6(2):480-487), was also induced in the 786-0 VHL⁻Nor. cells. Overexpression of these genes is consistent with the role of VHL in negatively regulating HIF-responsive genes.

468 genes are exclusively up-regulated in the 786-0 cells that have been engineered to re-express VHL, with respect to their expression in RPTECs. Some genes were previously implicated in playing a functional role in angiogenesis and tumorigenesis (Table 2d). These include syndecan 2 (SDC2) (Iozzo and San Antonio (2001) J. Clin. Invest. 108(3):349-355), neuropilin 1 (NRP1) (Soker, S. et al. (1990) Cell 92(6):735-745), plasminogen activator urokinase receptor (PLAUR) (Roland, et al. (1990) EMBO J. 9(2):467-474; Graham, et al. (1998) Blood 91(9):3300-3307; Graham, et al. (1999) Int. J. Cancer 80(4):617-623) and integrin beta 3 binding protein (ITGB3BP) (Shattil, et al. (1995) J. Cell Biol. 131(3):807-816 and Li, et al. (1999) Mol. Cell Biol. 19(10):7191-7202). The role of VHL in negatively regulating genes involved in angiogenesis has now been extensively studied. This data shows that VHL also positively regulates genes that promote angiogenesis, at least in the context of a cancer cell.

This analysis also identified 371 genes that are constitutively over-expressed in the 786-0 cells with respect to RPTECs, regardless of VHL expression (FIG. 2e). This set reflects genetic differences between a malignant cell line and normal cells. Some of the genes represent potential biomarkers or tumor specific antigens for renal cell carcinoma since their overexpression is independent of the genetic status of VHL. Indeed, cyclin B1 (CCNB1), a protein that regulates cell growth and proliferation, is induced about 7 to 10-fold, by SAGE, in the 786-0 cells. CCNB1 was recently identified as an epithelial tumor-specific antigen and is a candidate for cancer vaccine therapy (Kao, et al. (2001) J. Exp. Med. 194(9):1313-1324).

Although 371 genes are induced at least 4-fold in both the presence and absence of VHL, many of these genes are more highly expressed in the VHL⁻ cells. This shows that VHL plays a role in their transcriptional regulation. Many of these genes are involved in regulating tumor proliferation, differentiation, and angiogenesis.

HIF-α is regulated through prolyl hydroxylation by PHD (PH domain-containing) proteins 1, 2 and 3 (Bruick and McKnight (2001) Science 294:1337-1340 and Epsteen et al. (2001) Cell 107(1):43-54). In vitro studies using recombinant PHD proteins suggest that PHDs may be oxygen sensors (Epstein et al. (2001) supra, and Semenza (2001) Cell 107(1):1-3). Importantly, PHD-1 is up-regulated in the cancer cells grown under normoxia compared to RPTECs (Table 2d). Conceivably, alterations in PHD-1 expression impact the response of the cancer cells to oxygen changes.

Overall, more than 1000 genes showed induced expression in the 786-0 cells (relative to their expression in RPTECS), with and without VHL, suggesting that the cancer cells have undergone significant changes compared to non-transformed renal epithelial cells. These changes have resulted in the induction of genes that promote and sustain tumor growth.

Genes that are hypoxia-inducible in the presence of VHL. Hypoxia within a tumor is primarily a pathophysiologic consequence of structurally and functionally disturbed microcirculation, accompanied by the deterioration of diffusion conditions. Tumor hypoxia is a central issue in tumor physiology and cancer treatment, since it is associated with adverse outcomes. The identification of genes specifically induced under hypoxic conditions may be of functional importance in a variety of critical cellular pathways, including cell cycle arrest, apoptosis, metabolism, tissue remodeling and angiogenesis. The role of VHL in regulating gene expression in tumors and in response to hypoxic conditions is under intense study. Because the renal cell carcinoma cell line 786-0 carries a defective VHL gene, it is an ideal model system to delineate the role of VHL in both hypoxia signaling and tumorigenesis. By comparing the genes that are hypoxia-inducible in the 786-0 VHL⁺ cells with the hypoxia-inducible genes in RPTECs, genes were identified that are uniquely hypoxia-regulated in a tumor cell environment. Many of these genes have not previously been shown to be hypoxia-inducible in other tumor or normal cells. 160 genes are up-regulated by hypoxia in 786-0 VHL⁺ cells, whereas 414 genes are induced by hypoxia in the RPTECs. It is noteworthy that only seven genes are hypoxia-inducible at least 4-fold in both RPTECs and in the 786-0 VHL⁺ cells. The vast majority of genes (153 out of 160) that are hypoxia-inducible in the 786-0 VHL⁺ cells are not induced at least 4-fold in RPTECs grown under hypoxia. As discussed above, VEGF is induced more than 5-fold in RPTECs grown under hypoxia, but because basal expression of VEGF is elevated in 786-0 VHL⁺ cells, the hypoxic induction is not as dramatic in these cells. The observation that greater than 95% of the hypoxia-inducible genes differ between the malignant cells and the normal renal cells suggests the establishment of an alternative hypoxia sensitive pathway(s) in these tumor cells. The TGFβ-induced gene (TGFBI; Table 2f) is among the 153 hypoxia-inducible genes in 786-0 VHL⁺ cells but not in the RPTECs. TGFBI was also induced in the 786-0 cells regardless of VHL status (Table 2e), although the SAGE tags identified under these different conditions were distinct. Both tags are located in the 3′ UTR of the gene and therefore correspond to the same protein, although the alternative mRNAs may be regulated differently. TGFBI was previously reported to be elevated in adenomas and colorectal cancer (Zhang et al. (1997) Science 276(5316):1268-1272 and Buckhaults et al. (2001) Cancer Res. 61(19):6996-7001). TGFBI is known to bind to collagens, and is believed to play an important role in cell-collagen interactions (Munier (1997) Nature Genetics 15(3):247-25 1). Overexpression of this gene may therefore be a critical component in the tissue remodeling that accompanies tumorigenesis.

Genes in this group also include zinc finger protein 36 (ZFP36L1) which is thought to be involved in regulating the response to growth factors (Barnard et al. (1993) Nucleic Acids Res 21:3580; (Bustin et al. (1994) DNA Cell Biol. 13:449-459) and p53-induced protein PIGPC1, also called the THW tumor suppressor (Hildebrandt et al. (2000) Anticancer Research 20:2801-2809). THW is a putative transmembrane receptor that was initially identified as a gene that was down-regulated in a melanoma cell line capable of metastasis when compared to a non-metastasizing line. It was also down-regulated in mammary carcinoma cell lines compared with cell lines derived from non-malignant mammary epithelium, and in pancreas cell lines derived from metastases compared with a cell line derived from a primary pancreatic tumor (Hildebrandt et al. (2000) supra). Indeed, it was genetic data that led to THW's assignment as a tumor suppressor gene; the THW gene is located on chromosome 6q, a frequent site of loss of heterozygosity (LOH) associated with many malignancies (Hildebrandt et al. (2000) supra). Consistent with previous data, THW was found to be down-regulated in the cancer cell line, 786-0 VHL⁺ Nor., compared with normal RPTECs grown under normoxic conditions (8.22-fold). Interestingly, the down-regulation of THW is less dramatic in 786-0 VHL⁻ Nor. compared with normal RPTECs (3.79-fold,). Thus, VHL plays a role in THW expression, and establishes a pathway of regulation for tumor suppressor genes.

Recently, a set of hypoxia-overexpressed genes (HOGs) was identified in human glioblastoma cells using SAGE (Lal et al. (2001) J. Natl. Cancer Inst. 93(17):1337-1343), and six of the HOGs were found to be controlled by HIF-α (Lal et al. (2001) supra). Interestingly, none of the 6 HOGs was induced in the 786-0 VHL⁺ cells grown under in this study. As discussed above, the 786-0 cells lack HIF-1α expression so the failure to see HOG induction here may be explained by exclusive HIF-1α regulation of the HOGs.

The seven genes that are hypoxia-inducible in both RPTECs and in the 786-0 VHL⁺ cells may be regulated by HIF-α proteins. Since HIF-2α is constitutively expressed in the 786-0 VHL⁻ cells, regardless of oxygen pressure, higher expression of these seven genes is expected in the 786-0 VHL⁻Nor. cells with respect to RPTECs grown under normoxia. Interestingly, this is only true for two of the seven genes, PAI-1 and a novel gene (no match for the SAGE tag in the public databases). Thus the other 5 genes are controlled through proteins other than HIF, and again separates the VHL and HIF-α gene-induction pathways. Glycolytic enzyme enolase 2 (ENO2), which was previously found in nervous tissue (Oliva et al. (1991) Genomics 10:157-165), is among the seven hypoxic genes. Interestingly, the α-, or non-neuronal enolase (ENO1), is ubiquitously expressed and is known to be induced by hypoxia in an HIF-1α dependent manner (Iyer et al. (1998) Genes Dev 12:149-162).

414 genes were induced at least 4-fold in the RPTECs grown under hypoxic conditions. As expected, this group includes genes previously identified as hypoxic genes, such as GLUT 1, insulin-like growth factor binding protein 3 (IGFBP3), adenylate kinase 3 (ADK3), and VEGF (Table 2c, 2c′) (Semenza (1999) supra). HIF-1α mRNA was also detected at induced levels in the hypoxia treated RPTECs, which confirms that HIF-1α can be regulated at a transcriptional level as well (Semenza (2000) supra; Wenger (2000) supra). In addition to the genes known to be hypoxia-inducible, many of the detected genes are not well characterized and/or were not previously known to be regulated by hypoxia. Examples include retinoic acid induced gene 3 (RAI3) which encodes a putative G protein-coupled receptor (Cheng and Lotan (1998) J. Biol. Chem. 273:35008-35015), lysyl oxidaselike gene 2 (LOXL2) which encodes an extracellular protein that may be specifically involved in cell adhesion and senescence (Csiszar (2001) Prog Nucleic Acid Res Mol Biol. 70:1-32); Jourdan-Le Saux et al. (1998) Genomics 51:305-307); Saito et al. (1997) J Biol Chem. 272:8157-8160) and a presumably novel gene since the SAGE tag does not match any known genes or ESTs in the public databases.

VHL-independent Gene Expression in 786-0 Cells under Hypoxic Conditions. VHL is a known regulator of the hypoxia pathway through HIF. A defect in VHL will result in the constitutive activation of the HIF-α pathway and lead to up-regulation of hypoxia-inducible genes, even under normoxic condition. Interestingly, there are still 91 genes that are inducible by hypoxia in 786-0 VHL⁻ cells. 87 genes are exclusively hypoxia-inducible in 786-0 in the absence of VHL, whereas 156 are only induced by hypoxia in the presence of VHL. Thus, tumor cells evolve VHL-dependent and VHL-independent mechanisms to respond to hypoxic stress so that cells can survive in this adverse environment. Among the 87 genes which are uniquely hypoxia-inducible in 786-0 VHL⁻ cells are genes known to be involved in cell-cell interactions and vascular remodeling (Table 2h), including integrin α E (ITGAE) (Cerf-Bensussan et al. (1987) Eur. J. Immunol. 17(9):1279-1285) and endothelin 2 (EDN2) (Ohkubo et al. (1990) FEBS Lett. 274(1-2):136-140); Saida et al. (2000) Genomics 64(1):51-61). Interestingly, EDN2 was previously identified as a hypoxia-inducible gene in hypopharyngeal tumor cells (Koong et al. (2000) Cancer Res. 60(4):883-887). This list also includes genes involved in the stress response (stress-associated endoplasmic reticulum protein 1; SERP1) (Yamaguchi et al. (1999) J. Cell Biol. 147(6)1195-1204), cellular metabolism (phosphoglucomutase 1; PGM1) (Whitehouse et al. (1992) PNAS 89(1):411-415), and cell growth control (cell division cycle 25B; CDC25B) (Bulavin et al. (2001) Nature 411(6833):102-107). In addition, many of the genes encode proteins of unknown function that are candidate targets for regulators of hypoxia-responsive pathways.

Interestingly, 4 genes are induced by hypoxia in 786-0 cells regardless of VHL expression (Table 2i). These 4 genes are not among the 414 genes that are hypoxia inducible in normal kidney cells (RPTECs). The specificity of induction in renal cancer cells, regardless of VHL status, suggests that these four genes may be useful biomarkers for RCC and/or effective therapeutic targets. Among the 4 genes induced is HAX-1. The HAX-I gene product is known to interact with various proteins including the polycystic kidney disease protein (PKD2) (Gallagher et al. (2000) PNAS 97(8):4017-4022) and the F-actin binding protein, cortactin (Gallagher et al. (2000) supra). It has been proposed that HAX-1 connects PK D2 to the actin cytoskeleton. This study shows that expression of HAX-1 is increased in a hypoxic tumor environment suggests that this protein may contribute to the cell-matrix contact changes associated with tumor progression. Two of the remaining 3 genes induced in 786-0 cells, regardless of VHL, encode proteins with uncharacterized functions and the final SAGE tag in this group corresponds to multiple genes.

Genes which are negativelv regulated by VHL in 786-0 cells. It has been shown that VHL negatively regulates many hypoxia-inducible genes through the VHL-HIF pathway. In addition, VHL has been shown to regulate the mRNA stability of some hypoxia-inducible genes (Iliopoulos et al. (1996) Nature Med. 1(8):822-826). Thus, a defect in VHL is expected to result in the overexpression of VHL target genes. This is consistent with the observation that many genes which are induced by hypoxia in the VHL 786-0 VHL⁺ cells are also induced by the absence of VHL, under normoxia in the 786-0 VHL⁻ cells. 38 genes that are hypoxia-inducible in the 786-0 VHL⁺ cells are also up-regulated in 786-0 VHL⁻ cells under normoxic conditions, when compared to 786-VHL⁺ cells grown under normoxia. Again, the neuronal enolase, EN02, described above, is among these genes (Table 2b). Another interesting gene in this category is glia maturation factor beta (GMFB) (Table 2b) which is involved in the differentiation of brain cells and in the stimulation of neural regeneration (Kaplan et al. (1991) J Neurochem 57:483-490). Overexpression of genes involved in neuronal metabolism, growth and differentiation is intriguing since other manifestations of VHL disease are the development of hemangioblastomas of the retina and central nervous system and pheochromocytomas (Kondo and Kaelin (2001) Exp Cell Res 264:117-125; Maher and Kaelin (1997) Medicine (Baltimore) 76:381-391).

In the tumor cells, there are 154 genes that appear to be negatively regulated by VHL. These genes are up-regulated in 786-0 cells that lack VHL compared to those expressing VHL (Table 2a). Among those genes are matrix metalloprotease 1 (MMP1) which cleaves collagens of types I, II, III, VII and X (Templeton et al. (1990). Numerous studies on MMPs have demonstrated that these proteases play an important role in angiogenesis, morphogenesis, and tissue remodeling, processes which are all associated with the timely breakdown of extracellular matrix during tumorigenesis (Nagase and Woessner (1999) J Biol Chem 274:21491-21494; Stetler-Stevenson (1999) J Clinic Invest 103:1237-1241; Vu and Werb (2000) Genes Dev 14:2123-2133). This group also contains genes that are involved in cell growth and differentiation, including insulin-like growth factor binding protein 3 (IGFBP3) (Cubbage et al. (1990) J Biol Chem 265:12642-12649). This shows that the VHL tumor suppressor functions by directly regulating genes that control cell growth and differentiation, as well as angiogenesis.

In summary, the gene expression profile analyses of the renal cell carcinoma line 786-0 and the normal kidney cells, grown under different environmental conditions, demonstrated that 786-0 cells have evolved genetic alterations that affect cell growth and differentiation as well as an altered response to hypoxia. The molecular profiles presented here identified genes in the alternative pathways that are either VHL-dependent or -independent. The lists include genes with potential importance in the physiological and pathologic regulation of tumor growth, metabolism, and angiogenesis. Since renal cell tumors are highly vascularized, this data shows that the tumor cells induce angiogenic factors, such as VEGF and TGF-β. Moreover, this analysis identified genes that are inducible by hypoxia specifically in the cancer cells that have lost VHL expression, such as SERP1 and HAX1, which identifies them as valuable targets specific for mutant tumor cells. Importantly, genes such as MMP-1, that are known to play a critical role in tissue remodeling and angiogenesis, are VHL-regulated in these cells.

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

TABLE 1
SAGE libraries of cancer cells (RCC 786-0) and normal cells

| Library | Cell type | VHL status | O₂ conditions | SAGE Tags |
|---|---|---|---|---|
| V1 | 786-O | +VHL | Normoxia | 54,224 |
| V2 | 786-O | −VHL | Normoxia | 51,759 |
| V3 | 786-O | +VHL | Hypoxia | 51,000 |
| V4 | 786-O | −VHL | Hypoxia | 55,368 |
| V5, 6 | RPTEC | +VHL | Normoxia | 51,073; 56,395 |
| V7, 8 | RPTEC | +VHL | Hypoxia | 30,760; 31,068 |

TABLE 2
Differentially expressed genes in renal cells grown under different conditions

| | Accession # or | | | |
|---|---|---|---|---|
| SAGE tag | UniGene ID | Gene symbol (name) | Ratio 1 | Ratio 2 |
| a | | | V2/V1 | V3/V1 |
| TAGAATTTTC | AK001183 | MMP1; metalloprotease 1 (pitrilysin family) | 4.28 | 3.28 |
| GGTGAATCTC | Hs.77326 | IGFBP3; insulin-like growth factor binding protein 3 | 4.28 | 1.11 |
| | | (EST) | | |
| b | | | V2/V1 | V3/V1 |
| CCACGTTCCA | Y00691 M27 | ENO2; enolase 2, (gamma, neuronal) | 8.61 | 5.44 |
| GTGCTTGTAC | AB001106 | GMFB; glia maturation factor, beta | 7.50 | 6.56 |
| c | | | V2/V5, V6 | V7, V8/V5, V6 |
| GAGACTCCTG | AF070544 | GLUT1; solute carrier family 2 (facilitated glucose | 5.25 | 14.02 |
| | | transporter), member 1 | | |
| ACTGAGGAAA | X64875 | IGFBP3; insulin-like growth factor binding protein 3 | 6.63 | 5.18 |
| CCAGAGTCTC | Hs.73793 | VEGF; vascular endothelial growth factor (EST) | 4.33 | 5.39 |
| GCCACGTTGT | AL137588 | HOG3; hypothetical protein DKFZp434K1210 | 12.89 | 14.39 |
| TGGCTTAAAT | AF144755 | HIG2; hypoxia-inducible protein 2 | 9.16 | 18.68 |
| c¹ | | | | |
| TGATGGTGAT | X60673 S41 | ADK3; adenylate kinase 3 | 2.11 | 7.17 |
| CCTTGAGTAC | AF054987 | ALDOC; aldolase C, fructose-bisphosphate | 0 | 5.51 |
| AAAAGATACT | AF109161 | P35srj; Cbp/p300-interacting transactivator | 0 | 4.85 |
| GTGGTGGCAG | AK001761 | RAI3; retinoic acid induced 3 | 3.90 | 17.70 |
| TGTCATCACA | U92015 | LOXL2; lysyl oxidase-like 2 | 10.82 | 9.99 |
| ACATTTCATC | Hs.5890 | hypothetical protein FLJ23306 (EST) | 2.11 | 12.56 |
| d | | | V2/V5, V6 | V1/V5, V6 |
| CCAAAAGTAA | Hs.1501 | SDC2; syndecan 2 (EST) | 0 | 7.40 |
| GTTGTAAATA | AK001043 | ILKAP; integrin-linked kinase-associated serine/ | 0 | 6.11 |
| | | threonine phosphatase 2C | | |
| TTTTGTACCA | AL121748 | NRP1; neuropilin 1; VEGF165 receptor | 0 | 4.11 |
| TAGCCCCAGC | U09347 S63 | PLAUR; Plasminogen activator, urokinase receptor | 0 | 4.11 |
| TATAAAAGTG | BC005301 | ITGB3BP; integrin beta 3 binding protein (beta3- | 0 | 4.11 |
| | | endonexin) | | |
| TAATTTGAAA | AJ227859 | PHD-1; EGL 9 (C. elegans) homolog 1 | 2.11 | 4.11 |
| e | | | V2/V5, V6 | V1/V5, V6 |
| GTGTGTTTGT | M77349 | TGFBI; transforming growth factor, beta-induced, 68kD | 24.74 | 15.98 |
| TGCCATCTGT | BC006510 | CCNB1; cyclin B1 | 9.64 | 7.38 |
| f | | | V3/V1 | |
| GGAAGTCCTG | M77349 | TGFBI; transforming growth factor, beta-induced, 68kD | 6.56 | |
| | | (EST) | | |
| CTTTCTTCCC | X79067 X71 | ZFP36L1; Zinc finger protein 36 | 5.44 | |
| CCACAGGAGA | AJ251830 | p53-induced protein PIGPC1; THW tumor suppressor | 7.61 | |
| g | | | V3/V1 | V7, V8/V5, V6 |
| CCACGTTCCA | Y00691M27 | ENO2; enolase 2, (gamma, neuronal) | 5.44 | 12.28 |
| h | | | V4/V2 | |
| GAGCTGTTGG | L25851 | ITGAE; integrin alpha E | 4.74 | |
| CCTCAGCCAG | X55177 | EDN2; endothelin 2 | 4.74 | |
| AATAAAGCAA | AB022427 | SERP1; stress-associated endoplasmic reticulum protein | 7.58 | |
| | | 1 | | |
| TAACCCAACA | M83088 | PGM1; phosphoglucomutase 1 | 8.58 | |
| ACAGCCTGCA | AL10980 | CDC25B; cell division cycle 25B | 6.63 | |
| i | | | V4/V2 | V3/V1 |
| TCAGTTTGTC | U68566 | HAX1; HS1 binding protein | 4.18 | 5.30 |
| CAGAGTTGTA | AB014733 | Hypothetical protein AF140225 | 4.74 | 5.44 |
| CCCAGCTAAT | AF339827 | ATP6A1; ATPase, H+ transporting or FHX; FOXJ2 forkhead | 6.63 | 6.56 |
| | | factor or CENPH; kinetochore protein CENP-H | | |
| TAGTAAGTCA | AL110153 | Homo sapiens mRNA; cDNA DKFZp586E0524 | 4.74 | 5.44 |

¹Unigene Cluster ID.

| | | | | | | | | | | | | |
|---|---|---|---|---|---|---|---|---|---|---|---|---|
| | Accession/ | | | | | | | | | | | |
| SAGE Tag | UniGene ID | Description | V1 | V2 | V3 | V4 | V5 | V6 | V7 | V8 | V2/V1 | V3/V1 |

116 genes in FIG. 1(a) (109 genes; 7 tages with no matches not shown)
SAGE data for genes up-regulated ≧ 4-fold in 786-0 VHL⁻ Nor. (V2) when compared to 786-0 VHL⁺ Nor. (V1).
Also shown, for comparison, is the SAGE data for each gene in the 786-0 VHL⁺ Hyp. (V3) with respect to 786-0 VHL⁺ Nor. (V1).
SAGE tag abundances were normalized before calculating ratios, as shown. The table is presented in descending order of V2/V1.

| | | | | | | | | | | | | |
|---|---|---|---|---|---|---|---|---|---|---|---|---|
| AGGGCCGACT | X65550 | antigen identified by monoclonal antibody Ki-67 | 0.18 | 1.55 | 0.39 | 0.54 | 0.00 | 0.00 | 0.00 | 0.00 | 8.61 | 2.17 |
| ACTGCCACAG | Hs.298490 | ESTs | 0.18 | 1.35 | 0.00 | 0.18 | 0.00 | 0.00 | 0.00 | 0.00 | 7.50 | 0.00 |
| GGAAGAGGGT | Hs.5331 | Homo sapiens cDNA: FLJ22361 fis, clone HRC06524, highly similar to HSU15426 Human anonymous | 0.18 | 1.35 | 0.59 | 0.54 | 0.59 | 1.60 | 1.30 | 0.97 | 7.50 | 3.28 |
| | | mRNA | | | | | | | | | | |
| GGGAAGTCAC | U58766 | tissue specific transplantation antigen P35B | 0.18 | 1.35 | 0.59 | 0.72 | 0.59 | 0.71 | 0.00 | 0.00 | 7.50 | 3.28 |
| GGCTGTGCCA | X97261 | metallothionein 1L | 0.18 | 1.35 | 0.59 | 1.81 | 30.35 | 12.77 | 26.33 | 13.84 | 7.50 | 3.28 |
| ATTTTTTAAC | AF100747 | HSPCO34 protein | 0.18 | 1.16 | 0.39 | 0.00 | 0.20 | 0.00 | 0.00 | 0.00 | 6.44 | 2.17 |
| CCACACAAGC | AF155650 | goliath protein | 0.18 | 1.16 | 0.39 | 0.72 | 0.39 | 0.71 | 1.30 | 0.32 | 6.44 | 2.17 |
| CATACCAGGC | AK002050 | papillomavirus regulatory factor PRF-1 | 0.18 | 1.16 | 0.00 | 0.18 | 0.00 | 0.00 | 0.33 | 0.97 | 6.44 | 0.00 |
| CAAGCCCTGC | Hs.172035 | hypothetical protein similar to mouse HN1 (Hematological and Neurological expressed sequence 1) | 0.18 | 1.16 | 0.39 | 0.72 | 0.59 | 1.24 | 0.98 | 0.97 | 6.44 | 2.17 |
| TCTGACCACC | Hs.173205 | nucleophosmin (nucleolar phosphoprotein B23, numatrin) or ESTs | 0.18 | 1.16 | 0.00 | 0.18 | 0.20 | 0.35 | 0.00 | 0.00 | 6.44 | 0.00 |
| TTGAGGGGGT | Hs.76549 | ATPase, Na+/K+ transporting, alpha 1 polypeptide | 0.18 | 1.16 | 0.39 | 1.08 | 0.59 | 1.24 | 0.98 | 0.97 | 6.44 | 2.17 |
| TTAGTCTTCA | Hs.82712 | fragile X mental retardation, autosomal homolog 1 | 0.18 | 1.16 | 0.20 | 0.18 | 0.00 | 0.00 | 0.00 | 0.32 | 6.44 | 1.11 |
| CCTGTAAAGC | Hs.9691 | Homo sapiens cDNA: FLJ23249 fis, clone COL04196 | 0.18 | 1.16 | 0.59 | 0.72 | 0.20 | 0.18 | 1.63 | 3.86 | 6.44 | 3.28 |
| AAGAACCAAG | L08436 | carbohydrate (N-acetylglucosamine 6-O) sulfotransferase 6 (EST) | 0.18 | 1.16 | 0.39 | 0.36 | 0.78 | 0.00 | 0.33 | 0.32 | 6.44 | 2.17 |
| GACTTGGAGG | AF086057 | membrane-associated tyrosine- and threonine-specific cdc2-inhibitory kinase (EST) | 0.37 | 2.13 | 1.18 | 0.90 | 0.20 | 0.53 | 0.00 | 0.00 | 5.76 | 3.19 |
| AACCCTTGGG | AJ001258 | NIPSNAP, C. elegans, homolog 1 | 0.37 | 2.13 | 0.59 | 1.26 | 0.78 | 2.13 | 0.00 | 0.00 | 5.76 | 1.59 |
| CCTGCTCCCT | AB018009 | solute carrier family 7 (cationic amino acid transporter, y+ system), member 5 | 0.37 | 1.93 | 0.98 | 1.81 | 0.59 | 0.53 | 0.65 | 0.00 | 5.22 | 2.65 |
| GCAAGGTTGC | AB020708 | histone deacetylase 6 | 0.18 | 0.97 | 0.39 | 0.72 | 0.00 | 0.89 | 0.00 | 0.00 | 5.39 | 2.17 |
| AAGCGCTCTC | AF083420 | serine/threonine kinase 24 (Ste20, yeast homolog) | 0.18 | 0.97 | 0.20 | 0.36 | 1.17 | 0.35 | 0.33 | 0.97 | 5.39 | 1.11 |
| TGGAGGTGGG | AF086628 | VAMP (vesicle-associated membrane protein)-associated protein B and C | 0.18 | 0.97 | 0.39 | 0.36 | 0.00 | 0.18 | 1.30 | 0.32 | 5.39 | 2.17 |
| GCCCGTGCCA | AF289485 | MYG1 protein | 0.18 | 0.97 | 0.39 | 0.18 | 1.57 | 2.13 | 0.65 | 0.64 | 5.39 | 2.17 |
| TTTTACATCT | AJ000414 | thyroid hormone receptor interactor 10 | 0.18 | 0.97 | 0.00 | 0.54 | 0.00 | 0.00 | 0.00 | 0.00 | 5.39 | 0.00 |
| TCTGTGCTCA | AK000006 | hypothetical protein | 0.18 | 0.97 | 0.39 | 0.36 | 0.00 | 0.18 | 0.00 | 0.00 | 5.39 | 2.17 |
| GAGTGAAAGA | AK000474 | chromosome 21 open reading frame 59 | 0.18 | 0.97 | 0.20 | 0.18 | 0.00 | 0.18 | 0.33 | 0.32 | 5.39 | 1.11 |
| TGTTCTTTGC | AK001605 | hypothetical protein FLJ10743 | 0.18 | 0.97 | 0.20 | 0.18 | 0.00 | 0.00 | 0.00 | 0.00 | 5.39 | 1.11 |
| AAGCCCAGGC | AL080092 | DKFZP564N1362 protein | 0.37 | 1.93 | 1.37 | 2.17 | 1.37 | 2.31 | 0.00 | 0.97 | 5.22 | 3.70 |
| GAAGGATGTG | Hs.11169 | Gene 33/Mig-6 | 0.18 | 0.97 | 0.20 | 0.72 | 0.00 | 0.00 | 0.00 | 0.97 | 5.39 | 1.11 |
| TGTGTCAAAG | Hs.181163 | high-mobility group (nonhistone chromosomal) protein 17 | 0.18 | 0.97 | 0.59 | 0.90 | 0.20 | 0.00 | 0.00 | 0.00 | 5.39 | 3.28 |
| CACACAGTTT | Hs.204354 | ras homolog gene family, member B | 0.18 | 0.97 | 0.39 | 0.18 | 2.35 | 1.42 | 2.60 | 0.00 | 5.39 | 2.17 |
| ACCAGCTCCC | Hs.22546 | Homo sapiens cDNA: FLJ22217 fis, clone HRC01591 | 0.18 | 0.97 | 0.59 | 1.08 | 0.39 | 0.18 | 0.33 | 0.64 | 5.39 | 3.28 |
| CCCTGGCTGT | Hs.58127 | Homo sapiens cDNA FLJ13902 fis, clone THYRO1001793 or site-1 protease (subtilisin-like, | 0.18 | 0.97 | 0.59 | 0.72 | 0.20 | 0.18 | 0.00 | 0.32 | 5.39 | 3.28 |
| | | sterol-regulated, [symbol] | | | | | | | | | | |
| TTTGGAGCAT | Hs.7773 | ESTs | 0.18 | 0.97 | 0.00 | 0.36 | 0.39 | 1.06 | 0.65 | 0.64 | 5.39 | 0.00 |
| GGACGGCATT | Hs.78531 | transmembrane protein with EGF-like and two follistatin-like domains 1 | 0.18 | 0.97 | 0.20 | 0.18 | 0.20 | 0.18 | 0.00 | 0.64 | 5.39 | 1.11 |
| TCTGTTTACT | J04031 | methylenetetrahydrofolate dehydrogenase (NADP+ dependent), methenyltetrahydrofolate cyclohydrolase, | 0.18 | 0.97 | 0.59 | 0.36 | 0.20 | 0.89 | 0.00 | 0.00 | 5.39 | 3.28 |
| | | form | | | | | | | | | | |
| CTCACACATT | J04182 | lysosomal-associated membrane protein 1 | 0.18 | 0.97 | 0.20 | 0.72 | 0.59 | 0.71 | 0.33 | 0.32 | 5.39 | 1.11 |
| GGCGTGAACC | M15796 | proliferating cell nuclear antigen | 0.18 | 0.97 | 0.39 | 0.72 | 0.20 | 0.53 | 0.33 | 0.32 | 5.39 | 2.17 |
| CCTCACTTTC | M94859 | calnexin | 0.18 | 0.97 | 0.39 | 0.00 | 0.20 | 0.00 | 0.33 | 0.32 | 5.39 | 2.17 |
| GGACTGGGAG | U04810 | trophinin associated protein (tastin) | 0.18 | 0.97 | 0.20 | 0.18 | 0.00 | 0.00 | 0.00 | 0.00 | 5.39 | 1.11 |
| AAGCCAGTTT | U57059 | tumor necrosis factor (ligand) superfamily, member 10 | 0.18 | 0.97 | 0.00 | 0.72 | 0.00 | 0.00 | 0.00 | 0.00 | 5.39 | 0.00 |
| CTCAACCCCC | X13916 | low density lipoprotein-related protein 1 (alpha-2-macroglobulin receptor) | 0.18 | 0.97 | 0.00 | 0.54 | 0.00 | 0.18 | 1.63 | 0.97 | 5.39 | 0.00 |
| GGAGCTGGCC | AF120274 | artemin | 0.74 | 3.67 | 0.78 | 4.33 | 0.00 | 0.00 | 0.00 | 0.00 | 4.96 | 1.05 |
| AGCCACCACG | AF075097 | acetyl LDL receptor; SREC or phosphodiesterase 6B, cGMP-specific, rod, beta | 0.37 | 1.74 | 0.98 | 0.54 | 0.98 | 0.53 | 0.33 | 0.32 | 4.70 | 2.65 |
| GTGGCTGCTG | AF086034 | DC47 mRNA | 0.37 | 1.74 | 0.98 | 0.36 | 1.76 | 0.71 | 0.98 | 1.29 | 4.70 | 2.65 |
| TGGCTTAAAT | AF144755 | HIG2; hypoxia-inducible protein 2 | 0.37 | 1.74 | 0.78 | 2.53 | 0.20 | 0.18 | 1.63 | 5.47 | 4.70 | 2.11 |
| TACGCTCCTT | AB037850 | DKFZP434I116 protein | 0.18 | 0.77 | 0.00 | 0.18 | 0.00 | 0.35 | 0.00 | 0.64 | 4.28 | 0.00 |
| CTCTGTAATT | AF000231 | RAB11A, member RAS oncogene family | 0.18 | 0.77 | 0.39 | 0.36 | 0.20 | 0.18 | 0.98 | 0.32 | 4.28 | 2.17 |
| TGGAGAGTCG | AF047690 | ATP-binding cassette, sub-family B (MDR/TAP), member 8 | 0.18 | 0.77 | 0.59 | 0.18 | 0.78 | 0.53 | 0.65 | 0.32 | 4.28 | 3.28 |
| CCCATCCGCA | AF083248 | ribosomal protein L26 homolog | 0.18 | 0.77 | 0.20 | 0.36 | 0.59 | 0.71 | 0.00 | 0.00 | 4.28 | 1.11 |
| AATTCCCGTC | AF161494 | mitochondrial ribosomal protein L15 | 0.18 | 0.77 | 0.59 | 0.54 | 0.39 | 1.06 | 0.00 | 0.00 | 4.28 | 3.28 |
| GCCACTACCC | AF208861 | acid cluster protein 33 | 0.37 | 1.55 | 1.18 | 0.54 | 0.98 | 1.77 | 0.98 | 1.29 | 4.19 | 3.19 |
| GGCCCTGCAG | AF233396 | sirtuin (silent mating type information regulation 2, S. cerevisiae, homolog) 6 | 0.18 | 0.77 | 0.59 | 0.72 | 0.39 | 0.35 | 0.98 | 0.64 | 4.28 | 3.28 |
| AGGTTCTGCC | AF251040 | putative nuclear protein | 0.18 | 0.77 | 0.59 | 0.72 | 0.39 | 0.18 | 0.33 | 0.32 | 4.28 | 3.28 |
| TCAACGGTGT | AF306510 | RAN binding protein 9 | 0.18 | 0.77 | 0.20 | 0.90 | 0.78 | 0.53 | 0.00 | 0.32 | 4.28 | 1.11 |
| TTCTGAAGCA | AK000508 | uncharacterized hypothalamus protein HT012 | 0.18 | 0.77 | 0.00 | 0.18 | 0.20 | 0.18 | 0.33 | 0.32 | 4.28 | 0.00 |
| GTCTCCGGGA | AK000591 | hypothetical protein FLJ20584 | 0.18 | 0.77 | 0.20 | 0.36 | 0.20 | 0.18 | 0.00 | 0.00 | 4.28 | 1.11 |
| GAGGCGATCA | AK000857 | hypothetical protein FLJ20850 | 0.18 | 0.77 | 0.39 | 0.72 | 1.57 | 1.24 | 1.95 | 0.97 | 4.28 | 2.17 |
| TAGAATTTTC | AK001183 | MMP1; metallaprotease 1 (pitrilysin family) | 0.18 | 0.77 | 0.59 | 0.00 | 0.20 | 0.18 | 0.33 | 0.00 | 4.28 | 3.28 |
| ATCCCACTGC | AK001962 | hypothetical protein FLJ11100 | 0.18 | 0.77 | 0.00 | 0.54 | 0.00 | 0.18 | 0.00 | 0.00 | 4.28 | 0.00 |
| TGGTTCTATA | AL050348 | similar to synaptotagmin 1, ubiquitin-conjugating enzyme E2 (H10) and the TNNC2 gene | 0.18 | 0.77 | 0.00 | 0.18 | 0.20 | 0.35 | 0.00 | 0.00 | 4.28 | 0.00 |
| ACAAAATATG | AL080110 | hypothetical protein FLJ21031 | 0.18 | 0.77 | 0.20 | 0.18 | 0.00 | 0.00 | 0.65 | 0.00 | 4.28 | 1.11 |
| TGAGCAAGCC | AL096880 | zinc finger protein 278 | 0.18 | 0.77 | 0.00 | 0.18 | 0.20 | 0.18 | 0.00 | 0.00 | 4.28 | 0.00 |
| CTTTGATCAG | AL121903 | brefeldin A-inhibited guanine nucleotide-exchange protein 2 | 0.18 | 0.77 | 0.59 | 0.72 | 0.00 | 0.35 | 0.00 | 0.00 | 4.28 | 3.28 |
| AGGCCCTGCT | AL133069 | hypothetical protein FLJ14590 | 0.18 | 0.77 | 0.00 | 0.18 | 0.20 | 0.35 | 0.00 | 0.00 | 4.28 | 0.00 |
| CTGTGGTTAC | AL133581 | Similar to RIKEN cDNA 1100001L14 gene | 0.18 | 0.77 | 0.00 | 0.00 | 0.39 | 0.53 | 0.33 | 0.00 | 4.28 | 0.00 |
| CTGGTGATGG | AL136683 | hyphothetical protein DKFZp564D0478 | 0.18 | 0.77 | 0.20 | 0.00 | 0.78 | 0.35 | 0.65 | 0.97 | 4.28 | 1.11 |
| TACCCCCGAG | BC006173 | hypothetical protein MGC13251 | 0.18 | 0.77 | 0.59 | 0.18 | 0.20 | 0.89 | 0.00 | 0.32 | 4.28 | 3.28 |
| GTATAATTTG | BC007341 | small membrane protein 1 | 0.37 | 1.55 | 0.59 | 0.90 | 0.39 | 0.00 | 0.65 | 0.00 | 4.19 | 1.59 |
| CAGCCAGGAC | D63485 | IKK-related kinase epsilon; inducible IkappaB kinase | 0.18 | 0.77 | 0.20 | 0.18 | 0.20 | 0.35 | 0.33 | 0.00 | 4.28 | 1.11 |
| TCTTTCACCC | D88674 | antizyme inhibitor | 0.18 | 0.77 | 0.39 | 0.54 | 0.00 | 0.18 | 0.33 | 0.00 | 4.28 | 2.17 |
| GTGGGGGGGA | Hs.10700 | hypothetical protein | 0.18 | 0.77 | 0.00 | 0.54 | 0.00 | 0.18 | 0.00 | 0.32 | 4.28 | 0.00 |
| TGTAGCTGCA | Hs.107882 | hypothetical protein FLJ10659 | 0.18 | 0.77 | 0.39 | 0.00 | 0.00 | 0.00 | 0.00 | 0.00 | 4.28 | 2.17 |
| GCCAGCTGAC | Hs.118913 | ESTs | 0.18 | 0.77 | 0.20 | 0.36 | 0.39 | 0.53 | 0.00 | 0.32 | 4.28 | 1.11 |
| TTTTCCCCTG | Hs.12243 | hypothetical protein or Homo sapiens mRNA; cDNA DKFZp564C1216 (from clone DKFZp564C1216) | 0.18 | 0.77 | 0.39 | 0.18 | 0.00 | 0.00 | 0.00 | 0.00 | 4.28 | 2.17 |
| ACCCAATCCT | Hs.12420 | ESTs | 0.18 | 0.77 | 0.00 | 0.36 | 0.00 | 0.00 | 0.00 | 0.00 | 4.28 | 0.00 |
| CAAACTGGGG | Hs.13015 | hypothetical protein similar to mouse Dna [symbol] 1 | 0.18 | 0.77 | 0.00 | 0.18 | 0.00 | 0.00 | 0.00 | 0.00 | 4.28 | 0.00 |
| GGCTGGGGTC | Hs.145303 | ESTs | 0.18 | 0.77 | 0.20 | 0.90 | 0.78 | 0.53 | 0.33 | 0.32 | 4.28 | 1.11 |
| GCTCTGTCAC | Hs.168640 | Ank | 0.37 | 1.55 | 0.98 | 1.63 | 0.00 | 0.18 | 0.00 | 0.00 | 4.19 | 2.65 |
| CCTTTCAAAA | Hs.169921 | general transcription factor II, i, pseudogene 1 | 0.18 | 0.77 | 0.59 | 0.18 | 0.39 | 0.35 | 0.00 | 0.00 | 4.28 | 3.28 |
| TGCAATATGG | Hs.173912 | eukaryotic translation initiation factor 4A, isoform 2 or hypothetical protein DKFZp434D0412 | 0.18 | 0.77 | 0.39 | 0.36 | 0.00 | 0.18 | 0.00 | 0.32 | 4.28 | 2.17 |
| CCGTCCGGAA | Hs.180842 | ribosomal protein L13 or EST | 0.18 | 0.77 | 0.39 | 0.36 | 1.17 | 0.00 | 0.00 | 0.00 | 4.28 | 2.17 |
| AGCGGAGTCT | Hs.181013 | phosphoglycerate mutase 1 (brain) | 0.18 | 0.77 | 0.00 | 0.00 | 0.39 | 0.35 | 0.33 | 0.00 | 4.28 | 0.00 |
| ACCAGAGAGC | Hs.181185 | DKFZP564D116 protein | 0.18 | 0.77 | 0.20 | 0.18 | 0.20 | 0.18 | 0.98 | 0.32 | 4.28 | 1.11 |
| GGGGCTGGGG | Hs.183698 | ribosomal protein L29 or glucosidase, beta; acid (includes glucosylceramidase) or ESTs | 0.18 | 0.77 | 0.59 | 0.18 | 0.39 | 0.35 | 0.65 | 0.64 | 4.28 | 3.28 |
| GACAGCCTTA | Hs.187111 | ESTs | 0.18 | 0.77 | 0.20 | 0.54 | 0.00 | 0.18 | 0.00 | 0.32 | 4.28 | 1.11 |
| GCGGAACGCA | Hs.191290 | ESTs | 0.18 | 0.77 | 0.20 | 0.18 | 0.00 | 0.00 | 0.33 | 0.32 | 4.28 | 1.11 |
| TGGCAAAGCC | Hs.2186 | eukaryotic translation elongation factor 1 gamma | 0.18 | 0.77 | 0.00 | 0.00 | 0.59 | 0.71 | 0.00 | 0.32 | 4.28 | 0.00 |
| GGAACAACAG | Hs.22981 | DKFZP586M1523 protein | 0.18 | 0.77 | 0.39 | 0.54 | 1.17 | 0.18 | 1.30 | 1.29 | 4.28 | 2.17 |
| GTATTTGTGG | Hs.235475 | ESTs | 0.18 | 0.77 | 0.20 | 0.36 | 0.00 | 0.00 | 0.00 | 0.32 | 4.28 | 1.11 |
| CTGAGCAGAA | Hs.241427 | DKFZP434F1735 protein or ESTs | 0.18 | 0.77 | 0.00 | 0.00 | 0.00 | 0.00 | 0.00 | 0.00 | 4.28 | 0.00 |
| GCTTGCTGCC | Hs.278242 | tubulin, alpha, ubiquitous | 0.18 | 0.77 | 0.20 | 0.18 | 0.00 | 0.00 | 0.00 | 0.00 | 4.28 | 1.11 |
| GGGGGTTGGT | Hs.4779 | KIAA1150 protein | 0.18 | 0.77 | 0.39 | 0.90 | 1.17 | 0.71 | 0.65 | 0.97 | 4.28 | 2.17 |
| CTTCTGGTGC | Hs.75431 | fibrinogen, gamma polypeptide | 0.18 | 0.77 | 0.59 | 0.36 | 0.20 | 0.00 | 0.65 | 1.61 | 4.28 | 3.28 |
| GGATCCTCGG | Hs.78040 | KDEL (Lys-Asp-Glu-Leu) endoplasmic reticulum protein retention receptor 1 | 0.18 | 0.77 | 0.20 | 0.36 | 0.39 | 0.35 | 0.65 | 1.29 | 4.28 | 1.11 |
| GGTGAATCTC | Hs.77326 | IGFBP3; insulin-like growth factor binding protein 3 (EST) | 0.18 | 0.77 | 0.20 | 0.36 | 0.00 | 0.00 | 0.00 | 0.32 | 0.28 | 1.11 |
| GGCTGGGGCC | J03545 | (11nt = C) No; = G; PRTN3; proteinase 3 (serine proteinase, neutrophil, Wegener | 0.18 | 0.77 | 0.20 | 0.18 | 0.39 | 0.18 | 0.00 | 0.00 | 4.28 | 1.11 |
| | | granulomatosis autoantigen); | | | | | | | | | | |
| GCCTTTCTAA | L07597 | ribosomal protein S6 kinase, 90 kD, polypeptide 1 | 0.18 | 0.77 | 0.39 | 0.72 | 0.39 | 0.53 | 0.00 | 0.00 | 4.28 | 2.17 |
| AACTGTCCTT | L37385 | PEA15; phosphoprotein enriched in astrocytes 15 | 0.18 | 0.77 | 0.00 | 0.18 | 0.20 | 0.00 | 0.98 | 0.32 | 4.28 | 0.00 |
| TTTTTGTTTT | M21574 | PDGFRA; platelet-derived growth factor receptor, alpha (PDGF receptor alpha) polypeptide | 0.18 | 0.77 | 0.00 | 0.00 | 0.00 | 0.18 | 0.00 | 0.00 | 4.28 | 0.00 |
| TGCACCTTGG | M68520 | cyclin-dependent kinase 2 | 0.18 | 0.77 | 0.59 | 1.26 | 0.00 | 0.00 | 0.33 | 0.97 | 4.28 | 3.28 |
| TTCCTCCACG | S74620 | src homology 3 domain-containing protein HIP-55 | 0.37 | 1.55 | 0.59 | 0.90 | 1.57 | 1.42 | 0.33 | 0.00 | 4.19 | 1.59 |
| AGGTGCAGAG | U78310 | pescadillo (zebrafish) homolog 1, containing BRCT domain | 0.18 | 0.77 | 0.39 | 0.54 | 0.78 | 0.89 | 1.30 | 0.64 | 4.28 | 2.17 |
| CAGCCCAACC | U96074 | eukaryotic translation initiation factor 3, subunit 4 (delta, 44 kD) | 0.18 | 0.77 | 0.20 | 0.36 | 2.15 | 1.24 | 0.65 | 0.32 | 4.28 | 1.11 |
| GGCTCGGGAT | X04366 | calpain 1, (mu/l) large subunit | 0.18 | 0.77 | 0.39 | 0.00 | 2.74 | 1.42 | 0.33 | 0.97 | 4.28 | 2.17 |
| TGTGAACACA | X14454 | interferon regulatory factor 1 | 0.18 | 0.77 | 0.39 | 0.00 | 0.00 | 0.18 | 0.33 | 0.00 | 4.28 | 2.17 |
| ACAAAATAAA | X77366 | nuclear factor (erythroid-derived 2)-like 1 | 0.18 | 0.77 | 0.20 | 0.18 | 0.20 | 0.71 | 0.33 | 0.00 | 4.28 | 1.11 |
| AAACACTCTT | X80695 | oxidase (cytochrome c) assembly 1-like | 0.18 | 0.77 | 0.20 | 0.00 | 0.20 | 0.71 | 0.33 | 0.64 | 4.28 | 1.11 |
| TTGAATTGAA | X89985 | B-cell CLL/lymphoma 7B | 0.18 | 0.77 | 0.20 | 0.54 | 0.39 | 0.18 | 0.33 | 0.00 | 4.28 | 1.11 |
| ATGACTGTAC | X95592 | nuclear DNA-binding protein | 0.18 | 0.77 | 0.20 | 0.18 | 0.20 | 0.18 | 0.33 | 0.32 | 4.28 | 1.11 |
| CTCCCAGGTC | X98263 | M-phase phosphoprotein 6 | 0.18 | 0.77 | 0.39 | 0.72 | 0.00 | 0.00 | 0.00 | 0.00 | 4.28 | 2.17 |

38 genes in FIG. 1(b) (36 genes; 2 tags with no matches not shown)
SAGE data for genes up-regulated ≧ 4-fold in 786-0 VHL⁻ Nor. (V2) compared to 786-0 VHL⁺ Nor. (V1),
and induced by hypoxia in 786-0 VHL⁺ Hyp. (V3). compared to 786-0 VHL⁺ Nor. (V1). SAGE tag abundances were
normalized before calculating ratios, as shown. The table is presented in descending order of V2/V1.

| | | | | | | | | | | | | |
|---|---|---|---|---|---|---|---|---|---|---|---|---|
| CTGTTGGCAT | Hs.184108 | ribosomal protein L21 (gene or pseudogene) | 0.18 | 1.74 | 0.78 | 1.08 | 0.20 | 0.35 | 0.33 | 1.61 | 9.67 | 4.33 |
| AACGGGCCGG | Hs.26484 | HIRA-interacting protein 3 | 0.18 | 1.55 | 0.78 | 0.54 | 0.39 | 0.18 | 0.33 | 0.32 | 8.61 | 4.33 |
| CCACGTTCCA | NM_001975 | ENO2; enolase 2, (gamma, neuronal) | 0.18 | 1.55 | 0.98 | 1.63 | 0.78 | 1.06 | 5.85 | 16.74 | 8.61 | 5.44 |
| ATGCGCAAGG | Z36785 | ribosomal protein, large, P0 | 0.18 | 1.55 | 0.98 | 0.90 | 1.96 | 1.77 | 0.98 | 1.61 | 8.61 | 5.44 |
| GTGCTTGTAC | AB001106 | GMFB; glia maturation factor, beta | 0.18 | 1.35 | 1.18 | 1.08 | 0.20 | 0.35 | 0.33 | 0.97 | 7.50 | 6.56 |
| AGGAAGAGCC | AF125535 | pp21 homolog | 0.18 | 1.16 | 1.18 | 0.00 | 0.39 | 0.00 | 0.65 | 0.64 | 6.44 | 6.56 |
| ACCTGCTGGT | AK000447 | GTPase Rab14 | 0.18 | 1.16 | 1.18 | 0.00 | 0.20 | 0.35 | 0.00 | 0.97 | 6.44 | 6.56 |
| GTGGTGGGTG | X58236 | solute carrier family 14 (urea transporter), member 1 (Kidd blood group) | 0.18 | 1.16 | 0.78 | 0.90 | 0.39 | 0.89 | 0.65 | 0.32 | 6.44 | 4.33 |
| CCGGACCTGT | Hs.277777 | paired immunoglobulin-like receptor beta | 0.18 | 1.16 | 0.78 | 1.08 | 1.17 | 0.35 | 0.98 | 0.97 | 6.44 | 4.33 |
| GTGCCTAGGA | Hs.25999 | hypothetical protein FLJ22195 | 0.18 | 1.16 | 0.98 | 1.63 | 0.78 | 2.13 | 1.95 | 1.29 | 6.44 | 5.44 |
| CTCACTTCTT | Hs.165998 | PAI-1 mRNA-binding protein (EST) | 0.18 | 0.97 | 0.98 | 0.90 | 0.20 | 0.00 | 0.98 | 0.00 | 5.39 | 5.44 |
| CTAAGGTGGG | L76702 | protein phosphatase 2, regulatory subunit B (B56), delta isoform | 0.18 | 0.97 | 0.98 | 0.18 | 0.00 | 0.35 | 0.33 | 0.64 | 5.39 | 5.44 |
| GTTGTGATGT | NM_005243 | Ewing sarcoma breakpoint region 1 or EST | 0.18 | 0.97 | 0.18 | 1.08 | 0.00 | 0.35 | 0.00 | 0.00 | 5.39 | 6.56 |
| TTCTGGCACT | Hs.20237 | DKFZP566C134 protein or EST | 0.18 | 0.97 | 0.78 | 1.08 | 0.20 | 0.18 | 0.00 | 0.00 | 5.39 | 4.33 |
| GGGCGGGGGC | NM_002691 | 11nt = g; polymerase (DNA directed), delta 1, catalytic subunit (125 kD) (11 g) or KIAA0596 protein | 0.18 | 0.97 | 0.98 | 1.99 | 0.39 | 0.18 | 0.00 | 0.00 | 5.39 | 5.44 |
| | | (11 g EST) or EST | | | | | | | | | | |
| TACTGTGGAT | Hs.21537 | protein phosphatase 1, catalytic subunit, beta isoform | 0.37 | 1.93 | 1.76 | 0.90 | 0.78 | 0.53 | 0.65 | 0.97 | 5.22 | 4.76 |
| CGGGATGCAG | Hs.155560 | calnexin | 0.18 | 0.97 | 0.78 | 1.08 | 0.59 | 0.18 | 1.95 | 1.29 | 5.39 | 4.33 |
| CGCTTTTGTA | AL110124 | DKFZP564A2416 protein | 0.55 | 2.90 | 2.75 | 1.81 | 0.98 | 1.77 | 1.95 | 1.61 | 5.27 | 5.00 |
| CTTATGATCA | AJ238095 | Lsm3 protein | 0.18 | 0.97 | 0.78 | 0.54 | 0.78 | 0.18 | 0.33 | 0.00 | 5.39 | 4.33 |
| CAGAAGAGGC | AK001528 | DiGeorge syndrome critical region gene 6 | 0.18 | 0.97 | 0.98 | 0.36 | 1.96 | 2.13 | 0.98 | 0.97 | 5.39 | 5.44 |
| GCTAGGTATT | AJ250865 | testin | 0.18 | 0.77 | 0.98 | 1.08 | 0.00 | 0.18 | 0.33 | 0.00 | 4.28 | 5.44 |
| GTAAAACCCT | AF232009 | HSA250303; peroxisomal trans 2-enoyl CoA reductase; putative short chain alcohol dehydrogenase | 0.18 | 0.77 | 0.78 | 0.00 | 0.20 | 0.00 | 0.00 | 0.32 | 4.28 | 4.33 |
| TGGAAATAAA | BC000547 | mitochondrial ribosomal protein S6 | 0.18 | 0.77 | 0.98 | 0.36 | 0.00 | 0.35 | 0.00 | 0.64 | 4.28 | 5.44 |
| GTGAAGTCTT | AF055031 | solute carrier family 3 (activators of dibasic and neutral amino acid transport), member 2 | 0.37 | 1.55 | 1.57 | 0.72 | 0.39 | 0.35 | 0.98 | 0.97 | 4.19 | 4.24 |
| GAGTAGAGAA | Hs.145279 | SET translocation (myeloid leukemia-associated) | 0.37 | 1.55 | 1.76 | 1.44 | 0.39 | 0.35 | 0.33 | 0.32 | 4.19 | 4.76 |
| AGGGAGGCAG | Hs.74368 | transmembrane protein (63 kD), endoplasmic reticulum/Golgi intermediate compartment | 0.18 | 0.77 | 1.18 | 0.90 | 0.39 | 0.00 | 0.33 | 0.00 | 4.28 | 6.56 |
| AGCAACAGTG | U10117 | SCYE1; small inducible cytokine subfamily E, member 1 (endothelial monocyte-activating) | 0.18 | 0.77 | 1.18 | 0.18 | 0.39 | 0.00 | 0.33 | 0.97 | 4.28 | 6.56 |
| GAGAATCTGC | Hs.179260 | 11nt = t; chromosome 14 open reading frame 4 (11t) or EST (11c or t) | 0.18 | 0.77 | 0.78 | 0.72 | 0.39 | 0.18 | 0.00 | 0.00 | 4.28 | 4.33 |
| AAAAACCATA | Hs.76362 | general transcription factor IIA, 2 (12 kD subunit) or EST | 0.18 | 0.77 | 0.78 | 0.54 | 0.59 | 0.00 | 0.00 | 0.00 | 4.28 | 4.33 |
| GTCTTTCTGG | AB046781 | uveal autoantigen with coiled coil domains and ankyrin repeats | 0.18 | 0.77 | 0.98 | 0.54 | 0.00 | 0.00 | 0.00 | 0.64 | 4.28 | 5.44 |
| GCTACTATTA | Hs.154443 | minichromosome maintenance deficient (S. cerevisiae) 4 | 0.18 | 0.77 | 0.98 | 0.54 | 0.00 | 0.00 | 0.00 | 0.00 | 4.28 | 5.44 |
| CAGCTGGGGC | Hs.172550 | 11nt = g; polypyrimidine tract binding protein (heterogeneous nuclear ribonucleoprotein I) or EST (11 g) | 0.18 | 0.77 | 1.18 | 0.18 | 1.37 | 0.71 | 0.00 | 0.00 | 4.28 | 6.56 |
| TTCTCTCAAC | AF089106 | unknown | 0.18 | 0.77 | 0.98 | 0.72 | 0.78 | 1.42 | 2.28 | 0.97 | 4.28 | 5.44 |
| ACGGCTCCGA | Hs.48563 | ESTs | 0.18 | 0.77 | 1.18 | 0.90 | 2.15 | 0.71 | 0.98 | 0.97 | 4.28 | 6.56 |
| TGGGCTCTGA | D12676 | CD36 antigen (collagen type I receptor, thrombospondin receptor)-like 2 (lysosomal integral membrane | 0.18 | 0.77 | 1.18 | 0.90 | 0.98 | 2.13 | 0.98 | 1.61 | 4.28 | 6.56 |
| | | protein II) | | | | | | | | | | |
| GGCGTCCTGG | Hs.44017 | SIR2 (silent mating type information regulation 2, S. cerevisiae, homolog)-like | 0.18 | 0.77 | 0.78 | 0.36 | 1.96 | 1.42 | 0.98 | 0.00 | 4.28 | 4.33 |

122 genes in FIG. 1 (105 genes; 17 tags with no matches not shown)
SAGE data for genes up-regulated ≧ 4-fold by hypoxia in 786-0 VHL⁺ Hyp. (V3) when compared to 786-0 VHL⁺ Nor. (V1).
Also shown, for comparison, is the SAGE data for each gene in the 786-0 VHL⁻ Nor. (V2) with respect to 786-0 VHL⁺ Nor. (V1).
SAGE tag abundances were normalized before calculating ratios, as shown. The table is presented in descending order of V3/V1.

| | | | | | | | | | | | | |
|---|---|---|---|---|---|---|---|---|---|---|---|---|
| TATGTGCCAC | AK001323 | hypothetical protein FLJ10461 | 0.55 | 1.93 | 2.35 | 0.36 | 0.20 | 0.00 | 0.00 | 0.00 | 4.27 | 3.51 |
| TGTGACCTCT | AF061729 | dolichyl-phosphate mannosyltransferase polypeptide 2, regulatory subunit | 0.18 | 0.58 | 0.78 | 0.18 | 0.00 | 0.18 | 0.00 | 0.00 | 4.33 | 3.22 |
| AAGACAAGTG | Hs.184109 | ribosomal protein L37a | 0.18 | 0.58 | 0.78 | 0.18 | 0.00 | 0.18 | 0.00 | 0.00 | 4.33 | 3.22 |
| AGAATGTACG | AL049851 | hypothetical protein | 0.37 | 1.35 | 2.94 | 1.81 | 0.20 | 0.35 | 0.33 | 0.64 | 7.95 | 3.65 |
| GGTGCTCCCT | AB015633 | transmembrane protein 5 | 0.18 | 0.39 | 0.98 | 0.00 | 0.20 | 0.00 | 0.33 | 0.32 | 5.44 | 2.17 |
| GCGAGCAGCG | Hs.300141 | ribosomal protein L39 | 0.18 | 0.39 | 0.78 | 0.72 | 0.20 | 0.00 | 0.00 | 0.64 | 4.33 | 2.17 |
| CACTGAGCCA | AK000034 | DnaJ (Hsp40) homolog, subfamily B, member 12 | 0.37 | 1.35 | 1.57 | 1.08 | 0.00 | 0.71 | 0.33 | 0.32 | 4.24 | 3.65 |
| GCCAGGGGCC | BC002542 | KIAA1273 protein | 0.18 | 0.58 | 0.78 | 0.18 | 0.20 | 0.18 | 0.00 | 0.00 | 4.33 | 3.22 |
| CAGAGTGACT | L07758 | nuclear phosphoprotein similar to S. cerevisiae PWP1 | 0.18 | 0.58 | 0.78 | 1.26 | 0.39 | 0.00 | 0.33 | 0.32 | 4.33 | 3.22 |
| CTATATTTTT | X87212 | cathepsin C | 0.18 | 0.58 | 0.78 | 0.18 | 0.39 | 0.00 | 0.00 | 0.00 | 4.33 | 3.22 |
| ATGAAACCCC | U67806 | small inducible cytokine A5 (RANTES) or others | 0.37 | 1.16 | 2.35 | 0.72 | 0.59 | 0.35 | 0.00 | 0.64 | 6.35 | 3.14 |
| ATGTGAGGGA | AK000510 | tRNA selenocysteine associated protein | 0.18 | 0.39 | 0.78 | 0.36 | 0.00 | 0.35 | 0.65 | 0.97 | 4.33 | 2.17 |
| TAGTAAGTCA | AL110153 | ribosomal protein L22 | 0.18 | 0.19 | 0.98 | 0.90 | 0.00 | 0.18 | 0.00 | 0.00 | 5.44 | 1.06 |
| CCTGAATCTG | Hs.179779 | ribosomal protein L37 | 0.18 | 0.58 | 0.78 | 0.36 | 0.20 | 0.35 | 0.00 | 0.00 | 4.33 | 3.22 |
| GTGGTGTGTG | AB033023 | nuclear factor of activated T-cells, cytoplasmic, calcineurin-dependent 3 or others | 0.18 | 0.58 | 1.76 | 0.54 | 0.39 | 0.18 | 0.00 | 0.97 | 9.78 | 3.22 |
| CAGAAGTGTC | D13640 | KIAA0015 gene product | 0.18 | 0.58 | 0.78 | 0.54 | 0.39 | 0.18 | 0.65 | 0.00 | 4.33 | 3.22 |
| GCTCTGTAAG | AK000222 | putative methyltransferase | 0.18 | 0.39 | 0.78 | 0.72 | 0.20 | 0.18 | 0.00 | 0.32 | 4.33 | 2.17 |
| GTAAAAGTTC | D21262 | nucleolar and coiled-body phosphprotein 1 | 0.18 | 0.39 | 1.57 | 0.54 | 0.20 | 0.18 | 0.00 | 0.00 | 8.72 | 2.17 |
| TCTCTAGAAT | X75342 | SHB adaptor protein (a Src homology 2 protein) | 0.18 | 0.39 | 0.78 | 0.36 | 0.20 | 0.18 | 0.00 | 0.00 | 4.33 | 2.17 |
| AACTCCCAGT | AL050044 | GADD45B; growth arrest and DNA-damage-inducible, beta | 0.18 | 0.39 | 0.98 | 0.72 | 0.39 | 0.00 | 0.65 | 0.97 | 5.44 | 2.17 |
| TGCAAAAAAA | BC000610 | hypothetical protein FLJ10986 | 0.18 | 0.39 | 0.78 | 0.18 | 0.39 | 0.00 | 0.33 | 0.00 | 4.33 | 2.17 |
| GTTGTGGCCA | Hs.165563 | Homo sapiens cDNA FLJ13070 fis, clone NT2RP3001777 | 0.18 | 0.19 | 0.78 | 0.36 | 0.20 | 0.00 | 0.00 | 0.32 | 4.33 | 1.06 |
| TTTAATTGTG | Hs.145279 | SET translocation (myeloid leukemia-associated) or etoposide-induced mRNA | 0.18 | 0.19 | 0.78 | 0.00 | 0.20 | 0.00 | 0.33 | 0.00 | 4.33 | 1.06 |
| GAGGAGTGGG | Hs.206770 | zinc finger protein 297 | 0.18 | 0.19 | 0.98 | 0.36 | 0.20 | 0.00 | 0.00 | 0.00 | 5.44 | 1.06 |
| AATAGGGTCA | AF086183 | amyloid beta (A4) precursor-like protein 2 (EST) | 0.37 | 1.16 | 1.96 | 0.90 | 0.59 | 0.71 | 0.00 | 0.00 | 5.30 | 3.14 |
| CTGAGCTGTA | AK001080 | WD repeat domain 6 | 0.18 | 0.58 | 0.78 | 0.18 | 0.39 | 0.35 | 0.33 | 0.32 | 4.33 | 3.22 |
| TGTGTGGGGC | AK024450 | unknown | 0.18 | 0.58 | 0.78 | 0.00 | 0.39 | 0.35 | 0.00 | 0.00 | 4.33 | 3.22 |
| GTGGCGGGCG | D90313 | carcinoembryonic antigen-related cell adhesion molecule 1 (biliary glycoprotein) or others | 0.92 | 1.74 | 3.73 | 2.89 | 1.37 | 0.89 | 1.63 | 0.97 | 4.05 | 1.89 |
| TGTGAGCCCC | AF265209 | enigma (LIM domain protein) | 0.37 | 0.58 | 1.57 | 0.36 | 0.59 | 0.18 | 0.00 | 0.00 | 4.24 | 1.57 |
| CGTGAACAAT | AF226049 | GL004 protein | 0.18 | 0.39 | 0.78 | 0.18 | 0.20 | 0.35 | 0.00 | 0.32 | 4.33 | 2.17 |
| AGCCTCGGGC | Hs.75922 | brain protein I3 | 0.18 | 0.39 | 0.78 | 0.72 | 0.39 | 0.18 | 0.33 | 0.32 | 4.33 | 2.17 |
| GGCTGCAGTC | AL110253 | double ring-finger protein, Dorfin | 0.18 | 0.39 | 0.98 | 0.00 | 0.59 | 0.00 | 0.65 | 0.97 | 5.44 | 2.17 |
| TTCTCTCTGT | M57567 | ADP-ribosylation factor 5 | 0.18 | 0.58 | 0.78 | 0.72 | 0.20 | 0.71 | 0.00 | 0.00 | 4.33 | 3.22 |
| CTGCCAAAAA | AF112214 | mitochondrial ribosomal protein L13 | 0.18 | 0.39 | 0.78 | 0.36 | 0.20 | 0.53 | 0.00 | 0.00 | 4.33 | 2.17 |
| CTCTCAATAT | Hs.279518 | amyloid precursor protein homolog HSD-2 or amyloid beta (A4) precursor-like protein 2 | 0.18 | 0.39 | 0.78 | 0.36 | 0.20 | 0.53 | 0.33 | 0.00 | 4.33 | 2.17 |
| AAAATAAAGA | D90373 | APEX nuclease (multifunctional DNA repair enzyme) | 0.18 | 0.58 | 0.78 | 0.36 | 0.39 | 0.71 | 0.65 | 0.64 | 4.33 | 3.22 |
| ACCAAATTAA | AF016266 | tumor necrosis factor receptor superfamily, member 10b (TNF receptor) | 0.18 | 0.58 | 0.78 | 0.54 | 0.59 | 0.53 | 0.00 | 0.00 | 4.33 | 3.22 |
| AAATAAAAGC | X51521 | thyroid stimulating hormone receptor or villin 2 (ezrin) | 0.18 | 0.39 | 0.78 | 1.08 | 0.59 | 0.18 | 0.33 | 0.64 | 4.33 | 2.17 |
| CCCAGCTAAT | AF339827 | ATPase, H+ transporting, lysosomal (vacuolar proton pump), alpha polypeptide, 70 kD, isoform 1 (11nt) or | 0.18 | 0.19 | 1.18 | 1.26 | 0.20 | 0.18 | 0.33 | 0.32 | 6.56 | 1.06 |
| | | FOXJ2 forkhead factor (1 | | | | | | | | | | |
| GACCCACTAC | J03569 | SLC3A2; solute carrier family 3 (activators of dibasic and neutral amino acid transport), member 2 | 0.18 | 0.19 | 1.76 | 0.18 | 0.20 | 0.18 | 0.33 | 1.29 | 9.78 | 1.06 |
| CTGTTTGTTC | Hs.288965 | Homo sapiens cDNA: FLJ22300 fis, clone HRC04759 | 0.18 | 0.19 | 0.78 | 0.36 | 0.20 | 0.18 | 0.00 | 0.64 | 4.33 | 1.06 |
| GGAACGGATG | AJ131186 | nuclear matrix protein NMP200 related to splicing factor PRP19 | 0.18 | 0.39 | 0.98 | 0.00 | 0.78 | 0.18 | 0.65 | 0.00 | 5.44 | 2.17 |
| CTTTCTTCCC | X79067 X71 | ZFP36L1; Zinc finger protein 36, C3H type-like 1; butyrate response factor 1 (EGF-response factor 1) | 0.18 | 0.39 | 0.98 | 0.36 | 0.98 | 0.00 | 1.30 | 1.61 | 5.44 | 2.17 |
| GGCTATGCCA | AF147357 | cisplatin resistance-associated overexpressed protein (EST) or others | 0.37 | 0.77 | 1.57 | 2.17 | 0.00 | 0.00 | 0.00 | 0.00 | 4.24 | 2.08 |
| TAAACTTCAA | Hs.87627 | disrupter of silencing 10 | 0.18 | 0.19 | 0.98 | 0.54 | 0.20 | 0.35 | 0.65 | 0.00 | 5.44 | 1.06 |
| CTGCTGAGCC | Hs.1742 | IQ motif containing GTPase activating protein 1 | 0.18 | 0.39 | 0.78 | 0.54 | 0.78 | 0.53 | 0.33 | 1.29 | 4.33 | 2.17 |
| AACAACAGTG | AK001472 | anillin (Drosophila Scraps homolog), actin binding protein (EST) or others | 0.18 | 0.58 | 0.78 | 0.36 | 0.00 | 0.00 | 0.00 | 0.00 | 4.33 | 3.22 |
| TAGTCATCTT | U68758 | pyrroline-5-carboxylate synthetase (glutamate gamma-semialdehyde synthetase) | 0.18 | 0.58 | 1.18 | 0.18 | 0.00 | 0.00 | 0.00 | 0.00 | 6.56 | 3.22 |
| GTTTGGCTGC | Hs.154443 | minichromosome maintenance deficient (S. cerevisiae) 4 | 0.18 | 0.58 | 0.78 | 0.54 | 0.00 | 0.00 | 0.00 | 0.00 | 4.33 | 3.22 |
| GACTAAAAAA | NM_001019 | ribosomal protein S15a | 0.18 | 0.19 | 0.78 | 0.00 | 0.00 | 0.71 | 0.00 | 0.00 | 4.33 | 1.06 |
| GTGCAAAATG | Hs.247280 | HBV associated factor | 0.18 | 0.39 | 0.98 | 1.08 | 0.39 | 1.06 | 1.95 | 2.90 | 5.44 | 2.17 |
| TGTTGGGTTC | AB013385 | MAP4K4; mitogen-activated protein kinase kinase kinase kinase 4 | 0.18 | 0.19 | 0.78 | 0.18 | 0.20 | 0.53 | 0.65 | 0.00 | 4.33 | 1.06 |
| GTGGACCCTG | AF132959 | NOSIP (eNOS interacting protein) | 0.18 | 0.39 | 0.78 | 0.36 | 0.78 | 0.71 | 0.65 | 0.32 | 4.33 | 2.17 |
| GCTATGCTCC | BC003653 | presenilins associated rhombold-like protein | 0.18 | 0.39 | 0.98 | 0.72 | 0.78 | 0.71 | 0.33 | 0.97 | 5.44 | 2.17 |
| CAGAGTTGTA | AB014733 | hypothetical protein AF140225 | 0.18 | 0.19 | 0.98 | 0.90 | 0.59 | 0.18 | 0.33 | 0.32 | 5.44 | 1.06 |
| GAGCCGCCTC | AK001809 | HSPC182 protein | 0.18 | 0.39 | 1.37 | 0.90 | 0.59 | 1.06 | 0.00 | 0.32 | 7.61 | 2.17 |
| ACCCCAGCAA | Hs.164036 | glucosamine (N-acetyl)-6-sulfatase (Sanfilippo disease IIID) or putative zinc finger protein | 0.18 | 0.39 | 0.78 | 0.18 | 0.78 | 0.89 | 1.30 | 0.00 | 4.33 | 2.17 |
| GAGGTGCTCT | AF220050 | uncharacterized hematopoietic stem/progenitor cells protein MDS030 | 0.18 | 0.39 | 0.98 | 0.00 | 1.17 | 0.53 | 0.65 | 0.32 | 5.44 | 2.17 |
| AGGATGGCGG | Hs.182979 | ribosomal protein L12 | 0.55 | 0.58 | 2.35 | 0.90 | 1.37 | 1.42 | 0.98 | 1.29 | 4.27 | 1.05 |
| ATGGTCTACG | AK000254 | hypothetical protein | 0.18 | 0.39 | 0.98 | 0.18 | 1.17 | 0.71 | 2.60 | 0.97 | 5.44 | 2.17 |
| GAATGCAGTT | Hs.306155 | chorionic somatomammotropin hormone 2 or ESTs | 0.18 | 0.19 | 0.78 | 0.36 | 0.59 | 0.35 | 0.65 | 0.00 | 4.33 | 1.06 |
| GCCAAACTTG | Hs.119598 | ribosomal protein L3 or ALEX3 protein | 0.18 | 0.19 | 0.78 | 0.18 | 0.78 | 0.18 | 0.33 | 0.64 | 4.33 | 1.06 |
| TGGCAAAGTA | Hs.104480 | ESTs | 0.37 | 0.39 | 1.57 | 0.00 | 0.00 | 0.00 | 0.00 | 0.00 | 4.24 | 1.05 |
| TAATGACAAT | U60115 | four and a half LIM domains 1 | 0.18 | 0.39 | 0.98 | 0.36 | 0.00 | 0.00 | 0.00 | 0.00 | 5.44 | 2.17 |
| AAGCTGTTGT | X63692 | DNA (cytosine-5-)-methyltransferase 1 | 0.18 | 0.39 | 0.78 | 0.18 | 0.00 | 0.00 | 0.00 | 0.00 | 4.33 | 2.17 |
| TGTGGCCAGG | Hs.290357 | ESTs | 0.18 | 0.39 | 0.78 | 0.00 | 0.00 | 0.00 | 0.00 | 0.32 | 4.33 | 2.17 |
| TAACATTGAG | Hs.43756 | ESTs | 0.18 | 0.39 | 0.78 | 0.00 | 0.00 | 0.00 | 0.00 | 0.00 | 4.33 | 2.17 |
| GGAAGTCCTG | Hs.118787 | transforming growth factor, beta-induced, 68 kD | 0.18 | 0.39 | 1.18 | 1.08 | 0.00 | 0.00 | 0.33 | 0.00 | 6.56 | 2.17 |
| GCCTCCCGCC | BC001211 | kinesin family member C3 | 0.18 | 0.19 | 0.78 | 0.72 | 0.59 | 0.53 | 0.33 | 0.32 | 4.33 | 1.06 |
| AGAATTGTGT | BC004916 | hypothetical protein MGC4767 | 0.18 | 0.19 | 0.78 | 0.18 | 0.39 | 0.89 | 0.33 | 0.00 | 4.33 | 1.06 |
| TGACACCCAC | X17025 | isopentenyl-diphosphate delta isomerase | 0.18 | 0.19 | 0.78 | 0.36 | 0.78 | 0.53 | 1.30 | 0.00 | 4.33 | 1.06 |
| GGTTGAGTGT | Hs.20529 | Homo sapiens cDNA: FLJ21811 fis, clone HEP01037 | 0.18 | 0.39 | 0.78 | 0.36 | 1.78 | 1.06 | 1.95 | 0.32 | 4.33 | 2.17 |
| CCACAGGAGA | AJ251830 | p53-induced protein PIGPC1: THW tumor suppressor | 0.18 | 0.39 | 1.37 | 0.90 | 1.17 | 1.77 | 1.95 | 2.58 | 7.61 | 2.17 |
| AAGGAGTTTG | M33374 | NADH dehydrogenase (ubiquinone) 1 beta subcomplex, 7 (18 kD, B18) | 0.37 | 0.39 | 1.96 | 0.90 | 2.15 | 1.06 | 0.33 | 0.97 | 5.30 | 1.05 |
| GCGGCAGTTA | M55268 J02 | CSNK2A2; casein kinase 2, alpha prime polypeptide | 0.18 | 0.19 | 0.78 | 0.36 | 0.59 | 1.06 | 0.33 | 0.64 | 4.33 | 1.06 |
| TGAATACTAC | AB034747 | LPS-induced TNF-alpha factor | 0.18 | 0.19 | 0.78 | 0.18 | 0.00 | 0.00 | 0.33 | 0.00 | 4.33 | 1.06 |
| TGCATTGTTT | AB040915 | KIAA1482 protein | 0.18 | 0.19 | 0.78 | 0.18 | 0.00 | 0.00 | 0.00 | 0.00 | 4.33 | 1.06 |
| GTGTATATTG | Hs.292669 | ESTs | 0.18 | 0.19 | 0.78 | 0.18 | 0.00 | 0.00 | 0.33 | 0.00 | 4.33 | 1.06 |
| GATGCTGATT | Hs.26410 | ESTs | 0.18 | 0.19 | 0.78 | 0.18 | 0.00 | 0.00 | 0.00 | 0.00 | 4.33 | 1.06 |
| CCTTACTTTA | Hs.75415 | beta-2-microglobulin | 0.18 | 0.19 | 0.78 | 0.18 | 0.00 | 0.00 | 0.00 | 0.00 | 4.33 | 1.06 |
| CCACTTCCTC | Hs.77495 | KIAA0242 protein or ESTs | 0.18 | 0.19 | 0.78 | 0.36 | 0.00 | 0.00 | 0.00 | 0.00 | 4.33 | 1.06 |
| CATCCAAAAC | Hs.245710 | heterogeneous nuclear ribonucleoprotein H1 (H) | 0.18 | 0.19 | 0.78 | 0.72 | 0.00 | 0.00 | 0.00 | 0.00 | 4.33 | 1.06 |
| GTGGTATGGC | AF151076 | hypothetical protein | 0.18 | 0.19 | 0.98 | 0.36 | 0.98 | 1.06 | 0.33 | 0.97 | 5.44 | 1.06 |
| GCCAAGATGC | BC004944 | Similar to RIKEN cDNA 2310040G17 gene | 0.18 | 0.58 | 1.18 | 1.44 | 4.50 | 2.48 | 1.63 | 1.29 | 6.56 | 3.22 |
| TCAGTTTGTC | U68566 | HAX1; HS1 binding protein | 0.37 | 0.39 | 1.96 | 1.63 | 2.74 | 2.13 | 0.98 | 1.29 | 5.30 | 1.05 |
| TTGGGGTTCC | Hs.182771 | vitelliform macular dystrophy (Best disease, bestrophin) or retinoblastoma-binding protein [symbol] or | 0.18 | 0.19 | 0.78 | 0.72 | 0.78 | 1.77 | 1.63 | 1.29 | 4.33 | 1.06 |
| | | ferritin, heavy polypeptide 1 | | | | | | | | | | |
| GTATCTTAAT | AF155568 | NS1-associated protein 1 | 0.18 | 0.00 | 0.78 | 0.00 | 0.20 | 0.00 | 0.65 | 0.00 | 4.33 | 0.00 |
| TGGGGAAACT | X52333 | APMCF1 protein (EST) | 0.18 | 0.00 | 0.98 | 0.18 | 0.20 | 0.00 | 0.00 | 0.00 | 5.44 | 0.00 |
| ATGAATAAAA | X96650 | microtubule-associated protein, RP/EB family, member 2 or Rho GTPase activating protein 6 | 0.18 | 0.00 | 0.78 | 0.00 | 0.20 | 0.00 | 0.00 | 0.00 | 4.33 | 0.00 |
| GCTAAAAACA | Hs.8107 | Homo sapiens mRNA; cDNA DKFZp586B0918 (from clone DKFZp586B0918) | 0.18 | 0.00 | 0.78 | 0.54 | 0.00 | 0.35 | 0.00 | 0.00 | 4.33 | 0.00 |
| AAGTTGCATC | Hs.29131 | nuclear receptor coactivator 2 | 0.18 | 0.00 | 0.78 | 0.18 | 0.20 | 0.18 | 0.00 | 0.00 | 4.33 | 0.00 |
| TAACCAAACA | AJ243671 | KIAA0144 gene product | 0.18 | 0.00 | 0.78 | 0.18 | 0.39 | 0.00 | 0.00 | 0.00 | 4.33 | 0.00 |
| GGCAGCCTGG | U94836 | protein with polyglutamine repeat; calcium (ca2+) homeostasis endoplasmic reticulum protein | 0.18 | 0.00 | 0.78 | 0.18 | 0.39 | 0.00 | 0.65 | 0.64 | 4.33 | 0.00 |
| GCACAAGTAA | AF339775 | unknown | 0.18 | 0.00 | 0.98 | 0.90 | 0.20 | 0.35 | 0.65 | 0.00 | 5.44 | 0.00 |
| GTGAACTTAC | AB013803 | contactin 5; NB-2; hNB-2 | 0.18 | 0.00 | 1.37 | 0.36 | 0.59 | 0.18 | 0.98 | 0.00 | 7.61 | 0.00 |
| GCAAACAAAA | Hs.429 | ATP synthase, H+ transporting, mitochondrial F0 complex, subunit c (subunit 9) isoform 3 | 0.18 | 0.00 | 0.98 | 0.18 | 0.59 | 0.71 | 0.00 | 0.00 | 5.44 | 0.00 |
| ACAAATCCTT | M34539 | FK506-binding protein 1A (12 kD) | 0.18 | 0.00 | 0.98 | 0.72 | 1.96 | 0.00 | 2.28 | 0.64 | 5.44 | 0.00 |
| ATGCAAGAGA | AB051504 | KIAA1717 protein | 0.18 | 0.00 | 1.18 | 0.00 | 0.00 | 0.00 | 0.00 | 0.00 | 6.56 | 0.00 |
| GAGTGCAGGT | M25077 | Sjogren syndrome antigen A2 (60 kD, ribonucleoprotein autoantigen SS-A/Ro) | 0.18 | 0.00 | 0.98 | 0.18 | 0.00 | 0.00 | 0.00 | 0.32 | 5.44 | 0.00 |
| TATCACTTTT | Z11898 | POU domain, class 5, transcription factor 1 | 0.18 | 0.00 | 0.78 | 0.36 | 0.00 | 0.00 | 0.00 | 0.32 | 4.33 | 0.00 |
| CTACTGTCTA | Hs.108124 | ribosomal protein L41 | 0.18 | 0.00 | 0.78 | 0.00 | 0.00 | 0.00 | 0.65 | 0.64 | 4.33 | 0.00 |
| CCAGTGTGCA | Hs.249247 | heterogeneous nuclear protein similar to rat helix destabilizing protein | 0.18 | 0.00 | 1.18 | 0.36 | 0.00 | 0.00 | 0.00 | 0.00 | 6.56 | 0.00 |
| GTGCTATTCT | AL360136 | B7 homolog 3 | 0.18 | 0.00 | 0.78 | 0.90 | 1.17 | 1.06 | 0.98 | 0.00 | 4.33 | 0.00 |
| GAGCTGGTGA | NM_005830 | imogen 38 | 0.18 | 0.00 | 0.78 | 0.54 | 1.17 | 1.42 | 0.98 | 0.64 | 4.33 | 0.00 |
| GTCTGACCCC | Hs.173902 | protein phosphatase 2 (formerly 2A), regulatory subunit A (PR 65), alpha isoform | 0.37 | 0.00 | 1.57 | 0.54 | 3.13 | 2.48 | 0.98 | 1.29 | 4.24 | 0.00 |

| | | | | | | | | | | | | |
|---|---|---|---|---|---|---|---|---|---|---|---|---|
| | Accession/ | | | | | | | | | | V2/V5, | V1/V5, |
| SAGE Tag | UniGene ID | Description | V1 | V2 | V3 | V4 | V5 | V6 | V7 | V8 | V6 | V6 |

416 genes in FIG. 2(c) (369 genes; 47 tags with no matches not shown)
SAGE data for genes up-regulated > 4-fold in 786-0 VHL-Nor. (V2) when compared to the average of two independent cultures of
Renal Proximal Tubule cells (RPTECs) grown under nomoxia (V5, V6). Also shown, for comparison, is the SAGE data for each gene
in the 786-0 VHL⁺ Nor. (V1) with respect to its average expression in the two independent cultures of Renal Proximal Tubule
cells (RPTECs) grown under nomoxia (V5, V6). SAGE tag abundances were normalized before calculating ratios, as shown. The table
is presented in descending order of V2/V5, V6.

| | | | | | | | | | | | | |
|---|---|---|---|---|---|---|---|---|---|---|---|---|
| GCCACGTTGT | AL137588 | HOG3; hypothetical protein DKFZp434K1210 | 0.00 | 1.16 | 0.59 | 1.44 | 0.00 | 0.18 | 0.98 | 1.61 | 12.89 | 0.00 |
| ACCTAGCCAC | U11058 | potassium large conductance calcium-activated channel, subfamily M, alpha member 1 | 0.37 | 1.16 | 0.39 | 0.00 | 0.20 | 0.00 | 0.65 | 0.97 | 11.60 | 3.70 |
| ATTTTTTAAC | AF100747 | HSPCO34 protein | 0.18 | 1.16 | 0.39 | 0.00 | 0.20 | 0.00 | 0.00 | 0.00 | 11.60 | 1.80 |
| GACTGTTGCT | AK000109 | transporter-like protein (EST) | 0.00 | 1.16 | 0.20 | 0.36 | 0.20 | 0.00 | 0.33 | 0.32 | 11.60 | 0.00 |
| TGGAGGTGGG | AF086628 | VAMP (vesicle-associated membrane protein)-associated protein B and C | 0.18 | 0.97 | 0.39 | 0.36 | 0.00 | 0.18 | 1.30 | 0.32 | 10.78 | 2.00 |
| TCTGTGCTCA | AK000006 | hypothetical protein | 0.18 | 0.97 | 0.39 | 0.36 | 0.00 | 0.18 | 0.00 | 0.00 | 10.78 | 2.00 |
| GAGTGAAAGA | AK000474 | chromosome 21 open reading frame 59 | 0.18 | 0.97 | 0.20 | 0.18 | 0.00 | 0.18 | 0.33 | 0.32 | 10.78 | 2.00 |
| CTCAACCCCC | X13916 | low density lipoprotein-related protein 1 (alpha-2-macroglobulin receptor) | 0.18 | 0.97 | 0.00 | 0.54 | 0.00 | 0.18 | 1.63 | 0.97 | 10.78 | 2.00 |
| ATTAAAGTGC | AK000903 | EST | 0.00 | 0.97 | 0.00 | 0.18 | 0.00 | 0.18 | 0.33 | 0.00 | 10.78 | 0.00 |
| TTGCTTCTTT | AL096677 | NTF2-related export protein 1 | 0.00 | 0.97 | 0.20 | 0.36 | 0.00 | 0.18 | 0.00 | 0.00 | 10.78 | 0.00 |
| TACACAGAAC | Hs.24283 | ESTs, Moderately similar to reduced expression in cancer [H. sapiens] | 0.00 | 0.97 | 0.00 | 0.00 | 0.00 | 0.18 | 0.00 | 0.32 | 10.78 | 0.00 |
| CCCCTGCACT | Hs.155485 | huntingtin interacting protein 2 or enhancer of invasion 10 or transforming, acidic coiled-coil containing | 0.00 | 0.97 | 0.00 | 0.00 | 0.00 | 0.18 | 0.00 | 0.00 | 10.78 | 0.00 |
| | | protein 1 or ESTs | | | | | | | | | | |
| GTTTTTGCTT | Hs.79110 | nucleolin | 0.74 | 2.13 | 1.57 | 0.72 | 0.39 | 0.00 | 1.30 | 0.00 | 10.65 | 3.79 |
| CCAGTGTCTG | AF074993 | lysophospholipase I (EST) | 0.37 | 0.97 | 0.78 | 1.99 | 0.20 | 0.00 | 0.33 | 0.97 | 9.70 | 3.70 |
| TGAATAAAGT | U96094 | sarcolipin | 0.37 | 0.97 | 0.39 | 0.72 | 0.20 | 0.00 | 0.00 | 0.00 | 9.70 | 3.70 |
| ACTACAAATA | Hs.157850 | ribosomal protein L9 (EST) | 0.37 | 0.97 | 0.20 | 0.36 | 0.20 | 0.00 | 0.65 | 0.64 | 9.70 | 3.70 |
| AGCAAGCAAG | Hs.156764 | RAP1B, member of RAS oncogene family (EST) | 0.37 | 0.97 | 0.39 | 0.00 | 0.20 | 0.00 | 0.33 | 0.00 | 9.70 | 3.70 |
| CAGTAGCCTT | Hs.9589 | ubiquilin 1 (EST) | 0.37 | 0.97 | 0.00 | 0.00 | 0.20 | 0.00 | 0.00 | 0.00 | 9.70 | 3.70 |
| CTCACTTCTT | Hs.165998 | PAI-1 mRNA-binding protein (EST) | 0.18 | 0.97 | 0.98 | 0.90 | 0.20 | 0.00 | 0.98 | 0.00 | 9.70 | 1.80 |
| CCTCACTTTC | M94859 | calnexin | 0.18 | 0.97 | 0.39 | 0.00 | 0.20 | 0.00 | 0.33 | 0.32 | 9.70 | 1.80 |
| TGTGTCAAAG | Hs.181163 | hypothetical protein MGC5629 (EST) | 0.18 | 0.97 | 0.59 | 0.90 | 0.20 | 0.00 | 0.00 | 0.00 | 9.70 | 1.80 |
| CCCTTCTATT | Hs.12492 | CMP-N-acetylneuraminic acid synthase (EST) | 0.00 | 0.97 | 0.59 | 0.18 | 0.20 | 0.00 | 0.00 | 0.32 | 9.70 | 0.00 |
| GGAATCCAAT | AJ223953 | PTTG1; Pituitary tumor-transforming 1; tumor transforming protein 1 (TUTR1) | 0.55 | 1.74 | 0.20 | 1.99 | 0.20 | 0.18 | 0.00 | 0.32 | 9.16 | 2.89 |
| TGGCTTAAAT | AF144755 | HIG2; hypoxia-inducible protein 2 | 0.37 | 1.74 | 0.78 | 2.53 | 0.20 | 0.18 | 1.63 | 5.47 | 9.16 | 1.95 |
| TTGTAAATGC | AF332224 | testis protein mRNA | 0.55 | 1.74 | 1.57 | 0.90 | 0.39 | 0.00 | 0.33 | 0.32 | 8.70 | 2.82 |
| GCTAGGTATT | AJ250865 | testin | 0.18 | 0.77 | 0.98 | 1.08 | 0.00 | 0.18 | 0.33 | 0.00 | 8.56 | 2.00 |
| ATCCCACTGC | Hs.38114 | hypothetical protein FLJ11100 or EST | 0.18 | 0.77 | 0.00 | 0.54 | 0.00 | 0.18 | 0.00 | 0.00 | 8.56 | 2.00 |
| TCTTTCACCC | D88674 | antizyme inhibitor | 0.18 | 0.77 | 0.39 | 0.54 | 0.00 | 0.18 | 0.33 | 0.00 | 8.56 | 2.00 |
| TTTTTGTTTT | Hs.75772 | 11nt = c; EST (S checked) | 0.18 | 0.77 | 0.00 | 0.00 | 0.00 | 0.18 | 0.00 | 0.00 | 8.56 | 2.00 |
| TGTGAACACA | X14454 | interferon regulatory factor 1 | 0.18 | 0.77 | 0.39 | 0.00 | 0.00 | 0.18 | 0.33 | 0.00 | 8.56 | 2.00 |
| GACAGCCTTA | Hs.153221 | synovial sarcoma, translocated to X chromosome (EST) | 0.18 | 0.77 | 0.20 | 0.54 | 0.00 | 0.18 | 0.00 | 0.32 | 8.56 | 2.00 |
| TGCAATATGG | Hs.173912 | eukaryotic translation initiation factor 4A, isoform 2 (EST) | 0.18 | 0.77 | 0.39 | 0.36 | 0.00 | 0.18 | 0.00 | 0.32 | 8.56 | 2.00 |
| GTGGGGGGGA | Hs.10700 | hypothetical protein (EST) | 0.18 | 0.77 | 0.00 | 0.54 | 0.00 | 0.18 | 0.00 | 0.32 | 8.56 | 2.00 |
| GGCAACAGAG | AF009284 | EST | 0.00 | 0.77 | 0.20 | 0.36 | 0.00 | 0.18 | 0.98 | 0.64 | 8.56 | 0.00 |
| AAGACAGCGG | Hs.153792 | 5-methyltetrahydrofolate-homocysteine methyltransferase reductase (EST) | 0.00 | 0.77 | 0.20 | 0.18 | 0.00 | 0.18 | 0.65 | 0.00 | 8.56 | 0.00 |
| TCCTTATTAA | Hs.148589 | EST | 0.00 | 0.77 | 0.39 | 0.54 | 0.00 | 0.18 | 0.65 | 0.32 | 8.56 | 0.00 |
| AGTGCCCTGA | Hs.6823 | hypothetical protein FLJ10430 or nGAP-like protein (EST) | 0.00 | 0.77 | 0.00 | 0.72 | 0.00 | 0.18 | 0.00 | 0.00 | 8.56 | 0.00 |
| GATAGTTGTG | AF274949 | thyroid hormone receptor interactor 7 | 1.29 | 2.90 | 2.94 | 2.71 | 0.00 | 0.71 | 0.33 | 0.32 | 8.29 | 3.63 |
| GTATAATTTG | BC007341 | small membrane protein 1 | 0.37 | 1.55 | 0.59 | 0.90 | 0.39 | 0.00 | 0.65 | 0.00 | 7.75 | 1.90 |
| TTTGGAAATC | AF100755 | CGI-99 protein | 0.37 | 0.77 | 0.20 | 0.54 | 0.20 | 0.00 | 0.00 | 0.00 | 7.70 | 3.70 |
| CTGCCCTAGT | AL050346 | Novel human gene mapping to chomosome 22 | 0.37 | 0.77 | 0.20 | 0.36 | 0.20 | 0.00 | 0.98 | 0.32 | 7.70 | 3.70 |
| GATGTAGTGG | M81750 | myeloid cell nuclear differentiation antigen | 0.37 | 0.77 | 0.98 | 0.72 | 0.20 | 0.00 | 0.00 | 0.00 | 7.70 | 3.70 |
| GTAAAACCCT | AF232009 | HSA250303; peroxisomal trans 2-enoyl CoA reductase; putative short chain alcohol dehydrogenase | 0.18 | 0.77 | 0.78 | 0.00 | 0.20 | 0.00 | 0.00 | 0.32 | 7.70 | 1.80 |
| AACTGTCCTT | L37385 | PEA15; phosphoprotein enriched in astrocytes 15 | 0.18 | 0.77 | 0.00 | 0.18 | 0.20 | 0.00 | 0.98 | 0.32 | 7.70 | 1.80 |
| CTTCTGGTGC | Hs.75431 | fibrinogen, gamma polypeptide (EST) | 0.18 | 0.77 | 0.59 | 0.36 | 0.20 | 0.00 | 0.65 | 1.61 | 7.70 | 1.80 |
| CCTGTCATCC | AL133642 | RNB6 | 0.00 | 0.77 | 0.39 | 0.00 | 0.20 | 0.00 | 0.33 | 0.00 | 7.70 | 0.00 |
| GCCTTGCCTG | D87470 | KIAA0280 protein | 0.00 | 0.77 | 0.20 | 0.18 | 0.20 | 0.00 | 0.33 | 0.32 | 7.70 | 0.00 |
| GTAGCTTATG | Hs.76507 | PIG7; LPS-induced TNF-alpha factor (EST) | 0.00 | 0.77 | 0.00 | 0.18 | 0.20 | 0.00 | 0.33 | 0.00 | 7.70 | 0.00 |
| TGTTATTAAA | Hs.249247 | heterogeneous nuclear protein similar to rat helix destabilizing protein (EST) or glycerol-3-phosphate | 0.00 | 0.77 | 0.20 | 0.36 | 0.20 | 0.00 | 0.33 | 0.00 | 7.70 | 0.00 |
| | | dehydrogenase 2 (mitochondr | | | | | | | | | | |
| GTTCTGGGCG | Hs.286049 | solute carrier family 1 (glutamate/neutral amino acid transporter), member 4 (EST) or phosphoserine | 0.00 | 0.77 | 0.20 | 0.36 | 0.20 | 0.00 | 0.00 | 0.00 | 7.70 | 0.00 |
| | | aminotransferase (EST) | | | | | | | | | | |
| TCCTTGCTTC | AB037839 | hypothetical protein FLJ20297 | 1.48 | 2.90 | 0.98 | 1.99 | 0.59 | 0.18 | 0.33 | 0.00 | 7.63 | 3.84 |
| CTGTGTAAGC | D13892 | protein-L-isoaspartate (D-aspartate) O-methyltransferase | 0.74 | 1.35 | 1.37 | 1.26 | 0.20 | 0.18 | 0.33 | 0.00 | 7.11 | 3.89 |
| CAGCCGTGAT | M86707 | N-myristoyltransferase 1 | 0.74 | 1.35 | 1.37 | 0.54 | 0.20 | 0.18 | 0.65 | 0.97 | 7.11 | 3.89 |
| TCTTGAACAG | AF196186 | three-PDZ containing protein similar to C. elegans PAR3 (partitioning defect) | 1.11 | 1.93 | 1.57 | 1.44 | 0.39 | 0.18 | 0.00 | 0.00 | 6.89 | 3.89 |
| TTTTAAACTT | Hs.226770 | DKFZP566C0424 protein (EST) | 0.55 | 1.93 | 1.96 | 0.18 | 0.20 | 0.35 | 0.33 | 0.64 | 6.89 | 2.00 |
| GGATTCCAGT | AF127773 | ARP3 (actin-related protein 3, yeast) homolog | 0.55 | 1.35 | 0.59 | 0.36 | 0.39 | 0.00 | 0.33 | 0.00 | 6.75 | 2.82 |
| AGTGGGTATT | AF132968 | CGI-34 protein or hypothetical protein | 0.55 | 1.16 | 0.20 | 0.54 | 0.00 | 0.35 | 0.33 | 0.64 | 6.44 | 3.14 |
| TTCACTTATG | AL110280 | cDNA FLJ11651 fis, clone HEMBA1004460 | 0.55 | 1.16 | 0.59 | 0.36 | 0.00 | 0.35 | 0.65 | 0.32 | 6.44 | 3.14 |
| GTGTCAGATG | AB019572 | No | 0.18 | 0.58 | 0.20 | 0.00 | 0.00 | 0.18 | 0.33 | 0.32 | 6.44 | 2.00 |
| CCCCCAATGC | AC005263 | splicing factor 3a, subunit 2, 66 kD | 0.18 | 0.58 | 0.00 | 0.36 | 0.00 | 0.18 | 0.33 | 0.97 | 6.44 | 2.00 |
| TGTGACCTCT | AF061729 | dolichyl-phosphate mannosyltransferase polypeptide 2, regulatory subunit | 0.18 | 0.58 | 0.78 | 0.18 | 0.00 | 0.18 | 0.00 | 0.00 | 6.44 | 2.00 |
| AAAACAAAAC | AF185696 | oxysterol binding protein | 0.18 | 0.58 | 0.00 | 0.00 | 0.00 | 0.18 | 0.00 | 0.00 | 6.44 | 2.00 |
| AGAAGCAAGA | AF208110 | IL-17B receptor | 0.18 | 0.58 | 0.20 | 0.18 | 0.00 | 0.18 | 0.00 | 0.00 | 6.44 | 2.00 |
| AGTAAACCAT | AL050192 | cDNA DKFZp586C1723 (from clone DKFZp586C1723) | 0.37 | 1.16 | 0.00 | 0.72 | 0.00 | 0.35 | 0.65 | 0.00 | 6.44 | 2.11 |
| CTGACGGGTA | AL133609 | hypothetical protein DKFZp434L0117 | 0.18 | 0.58 | 0.20 | 0.36 | 0.00 | 0.18 | 0.00 | 0.00 | 6.44 | 2.00 |
| ATTTAAAAAA | BC008801 | cold shock domain protein A | 0.18 | 0.58 | 0.00 | 0.18 | 0.00 | 0.18 | 0.00 | 0.00 | 6.44 | 2.00 |
| TTTATTTAAG | D21235 | RAD23 (S. cerevisiae) homolog A | 0.18 | 0.58 | 0.59 | 0.36 | 0.00 | 0.18 | 0.00 | 0.00 | 6.44 | 2.00 |
| GAAGCAGGGC | Hs.180370 | cofilin 1 (non-muscle) (EST) | 0.18 | 0.58 | 0.20 | 0.54 | 0.00 | 0.18 | 0.00 | 0.32 | 6.44 | 2.00 |
| CCAACTCTCA | Hs.182447 | heterogeneous nuclear ribonucleoprtotein C (C1/C2) (EST) | 0.18 | 0.58 | 0.20 | 0.00 | 0.00 | 0.18 | 0.98 | 0.64 | 6.44 | 2.00 |
| GATGTGGACA | Hs.251653 | tubulin, beta, 2 (EST) or DR1-associated protein 1 (negative cofactor 2 alpha) (EST) | 0.18 | 0.58 | 0.39 | 0.36 | 0.00 | 0.18 | 0.00 | 0.00 | 6.44 | 2.00 |
| ATGGTCAGTA | Hs.287955 | EST | 0.18 | 0.58 | 0.39 | 0.54 | 0.00 | 0.18 | 0.98 | 0.00 | 6.44 | 2.00 |
| TATGCCCTAT | Hs.115740 | KIAA0210 gene product (EST) | 0.18 | 0.58 | 0.59 | 0.18 | 0.00 | 0.18 | 0.00 | 0.00 | 6.44 | 2.00 |
| CAAGTATACC | Hs.22026 | Weakly similar to A42926 L6 surface protein [H. sapiens] (EST) | 0.18 | 0.58 | 0.59 | 0.00 | 0.00 | 0.18 | 0.00 | 0.00 | 6.44 | 2.00 |
| AAGACAAGTG | Hs.296290 | ribosomal protein L37a (EST) | 0.18 | 0.58 | 0.78 | 0.18 | 0.00 | 0.18 | 0.00 | 0.00 | 6.44 | 2.00 |
| TGAGGGATGG | Hs.172740 | microtubule-associated protein, RP/EB family, member 3 (EST) or others | 0.18 | 0.58 | 0.00 | 0.18 | 0.00 | 0.18 | 0.33 | 0.32 | 6.44 | 2.00 |
| GCACCATAAT | Hs.43910 | CD164 antigen, sialomucin (EST) | 0.18 | 0.58 | 0.00 | 0.72 | 0.00 | 0.18 | 0.33 | 0.97 | 6.44 | 2.00 |
| AGTTCAAGAC | Hs.37558 | hypothetical protein FLJ11149 (EST) or F-box only protein 9 (EST) or others | 0.18 | 0.58 | 0.00 | 0.00 | 0.00 | 0.18 | 0.33 | 0.00 | 6.44 | 2.00 |
| GAGTTTGTGT | AB032969 | KIAA1143 protein | 0.00 | 0.58 | 0.00 | 0.36 | 0.00 | 0.18 | 0.00 | 0.00 | 6.44 | 0.00 |
| CAAATAAATG | AF035292 | KIAA0657 protein | 0.00 | 0.58 | 0.59 | 0.18 | 0.00 | 0.18 | 0.33 | 0.64 | 6.44 | 0.00 |
| CTGCAAGGAC | AF161413 | hypothetical protein MGC3036 or HSPC295 mRNA, partial cds | 0.00 | 0.58 | 0.20 | 0.72 | 0.00 | 0.18 | 0.33 | 0.00 | 6.44 | 0.00 |
| CTGGCCATCG | AK024451 | ITGB1; Integrin, beta 1 (fibronectin receptor, beta polypeptide, antigen CD29 includes MDF2, MSK12) | 0.00 | 0.58 | 0.00 | 0.00 | 0.00 | 0.18 | 0.00 | 0.00 | 6.44 | 0.00 |
| GGGCCAGGAA | BC007856 | MC1R; melanocortin 1 receptor (alpha melanocyte stimulating hormone receptor) | 0.00 | 0.58 | 0.00 | 0.18 | 0.00 | 0.18 | 0.33 | 0.00 | 6.44 | 0.00 |
| CTGGGATCAT | BC009111 | clone MGC: 17296, mRNA, complete cds | 0.00 | 0.58 | 0.59 | 0.00 | 0.00 | 0.18 | 0.00 | 0.00 | 6.44 | 0.00 |
| GCACCTTATT | NM_032822 | hypothetical protein FLJ14668 | 0.00 | 0.58 | 0.78 | 0.54 | 0.00 | 0.18 | 0.33 | 0.32 | 6.44 | 0.00 |
| GAAGCAATAA | X00033 K01 | major histocompatibility complex, class II, DQ alpha 1 | 0.00 | 0.58 | 0.20 | 0.90 | 0.00 | 0.18 | 0.00 | 0.00 | 6.44 | 0.00 |
| GGAAGAGCAC | X74570 | SIAT4C; sialyltransferase 4C (beta-galactosidase alpha-2,3-sialytransferase) | 0.00 | 0.58 | 0.00 | 0.18 | 0.00 | 0.18 | 0.00 | 0.32 | 6.44 | 0.00 |
| GTGGCAGTGG | Hs.16478 | EST | 0.00 | 0.58 | 0.20 | 0.18 | 0.00 | 0.18 | 0.00 | 0.32 | 6.44 | 0.00 |
| ACCCTGTAGA | Hs.16059 | HSPC009 protein (EST) | 0.00 | 0.58 | 0.00 | 0.18 | 0.00 | 0.18 | 0.33 | 0.00 | 6.44 | 0.00 |
| CCTGAAAACT | Hs.90286 | EST | 0.00 | 0.58 | 0.39 | 0.18 | 0.00 | 0.18 | 0.00 | 0.00 | 6.44 | 0.00 |
| CTGGGCAACA | Hs.105547 | NPDC1; neural proliferation, differentiation and control, 1 (EST) or KIAA1271 protein (EST) | 0.00 | 0.58 | 0.20 | 0.18 | 0.00 | 0.18 | 0.00 | 0.00 | 6.44 | 0.00 |
| GTTCATCCTT | Hs.236894 | Highly similar to S02392 alpha-2-macroglobulin receptor precursor [H. sapiens] (EST) | 0.00 | 0.58 | 0.20 | 0.36 | 0.00 | 0.18 | 0.00 | 0.32 | 6.44 | 0.00 |
| TCAGTGTGTG | Hs.27099 | hypothetical protein FLJ23293 similar to ARL-6 interacting protein-2 (EST) or lecithin retinol | 0.00 | 0.58 | 0.39 | 0.36 | 0.00 | 0.18 | 0.00 | 0.00 | 6.44 | 0.00 |
| | | acyltransferase (phosphatidylcholine-n | | | | | | | | | | |
| CTGGGCAAAC | Hs.77266 | qulescin Q6 (EST) | 0.00 | 0.58 | 0.00 | 0.36 | 0.00 | 0.18 | 0.00 | 0.00 | 6.44 | 0.00 |
| AAGAGAGCTT | Hs.155546 | KIAA1080 protein; Golgi-associated, gamma-adaptin ear containing, ARF-binding protein 2 (EST) | 0.00 | 0.58 | 0.00 | 0.54 | 0.00 | 0.18 | 0.00 | 0.32 | 6.44 | 0.00 |
| TGTAAGGCAC | Hs.77266 | quiescin Q6 (EST) | 0.00 | 0.58 | 0.20 | 0.00 | 0.00 | 0.18 | 0.00 | 0.32 | 6.44 | 0.00 |
| TTAGAAACAG | Hs.100748 | EST | 0.00 | 0.58 | 0.00 | 0.00 | 0.00 | 0.18 | 0.33 | 0.00 | 6.44 | 0.00 |
| GCCTTCCGTG | Hs.169476 | GAPD; glyceraldehyde-3-phosphate dehydrogenase (EST) | 0.00 | 0.58 | 0.39 | 0.00 | 0.00 | 0.18 | 0.33 | 1.29 | 6.44 | 0.00 |
| TACAATAAAC | AJ002030 | progesterone membrane binding protein | 1.29 | 2.90 | 2.55 | 3.43 | 0.39 | 0.53 | 1.30 | 0.64 | 6.30 | 2.80 |
| CAGGAACCAC | Hs.296290 | ribosomal protein L37a (EST) | 0.74 | 1.74 | 1.18 | 0.90 | 0.39 | 0.18 | 0.33 | 0.97 | 6.21 | 2.60 |
| CTGTTGGCAT | Hs.184108 | ribosomal protein L21 (gene or pseudogene) | 0.18 | 1.74 | 0.78 | 1.08 | 0.20 | 0.35 | 0.33 | 1.61 | 6.21 | 0.65 |
| AGGACAATGC | BC005809 | dual-specificity tyrosine-(Y)-phosphorylation regulated kinase 2 | 0.74 | 1.16 | 0.59 | 0.36 | 0.20 | 0.18 | 0.33 | 0.00 | 6.11 | 3.89 |
| CTCACAAGGG | Hs.129953 | Ewing sarcoma breakpoint region 1 (EST) | 0.55 | 1.16 | 0.20 | 0.54 | 0.20 | 0.18 | 0.33 | 0.32 | 6.11 | 2.89 |
| AGAGGTGGTG | Hs.272168 | tumor differentially expressed 1 (EST) | 0.55 | 1.16 | 0.98 | 1.44 | 0.20 | 0.18 | 0.00 | 1.29 | 6.11 | 2.89 |
| ATAAAGCTAC | NM_012151 | coagulation factor VIII-associated (Inronic transcript) | 0.37 | 1.16 | 1.18 | 0.18 | 0.20 | 0.18 | 0.33 | 0.00 | 6.11 | 1.95 |
| CCTGTAAAGC | Hs.9691 | Homo sapiens cDNA: FLJ23249 fis, clone COL04196 (EST) | 0.18 | 1.16 | 0.59 | 0.72 | 0.20 | 0.18 | 1.63 | 3.86 | 6.11 | 0.95 |
| GCCTCTTGAA | S72008 | hCDC10 = CDC10 homolog [human, fetal lung, mRNA, 231 | 1.11 | 1.74 | 1.37 | 1.99 | 0.59 | 0.00 | 0.98 | 0.32 | 6.00 | 3.76 |
| TTAAAAGCCT | X54941 X55 | CDC28 protein kinase 1 | 1.48 | 2.70 | 1.76 | 3.25 | 0.20 | 0.71 | 0.00 | 0.32 | 6.00 | 3.25 |
| TTCTAACATA | X03747 | ATPase, Na+/K+ transporting, beta 1 polypeptide | 0.92 | 2.13 | 0.78 | 3.43 | 0.20 | 0.53 | 0.33 | 1.29 | 5.92 | 2.52 |
| GACTTGGAGG | AF086057 | membrane-associated tyrosine- and threonine-specific cdc2-inhibitory kinase (EST) | 0.37 | 2.13 | 1.18 | 0.90 | 0.20 | 0.53 | 0.00 | 0.00 | 5.92 | 1.01 |
| GCAAAACTCC | AB007968 | CEACAM1; carcinoembryonic antigen-related cell adhesion molecule 1 (biliary glycoprotein) | 0.37 | 0.58 | 0.00 | 0.54 | 0.20 | 0.00 | 0.33 | 0.00 | 5.80 | 3.70 |
| TTTCTGTTAA | AF151043 | hypothetical protein | 0.37 | 0.58 | 0.20 | 0.54 | 0.20 | 0.00 | 0.00 | 0.00 | 5.80 | 3.70 |
| CAAGCCAAAA | AK001241 | Homo sapiens cDNA FLJ10379 fis, clone NT2RM2002014 | 0.37 | 0.58 | 0.20 | 0.00 | 0.20 | 0.00 | 0.00 | 0.00 | 5.80 | 3.70 |
| TTTAGGTAAA | AL136807 | SERP1; stress-associated endoplasmic reticulum protein 1; ribosome associated membrane protein 4 | 0.37 | 0.58 | 0.20 | 0.36 | 0.20 | 0.00 | 0.65 | 0.00 | 5.80 | 3.70 |
| ACCTTGCTGG | U35139 | necdln (mouse) homolog | 0.37 | 0.58 | 0.20 | 0.00 | 0.20 | 0.00 | 0.33 | 0.32 | 5.80 | 3.70 |
| CTCAAGTAAT | Hs.82916 | chaperonin containing TCP1, subunit 6A (zeta 1) (EST) | 0.37 | 0.58 | 0.20 | 0.18 | 0.20 | 0.00 | 0.00 | 0.00 | 5.80 | 3.70 |
| CGCCGAGCAC | Hs.123426 | serine/threonine kinase 22C (spermiogenesis associated) | 0.37 | 0.58 | 0.39 | 0.54 | 0.20 | 0.00 | 0.00 | 0.00 | 5.80 | 3.70 |
| CAGGGGAAGG | Hs.302435 | centaurin, gamma 1 | 0.37 | 0.58 | 0.39 | 0.00 | 0.20 | 0.00 | 0.33 | 0.32 | 5.80 | 3.70 |
| CTGAATTCAG | Hs.28739 | EST | 0.37 | 0.58 | 0.39 | 0.00 | 0.20 | 0.00 | 0.00 | 0.00 | 5.80 | 3.70 |
| GGAGACAGAG | Hs.210850 | KIAA1131 protein (EST) or other ESTs | 0.37 | 0.58 | 0.59 | 0.18 | 0.20 | 0.00 | 0.33 | 0.32 | 5.80 | 3.70 |
| GCACTGAAAA | Hs.279607 | calpastatin (EST) | 0.37 | 0.58 | 0.00 | 0.72 | 0.20 | 0.00 | 0.00 | 0.00 | 5.80 | 3.70 |
| GAAATGAGCA | D50917 | KIAA0127 gene product | 0.55 | 1.16 | 1.18 | 1.08 | 0.39 | 0.00 | 0.00 | 2.90 | 5.80 | 2.82 |
| ATGATGCCTC | AB018345 | KIAA0802 protein | 0.18 | 0.58 | 0.59 | 1.26 | 0.20 | 0.00 | 0.33 | 0.64 | 5.80 | 1.80 |
| TAACCTCAGG | AB020677 | KIAA0870 protein | 0.18 | 0.58 | 0.59 | 0.54 | 0.20 | 0.00 | 0.00 | 0.00 | 5.80 | 1.80 |
| TGCGTACCAC | AF132952 | CGI-18 protein | 0.18 | 0.58 | 0.39 | 0.00 | 0.20 | 0.00 | 0.00 | 0.00 | 5.80 | 1.80 |
| TCTGCATTTG | AF225981 | ATPase, Ca++ transporting, type 2C, member 1 | 0.18 | 0.58 | 0.00 | 0.00 | 0.20 | 0.00 | 0.00 | 0.00 | 5.80 | 1.80 |
| TTACAGAGCT | AF265215 | zinc finger protein 313 | 0.18 | 0.58 | 0.59 | 0.00 | 0.20 | 0.00 | 0.00 | 0.97 | 5.80 | 1.80 |
| ACACTTTTTT | AK001217 | hypothetical protein FLJ10355 | 0.18 | 0.58 | 0.20 | 0.18 | 0.20 | 0.00 | 0.00 | 0.00 | 5.80 | 1.80 |
| GACAGTGACG | D45213 | zinc finger protein | 0.18 | 0.58 | 0.20 | 0.36 | 0.20 | 0.00 | 0.00 | 0.64 | 5.80 | 1.80 |
| TTCTATTTTG | U82938 | CD27-binding (Siva) protein | 0.37 | 1.16 | 0.59 | 0.54 | 0.39 | 0.00 | 0.00 | 0.00 | 5.80 | 1.90 |
| GTTTAAATGG | Hs.203148 | EST | 0.18 | 0.58 | 0.20 | 0.18 | 0.20 | 0.00 | 0.00 | 0.00 | 5.80 | 1.80 |
| CTAGACAGTA | Hs.52526 | KIAA0669 gene product (EST) | 0.18 | 0.58 | 0.20 | 0.36 | 0.20 | 0.00 | 0.00 | 0.32 | 5.80 | 1.80 |
| CACCTAATTT | Hs.245035 | EST | 0.18 | 0.58 | 0.20 | 0.00 | 0.20 | 0.00 | 0.33 | 0.00 | 5.80 | 1.80 |
| CTTCTCTCTC | Hs.343211 | CD2-associated protein (EST) | 0.18 | 0.58 | 0.39 | 0.00 | 0.20 | 0.00 | 0.00 | 0.00 | 5.80 | 1.80 |
| ACACCCAGAA | Hs.164866 | hypothetical protein FLJ22558 (EST) | 0.18 | 0.58 | 0.39 | 0.00 | 0.20 | 0.00 | 0.00 | 0.00 | 5.80 | 1.80 |
| CCTTCTCACT | Hs.20013 | GCIP-interacting protein p29 (EST) | 0.18 | 0.58 | 0.59 | 0.00 | 0.20 | 0.00 | 0.33 | 0.97 | 5.80 | 1.80 |
| GGGGGGGGGG | Hs.108530 | hypothetical protein FLJ10856 (EST) or other ESTs | 0.18 | 0.58 | 0.00 | 0.00 | 0.20 | 0.00 | 0.00 | 0.00 | 5.80 | 1.80 |
| AGGAAGAGCC | AF125535 | pp21 homolog | 0.18 | 1.16 | 1.18 | 0.00 | 0.39 | 0.00 | 0.65 | 0.64 | 5.80 | 0.92 |
| TATAATAAAT | AF020351 | NDUFS4; NADH dehydrogenase (ubiquinone) Fe—S protein 4 (18 kD) (NADH-coenzyme Q reductase) | 0.00 | 0.58 | 0.20 | 0.00 | 0.20 | 0.00 | 0.33 | 0.00 | 5.80 | 0.00 |
| CGCCTCTCCA | AF030310 | UDP glycosyltransferase 1 family, polypeptide A3; B; A1; A9; A6 | 0.00 | 0.58 | 0.00 | 0.00 | 0.20 | 0.00 | 0.00 | 0.00 | 5.80 | 0.00 |
| CGGCCACAGA | D16990 | selenophosphate synthetase 2 | 0.00 | 0.58 | 0.00 | 0.18 | 0.20 | 0.00 | 0.33 | 0.32 | 5.80 | 0.00 |
| TAAACTTCTG | Y08612 | nucleoporin 88 kD | 0.00 | 0.58 | 0.20 | 0.72 | 0.20 | 0.00 | 0.00 | 0.00 | 5.80 | 0.00 |
| ATACCACTAA | Hs.153792 | 5-methyltetrahydrofolate-homocysteine methyltransferase reductase (EST) | 0.00 | 0.58 | 0.59 | 0.18 | 0.20 | 0.00 | 0.00 | 0.00 | 5.80 | 0.00 |
| ACAAAAATAA | Hs.347991 | nuclear receptor subfamily 2, group F, member 2 | 0.00 | 0.58 | 0.20 | 0.18 | 0.20 | 0.00 | 0.00 | 0.00 | 5.80 | 0.00 |
| CCTATAATAA | Hs.13885 | hypothetical protein MGC5309 (EST) | 0.00 | 0.58 | 0.78 | 0.36 | 0.20 | 0.00 | 0.33 | 0.32 | 5.80 | 0.00 |
| GGATGTAGAG | Hs.155485 | huntingtin interacting protein 2 (EST) | 0.00 | 0.58 | 0.59 | 0.72 | 0.20 | 0.00 | 0.33 | 0.00 | 5.80 | 0.00 |
| CCCCTTTGCA | Hs.75811 | N-acylsphingosine amidohydrolase (acid ceramidase) (EST) | 0.00 | 0.58 | 0.20 | 0.00 | 0.20 | 0.00 | 0.00 | 0.00 | 5.80 | 0.00 |
| CGGGGATCAT | Hs.55016 | hypothetical protein FLJ21935 (EST) | 0.00 | 0.58 | 0.00 | 0.00 | 0.20 | 0.00 | 0.00 | 0.00 | 5.80 | 0.00 |
| AGTTGTCCCG | AB007931 | KIAA04062 protein | 0.92 | 2.13 | 0.78 | 2.71 | 0.39 | 0.35 | 0.33 | 0.00 | 5.76 | 2.49 |
| CAACAGCTCT | Hs.118778 | KDEL (Lys-Asp-Glu-Leu) endoplasmic reticulum protein retention receptor 2 (EST) | 1.11 | 1.55 | 1.37 | 0.72 | 0.39 | 0.18 | 0.33 | 0.32 | 5.54 | 3.89 |
| CCACAGTAGA | AF046001 | Homo sapiens zinc finger transcription factor (ZNF | 0.74 | 1.55 | 0.98 | 0.90 | 0.39 | 0.18 | 0.65 | 0.32 | 5.54 | 2.60 |
| AACGGGCCGG | Hs.26484 | HIRA-interacting protein 3 | 0.18 | 1.55 | 0.78 | 0.54 | 0.39 | 0.18 | 0.33 | 0.32 | 5.54 | 0.63 |
| CAGCTTCACC | U90426 | nuclear RNA helicase, DECD variant of DEAD box family | 1.66 | 2.51 | 1.37 | 3.43 | 0.39 | 0.53 | 0.00 | 0.00 | 5.46 | 3.61 |
| CGGTCTTATG | D86550 | dual-specificity tyrosine-(Y)-phosphorylation regulated kinase 1A | 0.55 | 0.97 | 0.00 | 0.36 | 0.00 | 0.35 | 0.33 | 0.00 | 5.39 | 3.14 |
| CTAAGGTGGG | L76702 | protein phosphatase 2, regulatory subunit B (B56), delta isoform | 0.18 | 0.97 | 0.98 | 0.18 | 0.00 | 0.35 | 0.33 | 0.64 | 5.39 | 1.03 |
| GTTGTGATGT | NM_005243 | Ewing sarcoma breakpoint region 1 or EST | 0.18 | 0.97 | 1.18 | 1.08 | 0.00 | 0.35 | 0.00 | 0.00 | 5.39 | 1.03 |
| TGAAAGTGTG | D86956 | heat shock 105 kD | 1.29 | 1.93 | 1.76 | 1.63 | 0.20 | 0.53 | 0.00 | 0.00 | 5.36 | 3.53 |
| GGGTGCAAAA | Hs.249495 | heterogeneous nuclear ribonucleoprotein A1 | 2.03 | 3.86 | 2.16 | 2.35 | 0.20 | 1.24 | 0.00 | 0.00 | 5.36 | 2.82 |
| TTCTTGAACA | U41635 | acidic protein rich in leucines or amplified in osteosarcoma | 0.55 | 1.93 | 1.18 | 1.26 | 0.20 | 0.53 | 0.65 | 0.64 | 5.36 | 1.51 |
| CCAGTAGAAG | BC001451 | testin | 0.74 | 1.55 | 0.59 | 1.08 | 0.59 | 0.00 | 0.65 | 0.64 | 5.34 | 2.51 |
| GAGACTCCTG | AF070544 | GLUT1; solute carrier family 2 (facilitated glucose transporter), member 1, glucose transporter | 2.21 | 4.83 | 7.06 | 7.04 | 0.78 | 1.06 | 4.23 | 21.57 | 5.25 | 2.40 |
| GACGTGTGGG | X52317 X06 | H2A histone family, member Z | 2.40 | 4.25 | 3.92 | 2.53 | 0.78 | 0.89 | 0.65 | 0.64 | 5.12 | 2.87 |
| AATGACTGAA | BC006344 | ERP70; protein disulfide isomerase related protein (calcium-binding protein, intestinal-related | 0.74 | 0.97 | 0.20 | 0.36 | 0.20 | 0.18 | 0.98 | 0.00 | 5.11 | 3.89 |
| ATTTGAGAGT | Hs.146550 | myosin, heavy polypeptide 9, non-muscle (EST) | 0.74 | 0.97 | 1.18 | 0.90 | 0.20 | 0.18 | 2.60 | 0.64 | 5.11 | 3.89 |
| GCTGTTTTGT | AB023206 | Leman coiled-coil protein | 0.55 | 0.97 | 2.16 | 1.08 | 0.20 | 0.18 | 0.00 | 0.32 | 5.11 | 2.89 |
| AAGTGATTCT | D26120 | zinc finger protein 162 | 0.37 | 0.97 | 0.39 | 0.54 | 0.20 | 0.18 | 0.65 | 0.32 | 5.11 | 1.95 |
| TTCTGGCACT | Hs.20237 | DKFZP566C134 protein or EST | 0.18 | 0.97 | 0.78 | 1.08 | 0.20 | 0.18 | 0.00 | 0.00 | 5.11 | 0.95 |
| GGACGGCATT | Hs.78531 | Similar to RIKEN cDNA 5730528L13 gene, clone MGC: 17337, mRNA, complete cds (EST) | 0.18 | 0.97 | 0.20 | 0.18 | 0.20 | 0.18 | 0.00 | 0.64 | 5.11 | 0.95 |
| CCCTGGCTGT | Hs.58127 | hypothetical protein FLJ13902 or membrane-bound transcription factor protease, site 1 (EST) | 0.18 | 0.97 | 0.59 | 0.72 | 0.20 | 0.18 | 0.00 | 0.32 | 5.11 | 0.95 |
| AAATGGCTAA | BC000878 | CGI-83 protein | 0.92 | 1.93 | 0.59 | 0.72 | 0.59 | 0.18 | 0.00 | 0.00 | 5.08 | 2.39 |
| TCTGAAGTCA | Hs.180919 | inhibitor of DNA binding 2, dominant negative helix-loop-helix protein (EST) | 2.21 | 4.44 | 2.94 | 2.35 | 0.39 | 1.42 | 0.98 | 0.64 | 4.88 | 2.44 |
| TATTTATTGA | AB007930 | coat protein gamma-cop or others | 0.55 | 0.97 | 0.20 | 1.08 | 0.39 | 0.00 | 0.98 | 0.32 | 4.85 | 2.82 |
| CTTTATGTGA | U31814 | histone deacetylase 2 | 0.37 | 0.97 | 0.00 | 0.72 | 0.39 | 0.00 | 0.00 | 0.00 | 4.85 | 1.90 |
| AGGATATCCA | Hs.261285 | pleiotropic regulator 1 (PRL1, Arabidopsis homolog) | 0.00 | 0.97 | 0.20 | 0.00 | 0.39 | 0.00 | 0.00 | 0.00 | 4.85 | 0.00 |
| GCATATTAAA | D21090 | RAD23 (S. cerevisiae) homolog B | 1.11 | 1.74 | 2.35 | 1.63 | 0.20 | 0.53 | 0.33 | 0.32 | 4.83 | 3.04 |
| CAGCTCCGCT | AK000629 | dUTP pyrophosphatase | 0.74 | 1.74 | 0.20 | 1.08 | 0.20 | 0.53 | 0.00 | 0.97 | 4.83 | 2.03 |
| GGAGCCAGCT | AF147362 | PRO1659 protein or hypothetical protein DKFZp762A227 | 0.92 | 1.35 | 1.76 | 1.44 | 0.20 | 0.35 | 0.65 | 0.32 | 4.82 | 3.35 |
| CGGCAAAAAA | Hs.78019 | hypothetical protein 24432 (EST) or other EST | 0.92 | 1.35 | 0.39 | 0.36 | 0.20 | 0.35 | 0.00 | 0.00 | 4.82 | 3.35 |
| TTTGTTTCCA | Hs.194665 | DNA2 (DNA replication helicase, yeast, homolog)-like (EST) or others (EST) | 0.92 | 1.35 | 0.59 | 0.90 | 0.20 | 0.35 | 1.63 | 0.32 | 4.82 | 3.35 |
| AGAATGTACG | AL049851 | CARD10; caspase recruitment domain protein 10 | 0.37 | 1.35 | 2.94 | 1.81 | 0.20 | 0.35 | 0.33 | 0.64 | 4.82 | 1.35 |
| GTGCTTGTAC | AB001106 | GMFB: gila maturation factor, beta | 0.18 | 1.35 | 1.18 | 1.08 | 0.20 | 0.35 | 0.33 | 0.97 | 4.82 | 0.65 |
| TTCTGCACTG | BC007834 | hypothetical protein MGC14141 | 4.61 | 7.15 | 6.27 | 8.85 | 1.96 | 1.06 | 1.30 | 0.32 | 4.74 | 3.05 |
| GGTGGATGTG | AK001474 | methyl-CpG binding domain protein 3 | 4.06 | 5.60 | 7.25 | 4.15 | 0.98 | 1.42 | 2.28 | 0.00 | 4.67 | 3.38 |
| TGTGAACAAC | Hs.129771 | EST | 1.11 | 1.74 | 0.98 | 1.44 | 0.59 | 0.18 | 0.33 | 0.00 | 4.58 | 2.88 |
| TAAGCAGATG | AL050018 | DKFZP564B116 protein | 1.84 | 2.51 | 1.18 | 2.89 | 0.78 | 0.35 | 0.65 | 0.97 | 4.40 | 3.26 |
| AACTCTTGAA | U54559 | eukaryotic translation initiation factor 3, subunit 3 (gamma, 40 kD) | 1.84 | 2.51 | 2.16 | 0.72 | 0.78 | 0.35 | 0.65 | 1.29 | 4.40 | 3.26 |
| ACTTGATTTG | AB007963 | Homo sapiens mRNA for KIAA0494 protein, complete c | 0.18 | 0.39 | 0.39 | 0.72 | 0.00 | 0.18 | 0.33 | 0.00 | 4.33 | 2.00 |
| GTATTGAAGT | AB011170 | B cell RAG associated protein | 0.18 | 0.39 | 0.20 | 0.00 | 0.00 | 0.18 | 0.65 | 0.00 | 4.33 | 2.00 |
| AGGTCTAAGG | AB014562 | KIAA0662 gene product | 0.18 | 0.39 | 0.20 | 0.18 | 0.00 | 0.18 | 0.00 | 0.00 | 4.33 | 2.00 |
| AGAGACTCTT | AF261137 | DKFZP547E1010 protein | 0.18 | 0.39 | 0.39 | 0.00 | 0.00 | 0.18 | 0.33 | 0.00 | 4.33 | 2.00 |
| ATATTTCATT | AJ224144 | Homo sapiens mRNA variant beta for RNA polymerase | 0.18 | 0.39 | 0.00 | 0.00 | 0.00 | 0.18 | 0.00 | 0.00 | 4.33 | 2.00 |
| CAGGAGGAAA | AK000595 | KIAA0964 protein (EST) | 0.18 | 0.39 | 0.20 | 0.90 | 0.00 | 0.18 | 1.30 | 2.25 | 4.33 | 2.00 |
| TTTGACCTTT | AK001845 | Homo sapiens cDNA FLJ10983 fis, clone PLACE1001781 | 0.18 | 0.39 | 0.00 | 0.00 | 0.00 | 0.18 | 0.00 | 0.00 | 4.33 | 2.00 |
| TTGAATTTAT | AK002115 | intracellular membrane-associated calcium-Independent phospholipase A2 gamma | 0.18 | 0.39 | 0.00 | 0.18 | 0.00 | 0.18 | 0.00 | 0.00 | 4.33 | 2.00 |
| TTCTCTTTCA | AL133076 | hypothetical protein FLJ12716 | 0.18 | 0.39 | 0.00 | 0.18 | 0.00 | 0.18 | 0.00 | 0.32 | 4.33 | 2.00 |
| CAGCTCTTAG | AL133447 | hypothetical protein FLJ21128 | 0.18 | 0.39 | 0.39 | 0.36 | 0.00 | 0.18 | 0.00 | 0.00 | 4.33 | 2.00 |
| TGTGGGAACC | AL137764 | Novel human gene mapping to chomosome 1. | 0.18 | 0.39 | 0.20 | 0.18 | 0.00 | 0.18 | 0.00 | 0.00 | 4.33 | 2.00 |
| AAATCCTCAA | BC003606 | hypothetical protein FLJ10307 | 0.18 | 0.39 | 0.00 | 0.54 | 0.00 | 0.18 | 0.00 | 0.00 | 4.33 | 2.00 |
| GTAGAAAAGA | BC007617 | hypothetical protein MGC15729 | 0.18 | 0.39 | 0.00 | 0.36 | 0.00 | 0.18 | 0.65 | 0.00 | 4.33 | 2.00 |
| CAGACCCTGC | BC009435 | Similar to RIKEN cDNA 2010317E24 gene, clone IMAGE: 3502019, mRNA, partial cds | 0.18 | 0.39 | 0.59 | 0.36 | 0.00 | 0.18 | 0.00 | 0.00 | 4.33 | 2.00 |
| CTTGGCACCC | D13635 | ubiquitin-protein isopeptide ligase (E3) | 0.18 | 0.39 | 0.00 | 0.18 | 0.00 | 0.18 | 0.00 | 0.00 | 4.33 | 2.00 |
| GTACGCATTC | M76766 | general transcription factor IIB | 0.18 | 0.39 | 0.39 | 0.00 | 0.00 | 0.18 | 0.00 | 0.00 | 4.33 | 2.00 |
| TGAACACCCG | NM_031432 | uridine-cytidine kinase 1 | 0.18 | 0.39 | 0.20 | 0.00 | 0.00 | 0.18 | 0.00 | 0.00 | 4.33 | 2.00 |
| CAGTCTGGGA | Y09328 | interleukin 13 receptor, alpha 1 | 0.18 | 0.39 | 0.00 | 0.00 | 0.00 | 0.18 | 0.00 | 0.32 | 4.33 | 2.00 |
| CTGCTGATAA | Hs.3622 | procollagen-proline, 2-oxoglutarate 4-dioxygenase (proline 4-hydroxylase), alpha | 0.18 | 0.39 | 0.20 | 0.18 | 0.00 | 0.18 | 0.33 | 0.00 | 4.33 | 2.00 |
| | | polypeptide II (EST) or others | | | | | | | | | | |
| TAATGGATGT | Hs.13895 | EST | 0.18 | 0.39 | 0.20 | 0.36 | 0.00 | 0.18 | 0.33 | 0.00 | 4.33 | 2.00 |
| TCAATGGACA | Hs.1526 | ATPase, Ca++ transporting, cardiac muscle, slow twitch 2 (EST) | 0.18 | 0.39 | 0.20 | 0.36 | 0.00 | 0.18 | 0.00 | 0.00 | 4.33 | 2.00 |
| TCACGCCAAT | Hs.134177 | EST | 0.18 | 0.39 | 0.20 | 0.36 | 0.00 | 0.18 | 0.00 | 0.00 | 4.33 | 2.00 |
| CACAAACACA | Hs.69662 | EST | 0.18 | 0.39 | 0.20 | 0.36 | 0.00 | 0.18 | 0.00 | 0.00 | 4.33 | 2.00 |
| TGGAGTGTAC | Hs.159161 | Rho GDP dissociation inhibitor (GDI) alpha (EST) | 0.18 | 0.39 | 0.20 | 0.00 | 0.00 | 0.18 | 0.33 | 0.00 | 4.33 | 2.00 |
| ACTGCTCATT | Hs.177812 | uncharacterized bone marrow protein BM046 (EST) | 0.18 | 0.39 | 0.20 | 0.00 | 0.00 | 0.18 | 0.00 | 0.32 | 4.33 | 2.00 |
| TCCTGTGCTG | Hs.150644 | Weakly similar to RMS1_HUMAN REGULATOR OF MITOTIC SPINDLE | 0.18 | 0.39 | 0.20 | 0.00 | 0.00 | 0.18 | 0.00 | 0.00 | 4.33 | 2.00 |
| | | ASSEMBLY 1 [H. sapiens] (EST) | | | | | | | | | | |
| GCAAAAACAG | Hs.277401 | bromodomain adjacent to zinc finger domain, 2A (EST) | 0.18 | 0.39 | 0.20 | 0.00 | 0.00 | 0.18 | 0.00 | 0.00 | 4.33 | 2.00 |
| AGTTGTACTT | Hs.181357 | LAMR1; laminin receptor 1 (67 kD, ribosomal protein SA) (EST) | 0.18 | 0.39 | 0.39 | 0.18 | 0.00 | 0.18 | 0.00 | 0.32 | 4.33 | 2.00 |
| GCAACAGAAA | Hs.301724 | hypothetical protein FLJ11301 (EST) | 0.18 | 0.39 | 0.39 | 0.54 | 0.00 | 0.18 | 0.00 | 0.00 | 4.33 | 2.00 |
| AGGCTACCGG | Hs.119122 | ribosomal protein L 13a (EST) | 0.18 | 0.39 | 0.39 | 0.00 | 0.00 | 0.18 | 0.00 | 0.00 | 4.33 | 2.00 |
| CAGGCAACTT | Hs.282235 | EST | 0.18 | 0.39 | 0.39 | 0.00 | 0.00 | 0.18 | 0.00 | 0.00 | 4.33 | 2.00 |
| CTGGGAAGCA | Hs.288433 | neurotrimin (EST) | 0.18 | 0.39 | 0.59 | 0.18 | 0.00 | 0.18 | 0.00 | 0.00 | 4.33 | 2.00 |
| GAGTATGGAG | Hs.172089 | pro-oncosis receptor inducing membrane injury gene | 0.18 | 0.39 | 0.59 | 0.18 | 0.00 | 0.18 | 0.00 | 0.00 | 4.33 | 2.00 |
| CAAATATCTT | Hs.315482 | EST | 0.18 | 0.39 | 0.59 | 0.36 | 0.00 | 0.18 | 0.33 | 0.00 | 4.33 | 2.00 |
| ATCACAAAAA | Hs.2556 | TNFRSF17; tumor necrosis factor receptor superfamily, member 17 (EST) | 0.18 | 0.39 | 0.59 | 0.00 | 0.00 | 0.18 | 0.00 | 0.00 | 4.33 | 2.00 |
| GCCACAGTTG | Hs.77326 | IGFBP3; insulin-like growth factor binding protein 3 (EST) | 0.18 | 0.39 | 0.00 | 0.18 | 0.00 | 0.18 | 0.32 | 0.00 | 4.33 | 2.00 |
| CCACACCCTG | Hs.182426 | ribosomal protein S2 (EST) or others | 0.18 | 0.39 | 0.00 | 0.18 | 0.00 | 0.18 | 0.00 | 0.00 | 4.33 | 2.00 |
| TAAGACTATA | Hs.350424 | hypothetical protein DKFZp564K142 (EST) | 0.18 | 0.39 | 0.00 | 0.18 | 0.00 | 0.18 | 0.00 | 0.00 | 4.33 | 2.00 |
| GCCCAGCCAG | Hs.348987 | EST | 0.18 | 0.39 | 0.00 | 0.18 | 0.00 | 0.18 | 0.00 | 0.00 | 4.33 | 2.00 |
| CAGTTCTTGA | Hs.284217 | serologically defined colon cancer antigen 33 (EST) | 0.18 | 0.39 | 0.00 | 0.18 | 0.00 | 0.18 | 0.00 | 0.00 | 4.33 | 2.00 |
| GAGAAGACTG | Hs.118281 | zinc finger 266 | 0.18 | 0.39 | 0.00 | 0.36 | 0.00 | 0.18 | 0.00 | 0.00 | 4.33 | 2.00 |
| GAAGGGACCG | Hs.182485 | actinin, alpha 4 (EST) | 0.18 | 0.39 | 0.00 | 0.54 | 0.00 | 0.18 | 0.00 | 0.00 | 4.33 | 2.00 |
| TCAGAAACTG | Hs.193482 | cDNA FLJ11903 fis, clone HEMBB1000030 (EST) | 0.18 | 0.39 | 0.00 | 0.72 | 0.00 | 0.18 | 0.00 | 0.00 | 4.33 | 2.00 |
| CCAACAACTA | Hs.301226 | KIAA1085 protein (EST) | 0.18 | 0.39 | 0.00 | 0.00 | 0.00 | 0.18 | 0.33 | 0.00 | 4.33 | 2.00 |
| GCTGCTGCCT | AF396651 | tripartite motif-containing 7 | 0.18 | 0.39 | 0.00 | 0.00 | 0.00 | 0.18 | 0.33 | 0.32 | 4.33 | 2.00 |
| GTAAAAAAAG | Hs.13503 | inactivation escape 2 | 0.18 | 0.39 | 0.00 | 0.00 | 0.00 | 0.18 | 0.00 | 0.00 | 4.33 | 2.00 |
| GGCATTTTTG | Hs.11482 | splicing factor, arginine/serine-rich 11 (EST) | 0.18 | 0.39 | 0.00 | 0.00 | 0.00 | 0.18 | 0.00 | 0.00 | 4.33 | 2.00 |
| AGTTCAGGAG | Hs.314024 | Weakly similar to ZN91_HUMAN ZINC FINGER PROTEIN 91 [H. sapiens] (EST) | 0.18 | 0.39 | 0.00 | 0.00 | 0.00 | 0.18 | 0.00 | 0.00 | 4.33 | 2.00 |
| AGACGCTTTC | Hs.203772 | FSHD region gens 1 (EST) or others | 0.18 | 0.39 | 0.00 | 0.00 | 0.00 | 0.18 | 0.00 | 0.00 | 4.33 | 2.00 |
| TTGAAGCACC | Hs.11463 | UMP-CMP kinase (EST) or vanin 1 (EST) | 0.18 | 0.39 | 0.00 | 0.00 | 0.00 | 0.18 | 0.00 | 0.00 | 4.33 | 2.00 |
| CTCAGAAAAA | Hs.80120 | UDP-N-acetyl-alpha-D-galaclosamine: polypeptide N-acetylgalactosaminyltransferase | 0.18 | 0.39 | 0.00 | 0.00 | 0.00 | 0.18 | 0.00 | 0.00 | 4.33 | 2.00 |
| | | 1 (GalNac-T1) (EST) | | | | | | | | | | |
| CAGTGAAAAA | Hs.114765 | myeloid/lymphoid or mixed-lineage leukemia (trithorax (Drosophila) homolog); tanslocated to, | 0.18 | 0.39 | 0.00 | 0.00 | 0.00 | 0.18 | 0.00 | 0.00 | 4.33 | 2.00 |
| | | 2 (EST) or others | | | | | | | | | | |
| GAGCTTACAT | Hs.278672 | membrane component, chromosome 11, surface marker 1 (EST) or others | 0.18 | 0.39 | 0.00 | 0.00 | 0.00 | 0.18 | 0.00 | 0.00 | 4.33 | 2.00 |
| ACGCTCATCG | AB017364 | frizzled (Drosophila) homolog 2 | 0.00 | 0.39 | 0.20 | 0.00 | 0.00 | 0.18 | 0.00 | 0.00 | 4.33 | 0.00 |
| TTTTCTTCAT | AB017642 | OSR1; oxidative-stress rasponsive 1 | 0.00 | 0.39 | 0.20 | 0.18 | 0.00 | 0.18 | 0.00 | 0.32 | 4.33 | 0.00 |
| TACTGCCTCT | AF049105 | centrosomal protein 2 | 0.00 | 0.39 | 0.00 | 0.00 | 0.00 | 0.18 | 0.33 | 0.00 | 4.33 | 0.00 |
| CGGCAGGAGA | AF070535 | hypothetical protetin 24432 | 0.00 | 0.39 | 0.20 | 0.18 | 0.00 | 0.18 | 0.00 | 0.00 | 4.33 | 0.00 |
| AACATTGACA | AF130104 | hypothetical protein DKFZp564K142 | 0.00 | 0.39 | 0.20 | 0.36 | 0.00 | 0.18 | 0.33 | 0.64 | 4.33 | 0.00 |
| GGAAGTGCCA | AF154005 | junctional adhesion molecule 1 | 0.00 | 0.39 | 0.00 | 0.00 | 0.00 | 0.18 | 0.00 | 0.00 | 4.33 | 0.00 |
| AGGCCACCTC | AF186409 | frequenin (Drosophila) homolog | 0.00 | 0.39 | 0.00 | 0.36 | 0.00 | 0.18 | 0.33 | 0.00 | 4.33 | 0.00 |
| AACTTTCTGG | AF250136 | peroxisomal membrane protein 2 (22 kD) | 0.00 | 0.39 | 0.20 | 0.18 | 0.00 | 0.18 | 0.00 | 0.00 | 4.33 | 0.00 |
| TGGAAGCTAG | AJ224358 | surfelt 5 | 0.00 | 0.39 | 0.00 | 1.08 | 0.00 | 0.18 | 0.00 | 0.00 | 4.33 | 0.00 |
| GATAGAGGGA | AK000264 | hypothetical protein FLJ20257 | 0.00 | 0.39 | 0.20 | 0.72 | 0.00 | 0.18 | 0.00 | 0.00 | 4.33 | 0.00 |
| GTTCCTTTGG | AK000553 | hypothetical protein FLJ20546 | 0.00 | 0.39 | 0.20 | 0.36 | 0.00 | 0.18 | 0.33 | 0.00 | 4.33 | 0.00 |
| TGACTGGCTT | AK002127 | death associated transcription factor 1 | 0.00 | 0.39 | 0.00 | 0.00 | 0.00 | 0.18 | 0.00 | 0.00 | 4.33 | 0.00 |
| AACAGTCAAA | BC000081 | plakophilin 3 | 0.00 | 0.39 | 0.39 | 0.00 | 0.00 | 0.18 | 0.00 | 0.00 | 4.33 | 0.00 |
| CAGTTGCTGG | BC000808 | hypothetical protein MGC5499 | 0.00 | 0.39 | 0.00 | 0.00 | 0.00 | 0.18 | 0.33 | 0.00 | 4.33 | 0.00 |
| CGCCGTCGCA | BC001112 | moesin | 0.00 | 0.39 | 0.00 | 0.38 | 0.00 | 0.18 | 0.00 | 0.00 | 4.33 | 0.00 |
| CTAGTGAAAC | BC001771 | generel transcription factor IIF, polypeptide 2 (30 kD Subunit) | 0.00 | 0.39 | 0.20 | 0.00 | 0.00 | 0.18 | 0.00 | 0.00 | 4.33 | 0.00 |
| GTGATGTCTG | BC005075 | clone IMAGE: 3534875, mRNA, partial cds | 0.00 | 0.39 | 0.39 | 0.36 | 0.00 | 0.18 | 0.00 | 0.32 | 4.33 | 0.00 |
| GCAGGCCAAG | K01566 | B-factor, properdin or MHC class III serum complement factor B, mRNA or RAB30, member RAS | 0.00 | 0.39 | 0.00 | 0.00 | 0.00 | 0.18 | 0.33 | 0.32 | 4.33 | 0.00 |
| | | oncogene family | | | | | | | | | | |
| TTCTTTTCAT | L18960 | eukaryotic translation initiation factor 1A | 0.00 | 0.39 | 0.39 | 0.54 | 0.00 | 0.18 | 0.00 | 0.00 | 4.33 | 0.00 |
| ATCCTCTGCG | L41816 | cslcium/calmodulin-dependent protein kinase I | 0.00 | 0.39 | 0.20 | 0.18 | 0.00 | 0.18 | 0.00 | 0.00 | 4.33 | 0.00 |
| GCCTTTTACT | U00238 | Homo sapiens glutamine PRPP amidotransferase (GPAT | 0.00 | 0.39 | 0.00 | 0.18 | 0.00 | 0.18 | 0.00 | 0.00 | 4.33 | 0.00 |
| GCTGGGAACC | X60708 S40 | dipeptidylpeptidase IV (CD26, adenosine deaminase complexing protein 2) | 0.00 | 0.39 | 0.39 | 0.54 | 0.00 | 0.18 | 0.98 | 2.25 | 4.33 | 0.00 |
| GCCAGCAAAT | X80692 | mitogen-activated protein kinase 6 | 0.00 | 0.39 | 0.78 | 0.00 | 0.00 | 0.18 | 0.00 | 0.64 | 4.33 | 0.00 |
| CAGCCCCTCT | Y09836 | mRNA for 3′UTR of unknown protein | 0.00 | 0.39 | 0.00 | 0.00 | 0.00 | 0.18 | 0.00 | 0.00 | 4.33 | 0.00 |
| TAGGTGTATG | Hs.139105 | Weakly similar to I38022 hypothetical protein [H. sapiens] (EST) | 0.00 | 0.39 | 0.20 | 0.18 | 0.00 | 0.18 | 0.33 | 0.00 | 4.33 | 0.00 |
| TCACTGCTCT | Hs.54413 | retinoic acid responsive (EST) or homeo box D8 (EST) | 0.00 | 0.39 | 0.00 | 0.18 | 0.00 | 0.18 | 0.33 | 0.00 | 4.33 | 0.00 |
| GGCATTTATT | Hs.12705 | Homo sapiens, clone MGC: 4710 IMAGE: 3534806 mRNA complete cds | 0.00 | 0.39 | 0.20 | 0.18 | 0.00 | 0.18 | 0.00 | 0.64 | 4.33 | 0.00 |
| AATCCATATG | Hs.179661 | FK506-binding protein 1A (12 kD) (EST) | 0.00 | 0.39 | 0.00 | 0.18 | 0.00 | 0.18 | 0.33 | 0.32 | 4.33 | 0.00 |
| CCAGAGTCTC | Hs.73793 | VEGF; vascular endothelial growth factor (EST) | 0.00 | 0.39 | 0.39 | 0.18 | 0.00 | 0.18 | 0.65 | 0.32 | 4.33 | 0.00 |
| TTACTTATAC | Hs.159 | TNFRSF1A; tumor necrosia factor receptor superfamily, member 1A (EST) | 0.00 | 0.39 | 0.78 | 0.18 | 0.00 | 0.18 | 1.30 | 0.00 | 4.33 | 0.00 |
| ATCTTGGCTC | Hs.91622 | neuronal pentraxin receptor (EST) or others | 0.00 | 0.39 | 0.39 | 0.18 | 0.00 | 0.18 | 0.00 | 0.00 | 4.33 | 0.00 |
| AGCTCAGCGT | Hs.10450 | EST | 0.00 | 0.39 | 0.39 | 0.18 | 0.00 | 0.18 | 0.00 | 0.00 | 4.33 | 0.00 |
| GGCCCGCTGC | Hs.3745 | MFGE8; milk fat globule-EGF factor 8 protein (EST) | 0.00 | 0.39 | 0.20 | 0.18 | 0.00 | 0.18 | 1.30 | 0.64 | 4.33 | 0.00 |
| CCTAGTCCAA | Hs.2795 | lactate dehydrogenase A (EST) | 0.00 | 0.39 | 0.20 | 0.18 | 0.00 | 0.18 | 0.00 | 0.00 | 4.33 | 0.00 |
| GGGCTAACTC | Hs.66881 | dynein, cytoplasmic intermediate polypeptide 2 (EST) | 0.00 | 0.39 | 0.20 | 0.18 | 0.00 | 0.18 | 0.00 | 0.00 | 4.33 | 0.00 |
| GCACTACAGA | Hs.5947 | mel transforming oncogene (derived from cell line NK14)-RAB8 homolog (EST) | 0.00 | 0.39 | 0.20 | 0.18 | 0.00 | 0.18 | 0.00 | 0.00 | 4.33 | 0.00 |
| CACAAGGTAA | Hs.283978 | PRO2751 mRNA complete cds (EST) | 0.00 | 0.39 | 0.00 | 0.18 | 0.00 | 0.18 | 0.00 | 0.00 | 4.33 | 0.00 |
| GATGTGGCTG | Hs.275959 | FK506-binding protein 6 (36 kD) (EST) or eukaryotic translation elongation factor 1 beta 2 (EST) | 0.00 | 0.39 | 0.20 | 0.36 | 0.00 | 0.18 | 0.00 | 0.32 | 4.33 | 0.00 |
| CATCCTGTGC | Hs.333513 | small inducible cytokine subfamily E, member 1 (endothelial monocyte-activating) (EST) | 0.00 | 0.39 | 0.00 | 0.36 | 0.00 | 0.18 | 0.33 | 0.00 | 4.33 | 0.00 |
| GGTGGGACCA | Hs.277585 | KIAA0170 gene product (EST) | 0.00 | 0.39 | 0.00 | 0.36 | 0.00 | 0.18 | 0.00 | 0.32 | 4.33 | 0.00 |
| GTGGGACCAT | Hs.11774 | protein (peptidyl-prolyl cis/trans isomerase) NIMA-interacting, 4 (parvulin) (EST) | 0.00 | 0.39 | 0.59 | 0.36 | 0.00 | 0.18 | 0.00 | 0.97 | 4.33 | 0.00 |
| GCAACACCCC | Hs.75887 | coatomer protein complex, subunit alpha (EST) | 0.00 | 0.39 | 0.00 | 0.36 | 0.00 | 0.18 | 0.00 | 0.00 | 4.33 | 0.00 |
| GGAAGGAGGG | Hs.118638 | non-metastatic cells 1, protein NM23A expressed in (EST) | 0.00 | 0.39 | 0.00 | 0.36 | 0.00 | 0.18 | 0.00 | 0.00 | 4.33 | 0.00 |
| TCATTCTTAA | Hs.89104 | EST | 0.00 | 0.39 | 0.00 | 0.36 | 0.00 | 0.18 | 0.00 | 0.00 | 4.33 | 0.00 |
| GCAAAGATTG | Hs.28631 | EST | 0.00 | 0.39 | 0.20 | 0.54 | 0.00 | 0.18 | 0.65 | 0.97 | 4.33 | 0.00 |
| CACCCCCTCG | Hs.91246 | hypothetical protein DKFZp547O146 (EST) | 0.00 | 0.39 | 0.20 | 0.00 | 0.00 | 0.18 | 0.33 | 0.00 | 4.33 | 0.00 |
| CAAGAAGGCC | Hs.42129 | hypothetical protein MGC11102 (EST) | 0.00 | 0.39 | 0.00 | 0.00 | 0.00 | 0.18 | 0.33 | 0.00 | 4.33 | 0.00 |
| TACAGAAAGG | Hs.2994 | PCTAIRE protein kinase 3 (EST) | 0.00 | 0.39 | 0.00 | 0.00 | 0.00 | 0.18 | 0.33 | 0.00 | 4.33 | 0.00 |
| AAACAAGGCA | Hs.181013 | phosphoglycerate mutase 1 (brain) (EST) | 0.00 | 0.39 | 0.59 | 0.00 | 0.00 | 0.18 | 0.65 | 0.00 | 4.33 | 0.00 |
| AGCAGCGGGG | Hs.170285 | nucleoporin 214 KD (CAIN) (EST) | 0.00 | 0.39 | 0.00 | 0.00 | 0.00 | 0.18 | 0.33 | 0.32 | 4.33 | 0.00 |
| GCAGACATTG | Hs.179662 | nucleosome assembly protein 1-like 1 (EST) or others (EST) | 0.00 | 0.39 | 0.20 | 0.00 | 0.00 | 0.18 | 0.00 | 1.61 | 4.33 | 0.00 |
| CAGTCCCACA | Hs.324406 | ribosomal protein L41 (EST) | 0.00 | 0.39 | 0.78 | 0.00 | 0.00 | 0.18 | 0.00 | 0.00 | 4.33 | 0.00 |
| TCAGCTGTAC | Hs.15020 | homolog of mouse quaking QKI (KH domain RNA binding protein) (EST) | 0.00 | 0.39 | 0.39 | 0.00 | 0.00 | 0.18 | 0.00 | 0.00 | 4.33 | 0.00 |
| TAGGTATAGG | Hs.132071 | ovarian carcinoma Immunoreactive antigen (EST) | 0.00 | 0.39 | 0.20 | 0.00 | 0.00 | 0.18 | 0.00 | 0.00 | 4.33 | 0.00 |
| AAACTGGACA | Hs.5094 | ring finger protein 10 (EST) | 0.00 | 0.39 | 0.20 | 0.00 | 0.00 | 0.18 | 0.00 | 0.00 | 4.33 | 0.00 |
| AACTTTGTGG | Hs.301064 | arfaptin 1 (EST) | 0.00 | 0.39 | 0.00 | 0.00 | 0.00 | 0.18 | 0.00 | 0.00 | 4.33 | 0.00 |
| TGTCTGATGC | Hs.4437 | ribosomal protein L28 (EST) | 0.00 | 0.39 | 0.00 | 0.00 | 0.00 | 0.18 | 0.00 | 0.00 | 4.33 | 0.00 |
| CCCAGTTAAA | Hs.7844 | golgl autoantigen, golgin subfamily b, macrogolgin (with transmembrane signal), 1 (EST) | 0.00 | 0.39 | 0.00 | 0.00 | 0.00 | 0.18 | 0.00 | 0.00 | 4.33 | 0.00 |
| TAACCATCAC | Hs.44307 | EST | 0.00 | 0.39 | 0.00 | 0.00 | 0.00 | 0.18 | 0.00 | 0.00 | 4.33 | 0.00 |
| GAACAGATAG | Hs.108301 | nuclear raceptor subfamily 2, group C, member 1 (EST) | 0.00 | 0.39 | 0.00 | 0.00 | 0.00 | 0.18 | 0.00 | 0.00 | 4.33 | 0.00 |
| GCAAATTGTT | Hs.1645 | cytochrome P450, subfamily IVA, polypeptide 11 (EST) | 0.00 | 0.39 | 0.00 | 0.00 | 0.00 | 0.18 | 0.00 | 0.00 | 4.33 | 0.00 |
| CAGATGTGGA | Hs.6994 | cDNA: FLJ22044 fis, clone HEP09141 | 0.00 | 0.39 | 0.00 | 0.00 | 0.00 | 0.18 | 0.00 | 0.00 | 4.33 | 0.00 |
| CTACACAGTA | Hs.180610 | splicing factor proline/glutamine rich (polypyrimidine tract-binding protein-associated) (EST) | 0.00 | 0.39 | 0.00 | 0.00 | 0.00 | 0.18 | 0.00 | 0.00 | 4.33 | 0.00 |
| CTCATTCAGC | Hs.180139 | SMT3 (suppresssor of mif two 3, yeast) homolog 2 (EST) or C9orf10 protein (EST) | 1.29 | 1.55 | 2.75 | 2.17 | 0.20 | 0.53 | 0.98 | 0.00 | 4.31 | 3.53 |
| TGCAGATGGT | AB014587 | mitogen-activated protein kinase kinase kinase kinase 4 | 1.11 | 1.55 | 0.98 | 0.72 | 0.20 | 0.53 | 1.30 | 0.64 | 4.31 | 3.04 |
| GCCTGATTTT | D82060 | HLA class II region expressed gene KE4 or syndecan binding protein (syntenin) (EST) | 0.92 | 1.55 | 0.39 | 0.90 | 0.20 | 0.53 | 0.00 | 0.32 | 4.31 | 2.52 |
| TGTGCTAATA | S54711 | TSE1 = protein kinase A regulatory subunit gene [hum | 0.92 | 1.16 | 0.20 | 0.72 | 0.00 | 0.53 | 1.30 | 0.64 | 4.30 | 3.47 |
| CCTGAAATTT | U23803 | HNRPA0; heterogeneous nuclear ribonucleoprotein A0 | 1.29 | 1.93 | 0.39 | 1.26 | 0.20 | 0.71 | 0.00 | 0.32 | 4.29 | 2.84 |
| CCTTACCTAC | AF151885 | CGI-127 protein | 0.55 | 0.77 | 0.78 | 0.90 | 0.00 | 0.35 | 0.33 | 0.00 | 4.28 | 3.14 |
| TGGTGACATT | U57627 | oculocerebrorenal syndrome of Lowe | 0.37 | 0.77 | 0.00 | 0.18 | 0.00 | 0.35 | 0.00 | 0.00 | 4.28 | 2.11 |
| CGTTTAATGT | Hs.11637 | EST | 0.37 | 0.77 | 0.20 | 0.00 | 0.00 | 0.35 | 1.63 | 2.25 | 4.28 | 2.11 |
| GCGAGACCCC | Hs.4188 | EST | 0.37 | 0.77 | 0.39 | 0.54 | 0.00 | 0.35 | 0.33 | 0.64 | 4.28 | 2.11 |
| CCATACAGAC | Hs.289088 | heat shock 90 kD protein 1, alpha (EST) | 0.37 | 0.77 | 0.39 | 0.72 | 0.00 | 0.35 | 0.33 | 0.00 | 4.28 | 2.11 |
| TACGCTCCTT | AB037850 | DKFZP434I116 protein | 0.18 | 0.77 | 0.00 | 0.18 | 0.00 | 0.35 | 0.00 | 0.64 | 4.28 | 1.03 |
| CTTTGATCAG | AL121903 | brefeldin A-inhibited guanine nucleotide-exchange protein 2 | 0.18 | 0.77 | 0.59 | 0.72 | 0.00 | 0.35 | 0.00 | 0.00 | 4.28 | 1.03 |
| TGGAAATAAA | BC000547 | mitochondrial ribosomal protein S6 | 0.18 | 0.77 | 0.98 | 0.36 | 0.00 | 0.35 | 0.00 | 0.64 | 4.28 | 1.03 |
| GCTGGGCGGC | AK001002 | hypothetical protein FLJ10141 | 0.00 | 0.77 | 0.20 | 0.36 | 0.00 | 0.35 | 0.00 | 0.97 | 4.28 | 0.00 |
| GAAGAACAGA | AL442086 | hypothetical protein FLJ22376 | 0.00 | 0.77 | 0.59 | 0.18 | 0.00 | 0.35 | 0.65 | 0.32 | 4.28 | 0.00 |
| CTCATACACC | Hs.191146 | EST | 0.00 | 0.77 | 0.20 | 0.54 | 0.00 | 0.35 | 0.33 | 0.00 | 4.28 | 0.00 |
| GACAAGATGC | Hs.342389 | peptidylprolyl isomerase A (cyclophilin A) (EST) | 0.00 | 0.77 | 0.98 | 0.72 | 0.00 | 0.35 | 0.33 | 0.00 | 4.28 | 0.00 |
| TCCAAAAAAA | Hs.82985 | collagen, type V, alpha 2 (EST) or homocysteine-inducible, endoplasmic reticulum stress-inducible, | 0.00 | 0.77 | 0.20 | 0.00 | 0.00 | 0.35 | 0.00 | 0.00 | 4.28 | 0.00 |
| | | ubiquitin-like domain member 1 | | | | | | | | | | |
| GTGGCACGTG | AK000715 | polymerase I and transcript release factor | 2.21 | 3.67 | 2.16 | 2.17 | 1.37 | 0.35 | 1.63 | 0.64 | 4.27 | 2.57 |
| TGGGCTGGGG | Hs.3416 | adipose differentiation-related protein (EST) | 4.61 | 5.41 | 2.94 | 2.17 | 0.59 | 1.95 | 7.15 | 42.81 | 4.26 | 3.63 |
| TCGCCGCGAC | Hs.296290 | ribosomal protein L37a (EST) | 3.14 | 5.22 | 3.53 | 3.61 | 1.57 | 0.89 | 0.65 | 3.54 | 4.24 | 2.55 |
| TCTTCTGCCA | Hs.86392 | KIAA1402 protein (EST) | 1.84 | 3.09 | 2.16 | 1.08 | 0.39 | 1.06 | 0.65 | 0.00 | 4.23 | 2.54 |
| TACTGTAGTC | AK001362 | cDNA FLJ10500 fis, clone NT2RP2000369 | 1.66 | 1.93 | 1.76 | 1.26 | 0.39 | 0.53 | 1.63 | 0.97 | 4.20 | 3.61 |
| TGGTGACAGT | BC000098 | purine-rich element binding protein B | 2.21 | 3.48 | 4.71 | 2.89 | 0.59 | 1.06 | 0.65 | 0.32 | 4.19 | 2.68 |
| GTGAAGTCTT | AF055031 | solute carrier family 3 (activators of dibasic and neutral amino acid transport), member 2 | 0.37 | 1.55 | 1.57 | 0.72 | 0.39 | 0.35 | 0.98 | 0.97 | 4.19 | 1.00 |
| GAGTAGAGAA | Hs.145279 | SET translocation (myeloid leukemia-associated) | 0.37 | 1.55 | 1.76 | 1.44 | 0.39 | 0.35 | 0.33 | 0.32 | 4.19 | 1.00 |
| AACCCAGGAG | AF007137 | hypothetical protein FLJ22814 | 0.74 | 1.16 | 0.59 | 1.44 | 0.39 | 0.18 | 0.33 | 0.64 | 4.14 | 2.60 |
| TGCAATAAGA | Hs.7960 | DnaJ (Hsp40) homolog, subfamily B, member 12 (EST) | 0.74 | 1.16 | 0.39 | 0.18 | 0.39 | 0.18 | 0.33 | 0.00 | 4.14 | 2.60 |
| CACAGTCAAA | Hs.250712 | calcium channel, voltage-dependent, beta 3 subunit (EST) or desmoplakin (DPI, DPII) (EST) | 0.55 | 1.16 | 1.18 | 0.72 | 0.20 | 0.35 | 0.00 | 0.32 | 4.14 | 2.00 |
| CCTGCACACT | Hs.284161 | hypothetical protein from EUROIMAGE 42353 (EST) | 0.55 | 1.16 | 0.59 | 0.90 | 0.39 | 0.18 | 0.33 | 0.32 | 4.14 | 1.93 |
| CTTGCAAACC | Hs.127799 | baculoviral IAP repeat-containing 3 (EST) | 0.37 | 1.16 | 0.20 | 0.90 | 0.20 | 0.35 | 0.00 | 0.00 | 4.14 | 1.35 |
| CGGCAGAGCT | UB3993 | purinergic receptor P2X, ligand-gated ion channel, 4 | 0.37 | 1.16 | 0.59 | 0.54 | 0.39 | 0.18 | 0.98 | 0.00 | 4.14 | 1.30 |
| ACCTGCTGGT | AK000447 | GTPase Rab14 | 0.18 | 1.16 | 1.18 | 0.00 | 0.20 | 0.35 | 0.00 | 0.97 | 4.14 | 0.65 |
| TCTGACCACC | Hs.9614 | 11 nt = g; nucleophosmin (nucleolar phoshoprotein B23, numatrin(11 g, EST) | 0.18 | 1.16 | 0.00 | 0.18 | 0.20 | 0.35 | 0.00 | 0.00 | 4.14 | 0.65 |
| TAAGTGGAAT | BC003623 | tyrosine 3-monooxygenase/tryptophan 5-monooxygenase activation protein, zeta polypeptide | 2.03 | 4.25 | 1.57 | 1.99 | 1.17 | 0.89 | 0.65 | 0.00 | 4.13 | 1.97 |
| TTAAAGATTT | AJ000147 | tropomyosin 1 (alpha) | 2.03 | 3.09 | 1.78 | 5.42 | 0.78 | 0.71 | 0.65 | 0.32 | 4.12 | 2.72 |
| GCGGCAGCGG | Hs.326249 | ribosomal protein L22 (EST) | 1.29 | 1.55 | 0.39 | 1.26 | 0.59 | 0.18 | 0.33 | 1.61 | 4.08 | 3.35 |
| AAAGCAAACC | AK000414 | E2F transcription factor 5, p130-binding (EST) | 2.21 | 2.32 | 1.57 | 2.17 | 0.78 | 0.35 | 0.33 | 0.64 | 4.07 | 3.91 |
| TGACTTTTCT | BC000962 | zinc finger protein 207 | 1.29 | 2.32 | 0.59 | 0.36 | 0.78 | 0.35 | 0.00 | 0.32 | 4.07 | 2.28 |
| ATGTAGAGTG | X02308 | thymidytate synthetase | 3.50 | 4.06 | 4.71 | 2.53 | 0.00 | 0.00 | 0.33 | 0.64 | 4.06 | 3.50 |
| ATTTCACATT | AL137663 | DKFZp434G227 | 0.74 | 0.77 | 0.00 | 0.54 | 0.20 | 0.18 | 0.00 | 0.00 | 4.05 | 3.89 |
| GGCTGAGAAT | Hs.351680 | EST | 0.74 | 0.77 | 0.98 | 1.26 | 0.20 | 0.18 | 0.33 | 0.97 | 4.05 | 3.89 |
| CAAGGAAGCA | AJ420488 | eukaryotic translation elongation factor 1 alpha 1 | 0.74 | 0.77 | 0.00 | 0.18 | 0.20 | 0.18 | 0.65 | 0.00 | 4.05 | 3.89 |
| CCTGTGGTCC | AK000470 | KIAA1609 protein | 0.55 | 0.77 | 0.59 | 0.54 | 0.20 | 0.18 | 0.65 | 1.61 | 4.05 | 2.89 |
| CCTACAGATA | AK000513 | hypothetical protein FLJ20506 | 0.55 | 0.77 | 0.00 | 0.90 | 0.20 | 0.18 | 0.00 | 0.32 | 4.05 | 2.89 |
| GGGGAAAAAA | X75913 | complement component 1, q subcomponent binding protein | 0.55 | 0.77 | 0.59 | 0.54 | 0.20 | 0.18 | 0.00 | 0.32 | 4.05 | 2.89 |
| ACGTAATTAG | Hs.24341 | transcriptional co-activator with PDZ-binding motif (TAZ) (EST) | 0.55 | 0.77 | 0.59 | 0.18 | 0.20 | 0.18 | 0.00 | 0.97 | 4.05 | 2.89 |
| CCCGGCTCTT | BC010009 | Homo sapiens, clone IMAGE: 3355383, mRNA, partial cds | 0.55 | 0.77 | 0.59 | 0.54 | 0.20 | 0.18 | 0.00 | 0.00 | 4.05 | 2.89 |
| CCTGGATAAA | Hs.321130 | melanophilin (EST) or other EST | 0.55 | 0.77 | 1.18 | 0.90 | 0.20 | 0.18 | 0.33 | 0.00 | 4.05 | 2.89 |
| TAACCTGCTA | Hs.14541 | cullin 1 (EST) | 0.55 | 0.77 | 1.37 | 1.08 | 0.20 | 0.18 | 0.00 | 0.00 | 4.05 | 2.89 |
| TGATGTTCCA | AB032254 | bromodomain adjacent to zinc finger domain, 2A | 0.37 | 0.77 | 0.39 | 1.44 | 0.20 | 0.18 | 0.00 | 0.00 | 4.05 | 1.95 |
| CTGCCTGGCA | AK000719 | X-ray repair complementing defective repair in Chinese hamster cells 3 or hypothetical protein FLJ20712 | 0.37 | 0.77 | 0.39 | 0.54 | 0.20 | 0.18 | 0.00 | 0.32 | 4.05 | 1.95 |
| CAGGTCAAGA | X62153 | minichromosome maintenance deficient (S. cerevisiae) 3 | 0.37 | 0.77 | 0.98 | 0.54 | 0.20 | 0.18 | 0.00 | 0.00 | 4.05 | 1.95 |
| ACTTGCCATT | X76302 | putative nucleic acid binding protein RY-1 | 0.37 | 0.77 | 0.59 | 0.18 | 0.20 | 0.18 | 0.65 | 0.00 | 4.05 | 1.95 |
| GGAGAGACAG | Hs.46366 | zinc finger protein homologous to Zfp91 in mouse (EST) or other ESTs | 0.37 | 0.77 | 0.39 | 1.44 | 0.20 | 0.18 | 0.98 | 0.64 | 4.05 | 1.95 |
| GCTTTCATTG | Hs.118064 | similar to rat nuclear ubiquitous casein kinase 2 (EST) or chromosome 1 open reading frame 8 (EST) | 0.37 | 0.77 | 0.98 | 0.90 | 0.20 | 0.18 | 0.00 | 0.64 | 4.05 | 1.95 |
| CTCTGTAATT | AF000231 | RAB11A, member RAS oncogene family | 0.18 | 0.77 | 0.39 | 0.36 | 0.20 | 0.18 | 0.98 | 0.32 | 4.05 | 0.95 |
| TTCTGAAGCA | AK000508 | uncharacterized hypothalamus protein HT012 | 0.18 | 0.77 | 0.00 | 0.18 | 0.20 | 0.18 | 0.33 | 0.32 | 4.05 | 0.95 |
| GTCTCCGGGA | AK000591 | hypothetical protein FLJ20584 | 0.18 | 0.77 | 0.20 | 0.36 | 0.20 | 0.18 | 0.00 | 0.00 | 4.05 | 0.95 |
| TAGAATTTTC | AK001183 | MMP1; metalloprotease 1 (pitrilysin family) | 0.18 | 0.77 | 0.59 | 0.00 | 0.20 | 0.18 | 0.33 | 0.00 | 4.05 | 0.95 |
| TGAGCAAGCC | Hs.27801 | zinc finger protein 278 or EST | 0.18 | 0.77 | 0.00 | 0.18 | 0.20 | 0.18 | 0.00 | 0.00 | 4.05 | 0.95 |
| ATGACTGTAC | X95592 | nuclear DNA-binding protein | 0.18 | 0.77 | 0.20 | 0.18 | 0.20 | 0.18 | 0.33 | 0.32 | 4.05 | 0.95 |
| ACCAGAGAGC | Hs.181185 | DKFZP564D116 protein (EST) | 0.18 | 0.77 | 0.20 | 0.18 | 0.20 | 0.18 | 0.98 | 0.32 | 4.05 | 0.95 |
| ATTGTGCCAC | AL137457 | neurolysin (metallopeptidase M3 family) or cDNA DKFZp566G231 (from clone DKFZp566G231) | 0.00 | 0.77 | 0.20 | 0.36 | 0.20 | 0.18 | 0.00 | 0.00 | 4.05 | 0.00 |
| CGGCTTTTCT | Hs.324648 | EST | 0.00 | 0.77 | 0.98 | 0.54 | 0.20 | 0.18 | 0.65 | 0.32 | 4.05 | 0.00 |
| GATCTCGCAA | Hs.250773 | signal sequence receptor, alpha (translocon-associated protein alpha) (EST) | 1.29 | 1.93 | 1.18 | 1.99 | 0.78 | 0.18 | 0.00 | 0.64 | 4.02 | 2.69 |
| TGTGGCGTAT | Hs.288965 | cDNA: FLJ22300 fis, clone HRC04759 (EST) | 0.74 | 1.16 | 0.39 | 0.36 | 0.59 | 0.00 | 0.00 | 0.00 | 4.00 | 2.51 |
| CGCTGTTTTT | Hs.4756 | flap structure-specific endonuclease 1 | 0.74 | 1.16 | 0.59 | 1.99 | 0.59 | 0.00 | 0.33 | 0.00 | 4.00 | 2.51 |

468 genes in FIG. 2(d) (421 genes; 47 tags with no matches not shown)
SAGE data for genes up-regulated ≧ 4-fold in 786-0 VHL⁺ Nor. (V1) when compared to the average of two independent cultures of
Renal Proximal Tubule cells (RPTECs) grown under nomoxia (V5, V6). Also shown, for comparison, is the SAGE data for each gene in the 786-0
VHL⁻ Nor. (V2) with respect to its average expression in the two independent cultures of Renal Proximal Tubule cells (RPTECs) grown under
nomoxia (V5, V6). SAGE tag abundances were normalized before calculating ratios, as shown. The table is presented in descending order of V1/V5, V6.

| | | | | | | | | | | | | |
|---|---|---|---|---|---|---|---|---|---|---|---|---|
| TGCCCTCAGG | Hs.204238 | lipocalin 2 (oncogene 24p3) | 1.66 | 0.19 | 0.39 | 0.18 | 0.20 | 0.00 | 0.00 | 0.32 | 1.90 | 16.60 |
| ATGAAAAGAA | AL117612 | cDNA DKFZp564B1264 (from clone DKFZp564B1264) | 1.48 | 0.00 | 0.78 | 0.54 | 0.20 | 0.00 | 0.65 | 0.00 | 0.00 | 14.80 |
| GTAACAAGCT | AB007898 | KIAA0438 gene product | 1.11 | 0.19 | 0.59 | 0.18 | 0.00 | 0.18 | 0.98 | 0.32 | 2.11 | 12.33 |
| CTCCCAAGCT | AL117456 | KIAA0770 protein | 1.11 | 0.19 | 0.39 | 0.72 | 0.00 | 0.18 | 0.33 | 0.32 | 2.11 | 12.33 |
| AAAGAGAAAA | AL117492 | KIAA1007 protein | 1.11 | 0.39 | 0.20 | 0.90 | 0.20 | 0.00 | 0.00 | 0.00 | 3.90 | 11.10 |
| TGCAGACCCA | AK000098 | Tax1 (human T-cell leukemia virus type I) binding protein 1 | 2.03 | 0.39 | 0.98 | 1.08 | 0.20 | 0.18 | 0.65 | 0.00 | 2.05 | 10.68 |
| CAGGAGCCCC | AB023156 | KIAA0939 protein | 0.92 | 0.19 | 0.00 | 0.18 | 0.00 | 0.18 | 0.00 | 0.64 | 2.11 | 10.22 |
| GAGAATATCC | AF161386 | Similar to RIKEN cDNA 1110001J03 gene | 0.92 | 0.19 | 0.59 | 0.18 | 0.00 | 0.18 | 0.00 | 0.00 | 2.11 | 10.22 |
| GCTGCAAAGG | NM_032140 | hypothetical protein DKFZp434A1319 | 0.92 | 0.19 | 0.39 | 0.00 | 0.00 | 0.18 | 0.33 | 0.00 | 2.11 | 10.22 |
| TTTACTGGTA | AF155104 | RNA polymerase II tranacriptional regulation mediator (Med6, S. cerevisiae, homolog of) | 0.92 | 0.00 | 0.20 | 0.18 | 0.00 | 0.18 | 0.33 | 0.32 | 0.00 | 10.22 |
| GCGGTAAAAA | Hs.4746 | hypothetical protein FLJ21324 (EST) | 0.92 | 0.00 | 0.20 | 0.54 | 0.00 | 0.18 | 0.00 | 0.32 | 0.00 | 10.22 |
| TACTCGGTTG | Hs.213628 | Homo sapiens cDNA FLJ32269 fis, clone PROST1000526 (EST) | 0.92 | 0.00 | 0.59 | 0.36 | 0.00 | 0.18 | 0.00 | 0.00 | 0.00 | 10.22 |
| GCTTTTATTC | AF218024 | HT014 | 0.92 | 0.39 | 0.20 | 0.72 | 0.20 | 0.00 | 0.00 | 0.00 | 3.90 | 9.20 |
| GAAAGAGCTG | X14850 | H2A histone family, member X | 0.92 | 0.39 | 0.39 | 0.00 | 0.20 | 0.00 | 0.00 | 0.00 | 3.90 | 9.20 |
| AAGCAAAAGG | Hs.28505 | ubiquitin-conjugating enzyme E2H (homologous to yeast UBC8) (EST) | 0.92 | 0.39 | 0.00 | 0.36 | 0.20 | 0.00 | 0.33 | 0.32 | 3.90 | 9.20 |
| TGTCAAAAAA | Hs.55296 | HLA-B associated transcript 1 (EST) or other ESTs | 0.92 | 0.19 | 0.39 | 0.54 | 0.20 | 0.00 | 0.00 | 0.00 | 1.90 | 9.20 |
| TCAAATGCAA | AB020880 | KIAA0156 gene product | 0.74 | 0.19 | 0.20 | 0.18 | 0.00 | 0.18 | 0.00 | 0.00 | 2.11 | 8.22 |
| AACTCTGGAC | AK000518 | hypothetical protein FLJ20511 | 0.74 | 0.19 | 0.39 | 0.36 | 0.00 | 0.18 | 0.00 | 0.00 | 2.11 | 8.22 |
| ATTCCACAAA | Hs.21094 | RAB18, member RAS oncogene family (EST) | 0.74 | 0.19 | 0.20 | 0.36 | 0.00 | 0.18 | 0.33 | 0.00 | 2.11 | 8.22 |
| TGAACACGTT | Hs.118531 | EST | 0.74 | 0.19 | 0.20 | 0.00 | 0.00 | 0.18 | 0.00 | 0.00 | 2.11 | 8.22 |
| GGCTGGGGGG | Hs.198265 | Matrix metalloproteinase 25 (EST) | 0.74 | 0.19 | 0.20 | 0.00 | 0.00 | 0.18 | 0.00 | 0.00 | 2.11 | 8.22 |
| AATAAATGCA | Hs.29882 | predicted osteoblast protein (EST) | 0.74 | 0.19 | 0.39 | 0.18 | 0.00 | 0.18 | 0.00 | 0.00 | 2.11 | 8.22 |
| TATTCCCCAC | Hs.199316 | Homo sapiens, clone IMAGE: 3534002, mRNA, partial cds (EST) | 0.74 | 0.19 | 0.00 | 0.00 | 0.00 | 0.18 | 0.00 | 0.64 | 2.11 | 8.22 |
| TCTCCAACAA | Hs.257585 | Homo sapiens Hermansky-Pudlak syndrome type-3 protein (HPS3) mRNA, complete cds (EST) | 0.74 | 0.19 | 1.37 | 1.26 | 0.00 | 0.18 | 0.00 | 0.64 | 2.11 | 8.22 |
| TGACCTATTT | AB007916 | KIAA0447 gene product | 0.74 | 0.00 | 0.39 | 0.18 | 0.00 | 0.18 | 0.00 | 0.00 | 0.00 | 8.22 |
| TTGGTGAGGG | AF241255 | Prader-Willi syndrome chromosome region 1 | 0.74 | 0.00 | 0.00 | 0.00 | 0.00 | 0.18 | 0.65 | 0.00 | 0.00 | 8.22 |
| ACATTCTGCA | AK002170 | sorting nexin 2 | 0.74 | 0.00 | 0.00 | 0.18 | 0.00 | 0.18 | 0.00 | 0.00 | 0.00 | 8.22 |
| GCAGTCTAGC | BC006388 | N-acylsphingosine amidohydrolase (acid ceramidase)-like | 0.74 | 0.00 | 0.00 | 0.18 | 0.00 | 0.18 | 0.00 | 0.00 | 0.00 | 8.22 |
| TGTAAGAAAA | D84557 | minichromosome maintenance deficient (mis5, S. pombe) 6 | 0.74 | 0.00 | 0.20 | 0.90 | 0.00 | 0.18 | 0.00 | 0.00 | 0.00 | 8.22 |
| GGTAAAATTA | L37936 | Ts translation elongation factor mitochondrial | 0.74 | 0.00 | 0.00 | 0.00 | 0.00 | 0.18 | 0.00 | 0.32 | 0.00 | 8.22 |
| ACCGCTTGTT | U01062 | inositol 1,4,5-triphosphate receptor, type 3 | 0.74 | 0.00 | 0.39 | 0.72 | 0.00 | 0.18 | 0.00 | 0.32 | 0.00 | 8.22 |
| GTTAAATATG | Hs.288158 | hypothetical protein FLJ23591 (EST) | 0.74 | 0.00 | 0.78 | 0.54 | 0.00 | 0.18 | 0.00 | 0.00 | 0.00 | 8.22 |
| GGTGGAAAAA | Hs.178728 | methyl-CpG binding domain protein 3 (EST) or non-metastatic cells 4, protein expressed in (EST) | 0.74 | 0.00 | 0.00 | 0.00 | 0.00 | 0.18 | 0.00 | 0.00 | 0.00 | 8.22 |
| AAAATGTATT | AF035752 | U caveolin 2 | 0.74 | 0.39 | 0.59 | 0.54 | 0.20 | 0.00 | 0.33 | 0.32 | 3.90 | 7.40 |
| CTAAAGTACT | Hs.171553 | Moderately similar to CLC3_HUMAN CHLORIDE CHANNEL PROTEIN 3 [H. sapiens] (EST) | 0.74 | 0.39 | 0.20 | 0.18 | 0.20 | 0.00 | 0.00 | 0.00 | 3.90 | 7.40 |
| TTGGCCTCTG | Hs.252259 | EST | 0.74 | 0.39 | 1.18 | 0.54 | 0.20 | 0.00 | 0.00 | 0.00 | 3.90 | 7.40 |
| TTCATTAAAA | Hs.287797 | ITGB1; integrin, beta 1 (fibronectin raceptor, beta polypeptide, antigen CD29 includes MDF2, | 1.48 | 0.77 | 0.59 | 1.44 | 0.39 | 0.00 | 0.98 | 0.32 | 3.95 | 7.59 |
| | | MSK12) (EST) | | | | | | | | | | |
| CAGGACTAAG | AB046820 | KIAA1600 protein | 0.74 | 0.19 | 0.20 | 0.54 | 0.20 | 0.00 | 0.00 | 0.00 | 1.90 | 7.40 |
| AACAGCTGGA | AF030424 | histone acetyltransferase 1 | 0.74 | 0.19 | 0.00 | 0.36 | 0.20 | 0.00 | 0.00 | 0.00 | 1.90 | 7.40 |
| GAAACAAAAT | AF052182 | DHHC1 protein | 0.74 | 0.19 | 0.39 | 0.18 | 0.20 | 0.00 | 0.00 | 0.00 | 1.90 | 7.40 |
| CATCATTAAT | AF075027 | Homo sapiens full length insert cDNA YI37C01. | 0.74 | 0.19 | 0.20 | 0.00 | 0.20 | 0.00 | 0.00 | 0.00 | 1.90 | 7.40 |
| TAAGATGGCA | AF119042 | transcriptional intermediary factor 1 | 0.74 | 0.19 | 0.20 | 0.36 | 0.20 | 0.00 | 0.00 | 0.00 | 1.90 | 7.40 |
| GGATGGGTGT | AL080088 | DKFZP564K2062 protein | 0.74 | 0.19 | 0.20 | 0.18 | 0.20 | 0.00 | 0.00 | 0.32 | 1.90 | 7.40 |
| GTGAAACGCC | NM_024345 | hypothetical protein MGC10765 or other ESTs | 0.74 | 0.19 | 0.00 | 0.18 | 0.20 | 0.00 | 0.00 | 0.32 | 1.90 | 7.40 |
| CAACCAACTA | Hs.82071 | Cbp/p300-interacting transactivator, with Glu/Asp-rich carboxy-terminal domain, | 0.74 | 0.19 | 0.20 | 0.36 | 0.20 | 0.00 | 0.00 | 0.00 | 1.90 | 7.40 |
| | | 2 (EST) or lactate dehydrogenase A (EST) | | | | | | | | | | |
| TATTAAATAG | Hs.155543 | proteasome (prosome, macropain) 26S subunit, non-ATPase, 7 (Mov34 homolog) or EST | 0.74 | 0.19 | 0.59 | 1.26 | 0.20 | 0.00 | 0.00 | 0.32 | 1.90 | 7.40 |
| CAAACTGAAG | Hs.334826 | splicing factor 3b, subunit 1, 155 kD (EST) | 0.74 | 0.19 | 0.00 | 0.18 | 0.20 | 0.00 | 0.33 | 0.00 | 1.90 | 7.40 |
| CTGTACTAGG | AB026894 | nesca protein | 0.74 | 0.00 | 0.20 | 0.00 | 0.20 | 0.00 | 0.00 | 0.00 | 0.00 | 7.40 |
| ACCATTGTGT | AF088060 | CGI-203 protein | 0.74 | 0.00 | 0.00 | 0.36 | 0.20 | 0.00 | 0.00 | 0.00 | 0.00 | 7.40 |
| ACACTTCTTG | AF309777 | elongation factor G (EFG) mRNA, complete cds; nuclear gene for mitochondrial product | 0.74 | 0.00 | 0.39 | 0.18 | 0.20 | 0.00 | 0.65 | 0.00 | 0.00 | 7.40 |
| GTTGAAACTC | AL080113 | DEAD/H (Asp-Glu-Ala-Asp/His) box polypeptide 17 (72 kD) | 0.74 | 0.00 | 0.39 | 0.00 | 0.20 | 0.00 | 0.00 | 0.00 | 0.00 | 7.40 |
| TCATTTCCTG | U18932 | N-deacetylase/N-sulfotransferase (heparan glucosaminyl) 1 | 0.74 | 0.00 | 0.20 | 0.00 | 0.20 | 0.00 | 0.00 | 0.00 | 0.00 | 7.40 |
| GGCCTTCCTT | Hs.75789 | N-myc downstream regulated (EST) or other ESTs | 0.74 | 0.00 | 0.20 | 0.54 | 0.20 | 0.00 | 0.33 | 0.00 | 0.00 | 7.40 |
| ATTAAAAAAA | Hs.78713 | EST | 0.74 | 0.00 | 0.20 | 1.44 | 0.20 | 0.00 | 0.00 | 0.00 | 0.00 | 7.40 |
| GTTTTTACTT | Hs.4248 | vav 2 oncogene (EST) | 0.74 | 0.00 | 0.39 | 0.90 | 0.20 | 0.00 | 0.00 | 0.32 | 0.00 | 7.40 |
| ATTTTAGAAT | Hs.302649 | nucleosome assembly protein 1-like 1 (EST) | 0.74 | 0.00 | 0.39 | 0.00 | 0.20 | 0.00 | 0.00 | 0.00 | 0.00 | 7.40 |
| CCAAAAGTAA | Hs.1501 | SDC2; syndecan 2 (heparan sulfate proteoglycan 1, cell surface-associated, fibroglycan) (EST) | 0.74 | 0.00 | 0.39 | 0.00 | 0.20 | 0.00 | 0.00 | 0.00 | 0.00 | 7.40 |
| TAGGATGGGG | U51478 | ATPase, Na+/K+ transporting, beta 3 polypeptide. | 1.29 | 0.58 | 0.78 | 1.08 | 0.00 | 0.35 | 0.65 | 0.97 | 3.31 | 7.37 |
| TGCATATCAT | Y08614 | Homo sapiens mRNA for CRM1 protein. | 1.29 | 0.58 | 0.59 | 0.00 | 0.00 | 0.35 | 0.00 | 0.32 | 3.31 | 7.37 |
| TGTTAATGTT | AK001730 | Homo sapiens cDNA FLJ1086 fis, clone NT2RP4001644 | 1.29 | 0.58 | 0.78 | 0.36 | 0.20 | 0.18 | 0.00 | 0.00 | 3.05 | 6.79 |
| TACATAATTA | AF080092 | Homo sapiens trophoblast STAT utron mRNA. | 1.29 | 0.39 | 1.18 | 0.36 | 0.20 | 0.18 | 0.00 | 0.00 | 2.05 | 6.79 |
| TACTTGTGTG | AF035287 | Homo sapiens clone 23742 mRNA, partial cds. | 2.40 | 0.77 | 1.76 | 1.44 | 0.00 | 0.71 | 0.98 | 0.00 | 2.17 | 6.76 |
| AAAGTGCATC | AJ277437 | FGFRL1; fibroblast growth factor receptor-like 1 | 2.40 | 1.35 | 1.37 | 1.44 | 0.39 | 0.35 | 0.65 | 0.64 | 3.65 | 6.49 |
| TTTGGAATGT | AB018266 | matrin 3 | 2.40 | 0.58 | 0.98 | 1.63 | 0.39 | 0.35 | 0.65 | 0.64 | 1.57 | 6.49 |
| CAGAGTGCTG | Hs.9006 | VAMP (vesicle-associated membrane protein)-associated protein A (33 kD) (EST) | 1.11 | 0.58 | 0.59 | 0.72 | 0.00 | 0.35 | 0.98 | 0.32 | 3.31 | 6.34 |
| CAGCCATAAA | AF257241 | BTB (POZ) domain containing 1 | 1.11 | 0.39 | 0.20 | 0.54 | 0.00 | 0.35 | 0.00 | 0.32 | 2.23 | 6.34 |
| TATTTTACGT | L34587 | transcription elongation factor B (SIII), polypeptide 1 (15 kD, elongin C) | 1.11 | 0.39 | 0.78 | 0.36 | 0.00 | 0.35 | 0.33 | 0.64 | 2.23 | 6.34 |
| TTTCTTAAAG | AB002322 | RNA binding protein; AT-rich element binding factor | 0.55 | 0.19 | 0.59 | 0.00 | 0.00 | 0.18 | 0.00 | 1.29 | 2.11 | 6.11 |
| CATTTGTAAA | AB023188 | KIAA0971 protein or other ESTs | 0.55 | 0.19 | 0.39 | 0.54 | 0.00 | 0.18 | 0.00 | 0.00 | 2.11 | 6.11 |
| TCTACTGTTA | AB044943 | N-myc downstream-regulated gene 3 | 0.55 | 0.19 | 0.00 | 0.00 | 0.00 | 0.18 | 0.33 | 0.00 | 2.11 | 6.11 |
| CTTATTCCTT | AF047472 | BUB3 (budding uninhibited by benzimidazoles 3, yeast) homolog | 0.55 | 0.19 | 0.59 | 0.54 | 0.00 | 0.18 | 0.00 | 0.00 | 2.11 | 6.11 |
| TCTTTTCAAA | AF163324 | CS box-containing WD protein (+A) or membrane-bound transcription factor protease, site 2 (EST) | 0.55 | 0.19 | 0.20 | 0.00 | 0.00 | 0.18 | 0.00 | 0.00 | 2.11 | 6.11 |
| TAAACATTGT | AL049943 | integral inner nuclear membrane protein | 0.55 | 0.19 | 0.59 | 0.72 | 0.00 | 0.18 | 0.33 | 0.00 | 2.11 | 6.11 |
| TATATACATT | AL050216 | peptidase (mitochondrial processing) beta | 0.55 | 0.19 | 0.39 | 0.36 | 0.00 | 0.18 | 0.00 | 0.00 | 2.11 | 6.11 |
| GAAGCAAAAA | AL110260 | DKFZP586B1621 protein | 0.55 | 0.19 | 0.20 | 0.00 | 0.00 | 0.18 | 0.33 | 0.00 | 2.11 | 6.11 |
| GTCTCAGTCA | BC003616 | bleomycin hydrolase | 0.55 | 0.19 | 0.59 | 0.00 | 0.00 | 0.18 | 0.00 | 0.00 | 2.11 | 6.11 |
| CTCTTATTTC | BC005158 | hypothetical protein MGC4618 | 0.55 | 0.19 | 0.00 | 0.18 | 0.00 | 0.18 | 0.00 | 0.00 | 2.11 | 6.11 |
| CAGTATGTCC | BC008752 | clone MGC: 2485, mRNA, complete cds | 0.55 | 0.19 | 0.00 | 0.18 | 0.00 | 0.18 | 0.33 | 0.00 | 2.11 | 6.11 |
| TATCCATACC | D00723 | glycine cleavage system protein H (aminomethyl carrier) | 0.55 | 0.19 | 1.18 | 0.72 | 0.00 | 0.18 | 0.33 | 0.00 | 2.11 | 6.11 |
| CACTGTGTGT | NM_022458 | chromosome 7 open reading frame 2 or hypothetical protein FLJ21172 | 0.55 | 0.19 | 0.59 | 0.00 | 0.00 | 0.18 | 0.00 | 0.00 | 2.11 | 6.11 |
| GGGGCTGTGG | U02619 | general transcription factor IIIC, polypeptide 1 (alpha subunit, 220 kD) or another EST | 0.55 | 0.19 | 0.59 | 0.54 | 0.00 | 0.18 | 0.00 | 0.00 | 2.11 | 6.11 |
| TGGATGTACT | U86782 | 26S proteasome-associated pad1 homolog | 0.55 | 0.19 | 0.39 | 0.18 | 0.00 | 0.18 | 0.00 | 0.00 | 2.11 | 6.11 |
| GGGCCCCTGG | X57205 | fibroblast growth factor receptor 4 | 0.55 | 0.19 | 0.00 | 0.18 | 0.00 | 0.18 | 0.00 | 0.00 | 2.11 | 6.11 |
| TATCCTGGCT | X85116 | erythrocyte membrane protein band 7.2 (stomatin) | 0.55 | 0.19 | 0.20 | 0.54 | 0.00 | 0.18 | 0.65 | 0.64 | 2.11 | 6.11 |
| GTGGTGTCTG | Hs.27048 | EST | 0.55 | 0.19 | 0.20 | 0.18 | 0.00 | 0.18 | 0.33 | 0.00 | 2.11 | 6.11 |
| ATAGATGGGG | Hs.183684 | eukaryotic translation initiation factor 4 gamma, 2 (EST) or mitogen-activated protein kinase kinase 1 | 0.55 | 0.19 | 0.20 | 0.18 | 0.00 | 0.18 | 0.33 | 0.32 | 2.11 | 6.11 |
| | | (EST) | | | | | | | | | | |
| GCCCCTTGCA | Hs.351593 | fibrinogen, A alpha polypeptide (EST) | 0.55 | 0.19 | 0.39 | 0.18 | 0.00 | 0.18 | 0.00 | 0.00 | 2.11 | 6.11 |
| AGAAGGATGC | Hs.90458 | serine palmitoyltransferase, long chain base subunit 1 (EST) | 0.55 | 0.19 | 0.39 | 0.54 | 0.00 | 0.18 | 0.00 | 0.00 | 2.11 | 6.11 |
| AATCTGGTTG | Hs.103804 | heterogeneous nuclear ribonucleoprotein U (scaffold attachment factor A) (EST) | 0.55 | 0.19 | 0.39 | 0.72 | 0.00 | 0.18 | 0.65 | 0.32 | 2.11 | 6.11 |
| AGTTTAAGCA | Hs.7885 | phosphatidylinositol binding clathrin assembly protein (EST) | 0.55 | 0.19 | 0.39 | 0.00 | 0.00 | 0.18 | 0.33 | 0.32 | 2.11 | 6.11 |
| TCATCATCTG | Hs.351937 | ribosomal protein, large P2 (EST) | 0.55 | 0.19 | 0.59 | 0.54 | 0.00 | 0.18 | 0.65 | 0.00 | 2.11 | 6.11 |
| GTCCCTGGGT | Hs.284296 | surfeit 4 (EST) or RAB14, member RAS oncogene family (EST) | 0.55 | 0.19 | 0.59 | 0.54 | 0.00 | 0.18 | 0.00 | 0.00 | 2.11 | 6.11 |
| ATGTGTTTCA | Hs.80887 | v-yes-1 Yamaguchi sarcoma viral related oncogene homolog (EST) | 0.55 | 0.19 | 0.78 | 0.18 | 0.00 | 0.18 | 0.00 | 0.00 | 2.11 | 6.11 |
| TACCAAGGAT | Hs.347286 | hypothetical protein FLJ22427 (EST) | 0.55 | 0.19 | 0.78 | 0.54 | 0.00 | 0.18 | 0.00 | 0.00 | 2.11 | 6.11 |
| ATTTTAACAA | Hs.155218 | E1B-55 kDa-associated protein 5 (EST) or other EST | 0.55 | 0.19 | 0.00 | 0.00 | 0.00 | 0.18 | 0.00 | 0.00 | 2.11 | 6.11 |
| AAAAAATAAA | Hs.151242 | serine (or cysteine) proteinase inhibitor, clade G (C1 inhibitor), member 1 (EST) or brain-specific | 0.55 | 0.19 | 0.00 | 0.00 | 0.00 | 0.18 | 0.00 | 0.00 | 2.11 | 6.11 |
| | | angiogenesis inhibitor 3 (EST) or pin | | | | | | | | | | |
| GTAAATTTAA | BC003635 | MMP7; Matrix metelloproteinase 7 (matrilysin, uterine) | 1.11 | 0.19 | 0.20 | 0.54 | 0.00 | 0.35 | 0.00 | 0.00 | 1.09 | 6.34 |
| GGCTGAATTT | AB014548 | KIAA0648 protein | 0.55 | 0.00 | 0.20 | 0.36 | 0.00 | 0.18 | 0.00 | 0.00 | 0.00 | 6.11 |
| TTTCTGAAAA | AF035840 | NADH dehydrogenase (ubiquinone) 1 beta subcomplex, 6 (17 kD, B17) | 0.55 | 0.00 | 0.00 | 0.00 | 0.00 | 0.18 | 0.00 | 0.00 | 0.00 | 6.11 |
| CTTCCTATAC | AF044323 | COX15 (yeast) homolog, cytochrome c oxidase assembly protein | 0.55 | 0.00 | 0.39 | 0.54 | 0.00 | 0.18 | 0.00 | 0.00 | 0.00 | 6.11 |
| CTCAGCTGCC | AF086378 | hypothetical protein FLJ13852 | 0.55 | 0.00 | 0.59 | 0.72 | 0.00 | 0.18 | 0.00 | 0.00 | 0.00 | 6.11 |
| GGGGGCAAGC | AF219137 | PODLX2; endoglycan | 0.55 | 0.00 | 0.00 | 0.00 | 0.00 | 0.18 | 0.00 | 0.00 | 0.00 | 6.11 |
| TCTTAATGGG | AF359563 | inner mitochondrial membrane peptidase 2 like | 0.55 | 0.00 | 0.20 | 0.00 | 0.00 | 0.18 | 0.98 | 0.32 | 0.00 | 6.11 |
| GTTGTAAATA | AK001043 | ILKAP; integrin-linked kinase-associated serine/threonine phosphatase 2C | 0.55 | 0.00 | 0.39 | 0.54 | 0.00 | 0.18 | 0.00 | 0.00 | 0.00 | 6.11 |
| AAATGCCCTC | J02645 | eukaryotic translation initiation factor 2, subunit 1 (alpha, 35 kD) | 0.55 | 0.00 | 0.39 | 0.00 | 0.00 | 0.18 | 0.33 | 0.00 | 0.00 | 6.11 |
| CTATGAAGAT | M15841 | small nuclear ribonucleoprotein polypeptide B″ | 0.55 | 0.00 | 0.20 | 0.36 | 0.00 | 0.18 | 0.00 | 0.64 | 0.00 | 6.11 |
| TTTACAAACC | M97936 | signal transducer and activator of transcription 1.91 kD | 0.55 | 0.00 | 0.39 | 0.72 | 0.00 | 0.18 | 0.00 | 0.00 | 0.00 | 6.11 |
| TAGGACTTGG | Hs.78915 | GA-binding protein transcription factor, beta subunit 1 (53 kD) (EST) | 0.55 | 0.00 | 0.20 | 0.36 | 0.00 | 0.18 | 0.00 | 0.00 | 0.00 | 6.11 |
| AGCTCAGTGA | Hs.351706 | Homo sapiens cDNA FLJ32203 fis, clone PLACE6003038, weakly similar to ZINC | 0.55 | 0.00 | 0.20 | 0.00 | 0.00 | 0.18 | 0.00 | 0.00 | 0.00 | 6.11 |
| | | FINGER PROTEIN 84 | | | | | | | | | | |
| CGTGTCGATA | Hs.4437 | ribosomal protein L28 (EST) | 0.55 | 0.00 | 0.20 | 0.00 | 0.00 | 0.18 | 0.00 | 0.00 | 0.00 | 6.11 |
| ATCAAGAATC | Hs.14623 | IFI30; interferon, gamma-inducible protein 30 (EST) | 0.55 | 0.00 | 0.39 | 0.00 | 0.00 | 0.18 | 0.00 | 0.97 | 0.00 | 6.11 |
| TTAGCACTGT | Hs.94308 | RAB35, member RAS oncogene family (EST) | 0.55 | 0.00 | 0.39 | 0.18 | 0.00 | 0.18 | 0.00 | 0.00 | 0.00 | 6.11 |
| CTTGGTGCTG | Hs.180842 | ribosomal protein L13 (EST) | 0.55 | 0.00 | 0.78 | 0.36 | 0.00 | 0.18 | 0.00 | 0.32 | 0.00 | 6.11 |
| TCAACACAGT | Hs.218710 | ESTs, Weakly similar to I38022 hypothetical protein [H. sapiens] (EST) | 0.55 | 0.00 | 0.00 | 0.54 | 0.00 | 0.18 | 0.00 | 0.64 | 0.00 | 6.11 |
| TCTGGACTTT | Hs.50730 | EST | 0.55 | 0.00 | 0.00 | 0.18 | 0.00 | 0.18 | 0.00 | 0.00 | 0.00 | 6.11 |
| AGCCAATTCC | Hs.3192 | 6-pyruvoyl-tetrahydropterin synthase/dimerization cofactor of hepatocyte nuclear factor 1 alpha | 0.55 | 0.00 | 0.00 | 0.00 | 0.00 | 0.18 | 0.00 | 0.00 | 0.00 | 6.11 |
| | | (TCF1) (EST) | | | | | | | | | | |
| GAATGAGGAC | D42073 | reticulocalbin 1, EF-hand calcium binding domain | 3.87 | 1.93 | 3.14 | 2.53 | 0.20 | 1.06 | 2.93 | 0.32 | 3.06 | 6.14 |
| GCAAAGAAAA | AF161481 | LIM domain only 4 or others | 2.21 | 0.39 | 1.96 | 1.26 | 0.20 | 0.53 | 0.65 | 0.00 | 1.07 | 6.05 |
| TACTAAAAAA | AF013160 | NADH dehydrogenase (ubiquinone) Fe-S protein 2 (49 kD) (NADH-coenzyme Q reductase | 1.68 | 0.39 | 0.59 | 1.08 | 0.20 | 0.35 | 0.98 | 0.00 | 1.42 | 6.04 |
| CAGATTTCCA | Hs.21893 | hypothetical protein DKFZp761N0624 | 1.11 | 0.19 | 1.37 | 0.36 | 0.20 | 0.18 | 0.00 | 0.00 | 1.00 | 5.84 |
| TTACACTGGA | AB049127 | MAP/microtubule affinity-regulating kinase like 1 or other ESTs | 0.55 | 0.39 | 0.39 | 0.18 | 0.20 | 0.00 | 0.00 | 0.64 | 3.90 | 5.50 |
| CCAAGAATGT | AF151819 | SH3-domain, GRB2-like, endophilin B1 | 0.55 | 0.39 | 0.00 | 0.18 | 0.20 | 0.00 | 0.33 | 0.00 | 3.90 | 5.50 |
| CTTGCTCTCC | AF208845 | hypothetical protein | 0.55 | 0.39 | 0.39 | 0.54 | 0.20 | 0.00 | 0.00 | 0.00 | 3.90 | 5.50 |
| TAAGTTCCTT | AF210430 | hypothetical protein MGC5627 | 0.55 | 0.39 | 0.20 | 0.18 | 0.20 | 0.00 | 0.00 | 0.00 | 3.90 | 5.50 |
| CAGTGGGGTT | AK000310 | hypothetical protein FLJ20303 | 0.55 | 0.39 | 0.98 | 0.00 | 0.20 | 0.00 | 0.00 | 0.00 | 3.90 | 5.50 |
| TGGGAGCTTT | AK002165 | DKFZP564O123 protein (EST) | 0.55 | 0.39 | 0.20 | 0.72 | 0.20 | 0.00 | 0.33 | 0.00 | 3.90 | 5.50 |
| TGATCAAAAT | BC000686 | UCC1 protein | 0.55 | 0.39 | 0.78 | 0.72 | 0.20 | 0.00 | 0.00 | 0.00 | 3.90 | 5.50 |
| GGCAACAAAA | BC003572 | karyopherin (importin) beta 1 | 0.55 | 0.39 | 0.78 | 0.54 | 0.20 | 0.00 | 0.98 | 0.00 | 3.90 | 5.50 |
| TTAAAACAAA | BC004258 | hypothetical protein MGC10753 | 0.55 | 0.39 | 0.39 | 0.00 | 0.20 | 0.00 | 0.33 | 0.00 | 3.90 | 5.50 |
| AAGGTCCTAG | L49054 | myeloid leukemia factor 1 | 0.55 | 0.39 | 0.59 | 0.54 | 0.20 | 0.00 | 0.65 | 0.32 | 3.90 | 5.50 |
| TGTTAGATTT | U35451 | chromobox homolog 1 (Drosophila HP1 beta) | 0.55 | 0.39 | 1.18 | 1.08 | 0.20 | 0.00 | 0.00 | 0.32 | 3.90 | 5.50 |
| GCGTCGGGGA | X89814 | interferon (alpha, beta and omega) receptor 2 | 0.55 | 0.39 | 0.98 | 1.44 | 0.20 | 0.00 | 0.65 | 0.00 | 3.90 | 5.50 |
| ACCTCCCCAG | Y09305 | dual-specificity tyrosine-(Y)-phosphorylation regulated kinase 4 | 0.55 | 0.39 | 0.00 | 0.18 | 0.20 | 0.00 | 0.00 | 0.64 | 3.90 | 5.50 |
| TGCTACGATC | Hs.344911 | EST | 0.55 | 0.39 | 0.20 | 0.18 | 0.20 | 0.00 | 0.00 | 0.32 | 3.90 | 5.50 |
| TGTACCTTGT | Hs.215160 | EST | 0.55 | 0.39 | 0.20 | 0.18 | 0.20 | 0.00 | 0.00 | 0.00 | 3.90 | 5.50 |
| GAGAGCTTAA | Hs.278682 | Phosphatidylglycerophosphate Synthase (EST) | 0.55 | 0.39 | 0.20 | 0.36 | 0.20 | 0.00 | 0.00 | 0.00 | 3.90 | 5.50 |
| TATAAACCTT | Hs.22120 | EST | 0.55 | 0.39 | 0.20 | 0.00 | 0.20 | 0.00 | 0.33 | 0.00 | 3.90 | 5.50 |
| CTTTTTGGAA | Hs.77558 | thyroid hormone receptor interactor 7 (EST) | 0.55 | 0.39 | 0.20 | 0.00 | 0.20 | 0.00 | 0.00 | 0.00 | 3.90 | 5.50 |
| GAGATGTCTG | Hs.74861 | activated RNA polymerase II transcription cofactor 4 (EST) or other ESTs | 0.55 | 0.39 | 0.59 | 0.54 | 0.20 | 0.00 | 0.00 | 0.00 | 3.90 | 5.50 |
| GTAAGATTAG | Hs.250705 | mitochondrial carrier homolog 1 (EST) or Homo sapiens cDNA FLJ11685 fis, clone | 0.55 | 0.39 | 0.00 | 0.18 | 0.20 | 0.00 | 0.33 | 0.32 | 3.90 | 5.50 |
| | | HEMBA1004934 (EST) | | | | | | | | | | |
| CTCGTTAAGA | Hs.106061 | RD RNA-binding protein or other ESTs | 0.55 | 0.39 | 0.00 | 0.54 | 0.20 | 0.00 | 0.33 | 0.32 | 3.90 | 5.50 |
| CTGAAAATTC | AB067502 | KIAA1915 protein | 0.55 | 0.39 | 0.00 | 1.08 | 0.20 | 0.00 | 0.00 | 0.00 | 3.90 | 5.50 |
| CTACTGTACC | Hs.80120 | UDP-N-acetyl-alpha-D-galactosamine: polypeptide N-acetylgalactosaminyltransferase 1 | 0.55 | 0.39 | 1.37 | 0.36 | 0.20 | 0.00 | 0.00 | 0.00 | 3.90 | 5.50 |
| | | (GalNAc-T1)(EST) | | | | | | | | | | |
| CTAGCAGAGC | D17223 | dyskeratosis congenita 1, dyskerin | 1.66 | 0.58 | 0.00 | 0.54 | 0.59 | 0.00 | 0.00 | 0.32 | 1.97 | 5.63 |
| GTCTTACCTA | NM_024586 | hypothetical protein FLJ12492 | 1.66 | 0.58 | 0.59 | 0.72 | 0.59 | 0.00 | 0.00 | 0.32 | 1.97 | 5.63 |
| GGTGTTGCCG | Hs.97656 | Weakly similar to ALU1_HUMAN ALU SUBFAMILY J SEQUENCE | 1.11 | 0.39 | 0.39 | 0.90 | 0.39 | 0.00 | 0.33 | 0.32 | 2.00 | 5.69 |
| | | CONTAMINATION WARNING ENTRY [H. sapiens] (EST) | | | | | | | | | | |
| ACATAAGATC | AB020980 | putative membrane protein | 0.55 | 0.19 | 0.20 | 0.54 | 0.20 | 0.00 | 0.00 | 0.00 | 1.90 | 5.50 |
| TGACCACCTA | AF151856 | DKFZP547E2110 protein | 0.55 | 0.19 | 0.00 | 0.00 | 0.20 | 0.00 | 0.33 | 0.00 | 1.90 | 5.50 |
| AAACATTAGC | AF208850 | protein tyrosine phosphatase type IVA, member 2 | 0.55 | 0.19 | 0.59 | 0.18 | 0.20 | 0.00 | 0.00 | 0.00 | 1.90 | 5.50 |
| GCCAAAGTGT | AF237952 | PAN2 protein | 0.55 | 0.19 | 0.39 | 0.36 | 0.20 | 0.00 | 0.65 | 0.64 | 1.90 | 5.50 |
| ACTTTAACAG | AL031588 | CELSR1; cadherin, EGF LAG seven-pass G-type receptor 1, flamingo (Drosophila) homolog | 0.55 | 0.19 | 0.39 | 0.00 | 0.20 | 0.00 | 0.33 | 0.00 | 1.90 | 5.50 |
| TTTATCCCAA | AL050260 | DKFZP547E1010 protein (EST) | 0.55 | 0.19 | 0.00 | 0.00 | 0.20 | 0.00 | 0.00 | 0.00 | 1.90 | 5.50 |
| GATTTTTCAT | AL050282 | like mouse brain protein E46 | 0.55 | 0.19 | 0.39 | 0.72 | 0.20 | 0.00 | 0.33 | 0.32 | 1.90 | 5.50 |
| GGATGTGTTT | AL137604 | tumor antigen SLP-8p | 0.55 | 0.19 | 0.20 | 0.00 | 0.20 | 0.00 | 0.00 | 0.32 | 1.90 | 5.50 |
| TTGATCGAAG | BC000927 | hypothetical protein MGC5378 | 0.55 | 0.19 | 0.00 | 0.00 | 0.20 | 0.00 | 0.33 | 0.32 | 1.90 | 5.50 |
| ATCATTTGTT | BC004568 | Arg/Abl-interacting protein ArgBP2 | 0.55 | 0.19 | 0.39 | 0.18 | 0.20 | 0.00 | 0.33 | 0.00 | 1.90 | 5.50 |
| TTTTAAAAAT | M65213 | catechol-O-methyltransferase | 0.55 | 0.19 | 0.20 | 0.54 | 0.20 | 0.00 | 0.00 | 0.00 | 1.90 | 5.50 |
| ATCTTAGCTT | M80783 | TNFAIP1; tumor necrosis factor, alpha-induced protein 1 (endothelial) | 0.55 | 0.19 | 0.00 | 0.00 | 0.20 | 0.00 | 0.00 | 0.32 | 1.90 | 5.50 |
| CCAAATGATG | NM_024699 | hypothetical protein FLJ14007 | 0.55 | 0.19 | 0.20 | 0.00 | 0.20 | 0.00 | 0.33 | 0.00 | 1.90 | 5.50 |
| CTTTTTAAAT | U17714 | suppression of tumorigenicity 13 (colon carcinoma) (Hsp70-interacting protein) | 0.55 | 0.19 | 0.39 | 0.54 | 0.20 | 0.00 | 0.33 | 0.00 | 1.90 | 5.50 |
| AGGCTTTATG | U68494 | hbc647 mRNA sequence | 0.55 | 0.19 | 0.39 | 0.36 | 0.20 | 0.00 | 0.65 | 0.32 | 1.90 | 5.50 |
| CTAGAACATC | Hs.81424 | ubiquitin-like 1 (sentrin) (EST) | 0.55 | 0.19 | 0.20 | 0.90 | 0.20 | 0.00 | 0.00 | 0.00 | 1.90 | 5.50 |
| GAGCTCTTGT | Hs.264153 | Fanconi anemia, complementation group A (EST) | 0.55 | 0.19 | 0.39 | 0.36 | 0.20 | 0.00 | 0.00 | 0.00 | 1.90 | 5.50 |
| GAGGCTTAAT | Hs.78851 | hypothetical protein (EST) | 0.55 | 0.19 | 0.39 | 0.00 | 0.20 | 0.00 | 0.00 | 0.00 | 1.90 | 5.50 |
| TGAGTTGGGT | BC014878 | surfelt 6 | 0.55 | 0.19 | 0.59 | 0.54 | 0.20 | 0.00 | 0.00 | 0.00 | 1.90 | 5.50 |
| AGTGAGGATA | Hs.150614 | Weakly similar to ALU4_HUMAN ALU SUBFAMILY SB2 SEQUENCE CONTAMINATION | 0.55 | 0.19 | 0.00 | 0.18 | 0.20 | 0.00 | 0.33 | 0.00 | 1.90 | 5.50 |
| | | WARNING ENTRY [H. sapiens] (EST) | | | | | | | | | | |
| AACTTAAAAA | Hs.108708 | protein phosphatase 3 (formerly 2B), catalytic subunit, alpha isoform (calcineurin A alpha) (EST) | 0.55 | 0.19 | 0.00 | 0.18 | 0.20 | 0.00 | 0.00 | 0.00 | 1.90 | 5.50 |
| | | or calcium/calmodulin-dependent pro | | | | | | | | | | |
| AGTAAAAAAA | Hs.9552 | binder of Arl Two (EST) or Huntingtin interacting protein M (EST) or others ESTs | 0.55 | 0.19 | 0.00 | 0.36 | 0.20 | 0.00 | 0.00 | 0.00 | 1.90 | 5.50 |
| AACCCGGAAG | U90144 | butyrophilin, subfamily 3, member A2 | 1.11 | 0.19 | 0.59 | 0.72 | 0.39 | 0.00 | 0.00 | 0.64 | 0.97 | 5.69 |
| AATTTAAAAA | AF035295 | acetyl-Coenzyme A acyltransferase 1 (peroxisomal 3-oxoacyl-Coenzyme A thiolase) or steroidogenic | 0.55 | 0.00 | 0.20 | 0.18 | 0.20 | 0.00 | 0.00 | 0.00 | 0.00 | 5.50 |
| | | acute regulatory protein related | | | | | | | | | | |
| CAAATTACAA | AF038554 | DENR; density-regulated protein | 0.55 | 0.00 | 0.00 | 0.36 | 0.20 | 0.00 | 0.00 | 0.00 | 0.00 | 5.50 |
| GGATCTGCAA | AF131218 | chromosome 16 open reading frame 5 | 0.55 | 0.00 | 0.00 | 0.00 | 0.20 | 0.00 | 0.00 | 0.00 | 0.00 | 5.50 |
| GTGGTGGACG | AK001322 | growth differentiation factor 1 or others | 0.55 | 0.00 | 0.00 | 0.00 | 0.20 | 0.00 | 0.00 | 0.00 | 0.00 | 5.50 |
| GCACAGAGCC | AK001431 | hypothetical protein FLJ10569 | 0.55 | 0.00 | 0.00 | 0.36 | 0.20 | 0.00 | 0.33 | 0.32 | 0.00 | 5.50 |
| TATCAAAACA | AL080116 | origin recognition complex, subunit 3 (yeast homolog)-like or another | 0.55 | 0.00 | 0.39 | 0.54 | 0.20 | 0.00 | 0.65 | 0.00 | 0.00 | 5.50 |
| GCCAAGTTTG | BC001053 | proteasome (prosome, macropain) 26S subunit, non-ATPase, 1 | 0.55 | 0.00 | 0.20 | 0.18 | 0.20 | 0.00 | 0.33 | 0.00 | 0.00 | 5.50 |
| AAGGAGATTA | BC003541 | hypothetical protein FLJ10488 | 0.55 | 0.00 | 0.00 | 0.18 | 0.20 | 0.00 | 0.33 | 0.32 | 0.00 | 5.50 |
| CAGCGCAAGT | J01416 | human mt (mitochondrial) cytochrome c oxidase subu | 0.55 | 0.00 | 0.00 | 0.18 | 0.20 | 0.00 | 0.00 | 0.00 | 0.00 | 5.50 |
| AATTCCCTTT | M55630 M3 | topoisomerase (DNA) I | 0.55 | 0.00 | 0.20 | 0.18 | 0.20 | 0.00 | 0.00 | 0.00 | 0.00 | 5.50 |
| TATTTTTGGC | NM_024116 | hypothetical protein MGC5306 | 0.55 | 0.00 | 0.59 | 0.18 | 0.20 | 0.00 | 0.00 | 0.00 | 0.00 | 0.50 |
| ATGATAAAAC | Z48796 | superkiller viralicidic activity 2 (S. ceravisiae homolog)-like | 0.55 | 0.00 | 0.00 | 0.00 | 0.20 | 0.00 | 0.00 | 0.00 | 0.00 | 5.50 |
| TTACGATGAA | Hs.6335 | phosphatidylinosaitol-4-phosphate 5-kinase, type II, beta (EST) | 0.55 | 0.00 | 0.20 | 0.54 | 0.20 | 0.00 | 0.33 | 0.64 | 0.00 | 5.50 |
| GGAGATGGAG | Hs.188757 | clone MGC: 5564, mRNA, complete cds (EST) | 0.55 | 0.00 | 0.20 | 0.54 | 0.20 | 0.00 | 0.00 | 0.00 | 0.00 | 5.50 |
| CAAAAAAAAT | Hs.108196 | HSPC037 protein (EST) or other ESTs | 0.55 | 0.00 | 0.20 | 0.18 | 0.20 | 0.00 | 0.00 | 0.00 | 0.00 | 5.50 |
| GCCTGGCACC | Hs.58167 | zinc finger protein 282 (EST) | 0.55 | 0.00 | 0.20 | 0.00 | 0.20 | 0.00 | 0.00 | 0.00 | 0.00 | 5.50 |
| CCCTATGTTT | Hs.6557 | zinc finger protein 161 (EST) | 0.55 | 0.00 | 0.39 | 0.36 | 0.20 | 0.00 | 0.00 | 0.00 | 0.00 | 5.50 |
| GGACAAGATA | Hs.111911 | Weakly similar to MUC2_HUMAN MUCIN 2 PRECURSOR [H. sapiens] | 0.55 | 0.00 | 0.39 | 0.00 | 0.20 | 0.00 | 0.00 | 0.00 | 0.00 | 5.50 |
| TGACTGCTGC | Hs.90063 | neurocalcin delta (EST) | 0.55 | 0.00 | 0.00 | 0.36 | 0.20 | 0.00 | 0.33 | 0.00 | 0.00 | 5.50 |
| TCCAGTATGA | Hs.173274 | Integrin cytoplasmic domain-associated protein 1 (EST) | 0.55 | 0.00 | 0.00 | 0.18 | 0.20 | 0.00 | 0.33 | 0.00 | 0.00 | 5.50 |
| ACTCCAAGAA | Hs.106346 | ratinoic acid repressible protein (EST) | 0.55 | 0.00 | 0.00 | 0.18 | 0.20 | 0.00 | 0.00 | 0.00 | 0.00 | 5.50 |
| GATACGTAAG | Hs.296356 | cDNA DKFZp434M162 (from clone DKFZp434M162) (EST) | 0.55 | 0.00 | 0.00 | 0.18 | 0.20 | 0.00 | 0.00 | 0.00 | 0.00 | 5.50 |
| ATGTGGCACA | Hs.67776 | Weakly similar to T22341 hypothetical protein F47B8.5 - Caeriorhabditis elegans [C. elegans] (EST) | 1.48 | 0.58 | 0.98 | 1.26 | 0.00 | 0.53 | 0.33 | 0.32 | 2.19 | 5.58 |
| CAGTTACTTA | X57346 | H. sapiensmRNA for HS1 protein. | 3.14 | 2.13 | 1.37 | 1.99 | 0.98 | 0.18 | 1.63 | 0.64 | 3.67 | 5.41 |
| GTGTCCCTGT | AF032456 | Homo sapiens ubiquitin conjugating enzyme G2 (UBE2 | 1.48 | 0.97 | 0.59 | 0.54 | 0.20 | 0.35 | 0.33 | 0.00 | 3.53 | 5.38 |
| TCTGCACCTC | X70940 | eukaryotic translation elongation factor 1 alpha 2 | 1.48 | 0.97 | 1.57 | 0.72 | 0.20 | 0.35 | 1.30 | 0.32 | 3.53 | 5.38 |
| CCAATTTACA | Hs.337757 | high-mobility group (nonhistone chromosomal) protein 1 (EST) | 1.48 | 0.97 | 0.98 | 0.72 | 0.20 | 0.35 | 0.33 | 0.00 | 3.53 | 5.38 |
| AGTGACCTTC | Hs.34447 | EST | 1.48 | 0.97 | 1.57 | 0.90 | 0.20 | 0.35 | 0.00 | 0.32 | 3.53 | 5.38 |
| ATTTTGTGCA | AF217521 | uncharactarized bone marrow protein BM045 | 1.48 | 0.39 | 0.78 | 0.36 | 0.20 | 0.35 | 0.00 | 0.00 | 1.42 | 5.38 |
| CAAACCTTTA | Hs.75133 | transcription factor 6-like 1 (mitochondrial transcription factor 1-like) (EST) or | 1.48 | 0.39 | 0.98 | 1.63 | 0.20 | 0.35 | 0.33 | 0.00 | 1.42 | 5.38 |
| | | FT005 protein (EST) or another EST | | | | | | | | | | |
| GCATACCTGC | Hs.8258 | DKFZP434D1335 protein (EST) | 1.48 | 0.39 | 1.37 | 0.90 | 0.20 | 0.35 | 0.33 | 0.00 | 1.42 | 5.38 |
| TTGTTATATT | AF208844 | hypothetical protein | 1.48 | 0.19 | 1.37 | 0.90 | 0.20 | 0.35 | 0.33 | 0.32 | 0.69 | 5.38 |
| GAGTCAGGAG | AF151878 | CGI-120 protein | 4.98 | 3.67 | 5.88 | 4.88 | 0.98 | 0.89 | 0.98 | 0.64 | 3.93 | 5.33 |
| AGAGCCCTAG | L77701 | COX17 (yeast) homolog, cytochrome c oxidase assembly protein | 2.40 | 1.74 | 2.35 | 0.90 | 0.20 | 0.71 | 0.65 | 0.97 | 3.82 | 5.27 |
| ACCAAAGCCC | AF155654 | putative ribosomal protein S1 mRNA | 0.92 | 0.39 | 0.59 | 1.08 | 0.00 | 0.35 | 0.00 | 0.00 | 2.23 | 5.26 |
| TATGTATGTT | BC008717 | casein kinase 1, alpha 1 | 0.92 | 0.39 | 0.00 | 0.72 | 0.00 | 0.35 | 0.33 | 0.97 | 2.23 | 5.26 |
| GAAAAGAGAT | Hs.92145 | Weakly similar to SFR4_HUMAN SPLICING FACTOR, ARGININE/SERINE-RICH 4 [H. sapiens] | 0.92 | 0.39 | 0.20 | 0.90 | 0.00 | 0.35 | 0.00 | 1.29 | 2.23 | 5.26 |
| | | (EST) | | | | | | | | | | |
| TTAGTTAAGC | X81198 | archain 1 | 0.92 | 0.00 | 0.20 | 0.36 | 0.00 | 0.35 | 0.65 | 0.32 | 0.00 | 5.26 |
| GAAACTAGGA | AF043472 | potassium voltage-gated channel, delayed-rectifier, subfamily S, member 3 | 1.48 | 0.97 | 1.57 | 1.08 | 0.39 | 0.18 | 0.65 | 1.29 | 3.40 | 5.19 |
| TGTGCCACTA | Hs.181022 | CGI-07 protein | 1.48 | 0.97 | 0.39 | 0.72 | 0.39 | 0.18 | 0.33 | 0.97 | 3.40 | 5.19 |
| TTGCAATGCA | Hs.8127 | KIAA0144 gene product (EST) | 1.48 | 0.97 | 1.57 | 0.72 | 0.39 | 0.18 | 0.65 | 0.00 | 3.40 | 5.19 |
| ATCAGTGTGA | S80562 | acidic calponin [human, kidney, mRNA, 1607 nt]. | 1.48 | 0.97 | 0.59 | 0.90 | 0.59 | 0.00 | 0.33 | 0.32 | 3.29 | 5.02 |
| CCTTATATTT | D84294 | tetratricopeptide repeat domain 3 | 1.48 | 0.39 | 0.39 | 0.72 | 0.59 | 0.00 | 0.33 | 0.00 | 1.32 | 5.02 |
| TAAATTCACC | AB018262 | Homo sapiens mRNA for KIAA0719 protein, complete c | 0.92 | 0.58 | 0.98 | 0.36 | 0.20 | 0.18 | 0.00 | 0.64 | 3.05 | 4.84 |
| TTGTTCTTTG | J03804 | protein phosphatase 2 (formerly 2A), catalytic subunit, alpha isoform | 0.92 | 0.58 | 0.39 | 0.18 | 0.20 | 0.18 | 0.00 | 0.32 | 3.05 | 4.84 |
| TTGAAAAAAA | X61972 | proteasome (prosome, macropain) subunit, alpha type, 6 or collagen, type IV, alpha 3 | 0.92 | 0.58 | 0.39 | 0.72 | 0.20 | 0.18 | 0.00 | 0.32 | 3.05 | 4.84 |
| | | (Goodpasture antigen) binding protein | | | | | | | | | | |
| ATCATCCAAT | Hs.5888 | EST | 0.92 | 0.58 | 0.59 | 0.18 | 0.20 | 0.18 | 0.00 | 0.00 | 3.05 | 4.84 |
| CAGGCTACTG | AF102265 | N-acetylglucosamine phosphate mutase | 0.92 | 0.39 | 0.59 | 1.63 | 0.20 | 0.18 | 0.65 | 0.32 | 2.05 | 4.84 |
| TATTTTTGTT | X91247 | H. sapiens mRNA for thioredoxin reductase. | 0.92 | 0.39 | 0.00 | 0.54 | 0.20 | 0.18 | 0.00 | 0.32 | 2.05 | 4.84 |
| TCAGCACCTG | BC005104 | Similar to RIKEN cDNA 5830420C20 gene | 1.84 | 0.58 | 0.20 | 0.36 | 0.39 | 0.35 | 0.98 | 0.32 | 1.57 | 4.97 |
| GAGTCAGCAT | BC003621 | heterogeneous nuclear ribonucleoprotein U (scaffold attachment factor A) | 0.92 | 0.19 | 0.59 | 0.36 | 0.20 | 0.18 | 0.00 | 0.00 | 1.00 | 4.84 |
| TCTGTTGAGT | Hs.46679 | hypothetical protein FLJ20739 | 0.92 | 0.19 | 0.20 | 0.00 | 0.20 | 0.18 | 0.98 | 0.32 | 1.00 | 4.84 |
| TCTGTTATTG | Hs.182698 | mitochondrial ribosomal protein L20 | 0.92 | 0.19 | 0.00 | 0.00 | 0.20 | 0.18 | 0.33 | 0.00 | 1.00 | 4.84 |
| GAAGAGCTGG | BC008976 | EAP30 subunit of ELL complex | 0.92 | 0.00 | 0.00 | 0.18 | 0.20 | 0.18 | 0.33 | 0.00 | 0.00 | 4.84 |
| AAGAGTTTTG | J05017 | Human aldose reductase mRNA, complete cds. | 5.90 | 2.90 | 4.51 | 4.15 | 1.37 | 1.06 | 1.95 | 0.32 | 2.39 | 4.86 |
| GTTTCAGGTA | M23115 J04 | Homo sapiens calcium-ATPase (HK2) mRNA, complete c | 1.29 | 0.58 | 0.59 | 1.08 | 0.00 | 0.53 | 0.65 | 0.00 | 2.19 | 4.87 |
| CCTATAATCC | AL117415 | Homo sapiens mRNA; cDNA DKFZp434K0521 (from clone | 1.84 | 1.35 | 1.37 | 1.08 | 0.59 | 0.18 | 1.30 | 0.64 | 3.51 | 4.78 |
| TTCATTGTAG | AJ011001 | Homo sapiens mRNA for TM7XN1 protein. | 2.21 | 0.97 | 2.16 | 1.26 | 0.39 | 0.53 | 0.65 | 0.64 | 2.11 | 4.80 |
| TCTTAATGAA | S79942 | eukaryotic translation initiation factor 4A, isoform 2 | 3.14 | 0.58 | 1.96 | 1.26 | 0.78 | 0.53 | 0.65 | 0.64 | 0.89 | 4.79 |
| CCTAAAGGAG | BC017168 | hypothetical protein PRO1855 | 0.92 | 0.19 | 0.20 | 0.54 | 0.39 | 0.00 | 0.33 | 0.00 | 0.97 | 4.72 |
| CATTTTTCCC | Hs.90336 | ATPase, H+ transporting, lysosomal (vacuolar proton pump). member J (EST) | 0.92 | 0.19 | 0.20 | 0.90 | 0.39 | 0.00 | 0.65 | 0.00 | 0.97 | 4.72 |
| TTAGATAAGC | M94083 | chaperonin containing TCP1, subunit 6A (zeta 1) | 2.21 | 1.35 | 2.55 | 1.63 | 0.59 | 0.35 | 0.33 | 0.00 | 2.87 | 4.70 |
| CAAGTTAGTG | AL050215 | Homo sapiens mRNA; cDNA DKFZp586I1023 (from clone | 1.29 | 0.77 | 0.98 | 0.72 | 0.20 | 0.35 | 0.00 | 0.00 | 2.80 | 4.69 |
| ACTGGAGTTT | AF002990 | hypothetical protein (EST) | 1.29 | 0.39 | 0.20 | 0.54 | 0.20 | 0.35 | 0.65 | 0.00 | 1.42 | 4.69 |
| AGGAATGCTT | M63180 | threonyl-tRNA synthetase | 2.21 | 0.58 | 1.37 | 1.08 | 0.78 | 0.18 | 0.33 | 0.97 | 1.21 | 4.60 |
| GAAATTTAAA | D63874 | Human mRNA for HMG-1, complete cds. | 1.29 | 0.77 | 0.98 | 1.08 | 0.39 | 0.18 | 0.33 | 0.32 | 2.70 | 4.53 |
| GGGGGGGTCT | Z69030 | H. sapiens mRNA for gamma 1 isoform of 61 kDa regula | 1.29 | 0.77 | 1.57 | 1.44 | 0.39 | 0.18 | 0.33 | 0.64 | 2.70 | 4.53 |
| GGCAGTTAAC | Hs.1390 | proteasome (prosome, macropain) subunit, beta type, 2 (EST) | 1.29 | 0.58 | 0.59 | 1.26 | 0.39 | 0.18 | 1.30 | 0.32 | 2.04 | 4.53 |
| CCACTGCGCT | AB058744 | KIAA1841 protein | 1.29 | 0.19 | 0.59 | 0.54 | 0.39 | 0.18 | 0.00 | 0.32 | 0.67 | 4.53 |
| ACAGTGGGGA | BC003005 | unactive progesterone receptor, 23 kD | 7.19 | 5.02 | 3.92 | 4.88 | 1.76 | 1.42 | 1.63 | 3.86 | 3.16 | 4.52 |
| GTCCGGAGTC | AK001381 | protein phosphatase methylesterase-1 | 1.66 | 0.19 | 0.78 | 1.26 | 0.39 | 0.35 | 0.65 | 0.97 | 0.51 | 4.49 |
| ATAGAGGCAA | D14812 | Human mRNA for KIAA0026 gene, complete cds. | 3.69 | 1.55 | 2.75 | 3.61 | 0.98 | 0.71 | 1.30 | 1.61 | 1.83 | 4.37 |
| GATTCCACTG | AF001601 | paraoxonase 2 | 2.95 | 1.74 | 3.33 | 2.17 | 1.17 | 0.18 | 0.00 | 0.32 | 2.58 | 4.37 |
| TACTAGTCCT | D87666 | Human heart mRNA for heat shock protein 90, partia | 2.40 | 1.16 | 0.78 | 1.08 | 0.39 | 0.71 | 0.65 | 0.00 | 2.11 | 4.36 |
| TGCCTGTGAA | Hs.7678 | Homo sapiens, Similar to RIKEN cDNA 2310010G13 gene, clone MGC: 9810 IMAGE: | 2.03 | 0.77 | 0.78 | 0.90 | 0.59 | 0.35 | 0.33 | 0.00 | 1.64 | 4.32 |
| | | 3860434, mRNA, complete cds (EST) | | | | | | | | | | |
| GTCAACAGTA | AB010887 | ATP-binding cassette, sub-family C (CFTR/MRP), member 3 | 1.11 | 0.97 | 0.78 | 1.44 | 0.00 | 0.53 | 0.00 | 0.32 | 3.66 | 4.19 |
| CAATTCCTTC | AB002370 | KIAA0372 gene product | 0.74 | 0.58 | 0.20 | 0.00 | 0.00 | 0.35 | 0.00 | 0.00 | 3.31 | 4.23 |
| TGTTGATTTT | AF016903 | clone IMAGE: 3544662, mRNA, partial cds | 0.74 | 0.58 | 0.78 | 0.18 | 0.00 | 0.35 | 0.00 | 0.00 | 3.31 | 4.23 |
| CCCTTAGCAA | Hs.95549 | EST | 0.74 | 0.58 | 0.20 | 0.54 | 0.00 | 0.35 | 0.00 | 0.00 | 3.31 | 4.23 |
| GGGAACCGGA | Hs.75313 | aldo-keto reductase family 1, member B1 (aldose reductase) | 0.74 | 0.58 | 0.78 | 0.36 | 0.00 | 0.35 | 0.00 | 0.00 | 3.31 | 4.23 |
| GTGTTGACTG | AF077599 | Homo sapiens hypothetical SBBI03 protein mRNA, com | 0.74 | 0.39 | 0.00 | 0.00 | 0.00 | 0.35 | 0.00 | 0.00 | 2.23 | 4.23 |
| GTGATATCCA | AF131773 | Homo sapiens clone 25007 mRNA sequence. | 0.74 | 0.39 | 0.20 | 0.00 | 0.00 | 0.35 | 0.00 | 0.32 | 2.23 | 4.23 |
| CCTAGTAAAA | X95073 | translin-associated factor X | 0.74 | 0.39 | 0.78 | 0.54 | 0.00 | 0.35 | 0.00 | 0.00 | 2.23 | 4.23 |
| ATGAGCTATG | AB015631 | TMEM4; transmembrane protein 4 | 1.11 | 0.58 | 1.37 | 0.90 | 0.00 | 0.53 | 0.00 | 0.00 | 2.19 | 4.19 |
| TAATGAACTA | AB014539 | kinesin family member 13B | 0.37 | 0.19 | 0.00 | 0.00 | 0.00 | 0.18 | 0.00 | 0.00 | 2.11 | 4.11 |
| TCCAATCTTG | AB014549 | KIAA0649 gene product | 0.37 | 0.19 | 0.20 | 0.00 | 0.00 | 0.18 | 0.33 | 0.00 | 2.11 | 4.11 |
| ATGTTTGCCA | AB019397 | topoisomerase (DNA) II binding protein | 0.37 | 0.19 | 0.00 | 0.72 | 0.00 | 0.18 | 0.00 | 0.64 | 2.11 | 4.11 |
| ATTCAAAAAA | AB037842 | Homo sapiens mRNA for KIAA1421 protein, partial cd | 0.37 | 0.19 | 0.20 | 0.18 | 0.00 | 0.18 | 0.00 | 0.00 | 2.11 | 4.11 |
| GCGCCTCAAC | AF017633 | CD3-epsilon-associated protein; antisense to ERCC-1 | 0.37 | 0.19 | 0.00 | 0.00 | 0.00 | 0.18 | 0.00 | 0.00 | 2.11 | 4.11 |
| TATCCTGTGT | AF022385 | programmed cell death 10 | 0.37 | 0.19 | 0.00 | 0.36 | 0.00 | 0.18 | 0.00 | 0.32 | 2.11 | 4.11 |
| TCTTCTGCTT | AF151054 | Homo sapiens HSPC220 mRNA, complete cds. | 0.37 | 0.19 | 0.00 | 0.00 | 0.00 | 0.18 | 0.00 | 0.00 | 2.11 | 4.11 |
| TTCCCCACCC | AF219624 | Matrix metalloproteinase 28 (+C) | 0.37 | 0.19 | 0.78 | 0.00 | 0.00 | 0.18 | 0.00 | 0.00 | 2.11 | 4.11 |
| TAATTTGAAA | AJ227859 | EGL nine (C. elegans) homolog 1; O2 sensor; PHD-1 | 0.37 | 0.19 | 0.00 | 0.18 | 0.00 | 0.18 | 0.33 | 0.32 | 2.11 | 4.11 |
| ATAAAAAAAA | AL110267 | osteoglycin (osteoinductive factor, mimecan) or LIM and cysteine-rich domains 1 | 0.37 | 0.19 | 0.39 | 0.54 | 0.00 | 0.18 | 0.00 | 0.32 | 2.11 | 4.11 |
| | | or natural killer cell receptor 2B4 or ZAP3 protein | | | | | | | | | | |
| TAGCTGTCTT | AL117476 | DKFZP727C091 protein | 0.37 | 0.19 | 0.20 | 0.00 | 0.00 | 0.18 | 0.00 | 0.00 | 2.11 | 4.11 |
| TGCACACACA | AL137748 | EGF-like-domain, multiple 4 or beta-catenin-interacting protein ICAT | 0.37 | 0.19 | 0.39 | 0.72 | 0.00 | 0.18 | 0.00 | 0.00 | 2.11 | 4.11 |
| GGTTCTGTAG | BC000861 | hypothetical protein MGC5585 | 0.37 | 0.19 | 0.39 | 0.36 | 0.00 | 0.18 | 0.00 | 0.32 | 2.11 | 4.11 |
| TATTAGACAA | BC004121 | hypothetical protein FLJ22709 | 0.37 | 0.19 | 0.00 | 0.00 | 0.00 | 0.18 | 0.33 | 0.00 | 2.11 | 4.11 |
| TTGTACAATT | BC006403 | NCK adaptor protein 1 | 0.37 | 0.19 | 0.00 | 0.18 | 0.00 | 0.18 | 0.00 | 0.00 | 2.11 | 4.11 |
| CTTAGTACAT | BC006429 | hypothetical protein MGC12981 | 0.37 | 0.19 | 0.00 | 0.00 | 0.00 | 0.18 | 0.00 | 0.00 | 2.11 | 4.11 |
| ACATTTCATC | Hs.5890 | hypothetical protein FLJ23306 (EST) | 0.37 | 0.19 | 0.00 | 0.00 | 0.00 | 0.18 | 0.33 | 1.93 | 2.11 | 4.11 |
| GATCCTATTA | M61199 | sperm specific antigen 2 | 0.37 | 0.19 | 0.00 | 0.00 | 0.00 | 0.18 | 0.00 | 0.00 | 2.11 | 4.11 |
| AATATTTCAA | M87546 | acid phosphatase 1, soluble | 0.37 | 0.19 | 0.39 | 0.54 | 0.00 | 0.18 | 0.33 | 0.00 | 2.11 | 4.11 |
| GTAGGGGCCT | NM_023924 | hypothetical protein FLJ13441 | 0.37 | 0.19 | 0.39 | 0.00 | 0.00 | 0.18 | 0.00 | 0.00 | 2.11 | 4.11 |
| GTGGTGCCGC | NM_024907 | hypothetical protein FLJ11798 | 0.37 | 0.19 | 0.39 | 1.26 | 0.00 | 0.18 | 0.00 | 0.32 | 2.11 | 4.11 |
| AACAACTGGC | S78771 | NAT = CpG island-aasociated gene [human, mRNA, 1741 | 0.37 | 0.19 | 0.00 | 0.54 | 0.00 | 0.18 | 0.33 | 0.32 | 2.11 | 4.11 |
| TCAGAACAGT | U07231 | G-rich RNA sequence binding factor 1 (GRSF-1) | 0.37 | 0.19 | 0.00 | 0.36 | 0.00 | 0.18 | 0.33 | 0.64 | 2.11 | 4.11 |
| AATATGGGTG | U46571 | DnaJ (Hsp40) homolog, subfamily C, member 7 | 0.37 | 0.19 | 0.39 | 0.00 | 0.00 | 0.18 | 0.00 | 0.00 | 2.11 | 4.11 |
| TTGTATCAGA | U88966 | FK506 binding protein 12-rapamycin associated protein 1 | 0.37 | 0.19 | 0.00 | 0.00 | 0.00 | 0.18 | 0.33 | 0.00 | 2.11 | 4.11 |
| GTGCTTTCCT | X65867 | adenylosuccinate lyase | 0.37 | 0.19 | 0.20 | 0.00 | 0.00 | 0.18 | 0.33 | 0.00 | 2.11 | 4.11 |
| AAAATGACTT | X76952 | membrane component, chromosome 17, surface marker 2 (ovarian carcinoma antigen CA125) | 0.37 | 0.19 | 0.00 | 0.00 | 0.00 | 0.18 | 0.00 | 0.00 | 2.11 | 4.11 |
| GTATAAACGA | BC013135 | ras-like protein | 0.37 | 0.19 | 0.20 | 0.18 | 0.00 | 0.18 | 0.33 | 0.00 | 2.11 | 4.11 |
| ACAAACAAAA | Hs.23964 | sin3-associated polypeptide, 18 kD (EST) or other ESTs | 0.37 | 0.19 | 0.20 | 0.18 | 0.00 | 0.18 | 0.65 | 0.00 | 2.11 | 4.11 |
| TACTGGTTTA | Hs.30299 | IGF-II mRNA-binding protein 2 | 0.37 | 0.19 | 0.20 | 0.18 | 0.00 | 0.18 | 0.65 | 0.32 | 2.11 | 4.11 |
| GTGAATGACA | Hs.8834 | ring finger protein 3 (EST) | 0.37 | 0.19 | 0.20 | 0.18 | 0.00 | 0.18 | 0.00 | 0.00 | 2.11 | 4.11 |
| GGTGAAACCC | Hs.180930 | TBP-associated factor 172 (EST) | 0.37 | 0.19 | 0.20 | 0.18 | 0.00 | 0.18 | 0.00 | 0.00 | 2.11 | 4.11 |
| TCCTGCTGGC | Hs.173884 | cysteine and histidine rich protein (EST) | 0.37 | 0.19 | 0.20 | 0.72 | 0.00 | 0.18 | 0.00 | 0.00 | 2.11 | 4.11 |
| TGCTAACTGC | Hs.236522 | DKFZP434P106 protein (EST) | 0.37 | 0.19 | 0.20 | 0.00 | 0.00 | 0.18 | 0.00 | 0.00 | 2.11 | 4.11 |
| TCTGCCTCGT | Hs.20159 | CGI-92 protein (EST) | 0.37 | 0.19 | 0.20 | 0.00 | 0.00 | 0.18 | 0.00 | 0.00 | 2.11 | 4.11 |
| GGTGAGGGAG | Hs.9071 | progesterone membrane binding protein (EST) | 0.37 | 0.19 | 0.39 | 0.18 | 0.00 | 0.18 | 0.98 | 1.29 | 2.11 | 4.11 |
| TGCCACATTT | Hs.177391 | EST | 0.37 | 0.19 | 0.39 | 0.36 | 0.00 | 0.18 | 0.00 | 0.00 | 2.11 | 4.11 |
| CAAGAGCGAG | Hs.268763 | Breakpoint cluster region protein, uterine leiomyoma, 1; barrier to autointegration factor (EST) | 0.37 | 0.19 | 0.39 | 0.36 | 0.00 | 0.18 | 0.00 | 0.00 | 2.11 | 4.11 |
| AGAATTTGCA | Hs.250655 | hypothetical protein MGC12250 (EST) | 0.37 | 0.19 | 0.39 | 0.36 | 0.00 | 0.18 | 0.00 | 0.00 | 2.11 | 4.11 |
| GATCTGATTG | Hs.9856 | EST | 0.37 | 0.19 | 0.39 | 0.00 | 0.00 | 0.18 | 0.00 | 0.00 | 2.11 | 4.11 |
| GTGACCAGGT | Hs.12272 | BECN1; beclin 1 (coiled-coil, myosin-like BCL2-interacting protein) (EST) | 0.37 | 0.19 | 0.59 | 0.00 | 0.00 | 0.18 | 0.00 | 0.00 | 2.11 | 4.11 |
| TCATCTACAA | Hs.23240 | EST | 0.37 | 0.19 | 0.78 | 0.54 | 0.00 | 0.18 | 0.00 | 0.00 | 2.11 | 4.11 |
| TTAAGTGGAA | Hs.200260 | EST | 0.37 | 0.19 | 0.00 | 0.18 | 0.00 | 0.18 | 0.00 | 0.00 | 2.11 | 4.11 |
| CTGCAGGAGT | Hs.184544 | clone IMAGE: 3355383, mRNA, partial cds (EST) | 0.37 | 0.19 | 0.00 | 0.18 | 0.00 | 0.18 | 0.00 | 0.00 | 2.11 | 4.11 |
| TTTGCTTCTG | Hs.139226 | replication factor C (activator 1) 2 (40 kD) (EST) | 0.37 | 0.19 | 0.00 | 0.18 | 0.00 | 0.18 | 0.00 | 0.00 | 2.11 | 4.11 |
| AAGCACTTCT | Hs.189658 | CGI-149 protein (EST) | 0.37 | 0.19 | 0.00 | 0.36 | 0.00 | 0.18 | 0.00 | 0.00 | 2.11 | 4.11 |
| ATGGACAGGC | Hs.74267 | ribosomal protein L15 (EST) | 0.37 | 0.19 | 0.00 | 0.54 | 0.00 | 0.18 | 0.33 | 0.32 | 2.11 | 4.11 |
| TTCTTGTAAG | Hs.285833 | EST | 0.37 | 0.19 | 0.00 | 0.54 | 0.00 | 0.18 | 0.00 | 0.00 | 2.11 | 4.11 |
| AAAATGACAT | Hs.9222 | estrogen raceptor binding site associated, antigen, 9 (EST) | 0.37 | 0.19 | 0.00 | 0.00 | 0.00 | 0.18 | 0.33 | 0.00 | 2.11 | 4.11 |
| CCAAGATTTG | Hs.319095 | EST | 0.37 | 0.19 | 0.00 | 0.00 | 0.00 | 0.18 | 0.00 | 0.32 | 2.11 | 4.11 |
| AGTTAGCAGT | Hs.19312 | EST | 0.37 | 0.19 | 0.00 | 0.00 | 0.00 | 0.18 | 0.00 | 0.64 | 2.11 | 4.11 |
| GTTTCAGCAC | Hs.77502 | methionine adenosyltransferase II, alpha (EST) | 0.37 | 0.19 | 0.00 | 0.00 | 0.00 | 0.18 | 0.65 | 0.00 | 2.11 | 4.11 |
| TGGAGAAGAG | Hs.179526 | upregulated by 1,25-dihydroxyvitamin D-3 (EST) or others | 0.37 | 0.19 | 0.00 | 0.00 | 0.00 | 0.18 | 0.98 | 0.00 | 2.11 | 4.11 |
| TATGCTGTAA | Hs.32944 | inositol polyphospate-4-phosphatase, type I, 107 kD (EST) | 0.37 | 0.19 | 0.00 | 0.00 | 0.00 | 0.18 | 0.00 | 0.00 | 2.11 | 4.11 |
| CAAGTTTACC | Hs.6580 | EST | 0.37 | 0.19 | 0.00 | 0.00 | 0.00 | 0.18 | 0.00 | 0.00 | 2.11 | 4.11 |
| TAACTTAAGC | AF135161 | CGI-127 protein | 0.74 | 0.19 | 0.59 | 0.18 | 0.00 | 0.35 | 1.63 | 0.00 | 1.09 | 4.23 |
| TATCGTTGCC | D44466 | proteasome (prosome, macropain) 26S subunit, non-ATPase, 1 | 0.74 | 0.19 | 0.00 | 0.72 | 0.00 | 0.35 | 0.00 | 0.00 | 1.09 | 4.23 |
| ATCACGCCAC | Hs.253070 | Similar to cytochrome c-like antigen, clone MGC: 2960, mRNA, complete cds (EST) or other ESTs | 0.74 | 0.19 | 0.20 | 0.00 | 0.00 | 0.35 | 0.33 | 0.64 | 1.09 | 4.23 |
| TCATAAGCAA | Hs.177486 | amyloid beta (A4) precursor protein (protease nexin-II, Alzheimer disease) (EST) | 0.74 | 0.19 | 0.39 | 0.54 | 0.00 | 0.35 | 0.00 | 0.64 | 1.09 | 4.23 |
| CAAGACGGGG | AB037729 | RALGDS; ral guanine nucleotide dissociation stimulator | 1.11 | 0.19 | 0.78 | 0.90 | 0.00 | 0.53 | 1.63 | 3.86 | 0.72 | 4.19 |
| CTCCCGGCGA | AK000334 | hypothetical protein FLJ20327 | 1.11 | 0.19 | 0.39 | 0.36 | 0.00 | 0.53 | 0.00 | 0.00 | 0.72 | 4.19 |
| AGAACAGAAA | AB011083 | adenylate cyclase 3 | 0.37 | 0.00 | 0.20 | 0.18 | 0.00 | 0.18 | 0.00 | 0.00 | 0.00 | 4.11 |
| TCCCAGCCCA | AB017548 | seplapterin reductase (7,8-dihydrobiopterin: NADP+ oxidoreductase) | 0.37 | 0.00 | 0.00 | 0.00 | 0.00 | 0.18 | 0.00 | 0.00 | 0.00 | 4.11 |
| GGAGAGGCCA | AB030304 | GT198, complete ORF | 0.37 | 0.00 | 0.00 | 0.00 | 0.00 | 0.18 | 0.00 | 0.00 | 0.00 | 4.11 |
| TTCCCTGGGA | AB040971 | KIAA1538 protein or Weakly similar to A36071 galectin 3 [H. sapiens] (EST) | 0.37 | 0.00 | 0.20 | 0.18 | 0.00 | 0.18 | 0.33 | 0.00 | 0.00 | 4.11 |
| AGCACAGAGG | AF047042 | citrete synthase | 0.37 | 0.00 | 0.20 | 0.00 | 0.00 | 0.18 | 0.00 | 0.00 | 0.00 | 4.11 |
| TTCACAACAA | AF060877 | regulator of G-protein signalling 20 | 0.37 | 0.00 | 0.39 | 0.00 | 0.00 | 0.18 | 0.00 | 0.00 | 0.00 | 4.11 |
| GGTCAGGGAA | AF070557 | hypothetical protein FLJ12552 (+A) or karyopherin alpha 6 (importin alpha 7) (EST) | 0.37 | 0.00 | 0.00 | 0.00 | 0.00 | 0.18 | 0.00 | 0.00 | 0.00 | 4.11 |
| CCACTGTGCT | AF070566 | clones 24611 and 24705 mRNA sequence | 0.37 | 0.00 | 0.59 | 0.54 | 0.00 | 0.18 | 0.00 | 0.00 | 0.00 | 4.11 |
| GGTTGCAGGG | AF086202 | keratin, hair, acidic, 4 | 0.37 | 0.00 | 0.00 | 0.00 | 0.00 | 0.18 | 0.00 | 0.00 | 0.00 | 4.11 |
| TTCCCAAGGC | AF089720 | smoothened (Drosophila) homolog (+C) or plectin 1, intermediate filament binding protein, 500 kD | 0.37 | 0.00 | 0.59 | 0.36 | 0.00 | 0.18 | 0.00 | 0.00 | 0.00 | 4.11 |
| | | (EST) or small inducible cytokine A3 | | | | | | | | | | |
| GCATTTAGTT | AF121861 | sorting nexin 11 | 0.37 | 0.00 | 0.39 | 0.72 | 0.00 | 0.18 | 0.00 | 0.32 | 0.00 | 4.11 |
| GTAATTATTG | AF212247 | hypothetical protein CDA08 | 0.37 | 0.00 | 0.39 | 0.18 | 0.00 | 0.18 | 0.33 | 0.00 | 0.00 | 4.11 |
| TGATGTGGAA | AJ271832 | protein phosphatase 1B (formerly 2C), magnesium-dependent, beta isoform or other ESTs | 0.74 | 0.00 | 0.00 | 0.36 | 0.00 | 0.35 | 0.00 | 0.00 | 0.00 | 4.23 |
| TTTTTGAAGG | AK000055 | hypothetical protein FLJ20048 | 0.37 | 0.00 | 0.00 | 0.00 | 0.00 | 0.18 | 0.00 | 0.00 | 0.00 | 4.11 |
| TTCTTGCCAG | AK000059 | NY-REN-62 antigen mRNA, partial cds (EST) | 0.37 | 0.00 | 0.00 | 0.18 | 0.00 | 0.18 | 0.00 | 0.00 | 0.00 | 4.11 |
| CTCATATTTT | AK000333 | down-regulated in metastasis | 0.37 | 0.00 | 0.20 | 0.18 | 0.00 | 0.18 | 0.00 | 0.00 | 0.00 | 4.11 |
| TTGGTCTTCC | AK001220 | glutaminase (EST) | 0.37 | 0.00 | 0.20 | 0.18 | 0.00 | 0.18 | 0.00 | 0.00 | 0.00 | 4.11 |
| TAAAGGCTTT | AK001360 | hypothetical protein FLJ10498 | 0.37 | 0.00 | 0.00 | 0.18 | 0.00 | 0.18 | 0.00 | 0.00 | 0.00 | 4.11 |
| CCAAGCTGCC | AK001706 | hypothetical protein FLJ10751 | 0.37 | 0.00 | 0.00 | 0.18 | 0.00 | 0.18 | 0.00 | 0.00 | 0.00 | 4.11 |
| CAGTCCCAGC | AK001729 | hypothetical protein FLJ10867 | 0.37 | 0.00 | 0.00 | 0.72 | 0.00 | 0.18 | 0.00 | 0.00 | 0.00 | 4.11 |
| AGCACTGTAC | AK001901 | uncharacterized hypothalamus protein HT010 | 0.37 | 0.00 | 0.00 | 0.36 | 0.00 | 0.18 | 0.00 | 0.32 | 0.00 | 4.11 |
| CTATGTAAAT | AK002102 | hypothetical protein FLJ11240 | 0.37 | 0.00 | 0.00 | 0.00 | 0.00 | 0.18 | 0.00 | 0.00 | 0.00 | 4.11 |
| TTGGAATCCA | AK002152 | staufen (Drosophila, RNA-binding protein) homolog 2 | 0.37 | 0.00 | 0.00 | 0.36 | 0.00 | 0.18 | 0.00 | 0.00 | 0.00 | 4.11 |
| GGAGCATCTG | AK002155 | GENX-3414; genethonin 1 | 0.37 | 0.00 | 0.00 | 0.00 | 0.00 | 0.18 | 0.65 | 0.64 | 0.00 | 4.11 |
| ATAGGTAGAG | AL031775 | hypothetical protein FLJ12619 | 0.37 | 0.00 | 0.39 | 0.18 | 0.00 | 0.18 | 0.65 | 0.00 | 0.00 | 4.11 |
| CCATTTTAAA | AL049989 | sorting nexin 7 or other ESTs | 0.74 | 0.00 | 0.00 | 0.36 | 0.00 | 0.35 | 0.00 | 0.00 | 0.00 | 4.23 |
| CTGGTGTGTT | AL050197 | DKFZP586D0623 protein | 0.37 | 0.00 | 0.39 | 0.36 | 0.00 | 0.18 | 0.33 | 0.00 | 0.00 | 4.11 |
| TTTTGTACCA | AL121748 | NRP1; neuropilin 1; VEGF165 receptor | 0.37 | 0.00 | 0.00 | 0.18 | 0.00 | 0.18 | 0.00 | 0.64 | 0.00 | 4.11 |
| GCATTGTGAC | AL157441 | downstream neighbor of SON | 0.37 | 0.00 | 0.59 | 0.18 | 0.00 | 0.18 | 0.33 | 0.00 | 0.00 | 4.11 |
| CCTCTATGTT | AL160111 | Isoform 1 of a novel human mRNA from chromosome 22 | 0.37 | 0.00 | 0.00 | 0.00 | 0.00 | 0.18 | 0.00 | 0.00 | 0.00 | 4.11 |
| GTAATTTAAA | AL359053 | mRNA full length insert cDNA clone EUROIMAGE 2005735 or others | 0.37 | 0.00 | 0.39 | 0.00 | 0.00 | 0.18 | 0.00 | 0.00 | 0.00 | 4.11 |
| TTTGCTTCAG | BC001910 | hypothetical protein PRO1580 | 0.37 | 0.00 | 0.00 | 0.00 | 0.00 | 0.18 | 0.00 | 0.00 | 0.00 | 4.11 |
| ATATGGGGTG | BC002981 | hypothetical protein MGC2508 | 0.37 | 0.00 | 0.20 | 0.00 | 0.00 | 0.18 | 0.33 | 0.00 | 0.00 | 4.11 |
| AAATCCTAGA | BC003114 | clone IMAGE: 2961368, mRNA, partial cds | 0.37 | 0.00 | 0.39 | 0.00 | 0.00 | 0.18 | 0.00 | 0.32 | 0.00 | 4.11 |
| TATAAAAGTG | BC005301 | ITGB3BP: integrin beta 3 binding protein (beta3-endonexin) | 0.37 | 0.00 | 0.78 | 0.00 | 0.00 | 0.18 | 0.00 | 0.00 | 0.00 | 4.11 |
| AGGCTGGGAG | BC012853 | GNPI; glucosamine-6-phosphate isomerase or hypothetical protein FLJ23360 | 0.37 | 0.00 | 0.20 | 0.00 | 0.00 | 0.18 | 0.65 | 0.64 | 0.00 | 4.11 |
| CTCTTCACGG | D32050 | alanyl-tRNA synthetase | 0.37 | 0.00 | 0.59 | 0.00 | 0.00 | 0.18 | 0.33 | 0.00 | 0.00 | 4.11 |
| ACATTGGTTA | D63477 | KIAA0143 protein | 0.37 | 0.00 | 0.20 | 0.36 | 0.00 | 0.18 | 0.00 | 0.00 | 0.00 | 4.11 |
| ACCGCACAAA | D67029 | SEC14 (S. cerevisiae)-like 1 | 0.37 | 0.00 | 0.00 | 0.00 | 0.00 | 0.18 | 0.00 | 0.32 | 0.00 | 4.11 |
| ATCAAGTGGA | D87071 | KIAA0233 gene product | 0.37 | 0.00 | 0.20 | 0.54 | 0.00 | 0.18 | 0.00 | 0.00 | 0.00 | 4.11 |
| GGAGCTCTTG | J03473 | ADP-ribosyltransferase (NAD+; poly (ADP-ribose) polymerase) | 0.74 | 0.00 | 0.98 | 0.00 | 0.00 | 0.35 | 0.00 | 0.00 | 0.00 | 4.23 |
| TATTTTAAAA | M28826 | CD1B; CD1B antigen, b polypeptide | 0.37 | 0.00 | 0.39 | 0.00 | 0.00 | 0.18 | 0.00 | 0.00 | 0.00 | 4.11 |
| AAGAAAAAAA | M84605 | putative opioid receptor, neuromedin K (neuroklnin B) receptor-like or others? | 0.37 | 0.00 | 0.39 | 0.00 | 0.00 | 0.18 | 0.00 | 0.32 | 0.00 | 4.11 |
| GGTACTTTTA | NM_020474 | UDP-N-acetyl-alpha-D-galactosamine:polypeptide N-acetylgalactosaminyltransferase 1 (GalNAc-T1) | 0.37 | 0.00 | 0.20 | 0.18 | 0.00 | 0.18 | 0.00 | 0.00 | 0.00 | 4.11 |
| TACTGATGAA | NM_024824 | hypothetical protein FLJ11806 | 0.37 | 0.00 | 0.20 | 0.18 | 0.00 | 0.18 | 0.00 | 0.00 | 0.00 | 4.11 |
| TAGCCCCAGC | U09347 S63 | PLAUR; Plasminogen activator, urokinase receptor | 0.37 | 0.00 | 0.00 | 0.00 | 0.00 | 0.18 | 0.00 | 0.32 | 0.00 | 4.11 |
| TGCAAATAAA | Y09703 | pinin, desmosome associated protein | 0.37 | 0.00 | 0.39 | 0.18 | 0.00 | 0.18 | 0.00 | 0.32 | 0.00 | 4.11 |
| CAGTAGACAG | Y17392 | PFDN1; prefoldin 1 | 0.37 | 0.00 | 0.20 | 0.54 | 0.00 | 0.18 | 0.00 | 0.97 | 0.00 | 4.11 |
| TAAATACAGT | Hs.259785 | camitine palmitoyltransferase I, liver (EST) | 0.74 | 0.00 | 0.39 | 0.18 | 0.00 | 0.35 | 0.00 | 0.00 | 0.00 | 4.23 |
| TAGAGTGTAA | Hs.25063 | PRO0461 protein | 0.74 | 0.00 | 0.00 | 0.00 | 0.00 | 0.35 | 0.65 | 0.00 | 0.00 | 4.23 |
| AAGCCTGTAG | Hs.13572 | calcium modulating ligand (EST) | 0.74 | 0.00 | 0.00 | 0.54 | 0.00 | 0.35 | 0.00 | 0.00 | 0.00 | 4.23 |
| CAGTCTCAGA | Hs.194673 | phosphoprotein enriched in astrocytes 15 (EST) or KIAA1554 protein (EST) | 0.37 | 0.00 | 0.20 | 0.36 | 0.00 | 0.18 | 0.33 | 0.00 | 0.00 | 4.11 |
| TTTCATTAAT | Hs.247043 | type 1 tumor necrosis factor receptor shedding aminopeptidase regulator (EST) or other ESTs | 0.37 | 0.00 | 0.20 | 0.18 | 0.00 | 0.18 | 0.33 | 0.00 | 0.00 | 4.11 |
| TTAATCGAAG | Hs.153 | ribosomal protein L7 (EST) | 0.37 | 0.00 | 0.20 | 0.18 | 0.00 | 0.18 | 0.33 | 0.00 | 0.00 | 4.11 |
| TAAGAAAGAA | Hs.102471 | KIAA0680 gene product (EST) or Weakly similar to TRHY_HUMAN TRICHOHYALI [H. sapiens] | 0.37 | 0.00 | 0.20 | 0.72 | 0.00 | 0.18 | 0.00 | 0.00 | 0.00 | 4.11 |
| | | (EST) | | | | | | | | | | |
| CTGCCCCACC | Hs.279861 | CGI-31 protein (EST) | 0.37 | 0.00 | 0.20 | 0.36 | 0.00 | 0.18 | 0.00 | 0.00 | 0.00 | 4.11 |
| TATTATTTCA | Hs.239489 | TIA1 cytotoxic granule-associated RNA-binding protein (EST) or other ESTs | 0.37 | 0.00 | 0.20 | 0.36 | 0.00 | 0.18 | 0.00 | 0.00 | 0.00 | 4.11 |
| GGATTTGGCT | Hs.351937 | ribosomal protein, large P2 (EST) | 0.37 | 0.00 | 0.20 | 0.18 | 0.00 | 0.18 | 0.00 | 0.00 | 0.00 | 4.11 |
| ACCTCACTTA | Hs.127240 | hypothetical protein FLJ10211 (EST) | 0.37 | 0.00 | 0.20 | 0.18 | 0.00 | 0.18 | 0.00 | 0.00 | 0.00 | 4.11 |
| CTGTGACATT | Hs.177584 | 3-oxoacid CoA transferase (EST) | 0.37 | 0.00 | 0.20 | 0.18 | 0.00 | 0.18 | 0.00 | 0.00 | 0.00 | 4.11 |
| CTGCTGCAAG | Hs.75243 | bromodomain-containing 2 (EST) or arylsulfatase D (EST) | 0.37 | 0.00 | 0.20 | 0.00 | 0.00 | 0.18 | 0.00 | 0.00 | 0.00 | 4.11 |
| CATAAAATAG | Hs.22941 | KIAA1363 protein (EST) | 0.37 | 0.00 | 0.20 | 0.00 | 0.00 | 0.18 | 0.00 | 0.00 | 0.00 | 4.11 |
| GATGCAATAT | Hs.239720 | CCR4-NOT transcription complex, subunit 2 (EST) | 0.37 | 0.00 | 0.20 | 0.00 | 0.00 | 0.18 | 0.00 | 0.00 | 0.00 | 4.11 |
| GTGCCCGGCA | Hs.163593 | ribosomal protein L18a (EST) or other ESTs | 0.37 | 0.00 | 0.20 | 0.00 | 0.00 | 0.18 | 0.00 | 0.00 | 0.00 | 4.11 |
| TCTGTAGGCT | Hs.38694 | EST | 0.37 | 0.00 | 0.20 | 0.00 | 0.00 | 0.18 | 0.00 | 0.00 | 0.00 | 4.11 |
| AGGGAATTAA | Hs.239499 | KIAA0185 protein (EST) | 0.37 | 0.00 | 0.20 | 0.00 | 0.00 | 0.18 | 0.00 | 0.00 | 0.00 | 4.11 |
| GGCCACGTAG | Hs.155597 | DF; D component of complement (adipsin) (EST) | 0.37 | 0.00 | 0.20 | 0.18 | 0.00 | 0.18 | 0.00 | 3.86 | 0.00 | 4.11 |
| GTAAATCCT | Hs.126043 | chromosome 21 open reading frame 51 | 0.37 | 0.00 | 0.39 | 0.36 | 0.00 | 0.18 | 0.33 | 0.00 | 0.00 | 4.11 |
| CCCGCAAGCA | Hs.131304 | EST | 0.37 | 0.00 | 0.39 | 0.00 | 0.00 | 0.18 | 0.33 | 0.00 | 0.00 | 4.11 |
| TCGTGTTTCA | Hs.183454 | Homo sapiens cDNA FLJ14883 fis, clone PLACE1003596, moderately similar to OLIGOSACCHARYL | 0.37 | 0.00 | 0.39 | 0.18 | 0.00 | 0.18 | 0.00 | 0.32 | 0.00 | 4.11 |
| | | TRANSFERASE STT3 SUBUN | | | | | | | | | | |
| GAGTGGAGAG | Hs.77578 | (11nt = T) ubiquitin specific protease 9, X chromosome (Drosophila fat facets related) (EST) | 0.37 | 0.00 | 0.39 | 0.18 | 0.00 | 0.18 | 1.30 | 0.32 | 0.00 | 4.11 |
| GGTGACAGTG | Hs.346875 | Homo sapiens leukemia susceptibility mRNA, 3′ region (EST) | 0.37 | 0.00 | 0.39 | 0.00 | 0.00 | 0.18 | 0.00 | 0.00 | 0.00 | 4.11 |
| ACTGGTGGTC | Hs.323342 | actin related protein 2/3 complex, subunit 4 (20 kD) (EST) | 0.37 | 0.00 | 0.39 | 0.00 | 0.00 | 0.18 | 0.00 | 0.00 | 0.00 | 4.11 |
| CGCGGTGCTT | Hs.100651 | goigi SNAP receptor complex member 2 (EST) or other ESTs | 0.37 | 0.00 | 0.39 | 0.00 | 0.00 | 0.18 | 0.00 | 0.00 | 0.00 | 4.11 |
| GCTGACACTG | Hs.174139 | chloride channel 3 (EST) or integral type I protein (EST) | 0.37 | 0.00 | 0.59 | 0.36 | 0.00 | 0.18 | 0.33 | 0.00 | 0.00 | 4.11 |
| GATGTTTGAA | Hs.14992 | hypothetical protein FLJ11151 (EST) | 0.37 | 0.00 | 0.00 | 0.18 | 0.00 | 0.18 | 0.33 | 0.00 | 0.00 | 4.11 |
| TCAAATAAAG | Hs.256916 | Weakly similar to A38224 protein-tyrosine kinase [H. sapiens] (EST) | 0.37 | 0.00 | 0.00 | 0.00 | 0.00 | 0.18 | 0.33 | 0.00 | 0.00 | 4.11 |
| TTTCTGTTTT | Hs.93659 | protein disulfide isomerase related protein (calcium-binding protein, intestinal-related) | 0.37 | 0.00 | 0.00 | 0.18 | 0.00 | 0.18 | 0.00 | 0.32 | 0.00 | 4.11 |
| | | (EST) or other ESTs | | | | | | | | | | |
| ATCTCTATCC | Hs.75102 | alanyl-tRNA synthetase (EST) | 0.37 | 0.00 | 0.00 | 0.18 | 0.00 | 0.18 | 0.00 | 0.32 | 0.00 | 4.11 |
| TTAGCCCATC | Hs.154718 | tumor protein D52-like 2 (EST) | 0.37 | 0.00 | 0.00 | 0.00 | 0.00 | 0.18 | 0.65 | 0.00 | 0.00 | 4.11 |
| GCAAAAGCAA | Hs.282804 | Hermansky-Pudlak syndrome 3 (EST) | 0.37 | 0.00 | 0.00 | 0.36 | 0.00 | 0.18 | 0.33 | 0.32 | 0.00 | 4.11 |
| TCAGTGGGGG | Hs.325732 | Weakly similar to I38022 hypothetical protein [H. sapiens] (EST) | 0.37 | 0.00 | 0.00 | 0.36 | 0.00 | 0.18 | 0.00 | 0.00 | 0.00 | 4.11 |
| AGAAGGCCTT | Hs.239114 | ADP-ribosylation factor domain protein 1, 64 kD (EST) or mannosidase, alpha, class 1A, member 2 (EST) | 0.37 | 0.00 | 0.00 | 0.36 | 0.00 | 0.18 | 0.00 | 0.00 | 0.00 | 4.11 |
| CTTCGACGAA | Hs.82689 | tumor rejection antigen (gp96) 1 | 0.37 | 0.00 | 0.00 | 0.18 | 0.00 | 0.18 | 0.00 | 0.00 | 0.00 | 4.11 |
| AGAACAAAAA | Hs.181165 | eukaryotic translation elongation factor 1 alpha 1 (EST) or other ESTs | 0.37 | 0.00 | 0.00 | 0.18 | 0.00 | 0.18 | 0.00 | 0.00 | 0.00 | 4.11 |
| ATTGAAAAAA | Hs.740 | PTK2 protein tyrosine kinase 2 (EST) or other ESTs | 0.37 | 0.00 | 0.00 | 0.18 | 0.00 | 0.18 | 0.00 | 0.00 | 0.00 | 4.11 |
| GACTTAAGGT | Hs.155595 | neural precursor cell expressed, developmentally down-regulated 5 (EST) | 0.37 | 0.00 | 0.00 | 0.00 | 0.00 | 0.18 | 0.00 | 0.00 | 0.00 | 4.11 |
| TTGCAAAGGG | Hs.155291 | KIAA0005 gene product (EST) | 0.37 | 0.00 | 0.00 | 0.00 | 0.00 | 0.18 | 0.00 | 0.00 | 0.00 | 4.11 |
| GACAAGGACA | Hs.96322 | hypothetical protein FLJ23560 (EST) | 0.37 | 0.00 | 0.00 | 0.00 | 0.00 | 0.18 | 0.00 | 0.00 | 0.00 | 4.11 |
| TGCTGTTGGA | Hs.3187 | nuclear transcription factor, X-box binding 1 | 0.37 | 0.00 | 0.00 | 0.00 | 0.00 | 0.18 | 0.00 | 0.00 | 0.00 | 4.11 |
| GTTCTTCTGT | Hs.106432 | cDNA FLJ13410 fis, clone PLACE1001720 (EST) | 0.37 | 0.00 | 0.00 | 0.00 | 0.00 | 0.18 | 0.00 | 0.00 | 0.00 | 4.11 |
| TGAAGAGGCA | Hs.181161 | weakly similar to RYANODINE RECEPTOR, CARDIAC MUSCLE (EST) | 0.37 | 0.00 | 0.00 | 0.00 | 0.00 | 0.18 | 0.00 | 0.00 | 0.00 | 4.11 |
| CAACAGCAAA | Hs.132071 | ovarian carcinoma immunoreactive antigen (EST) | 0.37 | 0.00 | 0.00 | 0.00 | 0.00 | 0.18 | 0.00 | 0.00 | 0.00 | 4.11 |
| AAGCCATCGT | Hs.291677 | EST | 0.37 | 0.00 | 0.00 | 0.00 | 0.00 | 0.18 | 0.00 | 0.00 | 0.00 | 4.11 |
| AAAGAAGACT | Hs.7719 | GABA(A) receptor-associated protein (EST) | 0.37 | 0.00 | 0.00 | 0.00 | 0.00 | 0.18 | 0.00 | 0.00 | 0.00 | 4.11 |
| ATTTTAAGGG | Hs.29706 | EST | 0.37 | 0.00 | 0.00 | 0.00 | 0.00 | 0.18 | 0.00 | 0.00 | 0.00 | 4.11 |
| GTAGGGCAAA | Hs.173179 | Homo sapiens, clone MGC: 3245 IMAGE: 3505639, mRNA, complete cds (EST) | 0.37 | 0.00 | 0.00 | 0.00 | 0.00 | 0.18 | 0.00 | 0.00 | 0.00 | 4.11 |
| TGGGTGTGTA | Hs.155313 | death associated transcription factor 1 (EST) or other EST | 0.37 | 0.00 | 0.00 | 0.00 | 0.00 | 0.18 | 0.00 | 0.00 | 0.00 | 4.11 |
| TGGACAGAAA | Hs.83419 | KIAA0252 protein (EST) or other ESTs | 0.37 | 0.00 | 1.18 | 0.00 | 0.00 | 0.18 | 0.33 | 0.00 | 0.00 | 4.11 |
| TCTCAATTCT | AL031281 | cell division cycle 42 (GTP-binding protein, 25 kD) or Sec23 (S. cerevisiae) homolog B | 1.84 | 0.77 | 0.78 | 1.81 | 0.20 | 0.71 | 0.00 | 0.64 | 1.69 | 4.04 |
| TATGAATGCT | Hs.81800 | CSPG2; chondroitin sulfate proteoglycan 2 (versican) (EST) | 3.32 | 3.09 | 1.76 | 2.71 | 0.39 | 1.24 | 0.65 | 1.61 | 3.79 | 4.07 |
| TTCCTGGTAG | AB018331 | KIAA0788 protein | 1.48 | 1.35 | 0.98 | 1.99 | 0.20 | 0.53 | 0.65 | 0.64 | 3.70 | 4.05 |
| GAGGACCCAA | Hs.77313 | cyclin-dependent kinase (CDC2-like) 10 (EST) | 1.48 | 0.77 | 0.59 | 1.08 | 0.20 | 0.53 | 0.00 | 0.32 | 2.11 | 4.05 |
| TCCTGAAATA | AF052130 | RAB6A, member RAS oncogene family or Rab6-like protein | 1.48 | 0.58 | 0.39 | 0.18 | 0.20 | 0.53 | 0.98 | 0.64 | 1.59 | 4.05 |
| CAGCCCTCCC | L07044 | calcium/calmodulin-dependent protein kinase (CaM kinase) II gamma | 1.11 | 0.97 | 0.20 | 0.18 | 0.20 | 0.35 | 0.00 | 0.00 | 3.53 | 4.04 |
| TAATAAATGC | AF055000 | TTS-2.2; transport-secretion protein 2.2 | 1.11 | 0.19 | 0.98 | 0.36 | 0.20 | 0.35 | 0.00 | 0.32 | 0.69 | 4.04 |
| TACAGAGGGA | AF062346 | zinc finger protein 216 | 6.27 | 4.44 | 4.51 | 4.15 | 1.17 | 1.95 | 1.95 | 2.25 | 2.85 | 4.02 |

371 genes in FIG. 2(e) (351 genes; 20 tags with no matches not shown)
SAGE data for genes up-regulated ≧ 4-fold in 786-0 cells, regardless of VHL status (VHL⁺, V1 and VHL⁻, V2)
with respect to the average of two independent cultures of Renal Proximal Tubule cells (RPTECs) grown
under nomoxia (V5, V6). The table is presented in descending order of V2/V5, V6.

| | | | | | | | | | | | | |
|---|---|---|---|---|---|---|---|---|---|---|---|---|
| CCTTTGGCTA | Hs.279556 | KIAA1432 protein or 40S ribosomal protein S27 isoform | 3.32 | 4.25 | 2.55 | 1.63 | 0.00 | 0.18 | 0.33 | 0.64 | 47.22 | 36.89 |
| CCGTGACTCT | AL050228 | FSTL1; follistatin-like 1 | 5.90 | 8.11 | 6.27 | 6.32 | 0.20 | 0.18 | 0.98 | 0.00 | 42.68 | 31.05 |
| TTGGCAGTAT | AJ000534 | (11nt = T) SGCE; sarcoglycan, epsilon | 2.03 | 2.90 | 1.37 | 0.54 | 0.00 | 0.18 | 0.98 | 0.64 | 32.22 | 22.56 |
| TGTTAGAAAA | U84573 | PLOD2; procollagen-lysine, 2-oxoglutarate 5-dioxygenase (lysine hydroxylase) 2 | 2.03 | 2.90 | 2.94 | 2.71 | 0.00 | 0.18 | 1.30 | 0.97 | 32.22 | 22.56 |
| GAGGAAGAAG | S74942 | (11nt = A) TRA1; tumor rejection antigen/endoplasmic reticular heat (EST) | 0.92 | 2.90 | 2.35 | 2.89 | 0.20 | 0.00 | 0.65 | 0.97 | 29.00 | 9.20 |
| TTCGGGTGTG | L19559 | polo (Drosophia)-like kinase | 1.48 | 2.32 | 1.37 | 2.53 | 0.00 | 0.18 | 0.00 | 0.00 | 25.78 | 16.44 |
| GATGCCCTCC | AF161416 | IRS2; insulin receptor substrate 2 | 1.11 | 2.32 | 3.33 | 4.52 | 0.00 | 0.18 | 0.33 | 0.00 | 25.78 | 12.33 |
| GTGTGTTTGT | M77349 | TGFBI; transforming growth factor, beta-induced, 68 kD | 22.13 | 34.39 | 31.96 | 52.56 | 1.17 | 1.60 | 2.28 | 1.61 | 24.74 | 15.98 |
| CTGCAGGGCC | Hs.278242 | hypothetical protein MGC12992 | 0.74 | 2.13 | 1.57 | 0.90 | 0.00 | 0.18 | 0.00 | 0.00 | 23.67 | 8.22 |
| GCACTTGCAT | D50840 | UDP-glucose ceramide glucosyltransferase | 2.21 | 4.25 | 3.53 | 2.89 | 0.00 | 0.35 | 1.30 | 1.29 | 23.61 | 12.63 |
| CACTCGTGTG | BC002711 | cell division cycle 42 (GTP-binding protein, 25 KD) | 1.29 | 2.32 | 0.98 | 1.26 | 0.20 | 0.00 | 0.33 | 1.29 | 23.20 | 12.90 |
| AAAGCAGTTT | AF019225 | apollpoprotein L | 4.79 | 7.92 | 8.43 | 10.84 | 0.20 | 0.53 | 0.00 | 0.32 | 22.00 | 13.12 |
| TCTGGGGACG | AA588847 | eukaryotic translation initiation factor 2, subunit 2 (beta, 38 KD) (EST) | 3.87 | 2.13 | 1.76 | 1.63 | 0.20 | 0.00 | 0.65 | 0.97 | 21.30 | 38.70 |
| GGTACTCGAT | AF289489 | aspartate beta-hydroxylase | 2.40 | 2.13 | 2.16 | 1.99 | 0.20 | 0.00 | 0.98 | 1.29 | 21.30 | 24.00 |
| TCTGCAAAGG | X62534 | high-mobility group (nonhistone chromosomal) protein 2 | 1.11 | 2.13 | 0.59 | 1.08 | 0.20 | 0.00 | 0.00 | 0.64 | 21.30 | 11.10 |
| TCCTGTAAAG | AF070648 | clone 24651 mRNA sequence. | 4.79 | 7.53 | 6.27 | 7.04 | 0.20 | 0.53 | 1.63 | 2.58 | 20.92 | 13.12 |
| CTGCACTTAC | X74796 | minichromosome maintenance deficient (S. cerevisiae) 7 | 2.03 | 3.67 | 3.14 | 3.79 | 0.00 | 0.35 | 0.33 | 0.00 | 20.39 | 11.60 |
| AACACAGAGA | D88667 | CST; cerebroside (3′-phosphoadenylylsulfate: galactosylceramide 3′) sulfotransferase | 1.84 | 1.74 | 3.14 | 3.07 | 0.00 | 0.18 | 0.98 | 5.79 | 19.33 | 20.44 |
| CACCATTCAG | AL162046 | hypothetical protein FLJ10856 | 1.29 | 1.74 | 1.37 | 1.26 | 0.00 | 0.18 | 0.33 | 0.64 | 19.33 | 14.33 |
| CTGGTGAGTG | AF108145 | MYLE protein | 1.11 | 1.74 | 0.78 | 0.18 | 0.00 | 0.18 | 0.65 | 0.00 | 19.33 | 12.33 |
| CTTAAGGATT | AY032853 | PAI-RBP1; PAI-1 mRNA-binding protein | 1.11 | 1.74 | 1.96 | 1.08 | 0.00 | 0.18 | 0.65 | 0.32 | 19.33 | 12.33 |
| TATGTTTCAG | M93425 | protein tyrosine phosphatase, non-receptor type 12 (PTP-PEST) | 1.66 | 1.93 | 0.59 | 1.08 | 0.20 | 0.00 | 0.98 | 0.64 | 19.30 | 16.60 |
| TATGTGCCAC | AK001323 | hypothetical protein FLJ10461 | 0.55 | 1.93 | 2.35 | 0.36 | 0.20 | 0.00 | 0.00 | 0.00 | 19.30 | 5.50 |
| CTGCCGAGCT | U73379 | ubiquitin carrier protein E2-C | 2.21 | 3.48 | 2.75 | 1.44 | 0.20 | 0.18 | 0.00 | 0.00 | 18.32 | 11.63 |
| AATATGTGGG | X13238 | cytochrome c oxidase subunit Vlc | 9.41 | 9.85 | 7.06 | 7.22 | 0.20 | 0.89 | 0.98 | 3.22 | 18.24 | 17.27 |
| AACAGAAGCA | Hs.292815 | ESTs, Weakly similar to T23482 hypothetical protein K08F4.5 - Caenorhabditis elegans [C. elegans] | 5.35 | 5.02 | 6.47 | 4.70 | 0.20 | 0.35 | 0.65 | 0.64 | 17.93 | 19.45 |
| GAAGATGTGG | Hs.278428 | progestin induced protein or similar to rat nuclear ubiquitous casein kinase 2 | 2.40 | 1.74 | 1.57 | 1.99 | 0.20 | 0.00 | 0.65 | 0.32 | 17.40 | 24.00 |
| TCTCCCTTCA | AF099644 | CDC20 (cell division cycle 20, S. cerevisiae, homolog) | 1.84 | 1.74 | 1.96 | 3.25 | 0.20 | 0.00 | 0.00 | 0.00 | 17.40 | 18.40 |
| TAAGATTTCA | AF000364 | heterogeneous nuclear ribonucleoprotein R | 0.74 | 1.74 | 1.57 | 1.63 | 0.20 | 0.00 | 0.00 | 0.00 | 17.40 | 7.40 |
| GAATTCCTCG | BC001972 | hypothetical protein MGC5306 | 1.48 | 1.55 | 1.18 | 0.36 | 0.00 | 0.18 | 0.00 | 0.32 | 17.22 | 16.44 |
| TGCAGATATT | L27711 | cyclin-dependent kinase inhibitor 3 (CDK2-associated dual specificity phosphatase) | 1.11 | 1.55 | 0.98 | 1.44 | 0.00 | 0.18 | 0.00 | 0.00 | 17.22 | 12.33 |
| CTGTGCCCAG | Hs.6456 | chaperonin containing TCP1, subunit 2 (beta) or ribosomal protein L30 or EST | 0.92 | 1.55 | 0.98 | 0.72 | 0.00 | 0.18 | 0.65 | 0.32 | 17.22 | 10.22 |
| GCTCTGTCAC | Hs.33287 | nuclear factor I/B or ankylosis, progressive (mouse) homolog | 0.37 | 1.55 | 0.98 | 1.63 | 0.00 | 0.18 | 0.00 | 0.00 | 17.22 | 4.11 |
| GTGGTTTGCT | Hs.119571 | collagen, type III, alpha 1 (Ehlers-Danlos syndrome type IV, autosomal dominant) or calumenin | 0.92 | 1.55 | 0.59 | 0.54 | 0.20 | 0.00 | 0.65 | 0.00 | 15.50 | 9.20 |
| TACCTTCCTT | AB023175 | KIAA0958 protein | 0.74 | 1.35 | 0.59 | 1.44 | 0.00 | 0.18 | 0.00 | 0.32 | 15.00 | 8.22 |
| GAATGTAAGT | AF061940 | staufen (Drosophila, RNA-binding protein) | 0.74 | 1.35 | 0.39 | 0.54 | 0.00 | 0.18 | 0.65 | 0.32 | 15.00 | 8.22 |
| GGGCTCACCT | AF070552 | interleukin 1, delta or DNA replication factor | 0.37 | 1.35 | 1.18 | 1.26 | 0.00 | 0.18 | 0.00 | 0.00 | 15.00 | 4.11 |
| ATAATAAAGC | AB015032 | retinoic acid receptor responder (tazarotene induced) 2 | 11.99 | 11.21 | 11.76 | 13.55 | 0.00 | 1.60 | 0.00 | 0.32 | 14.01 | 14.99 |
| ACCAGCATAG | Hs.155530 | interferon, gamma-inducible protein 16 or actin related protein ⅔ complex, subunit 1A (41 kD) | 1.66 | 1.35 | 0.78 | 0.36 | 0.20 | 0.00 | 0.33 | 0.00 | 13.50 | 16.60 |
| AAAGTCATTG | M19713 | tropomyosin 1 (alpha) | 1.11 | 1.35 | 0.98 | 1.26 | 0.20 | 0.00 | 0.00 | 0.32 | 13.50 | 11.10 |
| GTTCCAGCCG | Hs.153 | ribosomal protein L7 | 1.11 | 1.35 | 0.98 | 0.90 | 0.20 | 0.00 | 0.00 | 0.64 | 13.50 | 11.10 |
| TTAGGCAAGT | Hs.75103 | tyrosine 3-monooxygenase/tryptophan 5-monooxygenase activation protein, zeta polypeptide | 1.11 | 1.35 | 1.37 | 1.63 | 0.20 | 0.00 | 0.65 | 1.29 | 13.50 | 11.10 |
| CCGAGATGAA | AB018254 | KIAA0711 gene product | 0.74 | 1.35 | 0.59 | 0.54 | 0.20 | 0.00 | 0.33 | 0.00 | 13.50 | 7.40 |
| TAAATAATTT | U07550 | heat shock 10 kD protein 1 (chaperonin 10) | 2.58 | 2.32 | 2.35 | 3.25 | 0.00 | 0.35 | 0.33 | 0.32 | 12.89 | 14.74 |
| GGTTTACCTG | Hs.278554 | chromobox homolog 3 (Drosophila HP1 gamma) | 1.29 | 1.16 | 1.57 | 1.08 | 0.00 | 0.18 | 0.00 | 0.00 | 12.89 | 14.33 |
| TGACTGTTAT | D38551 | RAD21 (S. pombe) homolog | 1.11 | 1.16 | 0.78 | 0.36 | 0.00 | 0.18 | 0.00 | 0.32 | 12.89 | 12.33 |
| GCTCACCTGT | Hs.30701 | ESTs | 1.11 | 1.16 | 1.76 | 1.81 | 0.00 | 0.18 | 0.00 | 0.00 | 12.89 | 12.33 |
| AGGCTGGATG | AB014568 | KIAA0668 protein | 0.92 | 1.16 | 1.37 | 1.63 | 0.00 | 0.18 | 0.33 | 0.00 | 12.89 | 10.22 |
| TATAATCTTT | M94556 | single-stranded DNA-binding protein | 0.74 | 1.16 | 1.18 | 0.90 | 0.00 | 0.18 | 0.00 | 0.32 | 12.89 | 8.22 |
| AGGAATGTTA | Hs.82689 | tumor rejection antigen (gp96) 1 or zinc finger protein 258 | 0.74 | 1.16 | 0.78 | 0.90 | 0.00 | 0.18 | 0.98 | 0.00 | 12.89 | 8.22 |
| CTTAAGAAAA | AL137274 | Gene 33/Mig-6 | 0.37 | 1.16 | 1.37 | 0.54 | 0.00 | 0.18 | 0.00 | 0.00 | 12.89 | 4.11 |
| TGACTGGAAA | Hs.127513 | EST | 0.37 | 1.16 | 0.59 | 1.08 | 0.00 | 0.18 | 0.00 | 0.00 | 12.89 | 4.11 |
| CAGGGGTGAC | Hs.79748 | solute carrier family 3 (activators of dibasic and neutral amino acid transport), member 2 | 1.11 | 2.13 | 1.57 | 1.08 | 0.00 | 0.35 | 0.65 | 0.97 | 11.83 | 6.34 |
| AAGAGGTTTG | X69910 | transmembrane protein (63 kD), endoplasmic reticulum/Golgi intermediate compartment | 1.84 | 3.28 | 2.94 | 1.63 | 0.20 | 0.35 | 0.98 | 0.00 | 11.71 | 6.69 |
| AAGCTGAGTG | L03532 | heterogeneous nuclear ribonucleoprotein M | 0.92 | 1.16 | 0.98 | 1.26 | 0.20 | 0.00 | 0.65 | 0.00 | 11.60 | 9.20 |
| TTGGCATTGT | Hs.306117 | KIAA0306 protein | 0.92 | 1.16 | 1.37 | 0.54 | 0.20 | 0.00 | 0.00 | 0.00 | 11.60 | 9.20 |
| CAAGACAGAA | AK000504 | HSPC150 protein similar to ubiquitin-conjugating enzyme | 0.74 | 1.16 | 1.18 | 0.72 | 0.20 | 0.00 | 0.00 | 0.00 | 11.60 | 7.40 |
| TACTGGAGTA | AY007810 | mesoderm development candidate 1 | 0.74 | 1.16 | 0.00 | 0.18 | 0.20 | 0.00 | 0.98 | 0.00 | 11.60 | 7.40 |
| GTGAGACCCT | X17650 | myogenic factor 3 or polymerase (DNA directed) kappa | 0.74 | 1.16 | 1.57 | 0.54 | 0.20 | 0.00 | 0.33 | 1.29 | 11.60 | 7.40 |
| TTACCCTGAA | Hs.274448 | hypothetical protein FLJ11029 | 0.74 | 1.16 | 0.59 | 1.63 | 0.20 | 0.00 | 0.00 | 0.00 | 11.60 | 7.40 |
| GAACATAGCC | AB030251 | GTPase activating protein | 0.55 | 1.16 | 0.78 | 0.18 | 0.20 | 0.00 | 0.00 | 0.00 | 11.60 | 5.50 |
| TGTCATCACA | U92015 | LOXL2; lysyl oxidase-like 2 | 4.98 | 9.47 | 10.00 | 9.93 | 1.37 | 0.35 | 12.35 | 4.83 | 11.01 | 5.79 |
| TGAAGCAGTA | Hs.326248 | Homo sapiens cDNA: FLJ22071 fis, clone HEP11691 or hypothetical protein EDAG-1 or ESTs | 1.29 | 0.97 | 0.98 | 0.90 | 0.00 | 0.18 | 0.65 | 0.64 | 10.78 | 14.33 |
| AACCAAAAAA | AL050094 | isocitrate dehydrogenase 3 (NAD+) beta or ribosomal protein S27a | 1.11 | 0.97 | 1.18 | 0.54 | 0.00 | 0.18 | 0.00 | 0.00 | 10.78 | 12.33 |
| TACCACACTA | Hs.193063 | Homo sapiens cDNA FLJ14201 fis, clone NT2RP3002955 | 1.11 | 0.97 | 0.39 | 0.18 | 0.00 | 0.18 | 0.00 | 0.00 | 10.78 | 12.33 |
| GCGAGACCCT | Hs.323954 | postmelotic segregation increased 2-like 8 or putative L-type neutral amino acid transporter | 1.11 | 0.97 | 1.18 | 1.99 | 0.00 | 0.18 | 0.65 | 0.00 | 10.78 | 12.33 |
| AATCGCTAAT | Hs.47986 | hypothetical protein MGC10940 | 0.92 | 0.97 | 0.20 | 1.08 | 0.00 | 0.18 | 0.00 | 0.00 | 10.78 | 10.22 |
| CCTGGCCAGA | Hs.180680 | ESTs, Weakly similar to I38022 hypothetical protein [H. sapiens] or Homo sapiens cDNA: FLJ20895 fis, | 0.74 | 0.97 | 0.00 | 0.36 | 0.00 | 0.18 | 0.00 | 0.64 | 10.78 | 8.22 |
| | | clone ADKA03483 | | | | | | | | | | |
| TAAAATTGCT | U75679 | stem-loop (histone) binding protein | 0.55 | 0.97 | 1.96 | 1.63 | 0.00 | 0.18 | 0.00 | 0.64 | 10.78 | 6.11 |
| TGAACTTACA | Hs.253658 | EST | 0.55 | 0.97 | 0.20 | 0.18 | 0.00 | 0.18 | 0.33 | 0.00 | 10.78 | 6.11 |
| CAGAGGAACT | D83532 | cytokine inducible SH2-containing protein or aldo-keto reductase family 1, member B$$ (aldose reductase) | 0.37 | 0.97 | 0.00 | 0.18 | 0.00 | 0.18 | 0.33 | 0.00 | 10.78 | 4.11 |
| CACTGAACTC | Hs.172028 | a disintegrin and metalloproteinase domain 10 | 0.37 | 0.97 | 0.59 | 0.90 | 0.00 | 0.18 | 0.65 | 0.97 | 10.78 | 4.11 |
| TACAAAACCA | AK000221 | nucleolin | 1.84 | 1.93 | 0.98 | 2.53 | 0.00 | 0.35 | 1.30 | 0.32 | 10.72 | 10.51 |
| TACCAGCACA | AF123534 | nucleolar protein NOP5/NOP58 | 1.66 | 1.93 | 1.96 | 1.26 | 0.00 | 0.35 | 0.00 | 0.00 | 10.72 | 9.49 |
| TAAAGCTGTT | X53251 | ubiquitin-conjugating enzyme E2B (RAD6 homolog) | 1.66 | 1.93 | 0.78 | 1.44 | 0.00 | 0.35 | 0.33 | 0.64 | 10.72 | 9.49 |
| TGTACTACTT | AK001163 | multifunctional polypeptide similar to SAICAR synthetase and AIR carboxylase (EST) | 2.21 | 2.13 | 1.18 | 1.08 | 0.39 | 0.00 | 0.00 | 0.64 | 10.65 | 11.33 |
| TGCTGCCTGT | X81005 | bone marrow stromal cell antigen 2 or HCGIV-6 protein | 4.24 | 5.60 | 7.06 | 4.15 | 0.00 | 1.06 | 0.00 | 0.64 | 10.57 | 8.00 |
| TTTGCTGTAG | Hs.254646 | ESTs, Moderately similar to reduced expression in cancer [H. sapiens] | 1.66 | 2.90 | 1.57 | 3.25 | 0.20 | 0.35 | 0.00 | 0.32 | 10.36 | 6.04 |
| GGAGTGTGCT | AL050318 | myosin regulatory light chain 2, smooth muscle isoform | 5.72 | 8.11 | 5.49 | 6.68 | 0.78 | 0.89 | 0.65 | 0.64 | 9.77 | 6.85 |
| TCACAGCTGT | Hs.77054 | B-cell translocation gene 1, anti-proliferative | 1.48 | 0.97 | 1.18 | 1.08 | 0.20 | 0.00 | 0.33 | 4.83 | 9.70 | 14.80 |
| CTAACTTCGT | Hs.14838 | likely ortholog of mouse NPC derived proline rich protein 1 | 1.11 | 0.97 | 0.39 | 0.72 | 0.20 | 0.00 | 0.00 | 0.00 | 9.70 | 11.10 |
| TTTGTTGCTT | AF150734 | PC326 protein | 0.92 | 0.97 | 1.96 | 1.44 | 0.20 | 0.00 | 0.65 | 0.32 | 9.70 | 9.20 |
| CTGACAGTTC | Hs.182278 | calmodulin 2 (phosphorylase kinase, delta) or hypothetical protein HDCMC04P | 0.74 | 0.97 | 1.37 | 0.90 | 0.20 | 0.00 | 0.33 | 0.64 | 9.70 | 7.40 |
| TCACTACTGG | AF044588 | protein regulator of cytokinesis 1 | 0.55 | 0.97 | 0.20 | 0.54 | 0.20 | 0.00 | 0.00 | 0.00 | 9.70 | 5.50 |
| TGGACCCAAC | AF006621 | chromosome 4 open reading frame 1 | 0.74 | 1.74 | 0.59 | 0.00 | 0.00 | 0.35 | 0.33 | 1.93 | 9.67 | 4.23 |
| ATTATTTTTC | AB029156 | CGI-142 or ribosomal protein L7 | 4.43 | 3.67 | 4.31 | 6.14 | 0.59 | 0.18 | 1.95 | 0.32 | 9.66 | 11.51 |
| GAAAAGCCTT | U55206 | gamma-glutamyl hydrolase (conjugase, folylpolygammaglutamyl hydrolase) | 2.95 | 3.67 | 2.55 | 3.43 | 0.59 | 0.18 | 0.65 | 0.64 | 9.66 | 7.66 |
| TGCCATCTGT | BC006510 | CCNB1; cyclin B1 | 2.03 | 2.70 | 2.94 | 2.71 | 0.20 | 0.35 | 0.00 | 0.32 | 9.64 | 7.38 |
| TTGATGTACA | M74002 | splicing factor, arginine/serine-rich 11 | 2.21 | 1.74 | 1.96 | 1.44 | 0.20 | 0.18 | 0.33 | 0.64 | 9.16 | 11.63 |
| TAGTTTGTGG | U03911 | mutS (E. coli) homolog 2 (colon cancer, nonpolyposis type 1) (EST) | 1.29 | 1.74 | 1.76 | 2.17 | 0.20 | 0.18 | 0.33 | 0.32 | 9.16 | 6.79 |
| GTGCTCTTGA | Hs.100002 | HSPC162 protein (EST) | 0.92 | 1.74 | 0.59 | 1.26 | 0.20 | 0.18 | 0.33 | 0.00 | 9.16 | 4.84 |
| CCTCCTCTGA | Hs.28505 | ubiquitin-conjugating enzyme E2H (homologous to yeast UBC8) (EST) | 2.03 | 1.74 | 1.37 | 1.63 | 0.39 | 0.00 | 0.65 | 1.29 | 8.70 | 10.41 |
| TGAAGGATGC | M58459 | ribosomal protein S4, Y-linked | 3.69 | 4.25 | 7.06 | 3.97 | 0.98 | 0.00 | 1.63 | 0.00 | 8.67 | 7.53 |
| ACAGCGTCTG | AB033118 | KIAA1292 protein | 1.29 | 1.55 | 1.18 | 1.08 | 0.00 | 0.35 | 0.33 | 0.00 | 8.61 | 7.37 |
| GATGACCCCG | AK000963 | FLJ10101(EST) | 1.11 | 1.55 | 1.76 | 1.63 | 0.00 | 0.35 | 0.00 | 0.00 | 8.61 | 6.34 |
| GATTGGGGAT | Hs.103834 | hypothetical protein MGC5576 or ESTs | 1.11 | 1.55 | 1.76 | 1.08 | 0.00 | 0.35 | 0.00 | 0.32 | 8.61 | 6.34 |
| TATCTGTCTA | Hs.145279 | SET translocation (myeloid leukemia-associated) (EST) | 4.98 | 3.09 | 5.10 | 7.59 | 0.20 | 0.53 | 0.00 | 0.64 | 8.58 | 13.64 |
| TTAAACCTCA | AB017019 | heterogeneous nuclear ribonucleoprotein D-like | 3.32 | 3.86 | 2.94 | 3.79 | 0.20 | 0.71 | 0.00 | 0.00 | 8.58 | 7.30 |
| CCAGTACAAT | U03398 | TNFSF9; tumor necrosis factor (ligand) superfamily, member 9 | 1.48 | 0.77 | 0.39 | 0.00 | 0.00 | 0.18 | 0.33 | 0.00 | 8.56 | 16.44 |
| GCATATGAGC | Hs.7149 | EST | 1.48 | 0.77 | 0.78 | 0.90 | 0.00 | 0.18 | 0.65 | 0.64 | 8.56 | 16.44 |
| TAAACCGGAA | AF151036 | hypothetical protein | 1.11 | 0.77 | 1.76 | 0.72 | 0.00 | 0.18 | 0.65 | 0.32 | 8.56 | 12.33 |
| GGCATTTTAA | Hs.79353 | transcription factor Dp-1 (EST) | 1.11 | 0.77 | 1.18 | 1.44 | 0.00 | 0.18 | 0.00 | 0.00 | 8.56 | 12.33 |
| TAAATAAATA | AB011125 | amyloid beta (A4) precursor protein (protease nexin-II, Alzheimer disease) or others | 0.92 | 0.77 | 0.20 | 1.08 | 0.00 | 0.18 | 0.33 | 0.64 | 8.56 | 10.22 |
| TTTTAAAATA | AB051478 | KIAA1691 protein | 0.92 | 0.77 | 0.39 | 0.72 | 0.00 | 0.18 | 0.00 | 0.00 | 8.56 | 10.22 |
| CTTGCAGATG | Hs.91161 | prefoldin 4 (EST) | 0.92 | 0.77 | 0.20 | 0.54 | 0.00 | 0.18 | 0.00 | 0.32 | 8.56 | 10.22 |
| ACACTGCAGT | Hs.103755 | receptor-interacting serine-threonine kinase 2 (EST) | 0.92 | 0.77 | 0.59 | 0.36 | 0.00 | 0.18 | 0.00 | 0.00 | 8.56 | 10.22 |
| CACCTGTAGT | AB037798 | sorting nexin 4 or ribosomal protein L5 or others | 0.74 | 0.77 | 0.00 | 0.54 | 0.00 | 0.18 | 0.00 | 0.00 | 8.56 | 8.22 |
| TTTCTGTGTA | AF161445 | hypothetical protein | 0.74 | 0.77 | 0.20 | 0.18 | 0.00 | 0.18 | 0.33 | 0.00 | 8.56 | 8.22 |
| CCACTGCCCT | Hs.347865 | EST | 0.74 | 0.77 | 0.20 | 0.72 | 0.00 | 0.18 | 0.00 | 0.00 | 8.56 | 8.22 |
| TCATCATATT | AF131820 | PTD010 protein | 0.55 | 0.77 | 0.59 | 0.00 | 0.00 | 0.18 | 0.00 | 0.00 | 8.56 | 6.11 |
| CACATCTCTG | AK000174 | EST | 0.55 | 0.77 | 0.20 | 0.54 | 0.00 | 0.18 | 0.00 | 0.32 | 8.56 | 6.11 |
| TGGGCCCCTT | AK001519 | CGI-74 protein | 0.55 | 0.77 | 0.20 | 0.54 | 0.00 | 0.18 | 0.00 | 0.32 | 8.56 | 6.11 |
| TCATCTTTGT | BC003375 | mitochondrial ribosomal protein L3 | 0.55 | 0.77 | 0.59 | 0.18 | 0.00 | 0.18 | 0.00 | 0.00 | 8.56 | 6.11 |
| CAGATAACAT | D13641 | translocase of outer mitochondrial membrane 20 (yeast) homolog | 0.55 | 0.77 | 0.78 | 0.54 | 0.00 | 0.18 | 0.33 | 0.97 | 8.56 | 6.11 |
| TTTATTTGGC | L25931 | lamin B receptor | 0.55 | 0.77 | 0.59 | 0.90 | 0.00 | 0.18 | 0.00 | 0.00 | 8.56 | 6.11 |
| AGGAGGGTGG | M34458 | lamin B1 | 0.55 | 0.77 | 1.18 | 1.63 | 0.00 | 0.18 | 0.33 | 0.00 | 8.56 | 6.11 |
| TGGAAAAAAA | Y10812 | fructose-1,6-bisphosphatase 2 or upstream transcription factor 2, c-fos interacting | 0.55 | 0.77 | 0.59 | 0.72 | 0.00 | 0.18 | 0.00 | 0.00 | 8.56 | 6.11 |
| AGGCTGCCCA | AB023195 | KIAA0978 protein | 0.37 | 0.77 | 0.39 | 0.54 | 0.00 | 0.18 | 0.33 | 0.32 | 8.56 | 4.11 |
| GAGAACCACC | AF055377 | v-maf musculoaponeurotic fibrosarcoma (avian) oncogene homolog | 0.37 | 0.77 | 0.59 | 0.00 | 0.00 | 0.18 | 0.00 | 0.00 | 8.56 | 4.11 |
| TACTTGGGAG | AF061258 | LIM protein (similar to rat protein kinase C-binding enigma) | 0.37 | 0.77 | 0.00 | 0.72 | 0.00 | 0.18 | 0.33 | 0.00 | 8.56 | 4.11 |
| ATGTATGGGG | AF277187 | PNAS-129 mRNA | 0.37 | 0.77 | 0.00 | 0.90 | 0.00 | 0.18 | 0.65 | 0.00 | 8.56 | 4.11 |
| AATACACAGA | AF353720 | DEAD/H (Asp-Glu-Ala-Asp/His) box polypeptide 19 (Dbp5, yeast, homolog) | 0.37 | 0.77 | 0.00 | 0.18 | 0.00 | 0.18 | 0.65 | 0.32 | 8.56 | 4.11 |
| TTCCCTCGTG | BC000629 | aspartyl-tRNA synthetase | 0.37 | 0.77 | 0.20 | 0.36 | 0.00 | 0.18 | 0.33 | 0.32 | 8.56 | 4.11 |
| TTTACCTGCC | BC001727 | hypothetical protein FLJ20093 | 0.37 | 0.77 | 0.39 | 0.36 | 0.00 | 0.18 | 0.00 | 0.00 | 8.56 | 4.11 |
| TCAGAGTAAT | BC006329 | melanoma cell adhesion molecule | 0.37 | 0.77 | 1.37 | 1.08 | 0.00 | 0.18 | 0.00 | 0.00 | 8.56 | 4.11 |
| GGTTATTTAG | D67031 | ADD3; adducin 3 (gamma) | 0.37 | 0.77 | 0.59 | 0.36 | 0.00 | 0.18 | 0.65 | 0.32 | 8.56 | 4.11 |
| AGAAGCAGAC | Z29328 | ubiquitin-conjugating enzyme E2H (homologous to yeast UBC8) | 0.37 | 0.77 | 0.00 | 0.00 | 0.00 | 0.18 | 0.33 | 0.32 | 8.56 | 4.11 |
| ATGTACAGGT | Hs.70704 | hypothetical protein dJ616B8.3 | 0.37 | 0.77 | 0.00 | 0.54 | 0.00 | 0.18 | 0.00 | 0.00 | 8.56 | 4.11 |
| CAGCTCAGCT | AJ012737 | filamin C, gamma (actin-binding protein-280) | 2.58 | 2.32 | 2.16 | 2.53 | 0.39 | 0.18 | 0.33 | 0.00 | 8.29 | 9.05 |
| AGAAAGATGT | X05908 | annexin A1 (lipocortin) | 10.51 | 7.73 | 9.61 | 10.29 | 1.17 | 0.71 | 5.53 | 1.93 | 8.22 | 11.18 |
| AATGAGTTTG | Hs.283740 | DC6 protein | 2.21 | 1.55 | 1.18 | 2.53 | 0.20 | 0.18 | 0.00 | 0.00 | 8.16 | 11.63 |
| GGGAGCCTCA | AB027467 | chromosome 20 open reading frame 1 | 1.29 | 1.55 | 0.98 | 2.71 | 0.20 | 0.18 | 0.00 | 0.00 | 8.16 | 6.79 |
| TACCCCACCT | Hs.54468 | H2A histone family, member Y(EST) or tubby (mouse) homolog | 1.29 | 1.55 | 1.37 | 1.81 | 0.20 | 0.18 | 0.33 | 0.32 | 8.16 | 6.79 |
| TGCTGTGCAT | AF000982 | DEAD/H (Asp-Glu-Ala-Asp/His) box polypeptide 3 | 3.69 | 3.67 | 2.16 | 1.81 | 0.59 | 0.35 | 0.65 | 0.97 | 7.81 | 7.85 |
| GTGAGACCCC | AK001001 | hypothetical protein FLJ10139 or PTD012 protein | 1.29 | 0.77 | 0.39 | 1.26 | 0.20 | 0.00 | 0.98 | 0.97 | 7.70 | 12.90 |
| ATCTCAAAGA | Hs.343475 | cathepsin D (lysosomal aspartyl protease) (EST) | 1.29 | 0.77 | 1.76 | 1.26 | 0.20 | 0.00 | 0.00 | 0.00 | 7.70 | 12.90 |
| TCTGCTTGTC | K03000 | aldehyde dehydrogenase 1 family, member A1 | 1.11 | 0.77 | 0.59 | 0.72 | 0.20 | 0.00 | 0.00 | 0.32 | 7.70 | 11.10 |
| TAAGAAAAGG | U94316 | POP7 (processing of precursor, S. cerevisiae) homolog | 1.11 | 0.77 | 0.00 | 0.54 | 0.20 | 0.00 | 0.00 | 0.00 | 7.70 | 11.10 |
| TACTGGCCGC | Hs.35962 | EST | 0.92 | 0.77 | 0.78 | 1.44 | 0.20 | 0.00 | 0.00 | 0.00 | 7.70 | 9.20 |
| GTAGACACCT | U63785 | ribosomal protein L7(EST) | 0.74 | 0.77 | 1.57 | 1.81 | 0.20 | 0.00 | 0.33 | 0.32 | 7.70 | 7.40 |
| GCAGGTGGTT | Hs.145696 | splicing factor (CC1.3) (EST) | 0.74 | 0.77 | 0.59 | 0.90 | 0.20 | 0.00 | 0.00 | 0.32 | 7.70 | 7.40 |
| TAGGGCTCTC | AK001330 | hypothetical protein FLJ10468 | 0.55 | 0.77 | 1.18 | 0.72 | 0.20 | 0.00 | 0.00 | 0.00 | 7.70 | 5.50 |
| GACTGAGCTT | AL050265 | TAR DNA binding protein | 0.55 | 0.77 | 1.18 | 1.08 | 0.20 | 0.00 | 0.00 | 0.32 | 7.70 | 5.50 |
| GTTGACTTAC | D17219 | HTGN29 protein | 0.55 | 0.77 | 0.39 | 0.54 | 0.20 | 0.00 | 0.00 | 0.64 | 7.70 | 5.50 |
| GTGAGCCCAT | Hs.74335 | heat shock 90 kD protein 1, beta (EST) | 0.55 | 0.77 | 1.18 | 0.36 | 0.20 | 0.00 | 0.33 | 0.32 | 7.70 | 5.50 |
| TGTACCTGTA | Z36832 | tubulin, alpha, ubiquitous or tubulin alpha 1 | 42.79 | 47.72 | 44.90 | 56.71 | 5.87 | 6.56 | 5.20 | 0.64 | 7.67 | 6.88 |
| ATGCTAAAAA | Hs.129548 | heterogeneous nuclear ribonucleoprotein K (EST) | 1.66 | 1.35 | 0.78 | 1.08 | 0.00 | 0.35 | 0.00 | 0.32 | 7.50 | 9.49 |
| CCTGTGTGCA | AB037845 | TBP-associated factor 172 | 1.11 | 1.35 | 0.78 | 0.90 | 0.00 | 0.35 | 0.33 | 0.97 | 7.50 | 6.34 |
| GAAGGAGATA | AF047431 | cholinephosphotransferase 1 | 0.74 | 1.35 | 0.39 | 0.00 | 0.00 | 0.35 | 1.30 | 0.64 | 7.50 | 4.23 |
| CAAGAGGCAA | AB014579 | MGEA5; meningioma expressed antigen 5 (hyaluronidase) | 1.84 | 2.13 | 2.35 | 2.35 | 0.59 | 0.00 | 0.65 | 0.97 | 7.34 | 6.24 |
| ATTGTTTATG | AB037814 | KIAA1393 protein or high-mobility group (nonhistone chromosomal) protein 17 | 6.64 | 5.99 | 6.08 | 6.32 | 0.59 | 1.06 | 0.33 | 0.64 | 7.22 | 8.05 |
| TGTTTTCGCC | U64028 | NADH dehydrogenase (ubiquinone) 1 alpha subcomplex, 5 (13 kD, B13) | 2.40 | 1.35 | 2.35 | 2.53 | 0.20 | 0.18 | 0.00 | 0.32 | 7.11 | 12.63 |
| CAAGTTCTTT | Hs.75968 | thymosin, beta 4, X chromosome (EST) | 1.66 | 1.35 | 1.96 | 1.44 | 0.20 | 0.18 | 0.98 | 0.00 | 7.11 | 8.74 |
| CACCAAAAAA | Hs.132071 | ovarian carcinoma immunoreactive antigen (EST) or ADP-ribosylation factor-like 7 (EST) | 1.29 | 1.35 | 0.39 | 0.72 | 0.20 | 0.18 | 0.33 | 0.00 | 7.11 | 6.79 |
| ATTGATCTTG | L07493 | replication protein A3 (14 kD) | 0.92 | 1.35 | 0.78 | 0.54 | 0.20 | 0.18 | 0.00 | 0.00 | 7.11 | 4.84 |
| TTCTGTGAAT | AJ223812 | hypothetical protein FLJ12750 | 1.66 | 2.70 | 1.57 | 1.81 | 0.59 | 0.18 | 0.33 | 0.32 | 7.11 | 4.31 |
| TGTATAAAAA | X15187 | tumor rejection antigen (gp96) 1 | 2.58 | 1.93 | 4.31 | 1.44 | 0.20 | 0.35 | 0.98 | 0.64 | 6.89 | 9.38 |
| CGTTCCTGCG | X77956 | ID1; inhibitor of DNA binding 1, dominant negative helix-loop-helix protein | 2.40 | 1.93 | 2.55 | 2.35 | 0.20 | 0.35 | 0.00 | 0.32 | 6.89 | 8.73 |
| GCTTACCTTT | Hs.7753 | calumenin (EST) | 1.66 | 1.93 | 1.37 | 1.99 | 0.39 | 0.18 | 0.65 | 0.00 | 6.89 | 5.82 |
| TTGCCTGTAT | Hs.184693 | transcription elongation factor B (SIII), polypeptide 1 (15 kD, elongin C) (EST) | 2.21 | 3.09 | 1.37 | 1.44 | 0.20 | 0.71 | 0.98 | 0.64 | 6.87 | 4.86 |
| GCCTTCCAAT | X15729 | DEAD/H (Asp-Glu-Ala-Asp/His) box polypeptide 5 (RNA helicase, 68 kD) | 11.43 | 11.98 | 11.37 | 11.92 | 1.37 | 2.13 | 1.30 | 0.97 | 6.85 | 6.53 |
| CAAGTGGCAA | Hs.227835 | KIAA1049 protein (EST) | 1.84 | 2.51 | 0.78 | 2.35 | 0.39 | 0.35 | 1.30 | 0.00 | 6.78 | 4.97 |
| AAAGGAAAGT | AF060925 | hematological and neurological expressed 1 | 1.84 | 1.35 | 0.20 | 1.26 | 0.39 | 0.00 | 0.33 | 0.00 | 6.75 | 9.44 |
| ACTGAGGAAA | X64875 | IGFBP3; insulin-like growth factor binding protein 3 | 3.50 | 5.80 | 9.02 | 9.21 | 1.57 | 0.18 | 4.55 | 4.51 | 6.67 | 4.00 |
| TTAACCCTCT | Z48950 | H3 histone, family 3B (H3.3B) | 1.29 | 0.58 | 0.20 | 0.72 | 0.00 | 0.18 | 0.65 | 0.32 | 6.44 | 14.33 |
| AGCTCAGGAG | Hs.269177 | EST | 1.11 | 0.58 | 0.00 | 0.54 | 0.00 | 0.18 | 0.33 | 0.00 | 6.44 | 12.33 |
| ACCACCCATT | AB012911 | frizzled (Drosophila) homolog 6 | 0.92 | 0.58 | 0.78 | 0.36 | 0.00 | 0.18 | 0.33 | 0.00 | 6.44 | 10.22 |
| TTCCTTTTTA | L26339 | autoantigen | 0.74 | 0.58 | 0.00 | 0.18 | 0.00 | 0.18 | 0.00 | 0.32 | 6.44 | 8.22 |
| CCTGTAATCT | M34309 | v-erb-b2 avian erythroblastic leukemia viral oncogene homolog 3 or pyrimidinergic receptor P2Y, | 0.74 | 0.58 | 0.98 | 0.54 | 0.00 | 0.18 | 0.33 | 0.32 | 6.44 | 8.22 |
| | | G-protein coupled, 6 or T | | | | | | | | | | |
| TTTGCACTTG | X62048 | wee1+ (S. pombe) homolog or protein × 013 | 0.74 | 0.58 | 0.00 | 0.90 | 0.00 | 0.18 | 0.33 | 0.00 | 6.44 | 8.22 |
| TTACTGGGTT | Hs.170263 | tumor protein p53-binding protein, 1 (EST) | 0.74 | 0.58 | 0.20 | 0.18 | 0.00 | 0.18 | 0.00 | 0.00 | 6.44 | 8.22 |
| GTTTCTGCAA | AB002334 | RAN binding protein 2-like 1 or KIAA0336 gene product | 0.55 | 0.58 | 1.37 | 0.36 | 0.00 | 0.18 | 0.00 | 0.00 | 6.44 | 6.11 |
| TATAATGTGC | AF117108 | KOC1; IGF-II mRNA-binding protein 3 | 0.55 | 0.58 | 0.78 | 0.54 | 0.00 | 0.18 | 0.00 | 0.00 | 6.44 | 6.11 |
| TTTTCAAGAA | AL080135 | hypothetical protein DKFZp434I143 | 0.55 | 0.58 | 0.78 | 0.18 | 0.00 | 0.18 | 0.33 | 0.00 | 6.44 | 6.11 |
| TACAAAAAAA | BC000281 | PCTAIRE protein kinase 3 | 0.55 | 0.58 | 0.20 | 0.36 | 0.00 | 0.18 | 0.00 | 0.00 | 6.44 | 6.11 |
| CAAATAAAAT | D78130 | squalene epoxidase | 0.55 | 0.58 | 0.39 | 0.54 | 0.00 | 0.18 | 1.30 | 0.00 | 6.44 | 6.11 |
| TTCAGGAGGG | M17661 J02 | T-cell receptor alpha delta locus from bases 501613 to 752736 (section 3 of 5) | 0.55 | 0.58 | 0.59 | 0.18 | 0.00 | 0.18 | 0.98 | 1.61 | 6.44 | 6.11 |
| AACAAGCCAA | X79676 | protein tyrosine phosphatase, non-receptor type 13 (APO-1/CD95 (Fas)-associated phosphatase) or another | 0.55 | 0.58 | 0.00 | 0.72 | 0.00 | 0.18 | 0.00 | 0.00 | 6.44 | 6.11 |
| ATGTTTGCCC | X97795 | RAD54 (S. cerevisiae)-like | 0.55 | 0.58 | 0.20 | 0.72 | 0.00 | 0.18 | 0.00 | 0.00 | 6.44 | 6.11 |
| AGGAAAGCCA | Z97074 | Rab9 effector p40 | 0.55 | 0.58 | 0.00 | 0.54 | 0.00 | 0.18 | 0.00 | 0.64 | 6.44 | 6.11 |
| AAGAACGTAG | Hs.145696 | splicing factor (CC1.3) (EST) | 0.55 | 0.58 | 0.20 | 0.54 | 0.00 | 0.18 | 0.00 | 0.00 | 6.44 | 6.11 |
| TTGGTAAGAC | Hs.260024 | Cdc42 effector protein 3 (EST) | 0.55 | 0.58 | 0.20 | 0.00 | 0.00 | 0.18 | 0.00 | 0.00 | 6.44 | 6.11 |
| CAGATTTCTG | Hs.198891 | serine/threonine-protein kinase PRP4 homolog (EST) | 0.55 | 0.58 | 0.39 | 0.54 | 0.00 | 0.18 | 0.00 | 0.00 | 6.44 | 6.11 |
| TCTGCAGGTC | AB016839 | hypothetical protein FLJ10788 | 0.37 | 0.58 | 0.20 | 0.00 | 0.00 | 0.18 | 0.33 | 0.00 | 6.44 | 6.11 |
| GCTGGGACAG | AB023186 | phosphoinositol 3-phosphate-binding protein-2 | 0.37 | 0.58 | 0.39 | 1.08 | 0.00 | 0.18 | 0.00 | 0.32 | 6.44 | 4.11 |
| TGTCTGGATG | AF040704 | 101F6; putative tumor suppressor | 0.37 | 0.58 | 0.00 | 0.18 | 0.00 | 0.18 | 0.00 | 0.00 | 6.44 | 4.11 |
| TCCTAGTAGG | AF059524 | RTN3; reticulon 3 | 0.37 | 0.58 | 0.20 | 0.54 | 0.00 | 0.18 | 0.00 | 0.00 | 6.44 | 4.11 |
| GTGAAGCCCC | AK000702 | WW Domain-Containing Gene or hypothetical protein FLJ20695 | 0.37 | 0.58 | 0.39 | 0.18 | 0.00 | 0.18 | 0.65 | 0.64 | 6.44 | 4.11 |
| GTTTGTTGGG | AK001628 | KIAA0483 protein | 0.37 | 0.58 | 0.39 | 0.18 | 0.00 | 0.18 | 0.00 | 0.00 | 6.44 | 4.11 |
| TTCTGAAGAC | BC001625 | for protein disulfide isomerase-related | 0.37 | 0.58 | 0.20 | 0.36 | 0.00 | 0.18 | 0.00 | 0.00 | 6.44 | 4.11 |
| TATTCTCAAT | U79254 | asparaginyl-tRNA synthetase | 0.37 | 0.58 | 0.39 | 0.36 | 0.00 | 0.18 | 0.98 | 0.00 | 6.44 | 4.11 |
| GAGCAGCTGG | U83246 | copine I or SWAP-70 protein | 0.37 | 0.58 | 0.00 | 0.72 | 0.00 | 0.18 | 0.33 | 0.64 | 6.44 | 4.11 |
| GAACTGCCTC | U97075 | CFLAR; CASP8 and FADD-like apoptosis regulator | 0.37 | 0.58 | 0.98 | 0.72 | 0.00 | 0.18 | 0.00 | 0.64 | 6.44 | 4.11 |
| GGCAGACAAT | Hs.181163 | hypothetical protein MGC5629 (EST) | 0.74 | 1.16 | 0.20 | 0.36 | 0.00 | 0.35 | 0.00 | 0.00 | 6.44 | 4.23 |
| TTTTCCCTGT | Hs.178292 | KIAA0180 protein (EST) | 0.37 | 0.58 | 0.39 | 0.18 | 0.00 | 0.18 | 0.00 | 0.00 | 6.44 | 4.11 |
| CCAAAATTAG | Hs.239737 | C-terminal binding protein 1 (EST) | 0.37 | 0.58 | 0.39 | 0.90 | 0.00 | 0.18 | 0.65 | 0.00 | 6.44 | 4.11 |
| TTAAAATACA | Hs.11356 | EST | 0.37 | 0.58 | 0.59 | 0.18 | 0.00 | 0.18 | 0.00 | 0.00 | 6.44 | 4.11 |
| GGGCTGGGGG | Hs.185564 | EST | 0.37 | 0.58 | 1.37 | 0.00 | 0.00 | 0.18 | 1.30 | 0.32 | 6.44 | 4.11 |
| CAGCTATTTC | M94856 | fatty acid binding protein 5 (psoriasis-associated) | 3.32 | 2.32 | 1.57 | 1.99 | 0.39 | 0.35 | 0.00 | 0.64 | 6.27 | 8.97 |
| CAGGACAGTT | AF070629 | RAB2, member RAS oncogene family | 1.66 | 1.74 | 1.37 | 1.26 | 0.20 | 0.35 | 1.63 | 0.32 | 6.21 | 6.04 |
| CTGCTAGGAA | AF130055 | translocating chain-associating membrane protein | 12.17 | 13.33 | 14.71 | 17.34 | 2.35 | 1.95 | 2.28 | 0.64 | 6.20 | 5.66 |
| CCGGGGAGCA | AK000999 | GDP-mannose pyrophosphorylase A | 1.84 | 1.74 | 1.18 | 0.54 | 0.59 | 0.00 | 0.33 | 0.97 | 6.00 | 6.24 |
| TCTTGTAACT | AK001023 | nucleotide binding protein 2 (E. coli MinD like) | 1.29 | 0.58 | 1.57 | 0.72 | 0.20 | 0.00 | 0.33 | 0.00 | 5.80 | 12.90 |
| TCTGTTGTTC | Hs.289770 | EST | 1.29 | 0.58 | 0.20 | 0.36 | 0.20 | 0.00 | 0.00 | 0.00 | 5.80 | 12.90 |
| TGTGAATAAA | D17286 | EST | 1.11 | 0.58 | 0.20 | 0.72 | 0.20 | 0.00 | 0.33 | 0.00 | 5.80 | 11.10 |
| TGGCAAGATG | Hs.91728 | polymyositis/scleroderma autoantigen 1 (75 kD) (EST) | 1.11 | 0.58 | 0.20 | 1.44 | 0.20 | 0.00 | 0.00 | 0.00 | 5.80 | 11.10 |
| TCTAAAGAAT | AK001226 | hypothetical protein | 0.92 | 0.58 | 0.00 | 0.18 | 0.20 | 0.00 | 0.00 | 0.00 | 5.80 | 9.20 |
| GGCGTTAGAG | AJ277841 | ELG protein | 0.74 | 0.58 | 0.00 | 0.00 | 0.20 | 0.00 | 0.00 | 0.00 | 5.80 | 7.40 |
| TACCCCAGAA | AJ290393 | putative b,b-carotene-9′, 10′-dioxygenase | 0.74 | 0.58 | 0.59 | 0.18 | 0.20 | 0.00 | 0.00 | 0.00 | 5.80 | 7.40 |
| TGTACATTTT | J03198 | guanine nucleotide binding protein (G protein), alpha inhibiting activity polypeptide 3 | 0.74 | 0.58 | 0.20 | 0.00 | 0.20 | 0.00 | 0.33 | 0.00 | 5.80 | 7.40 |
| CAGCTCCAAA | Hs.121575 | cDNA FLJ12231 fis, clone MAMMA1001191 | 0.74 | 0.58 | 0.39 | 0.90 | 0.20 | 0.00 | 0.33 | 0.32 | 5.80 | 7.40 |
| GGAAACTGAT | Hs.83916 | NADH dehydrogenase (ubiquinone) 1 alpha subcomplex, 5 (13 kD, B13) (EST) | 0.74 | 0.58 | 0.59 | 0.18 | 0.20 | 0.00 | 0.33 | 0.64 | 5.80 | 7.40 |
| CTTCTGTTTT | Hs.293687 | EST | 0.74 | 0.58 | 0.59 | 0.72 | 0.20 | 0.00 | 0.00 | 0.00 | 5.80 | 7.40 |
| TGTCAGGAAC | Hs.66666 | Weakly similar to S19560 proline-rich protein MP4 - mouse [M. musculus] (EST) | 0.74 | 0.58 | 0.00 | 0.00 | 0.20 | 0.00 | 0.00 | 0.00 | 5.80 | 7.40 |
| AAAGGGGGCA | Hs.249247 | heterogeneous nuclear protein similar to rat helix destabilizing protein (EST) | 1.29 | 1.16 | 0.59 | 0.18 | 0.39 | 0.00 | 0.98 | 0.00 | 5.80 | 6.62 |
| TTCAACGAGG | AB012193 | cullin 4A | 0.55 | 0.58 | 0.78 | 0.72 | 0.20 | 0.00 | 0.00 | 0.00 | 5.80 | 5.50 |
| TAAGGATTTT | AL080084 | CGI-100 protein | 0.55 | 0.58 | 0.59 | 0.90 | 0.20 | 0.00 | 0.00 | 0.00 | 5.80 | 5.50 |
| TAAAAGACAA | AL110273 | spectrin, alpha, non-erythrocytic 1 (alpha-fodrin) | 0.55 | 0.58 | 1.96 | 1.44 | 0.20 | 0.00 | 0.33 | 0.32 | 5.80 | 5.50 |
| GGCTCTGTCA | Hs.107000 | hypothetical protein FLJ11294 (EST) or KIAA1856 protein (EST) | 0.55 | 0.58 | 0.20 | 0.36 | 0.20 | 0.00 | 0.00 | 0.32 | 5.80 | 5.50 |
| TGAACCCGTT | Hs.36927 | heat shock 105 kD (EST) | 0.55 | 0.58 | 0.39 | 0.18 | 0.20 | 0.00 | 0.65 | 0.00 | 5.80 | 5.50 |
| GCTGCATCCC | Hs.106673 | eukaryotic translation initiation factor 3, subunit 6 (48 kD) (EST) | 0.55 | 0.58 | 0.39 | 0.36 | 0.20 | 0.00 | 0.00 | 0.00 | 5.80 | 5.50 |
| TACTGGAAGT | Hs.9075 | serine/threonine kinase 17a (apoptosis-inducing) (EST) | 0.55 | 0.58 | 1.18 | 0.72 | 0.20 | 0.00 | 0.00 | 0.32 | 5.80 | 5.50 |
| GTTATAAGAT | U41515 | Deleted in split-hand/split-foot 1 region | 0.92 | 1.16 | 1.18 | 1.26 | 0.39 | 0.00 | 0.33 | 0.00 | 5.80 | 4.72 |
| GTACACATCC | L08096 | TNFSF7; tumor necrosis factor (ligand) superfamily, member 7 | 2.40 | 2.13 | 0.98 | 1.44 | 0.39 | 0.35 | 0.00 | 0.00 | 5.76 | 6.49 |
| TGAAATACTG | Hs.154545 | PDZ domain containing guanine nucleotide exchange factor(GEF)1 (EST) or other ESTs | 1.29 | 1.55 | 1.18 | 1.99 | 0.00 | 0.53 | 0.00 | 0.32 | 5.74 | 4.87 |
| GGCTTTGATT | X70476 | coatomer protein complex, subunit beta 2 (beta prime) | 4.61 | 3.09 | 2.75 | 2.17 | 0.20 | 0.89 | 0.33 | 0.00 | 5.72 | 8.46 |
| TAGCTTCTTC | Hs.76392 | aldehyde dehydrogenase 1 family, member A1 (EST) or other ESTs | 2.40 | 1.55 | 0.59 | 0.54 | 0.20 | 0.35 | 0.00 | 0.00 | 5.54 | 8.73 |
| CTAAAAGGAG | X12466 X13 | Human mRNA for snRNP E protein. | 1.84 | 1.55 | 1.37 | 0.72 | 0.20 | 0.35 | 0.98 | 0.32 | 5.54 | 6.69 |
| GAGGCTCAAT | AB030905 | chromobox homolog 3 (Drosophila HP1 gamma) or heterochromatin-like protein 1 | 2.40 | 2.90 | 3.33 | 3.25 | 0.00 | 1.06 | 0.33 | 0.00 | 5.47 | 4.53 |
| TATGCGTTTG | AF086023 | Weakly similar to I38022 hypothetical protein [H. sapiens] (EST) | 1.29 | 0.97 | 0.98 | 1.08 | 0.00 | 0.35 | 0.33 | 0.32 | 5.39 | 7.37 |
| ATGTACTAAA | Y07968 | TRK-fused gene | 1.29 | 0.97 | 1.18 | 1.99 | 0.00 | 0.35 | 0.65 | 1.29 | 5.39 | 7.37 |
| CAAATGCTGT | M97935 | signal transducer and activator of transcription 1, 91 kD | 5.35 | 1.93 | 3.14 | 1.63 | 0.20 | 0.53 | 0.65 | 0.32 | 5.36 | 14.66 |
| TCAGAAGTTT | AK000745 | EST | 1.66 | 1.55 | 1.96 | 2.17 | 0.59 | 0.00 | 0.33 | 0.00 | 5.34 | 5.63 |
| GGGGGTGGGT | AF086246 | tudor repeat associator with PCTAIRE 2 or mago-nashi (Drosophila) homolog, proliferation-associated | 2.03 | 1.93 | 1.37 | 1.81 | 0.39 | 0.35 | 0.33 | 0.00 | 5.22 | 5.49 |
| TGTAAGTCTG | M88108 | GAP-associated tyrosine phosphoprotein p62 (Sam68) | 2.77 | 2.32 | 2.16 | 2.35 | 0.20 | 0.71 | 0.98 | 0.97 | 5.16 | 6.09 |
| CTGTCATTTG | BC000914 | splicing factor, arginine/serine-rich 3 | 2.58 | 3.28 | 2.35 | 2.35 | 0.39 | 0.89 | 0.00 | 0.32 | 5.13 | 4.03 |
| CTCAGCAATG | L47277 | topoisomerase (DNA) II alpha (170 kD) (EST) | 1.66 | 0.97 | 1.76 | 1.08 | 0.20 | 0.18 | 0.00 | 0.00 | 5.11 | 8.74 |
| ACAAACTGTG | Hs.90370 | actin related protein 2/3 complex, subunit 1A (41 kD) (EST) | 1.29 | 0.97 | 0.78 | 0.36 | 0.20 | 0.18 | 0.98 | 0.64 | 5.11 | 6.79 |
| TAATAAAGCA | X76092 | regulatory factor X, 3 (influences HLA class II expression) or seryl-tRNA synthetase | 1.11 | 0.97 | 0.59 | 0.72 | 0.20 | 0.18 | 1.30 | 0.97 | 5.11 | 5.84 |
| GTAGACCCCA | Hs.236510 | uncharacterized hypothalamus protein HTMS (EST) | 0.92 | 0.97 | 0.59 | 0.90 | 0.20 | 0.18 | 0.00 | 0.00 | 5.11 | 4.84 |
| TGCCTGTAAT | AK001590 | islet amyloid polypeptide or others | 1.29 | 1.35 | 1.57 | 1.08 | 0.00 | 0.53 | 0.98 | 0.32 | 5.00 | 4.87 |
| TGCAATAAGC | AL122072 | heterogeneous nuclear ribonucleoprotein U (scaffold attachment factor A) | 1.48 | 0.97 | 0.39 | 1.26 | 0.39 | 0.00 | 0.98 | 0.32 | 4.85 | 7.59 |
| GTGTATCTTT | M90104 | splicing factor, arginine/serine-rich 2 | 0.92 | 0.97 | 1.18 | 1.99 | 0.39 | 0.00 | 0.65 | 0.00 | 4.85 | 4.72 |
| TGCCAAAAAA | Hs.19261 | dystonia 1, torsion (autosomal dominant; torsin A) (EST) or titin-cap (telethonin) (EST) | 0.92 | 0.97 | 0.78 | 1.63 | 0.39 | 0.00 | 0.00 | 0.00 | 4.85 | 4.72 |
| AGGACTTCTG | Hs.43847 | mRNA for MADP-1 protein, complete cds (EST) | 2.03 | 1.35 | 0.39 | 1.26 | 0.39 | 0.18 | 0.33 | 0.00 | 4.82 | 7.12 |
| ACTGGGGAAT | Hs.24763 | RAN binding protein 1 (EST) | 1.29 | 1.35 | 1.37 | 1.26 | 0.20 | 0.35 | 0.98 | 0.00 | 4.82 | 4.69 |
| CAGTTTGTAC | X52709 | pyruvate dehydrogenase (lipoamide) alpha 1 | 2.40 | 2.70 | 2.16 | 1.63 | 0.59 | 0.53 | 0.98 | 0.00 | 4.82 | 4.29 |
| GCAGCTAATT | AF226054 | GK001 protein | 1.11 | 1.35 | 0.59 | 0.90 | 0.20 | 0.35 | 0.00 | 0.97 | 4.82 | 4.04 |
| CCCCAGTCGG | M59371 M36 | EphA2 | 2.21 | 1.74 | 1.18 | 0.90 | 0.39 | 0.35 | 0.65 | 0.00 | 4.70 | 5.97 |
| TCAACAGCGT | BC005884 | enolase 1, (alpha) | 2.03 | 1.74 | 0.98 | 0.90 | 0.39 | 0.35 | 0.33 | 0.00 | 4.70 | 5.49 |
| TAGCTGAGAC | U09559 | karyopherin alpha 2 (RAG cohort 1, importin alpha 1) | 1.66 | 1.74 | 1.57 | 1.08 | 0.39 | 0.35 | 0.65 | 0.00 | 4.70 | 4.49 |
| GATTTTGTAG | Hs.84264 | acidic protein rich in leucines | 2.03 | 2.13 | 3.14 | 2.17 | 0.39 | 0.53 | 0.33 | 0.97 | 4.63 | 4.41 |
| TACCCTAAAA | AB014575 | KIAA0675 gene product or others | 6.09 | 4.06 | 3.14 | 3.79 | 0.59 | 1.24 | 1.95 | 0.32 | 4.46 | 6.66 |
| TACCAGTGTA | M34664 | heat shock 60 kD protein 1 (chaperonin) | 4.24 | 3.67 | 3.53 | 1.99 | 0.78 | 0.89 | 0.33 | 1.29 | 4.42 | 5.08 |
| TAACCATTTT | AL031320 | hypothetical protein MGC1314 similar to fucosidase, alpha-L-1, tissue | 1.66 | 0.39 | 0.39 | 0.36 | 0.00 | 0.18 | 0.00 | 0.00 | 4.33 | 18.44 |
| TTGCTATGAA | AB051495 | KIAAI708 protein | 1.29 | 0.39 | 0.39 | 0.36 | 0.00 | 0.18 | 0.00 | 0.00 | 4.33 | 14.33 |
| ACACTTGGAG | Hs.198726 | vasoactive intestinal peptide receptor 1 (EST) | 1.29 | 0.39 | 0.98 | 1.44 | 0.00 | 0.18 | 1.30 | 1.93 | 4.33 | 14.33 |
| ATTGCTTTTG | AF091071 | similar to S. cerevisiae RER1 | 1.11 | 0.39 | 0.20 | 1.08 | 0.00 | 0.18 | 0.98 | 0.64 | 4.33 | 12.33 |
| AGCTCTGCTG | AK024501 | Mad4 homolog | 1.11 | 0.39 | 0.59 | 0.72 | 0.00 | 0.18 | 0.33 | 0.00 | 4.33 | 12.33 |
| TAACTAACAA | D14663 | kynureninase (L-kynurenine hydrolase) | 0.92 | 0.39 | 0.20 | 0.00 | 0.00 | 0.18 | 0.33 | 0.64 | 4.33 | 10.22 |
| TACTGTATGT | AB020658 | suppressor of actin 1 | 0.74 | 0.39 | 0.20 | 0.54 | 0.00 | 0.18 | 0.33 | 0.00 | 4.33 | 8.22 |
| GTTCATAGGT | AB051486 | secretory protein SEC8; KIAA1699 protein or N-terminal acetyltransferase complex ard1subunit (EST) | 0.74 | 0.39 | 0.39 | 0.00 | 0.00 | 0.18 | 0.00 | 0.32 | 4.33 | 8.22 |
| TGGTCCACGG | AF125098 | Homo sapiens HSPC037 protein mRNA, complete cds. | 0.74 | 0.39 | 0.98 | 0.54 | 0.00 | 0.18 | 0.00 | 0.00 | 4.33 | 8.22 |
| TATGATTATT | L07956 | glucan (1,4-alpha-), branching enzyme 1 (glycogen branching enzyme, Andersen disease, | 0.74 | 0.39 | 0.00 | 0.18 | 0.00 | 0.18 | 0.00 | 0.00 | 4.33 | 8.22 |
| | | glycogen storage disease type | | | | | | | | | | |
| CCTCGGAGAT | L22253 | Homo sapiens 9G8 splicing factor mRNA, complete cd | 0.74 | 0.39 | 0.39 | 0.54 | 0.00 | 0.18 | 0.00 | 0.00 | 4.33 | 8.22 |
| TAGGTAGCTC | L36587 | clone IMAGE: 3457003, mRNA | 0.74 | 0.39 | 0.78 | 0.90 | 0.00 | 0.18 | 0.00 | 0.00 | 4.33 | 8.22 |
| TTCTTCTCGT | X99584 | SMT3 (suppressor of mif two 3, yeast) homolog 1 | 0.74 | 0.39 | 0.00 | 0.18 | 0.00 | 0.18 | 0.00 | 0.00 | 4.33 | 8.22 |
| ATGAACACGG | Hs.180799 | C3HC4-type zinc figer protein (EST) | 0.74 | 0.39 | 0.39 | 0.36 | 0.00 | 0.18 | 0.00 | 0.00 | 4.33 | 8.22 |
| GCTTAGAAGT | Hs.289088 | heat shock 90 kD protein 1, alpha (EST) | 0.74 | 0.39 | 0.59 | 0.72 | 0.00 | 0.18 | 0.00 | 0.32 | 4.33 | 8.22 |
| GAATGGATGC | Hs.292419 | EST | 0.74 | 0.39 | 1.18 | 0.54 | 0.00 | 0.18 | 0.00 | 0.00 | 4.33 | 8.22 |
| CTTGATTCCT | AB020710 | KIAA0903 protein | 0.55 | 0.39 | 0.00 | 0.36 | 0.00 | 0.18 | 0.33 | 0.32 | 4.33 | 6.11 |
| TGTTTAATGT | AB033075 | DDEF1; development and differentiation enhanching factor 1 | 0.55 | 0.39 | 0.00 | 0.90 | 0.00 | 0.18 | 0.00 | 0.00 | 4.33 | 6.11 |
| CATTTGAAAG | AF052159 | tumor protein p53-binding protein, 1 or clone 24416 mRNA sequence | 0.55 | 0.39 | 0.59 | 0.18 | 0.00 | 0.18 | 0.00 | 0.00 | 4.33 | 6.11 |
| CCCCTCAAAA | AK000301 | hypothetical protein FLJ20294 | 0.55 | 0.39 | 0.00 | 0.00 | 0.00 | 0.18 | 0.00 | 0.00 | 4.33 | 6.11 |
| TTCTGTGTAT | AK001286 | TLS-associated serine-arglnine protein 1 | 0.55 | 0.39 | 0.78 | 0.54 | 0.00 | 0.18 | 0.00 | 0.32 | 4.33 | 6.11 |
| TAGCCAAAAA | BC007067 | DKFZP566O1646 protein | 0.55 | 0.39 | 0.39 | 0.36 | 0.00 | 0.18 | 0.00 | 0.00 | 4.33 | 6.11 |
| GAGGATCACT | BC009471 | alpha-methylacyl-CoA racemase | 0.55 | 0.39 | 0.39 | 0.54 | 0.00 | 0.18 | 0.00 | 0.00 | 4.33 | 6.11 |
| AAGTGTGTTT | D25278 | KIAA0036 gene product | 0.55 | 0.39 | 0.20 | 0.00 | 0.00 | 0.18 | 0.33 | 0.00 | 4.33 | 6.11 |
| TTGTTTAATT | U03851 | capping protein (actin filament) muscle Z-line, alpha 2 | 0.55 | 0.39 | 0.39 | 0.18 | 0.00 | 0.18 | 0.00 | 0.00 | 4.33 | 6.11 |
| ACTGGTATAC | U10860 | guanine monphosphate synthetase | 0.55 | 0.39 | 0.39 | 0.54 | 0.00 | 0.18 | 0.33 | 0.00 | 4.33 | 6.11 |
| GCCGCTTCTA | U24497 U24 | PKD1; polycystic kidney disease 1 (autosomal dominant, protein | 0.55 | 0.39 | 1.18 | 0.18 | 0.00 | 0.18 | 0.33 | 0.64 | 4.33 | 6.11 |
| TGTGATCACA | Y13640 | proteasome (prosome, macropain) subunit, beta type, 10 | 0.55 | 0.39 | 0.39 | 0.18 | 0.00 | 0.18 | 0.98 | 0.00 | 4.33 | 6.11 |
| GCCCCCAATT | Hs.227751 | lectin, galactoside-binding, soluble, 1 (galectin 1) (EST) | 0.55 | 0.39 | 0.20 | 0.18 | 0.00 | 0.18 | 0.00 | 0.00 | 4.33 | 6.11 |
| TGAAGAGACT | Hs.240767 | Contains the 5′ end of the gene for a novel cyclophilin type peptidyl-prolyl cis-trans | 0.55 | 0.39 | 0.20 | 0.54 | 0.00 | 0.18 | 0.65 | 0.00 | 4.33 | 6.11 |
| | | isomerase (EST) or others | | | | | | | | | | |
| TGGGTTAATA | Hs.101047 | transcription factor 3 (E2A immunoglobulin enhancer binding factors E12/E47) (EST) | 0.55 | 0.39 | 0.39 | 0.18 | 0.00 | 0.18 | 0.33 | 0.00 | 4.33 | 6.11 |
| TAAGCCCTTT | Hs.90606 | 15 kDa selenoprotein | 0.55 | 0.39 | 0.39 | 0.18 | 0.00 | 0.18 | 0.00 | 0.00 | 4.33 | 6.11 |
| TGCCCCGGGC | Hs.278242 | hypothetical protein MGC12992 (EST) | 0.55 | 0.39 | 0.39 | 0.00 | 0.00 | 0.18 | 0.00 | 0.32 | 4.33 | 6.11 |
| CACATTAGGT | Hs.182625 | VAMP (vesicle-associated membrane protein)-associated protein B and C (EST) or another EST | 0.55 | 0.39 | 0.39 | 0.00 | 0.00 | 0.18 | 0.33 | 0.32 | 4.33 | 6.11 |
| TTTCTCAGTG | Hs.172280 | SWI/SNF related, matrix associated, actin dependent regulator of chromatin, subfamily c, member 1 (EST) | 0.55 | 0.39 | 0.59 | 0.00 | 0.00 | 0.18 | 0.00 | 0.32 | 4.33 | 6.11 |
| GAACACCACT | Hs.155595 | neural precursor cell expressed, developmentally down-regulated 5 (EST) | 0.55 | 0.39 | 0.98 | 0.36 | 0.00 | 0.18 | 0.98 | 0.00 | 4.33 | 6.11 |
| TATGGTCTGG | Hs.28309 | UDP-glucose dehydrogenase (EST) | 0.55 | 0.39 | 0.00 | 0.18 | 0.00 | 0.18 | 0.00 | 0.00 | 4.33 | 6.11 |
| AATGTCCAGT | Hs.26373 | cDNA: FLJ23449 fis, clone HSI05859 (EST) | 0.55 | 0.39 | 0.00 | 0.54 | 0.00 | 0.18 | 0.00 | 0.00 | 4.33 | 6.11 |
| TAACAAAGGA | Hs.4113 | S-adenosylhomocysteine hydrolase-like 1 (EST) | 0.55 | 0.39 | 1.18 | 1.08 | 0.00 | 0.18 | 0.00 | 0.00 | 4.33 | 6.11 |
| TACATTTCAA | AB019391 | proprotein convertase subtilisin/kexin type 5 | 0.37 | 0.39 | 0.39 | 0.36 | 0.00 | 0.18 | 0.00 | 0.32 | 4.33 | 4.11 |
| GTTTCAGGAG | AB023430 | proteine tyrosine phosphatase, non-receptor type substrate 1 | 0.37 | 0.39 | 0.59 | 1.26 | 0.00 | 0.18 | 1.63 | 1.29 | 4.33 | 4.11 |
| CAAAGGCCCT | AB033068 | Frz1 protein | 0.37 | 0.39 | 0.20 | 0.18 | 0.00 | 0.18 | 0.00 | 0.32 | 4.33 | 4.11 |
| GGACAGGGCA | AB058759 | KIAA1856 protein | 0.37 | 0.39 | 0.20 | 0.00 | 0.00 | 0.18 | 0.00 | 0.00 | 4.33 | 4.11 |
| GAGAACTCCC | AF054992 | PKD2: polycystic kidney disease 2(autosomal dominant) | 0.37 | 0.39 | 0.78 | 0.54 | 0.00 | 0.18 | 0.00 | 0.32 | 4.33 | 4.11 |
| GCACTTCAAA | AF070585 | topoisomerase (DNA) III beta or others | 0.37 | 0.39 | 0.78 | 0.18 | 0.00 | 0.18 | 0.00 | 0.00 | 4.33 | 4.11 |
| ACCCTGGGCA | AF070630 | chromosome 1 open reading frame 2 (=A) or hypothetical protein FLJ13052 (=C) | 0.37 | 0.39 | 0.20 | 0.00 | 0.00 | 0.18 | 0.00 | 0.00 | 4.33 | 4.11 |
| TTTATTCCCT | AF144094 | myosin XV or DC13 protein | 0.37 | 0.39 | 1.37 | 0.54 | 0.00 | 0.18 | 0.00 | 0.00 | 4.33 | 4.11 |
| TAACTGTCAA | AF161398 | hypothetical protein PRO2013 | 0.37 | 0.39 | 0.20 | 0.38 | 0.00 | 0.18 | 0.00 | 0.00 | 4.33 | 4.11 |
| AGACTATATT | AF195530 | XPNPEP1; X-prolyl aminopeptidase (aminopeptidase P) 1, soluble | 0.37 | 0.39 | 0.39 | 0.18 | 0.00 | 0.18 | 0.98 | 0.00 | 4.33 | 4.11 |
| TTATGCTTTC | AF208856 | hypothetical protein | 0.37 | 0.39 | 0.20 | 0.18 | 0.00 | 0.18 | 0.00 | 0.32 | 4.33 | 4.11 |
| GGACTTAGAA | AK000746 | hypothetical protein FLJ20739 | 0.37 | 0.39 | 0.00 | 0.00 | 0.00 | 0.18 | 0.00 | 0.32 | 4.33 | 4.11 |
| TTTTGTAATT | AL133583 | MBTPS1; membrane-bound transcription factor protease, site 1 | 0.37 | 0.39 | 0.20 | 0.00 | 0.00 | 0.18 | 0.00 | 0.00 | 4.33 | 4.11 |
| TTTTGTGCAT | AY004255 | cyclin-dependent kinase inhibitor 1B (p27, Kip1) | 0.37 | 0.39 | 0.00 | 0.36 | 0.00 | 0.18 | 0.65 | 0.00 | 4.33 | 4.11 |
| TCAAGGCCCC | BC002329 | hypothetical protein DKFZp547M136 similar to widely-interspaced zinc finger motifs | 0.37 | 0.39 | 0.00 | 0.00 | 0.00 | 0.18 | 0.00 | 0.00 | 4.33 | 4.11 |
| GGACCGAGGG | BC003353 | hypothetical protein MGC5309 | 0.37 | 0.39 | 0.20 | 0.18 | 0.00 | 0.18 | 0.00 | 0.97 | 4.33 | 4.11 |
| CTTGTGTGTA | D31883 | Human mRNA for KIAA0059 gene, complete cds. | 0.37 | 0.39 | 0.39 | 0.00 | 0.00 | 0.18 | 0.00 | 0.32 | 4.33 | 4.11 |
| CCCATTCACT | L25275 | sulfotransferase family, cytosolic, 1A, phenol-preferring, member 3 | 0.37 | 0.39 | 0.00 | 0.18 | 0.00 | 0.18 | 0.00 | 0.32 | 4.33 | 4.11 |
| TACACCCGCT | M31899 | excision repair cross-complementing rodent repair deficiency, complementation group 3 | 0.37 | 0.39 | 0.00 | 0.36 | 0.00 | 0.18 | 0.33 | 0.00 | 4.33 | 4.11 |
| CATCCTTGGG | Hs.119222 | suppression of tumorigenicity 13 (colon carcinoma) (Hsp70-interacting protein) (EST) | 0.37 | 0.39 | 0.20 | 0.18 | 0.00 | 0.18 | 0.33 | 0.00 | 4.33 | 4.11 |
| TGGGCCAGGC | Hs.175358 | Weakly similar to A47582 B-cell growth factor precursor [H. sapiens] (EST) or Weakly similar to | 0.37 | 0.39 | 0.20 | 0.18 | 0.00 | 0.18 | 0.00 | 0.00 | 4.33 | 4.11 |
| | | neuronal thread protein AD | | | | | | | | | | |
| GCTGGATTTT | Hs.172788 | ALEX3 protein (EST) or laminin, beta 1 (EST) or others | 0.37 | 0.39 | 0.20 | 0.18 | 0.00 | 0.18 | 0.00 | 0.00 | 4.33 | 4.11 |
| GCCAAGCCCC | Hs.283738 | casein kinase 1, alpha 1 (EST) | 0.37 | 0.39 | 0.20 | 0.36 | 0.00 | 0.18 | 0.00 | 0.32 | 4.33 | 4.11 |
| CTGAAACCCC | Hs.49760 | origin recognition complex, subunit 6 (yeast homolog)-like (EST) or other ESTs | 0.37 | 0.39 | 0.39 | 0.36 | 0.00 | 0.18 | 0.00 | 0.32 | 4.33 | 4.11 |
| GCATCCCTGA | Hs.211973 | homolog of Yeast RRP4 (ribosomal RNA processing 4), 3′-5′-exoribonuclease (EST) | 0.37 | 0.39 | 0.39 | 0.72 | 0.00 | 0.18 | 0.00 | 0.00 | 4.33 | 4.11 |
| CAAGTTTCCA | Hs.332405 | arginase, liver (EST) or four and a half LIM domains 1 (EST) | 0.37 | 0.39 | 0.59 | 0.36 | 0.00 | 0.18 | 0.00 | 0.00 | 4.33 | 4.11 |
| TCTGCTTTTG | Hs.350401 | solute carrier family 4 (anion exchanger), member 1 adapter protein (EST) | 0.37 | 0.39 | 0.59 | 0.00 | 0.00 | 0.18 | 0.33 | 0.00 | 4.33 | 4.11 |
| AGCTGACAGG | Hs.278589 | general transcription factor II, I (EST) | 0.37 | 0.39 | 0.78 | 0.36 | 0.00 | 0.18 | 0.00 | 0.32 | 4.33 | 4.11 |
| TCCATAGATT | Hs.75607 | myristoylated alanine-rich protein kinase C substrate (MARCKS, 80K-L) (EST) | 0.37 | 0.39 | 0.78 | 0.00 | 0.00 | 0.18 | 0.65 | 0.64 | 4.33 | 4.11 |
| CTCCTTAAGA | Hs.49760 | origin recognition complex, subunit 6 (yeast homolog)-like (EST) or others | 0.37 | 0.39 | 0.00 | 0.18 | 0.00 | 0.18 | 0.00 | 0.32 | 4.33 | 4.11 |
| GGACAGCTCA | Hs.187958 | solute carrier family 6 (neurotransmitter transporter, creatine), member 8 (EST, or zinc finger | 0.37 | 0.39 | 0.00 | 0.18 | 0.00 | 0.18 | 0.00 | 0.00 | 4.33 | 4.11 |
| | | protein homologous to Zfp91 | | | | | | | | | | |
| CAAATAAACT | Hs.301865 | dopachrome tautomerase (dopachrome dalta-isomerase, tyrosine-related protein 2) (EST) or others | 0.37 | 0.39 | 0.00 | 0.36 | 0.00 | 0.18 | 0.00 | 0.32 | 4.33 | 4.11 |
| CTCCTGGCCC | Hs.5321 | ARP3 (actin-related protein 3, yeast) homolog (EST) | 0.37 | 0.39 | 0.00 | 0.54 | 0.00 | 0.18 | 0.00 | 0.00 | 4.33 | 4.11 |
| GTTTGGGGCT | Hs.75725 | transgelin 2 (EST) | 0.37 | 0.39 | 0.00 | 0.00 | 0.00 | 0.18 | 0.00 | 0.00 | 4.33 | 4.11 |
| GCCTTAACAA | U02020 | pre-B-cell colony-enhancing factor | 1.48 | 1.16 | 1.76 | 0.90 | 0.00 | 0.53 | 0.00 | 1.29 | 4.30 | 5.58 |
| CTGTACAGAC | AK000560 | tubulin, beta, 2 | 12.72 | 13.33 | 13.92 | 15.71 | 1.96 | 4.26 | 1.30 | 0.97 | 4.29 | 4.09 |
| AAGAAAGTTC | AL050255 | Novel gene similar to C. elegens hypothetical 55.2 | 2.03 | 0.77 | 0.78 | 1.26 | 0.00 | 0.35 | 0.00 | 0.64 | 4.28 | 11.60 |
| GCTGGATGCG | AF043896 | chromosome 9 open reading frame 3 or another EST | 1.11 | 0.77 | 1.18 | 1.08 | 0.00 | 0.35 | 0.33 | 0.32 | 4.28 | 6.34 |
| TGGAAAGAGC | Hs.9043 | DKFZP564O092 protein (EST) | 0.92 | 0.77 | 0.39 | 0.36 | 0.00 | 0.35 | 0.33 | 0.00 | 4.28 | 5.26 |
| GCAAGCCCCA | Hs.334895 | Moderately similar to PC4259 ferritin associated protein [H. sapiens] (EST) or heat | 0.92 | 0.77 | 0.39 | 0.72 | 0.00 | 0.35 | 0.00 | 0.32 | 4.28 | 5.26 |
| | | shock 27 kD protein 1 (EST) | | | | | | | | | | |
| TGGAACTGTA | Hs.234839 | vacuolar sorting protein 4 (EST) or others | 0.92 | 0.77 | 0.78 | 0.18 | 0.00 | 0.35 | 0.33 | 0.00 | 4.28 | 5.26 |
| TGAATGTCAA | Hs.5297 | DKFZP564A2416 protein (EST) | 0.92 | 0.77 | 1.18 | 0.54 | 0.00 | 0.35 | 0.98 | 0.32 | 4.28 | 5.26 |
| CCTTTGTAAG | Hs.78465 | v-jun avian sarcoma virus 17 oncogene homolog (EST) | 0.74 | 0.77 | 0.39 | 0.18 | 0.00 | 0.35 | 1.63 | 0.64 | 4.28 | 4.23 |
| ATGGCGGCGA | Hs.165590 | ribosomal protein S13 (EST) | 0.74 | 0.77 | 0.39 | 0.54 | 0.00 | 0.35 | 0.00 | 0.00 | 4.28 | 4.23 |
| TGGACAAGCT | Hs.79516 | brain abundant, membrane attached signal protein 1 (EST) | 0.74 | 0.77 | 0.00 | 1.26 | 0.00 | 0.35 | 0.33 | 0.00 | 4.28 | 4.23 |
| AGCTGTATTC | X54942 X55 | CDC28 protein kinase 2 | 3.32 | 1.93 | 3.14 | 2.35 | 0.39 | 0.53 | 0.65 | 0.00 | 4.20 | 7.22 |
| ATGTGAAGAG | J03040 | secreted protein, acidic, cysteine-rich (osteonectin) (SPARC) | 6.82 | 6.18 | 6.08 | 8.31 | 1.57 | 1.42 | 2.93 | 1.61 | 4.15 | 4.56 |
| CCGAGGCTTG | K03200 | EST | 1.48 | 1.16 | 2.35 | 1.44 | 0.39 | 0.18 | 1.63 | 0.32 | 4.14 | 5.19 |
| CATACAGAAA | X84700 | CD97 antigen | 1.48 | 1.16 | 1.18 | 0.72 | 0.39 | 0.18 | 0.00 | 0.00 | 4.14 | 5.19 |
| CAGATTGCTG | Hs.21537 | protein phosphatase 1, catalytic subunit, beta isoform (EST) or serine/threonine-protein | 1.11 | 1.16 | 1.18 | 1.08 | 0.20 | 0.35 | 0.98 | 0.97 | 4.14 | 4.04 |
| | | kinase PRP4 homolog (EST) | | | | | | | | | | |
| TAGATAATGG | BC001002 | tubulin, beta 5 | 1.84 | 1.55 | 0.98 | 1.44 | 0.59 | 0.18 | 1.63 | 0.64 | 4.08 | 4.78 |
| TCAATCAAGA | S80794 | 14.3.3 eta chain = brain-specific tyrosine and trypt | 1.66 | 1.55 | 2.35 | 1.81 | 0.59 | 0.18 | 1.30 | 0.00 | 4.08 | 4.31 |
| ATGTAGTAGT | D55673 | heterogeneous nuclear ribonucleoprotein D (AU-rich element RNA-binding 1, 37 kD) | 3.14 | 2.32 | 2.55 | 2.89 | 0.78 | 0.35 | 0.65 | 0.32 | 4.07 | 5.56 |
| TCTGAAAACC | Hs.111554 | ADP-ribosylation factor-like 7 (EST) or others | 1.11 | 0.77 | 0.39 | 1.08 | 0.20 | 0.18 | 0.33 | 0.00 | 4.05 | 5.84 |
| TTACAACATT | AL049397 | cDNA DKFZp586C1019 (from clone DKFZp586C1019) | 0.92 | 0.77 | 1.57 | 0.90 | 0.20 | 0.18 | 0.33 | 0.32 | 4.05 | 4.84 |
| GATTGGCGGC | X82208 | BAF53 | 0.92 | 0.77 | 0.59 | 0.90 | 0.20 | 0.18 | 0.00 | 0.00 | 4.05 | 4.84 |
| CCACGTGTCC | Hs.239451 | Similar to zinc finger protein 16 (KOX 9) (EST) | 0.92 | 0.77 | 0.59 | 0.54 | 0.20 | 0.18 | 0.98 | 0.32 | 4.05 | 4.84 |
| TGCCTTACTT | AK001917 | PDCD6: programmed cell death 6 | 2.03 | 1.93 | 2.35 | 1.26 | 0.78 | 0.18 | 0.33 | 0.64 | 4.02 | 4.23 |

| | | | | | | | | | | | | |
|---|---|---|---|---|---|---|---|---|---|---|---|---|
| | | | | | | | | | | | | V7, |
| | Accession/ | | | | | | | | | | | V8/V5, |
| SAGE Tag | UniGene ID | Description | V1 | V2 | V3 | V4 | V5 | V6 | V7 | V8 | V3/V1 | V6 |

153 genes in FIG. 3(f) (135 genes; 18 tags with no mathches not shown)
SAGE data for genes up-regulated ≧ 4-fold by hypoxia in 786-0 VHL⁺ Hyp. (V3) when compared to 786-0 VHL⁺ Nor. (V1). Also shown,
for comparison, is the SAGE data for each gene as a ratio of its average expression in the two independent culture of RPTECs grown under hypoxia (V7, V8) with
respect to their average expression in the two independent cultures of RPTECs grown under nomoxia (V5, V6). SAGE tag abundances were normalized before
calculating ratios, as shown. The table is presented in descending order of V3/V1.

| | | | | | | | | | | | | |
|---|---|---|---|---|---|---|---|---|---|---|---|---|
| GTGGTGTGTG | AB033023 | nuclear factor of activated T-cells, cytoplasmic, calcineurin-dependent 3 or others | 0.18 | 0.58 | 1.76 | 0.54 | 0.39 | 0.18 | 0.00 | 0.97 | 9.78 | 1.70 |
| GTAAAAGTTC | D21262 | nucleolar and coiled-body phosphprotein 1 | 0.18 | 0.39 | 1.57 | 0.54 | 0.20 | 0.18 | 0.00 | 0.00 | 8.72 | 0.00 |
| AGAATGTACG | AL049851 | hypothetical protein | 0.37 | 1.35 | 2.94 | 1.81 | 0.20 | 0.35 | 0.33 | 0.64 | 7.95 | 1.76 |
| GAGCCGCCTC | AK001809 | HSPC182 protein | 0.18 | 0.39 | 1.37 | 0.90 | 0.59 | 1.06 | 0.00 | 0.32 | 7.61 | 0.19 |
| CCACAGGAGA | AJ251830 | p53-induced protein PIGPC1; THW tumor suppressor | 0.18 | 0.39 | 1.37 | 0.90 | 1.17 | 1.77 | 1.95 | 2.58 | 7.61 | 1.54 |
| GTGAACTTAC | AB013803 | contactin 5; NB-2; hNB-2 | 0.18 | 0.00 | 1.37 | 0.36 | 0.59 | 0.18 | 0.98 | 0.00 | 7.61 | 1.27 |
| AGGAAGAGCC | AF125535 | pp21 homolog | 0.18 | 1.16 | 1.18 | 0.00 | 0.39 | 0.00 | 0.65 | 0.64 | 6.56 | 3.31 |
| GTTGTGATGT | X66899 | Ewing sarcoma breakpoint region 1 | 0.18 | 0.97 | 1.18 | 1.08 | 0.00 | 0.35 | 0.00 | 0.00 | 6.56 | 0.00 |
| GTGCTTGTAC | AB001106 | GMFB; glia maturation factor, beta | 0.18 | 1.35 | 1.18 | 1.08 | 0.20 | 0.35 | 0.33 | 0.97 | 6.56 | 2.36 |
| ACCTGCTGGT | AK000447 | GTPase Rab14 | 0.18 | 1.16 | 1.18 | 0.00 | 0.20 | 0.35 | 0.00 | 0.97 | 6.56 | 1.76 |
| AGGGAGGCAG | Hs.74368 | transmembrane protein (63 kD), endoplasmic reticulum/Golgi intermediate compartment | 0.18 | 0.77 | 1.18 | 0.90 | 0.39 | 0.00 | 0.33 | 0.00 | 6.56 | 0.85 |
| AGCAACAGTG | U10117 | SCYE1; small inducible cytokine subfamily E, member 1 (endothelial monocyte-activating) | 0.18 | 0.77 | 1.18 | 0.18 | 0.39 | 0.00 | 0.33 | 0.97 | 6.56 | 3.33 |
| ATGAAACCCC | U67806 | small inducible cytokine A5 (RANTES) or others | 0.37 | 1.16 | 2.35 | 0.72 | 0.59 | 0.35 | 0.00 | 0.64 | 6.35 | 0.68 |
| CCCAGCTAAT | AF339827 | ATPase, H+ transporting, lysosomal (vacuolar proton pump), alpha polypeptide, 70 kD, isoform 1 | 0.18 | 0.19 | 1.18 | 1.26 | 0.20 | 0.18 | 0.33 | 0.32 | 6.56 | 1.71 |
| | | (11nt) or FOXJ2 forkhead factor (11 [symbol] | | | | | | | | | | |
| CAGCTGGGGC | X66975 S41 | polypyrimidine tract binding protein (heterogeneous nuclear ribonucleoprotein I) | 0.18 | 0.77 | 1.18 | 0.18 | 1.37 | 0.71 | 0.00 | 0.00 | 6.56 | 0.00 |
| TAGTCATCTT | U68758 | pyrroline-5-carboxylate synthetase (glutamate gamma-semialdehyde synthetase) | 0.18 | 0.58 | 1.18 | 0.18 | 0.00 | 0.00 | 0.00 | 0.00 | 6.56 | 0.00 |
| ACGGCTCCGA | Hs.48563 | ESTs | 0.18 | 0.77 | 1.18 | 0.90 | 2.15 | 0.71 | 0.98 | 0.97 | 6.56 | 0.68 |
| TGGGCTCTGA | D12676 | CD36 antigen (collagen type I receptor, thrombospondin receptor)-like 2 | 0.18 | 0.77 | 1.18 | 0.90 | 0.98 | 2.13 | 0.98 | 1.61 | 6.56 | 0.83 |
| | | (lysosomal integral membrane protein II) | | | | | | | | | | |
| GGAAGTCCTG | Hs.118787 | transforming growth factor, beta-induced, 68 kD | 0.18 | 0.39 | 1.18 | 1.08 | 0.00 | 0.00 | 0.33 | 0.00 | 6.56 | 0.17 |
| GCCAAGATGC | BC004944 | Similar to RIKEN cDNA 2310040G17 gene | 0.18 | 0.58 | 1.18 | 1.44 | 4.50 | 2.48 | 1.63 | 1.29 | 6.56 | 0.42 |
| ATGCAAGAGA | AB051504 | KIAA1717 protein | 0.18 | 0.00 | 1.18 | 0.00 | 0.00 | 0.00 | 0.00 | 0.00 | 6.56 | 0.00 |
| CCAGTGTGCA | Hs.249247 | heterogeneous nuclear protein similar to rat helix destabilizing protein | 0.18 | 0.00 | 1.18 | 0.36 | 0.00 | 0.00 | 0.00 | 0.00 | 6.56 | 0.00 |
| GCTAGGTATT | AJ250865 | testin | 0.18 | 0.77 | 0.98 | 1.08 | 0.00 | 0.18 | 0.33 | 0.00 | 5.44 | 1.83 |
| CTAAGGTGGG | L76702 | protein phosphatase 2, regulatory subunit B (B56), delta isoform | 0.18 | 0.97 | 0.98 | 0.18 | 0.00 | 0.35 | 0.33 | 0.64 | 5.44 | 2.77 |
| TGGAAATAAA | BC000547 | mitochondrial ribosomal protein S6 | 0.18 | 0.77 | 0.98 | 0.36 | 0.00 | 0.35 | 0.00 | 0.64 | 5.44 | 1.83 |
| GGTGCTCCCT | AB015633 | transmembrane protein 5 | 0.18 | 0.39 | 0.98 | 0.00 | 0.20 | 0.00 | 0.33 | 0.32 | 5.44 | 3.25 |
| GGGCGGGGGC | M81735 | polymerase (DNA directed), delta 1, catalytic subunit (125 kD) | 0.18 | 0.97 | 0.98 | 1.99 | 0.39 | 0.18 | 0.00 | 0.00 | 5.44 | 0.00 |
| TAGTAAGTCA | AL110153 | ribosomal protein L22 | 0.18 | 0.19 | 0.98 | 0.90 | 0.00 | 0.18 | 0.00 | 0.00 | 5.44 | 0.00 |
| GAGGAGTGGG | Hs.206770 | zinc finger protein 297 | 0.18 | 0.19 | 0.98 | 0.36 | 0.20 | 0.00 | 0.00 | 0.00 | 5.44 | 0.00 |
| AATAGGGTCA | AF086183 | amyloid beta (A4) precursor-like protein 2 (EST) | 0.37 | 1.16 | 1.96 | 0.90 | 0.59 | 0.71 | 0.00 | 0.00 | 5.30 | 0.00 |
| GGCTGCAGTC | AL110253 | double ring-finger protein, Dorfin | 0.18 | 0.39 | 0.98 | 0.00 | 0.59 | 0.00 | 0.65 | 0.97 | 5.44 | 2.75 |
| ATGCGCAAGG | Z36785 | ribosomal protein, large, P0 | 0.18 | 1.55 | 0.98 | 0.90 | 1.96 | 1.77 | 0.98 | 1.61 | 5.44 | 0.69 |
| GGAACGGATG | AJ131186 | nuclear matrix protein NMP200 related to splicing factor PRP19 | 0.18 | 0.39 | 0.98 | 0.00 | 0.78 | 0.18 | 0.65 | 0.00 | 5.44 | 0.68 |
| CTTTCTTCCC | X79067 X71 | ZFP36L1; Zinc finger protein 36, C3H type-like 1; butyrate response factor 1 (EGF-response factor 1) | 0.18 | 0.39 | 0.98 | 0.36 | 0.98 | 0.00 | 1.30 | 1.61 | 5.44 | 2.97 |
| GTGCCTAGGA | Hs.25999 | hypothetical protein FLJ22195 | 0.18 | 1.16 | 0.98 | 1.63 | 0.78 | 2.13 | 1.95 | 1.29 | 5.44 | 1.11 |
| GTCTTTCTGG | AB046781 | uveal autoantigen with coiled coil domains and ankyrin repeats | 0.18 | 0.77 | 0.98 | 0.54 | 0.00 | 0.00 | 0.00 | 0.64 | 5.44 | 0.32 |
| GCTACTATTA | Hs.154443 | minichromosome maintenance deficient (S. cerevisiae) 4 | 0.18 | 0.77 | 0.98 | 0.54 | 0.00 | 0.00 | 0.00 | 0.00 | 5.44 | 0.00 |
| TTCTCTCAAC | AF089106 | unknown | 0.18 | 0.77 | 0.98 | 0.72 | 0.78 | 1.42 | 2.28 | 0.97 | 5.44 | 1.48 |
| TAAACTTCAA | Hs.87627 | disrupter of silencing 10 | 0.18 | 0.19 | 0.98 | 0.54 | 0.20 | 0.35 | 0.65 | 0.00 | 5.44 | 1.18 |
| GTGCAAAATG | Hs.247280 | HBV associated factor | 0.18 | 0.39 | 0.98 | 1.08 | 0.39 | 1.06 | 1.95 | 2.90 | 5.44 | 3.34 |
| GCTATGCTCC | BC003653 | presenilins associated rhomboid-like protein | 0.18 | 0.39 | 0.98 | 0.72 | 0.78 | 0.71 | 0.33 | 0.97 | 5.44 | 0.87 |
| CAGAGTTGTA | AB014733 | hypothetical protein AF140225 | 0.18 | 0.19 | 0.98 | 0.90 | 0.59 | 0.18 | 0.33 | 0.32 | 5.44 | 0.84 |
| CAGAAGAGGC | AK001528 | DiGeorge syndrome critical region gene 6 | 0.18 | 0.97 | 0.98 | 0.36 | 1.96 | 2.13 | 0.98 | 0.97 | 5.44 | 0.48 |
| GAGGTGCTCT | AF220050 | uncharacterized hematopoietic stem/progenitor cells protein MDS030 | 0.18 | 0.39 | 0.98 | 0.00 | 1.17 | 0.53 | 0.65 | 0.32 | 5.44 | 0.57 |
| ATGGTCTACG | AK000254 | hypothetical protein | 0.18 | 0.39 | 0.98 | 0.18 | 1.17 | 0.71 | 2.60 | 0.97 | 5.44 | 1.90 |
| TAATGACAAT | U60115 | four and a half LIM domains 1 | 0.18 | 0.39 | 0.98 | 0.38 | 0.00 | 0.00 | 0.00 | 0.00 | 5.44 | 0.00 |
| AAGGAGTTTG | M33374 | NADH dehydrogenase (ubiquinone) 1 beta subcomplex, 7 (18 kD, B18) | 0.37 | 0.39 | 1.96 | 0.90 | 2.15 | 1.06 | 0.33 | 0.97 | 5.30 | 0.40 |
| GTGGTATGGC | AF151076 | hypothetical protein | 0.18 | 0.19 | 0.98 | 0.36 | 0.98 | 1.06 | 0.33 | 0.97 | 5.44 | 0.64 |
| TCAGTTTGTC | U68566 | HAX1; HS1 binding protein | 0.37 | 0.39 | 1.96 | 1.63 | 2.74 | 2.13 | 0.98 | 1.29 | 5.30 | 0.47 |
| TGGGGAAACT | X52333 | APMCF1 protein (EST) | 0.18 | 0.00 | 0.98 | 0.18 | 0.20 | 0.00 | 0.00 | 0.00 | 5.44 | 0.00 |
| GCACAAGTAA | AF339775 | unknown | 0.18 | 0.00 | 0.98 | 0.90 | 0.20 | 0.35 | 0.65 | 0.00 | 5.44 | 1.18 |
| GCAAACAAAA | Hs.429 | ATP synthase, H+ transporting, mitochondrial F0 complex, subunit c (subunit 9) isoform 3 | 0.18 | 0.00 | 0.98 | 0.18 | 0.59 | 0.71 | 0.00 | 0.00 | 5.44 | 0.00 |
| ACAAATCCTT | M34539 | FK506-binding protein 1A (12 kD) | 0.18 | 0.00 | 0.98 | 0.72 | 1.96 | 0.00 | 2.28 | 0.64 | 5.44 | 1.49 |
| GAGTGCAGGT | M25077 | Sjogren syndrome antigen A2 (60 kD, ribonucleoprotein autoantigen SS-A/Ro) | 0.18 | 0.00 | 0.98 | 0.18 | 0.00 | 0.00 | 0.00 | 0.32 | 5.44 | 0.16 |
| CGCTTTTGTA | AL110124 | DKFZP564A2416 protein | 0.55 | 2.90 | 2.75 | 1.81 | 0.98 | 1.77 | 1.95 | 1.61 | 5.00 | 1.29 |
| GAGTAGAGAA | Hs.145279 | SET translocation (myeloid leukemia-associated) | 0.37 | 1.55 | 1.76 | 1.44 | 0.39 | 0.35 | 0.33 | 0.32 | 4.76 | 0.88 |
| TACTGTGGAT | Hs.21537 | protein phosphatase 1, catalytic subunit, beta isoform | 0.37 | 1.93 | 1.76 | 0.90 | 0.78 | 0.53 | 0.65 | 0.97 | 4.76 | 1.24 |
| TATGTGCCAC | AK001323 | hypothetical protein FLJ10461 | 0.55 | 1.93 | 2.35 | 0.36 | 0.20 | 0.00 | 0.00 | 0.00 | 4.27 | 0.00 |
| GTAAAACCCT | AF232009 | HSA250303; peroxisomal trans 2-enoyl CoA reductase; putative short chain alcohol dehydrogenase | 0.18 | 0.77 | 0.78 | 0.00 | 0.20 | 0.00 | 0.00 | 0.32 | 4.33 | 1.60 |
| TGTGACCTCT | AF061729 | dolichyl-phosphate mannosyltransferase polypeptide 2, regulatory subunit | 0.18 | 0.58 | 0.78 | 0.18 | 0.00 | 0.18 | 0.00 | 0.00 | 4.33 | 0.00 |
| AAGACAAGTG | Hs.184109 | ribosomal protein L37a | 0.18 | 0.58 | 0.78 | 0.18 | 0.00 | 0.18 | 0.00 | 0.00 | 4.33 | 0.00 |
| CTGTTGGCAT | Hs.184108 | ribosomal protein L21 (gene or pseudogene) | 0.18 | 1.74 | 0.78 | 1.08 | 0.20 | 0.35 | 0.33 | 1.61 | 4.33 | 3.53 |
| AACGGGCCGG | Hs.26484 | HIRA-interacting protein 3 | 0.18 | 1.55 | 0.78 | 0.54 | 0.39 | 0.18 | 0.33 | 0.32 | 4.33 | 1.14 |
| TTCTGGCACT | AF161402 | DKFZP566C134 protein | 0.18 | 0.97 | 0.78 | 1.08 | 0.20 | 0.18 | 0.00 | 0.00 | 4.33 | 0.00 |
| GTGAAGTCTT | AF055031 | solute carrier family 3 (activators of dibasic and neutral amino acid transport), member 2 | 0.37 | 1.55 | 1.57 | 0.72 | 0.39 | 0.35 | 0.98 | 0.97 | 4.24 | 2.64 |
| GCGAGCAGCG | Hs.300141 | ribosomal protein L39 | 0.18 | 0.39 | 0.78 | 0.72 | 0.20 | 0.00 | 0.00 | 0.64 | 4.33 | 3.20 |
| CACTGAGCCA | AK000034 | DnaJ (Hsp40) homolog, subfamily B, member 12 | 0.37 | 1.35 | 1.57 | 1.08 | 0.00 | 0.71 | 0.33 | 0.32 | 4.24 | 0.92 |
| GCCAGGGGCC | BC002542 | KIAA1273 protein | 0.18 | 0.58 | 0.78 | 0.18 | 0.20 | 0.18 | 0.00 | 0.00 | 4.33 | 0.00 |
| CAGAGTGACT | L07758 | nuclear phosphoprotein similar to S. cerevisiae PWP1 | 0.18 | 0.58 | 0.78 | 1.26 | 0.39 | 0.00 | 0.33 | 0.32 | 4.33 | 1.67 |
| CTATATTTTT | X87212 | cathepsin C | 0.18 | 0.58 | 0.78 | 0.18 | 0.39 | 0.00 | 0.00 | 0.00 | 4.33 | 0.00 |
| GAGAATCTGC | AF075110 | chromosome 14 open reading frame 4 | 0.18 | 0.77 | 0.78 | 0.72 | 0.39 | 0.18 | 0.00 | 0.00 | 4.33 | 0.00 |
| AAAAACCATA | U21242 | general transcription factor IIA, 2 (12 kD subunit) | 0.18 | 0.77 | 0.78 | 0.54 | 0.59 | 0.00 | 0.00 | 0.00 | 4.33 | 0.00 |
| CCTGAATCTG | Hs.179779 | ribosomal protein L37 | 0.18 | 0.58 | 0.78 | 0.36 | 0.20 | 0.35 | 0.00 | 0.00 | 4.33 | 0.00 |
| CAGAAGTGTC | D13640 | KIAA0015 gene product | 0.18 | 0.58 | 0.78 | 0.54 | 0.39 | 0.18 | 0.65 | 0.00 | 4.33 | 1.14 |
| GCTCTGTAAG | AK000222 | putative methyltransferase | 0.18 | 0.39 | 0.78 | 0.72 | 0.20 | 0.18 | 0.00 | 0.32 | 4.33 | 0.84 |
| TCTCTAGAAT | X75342 | SHB adaptor protein (a Src homology 2 protein) | 0.18 | 0.39 | 0.78 | 0.36 | 0.20 | 0.18 | 0.00 | 0.00 | 4.33 | 0.00 |
| CTTATGATCA | AJ238095 | Lsm3 protein | 0.18 | 0.97 | 0.78 | 0.54 | 0.78 | 0.18 | 0.33 | 0.00 | 4.33 | 0.34 |
| TGCAAAAAAA | BC000610 | hypothetical protein FLJ10986 | 0.18 | 0.39 | 0.78 | 0.18 | 0.39 | 0.00 | 0.33 | 0.00 | 4.33 | 0.85 |
| GTTGTGGCCA | Hs.165563 | Homo sapiens cDNA FLJ13070 fis, clone NT2RP3001777 | 0.18 | 0.19 | 0.78 | 0.36 | 0.20 | 0.00 | 0.00 | 0.32 | 4.33 | 1.60 |
| TTTAATTGTG | Hs.145279 | SET translocation (myeloid leukemia-associated) or etoposide-induced mRNA | 0.18 | 0.19 | 0.78 | 0.00 | 0.20 | 0.00 | 0.33 | 0.00 | 4.33 | 1.65 |
| GTGGTGGGTG | X58236 | solute carrier family 14 (urea transporter), member 1 (Kidd blood group) | 0.18 | 1.16 | 0.78 | 0.90 | 0.39 | 0.89 | 0.65 | 0.32 | 4.33 | 0.76 |
| CTGAGCTGTA | AK001080 | WD repeat domain 6 | 0.18 | 0.58 | 0.78 | 0.18 | 0.39 | 0.35 | 0.33 | 0.32 | 4.33 | 0.88 |
| TGTGTGGGGC | AK024450 | unknown | 0.18 | 0.58 | 0.78 | 0.00 | 0.39 | 0.35 | 0.00 | 0.00 | 4.33 | 0.00 |
| TGTGAGCCCC | AF265209 | enigma (LIM domain protein) | 0.37 | 0.58 | 1.57 | 0.36 | 0.59 | 0.18 | 0.00 | 0.00 | 4.24 | 0.00 |
| CCGGACCTGT | Hs.277777 | paired immunoglobulin-like receptor beta | 0.18 | 1.16 | 0.78 | 1.08 | 1.17 | 0.35 | 0.98 | 0.97 | 4.33 | 1.28 |
| CGTGAACAAT | AF226049 | GL004 protein | 0.18 | 0.39 | 0.78 | 0.18 | 0.20 | 0.35 | 0.00 | 0.32 | 4.33 | 0.58 |
| AGCCTCGGGC | Hs.75922 | brain protein I3 | 0.18 | 0.39 | 0.78 | 0.72 | 0.39 | 0.18 | 0.33 | 0.32 | 4.33 | 1.14 |
| TTCTCTCTGT | M57567 | ADP-ribosylation factor 5 | 0.18 | 0.58 | 0.78 | 0.72 | 0.20 | 0.71 | 0.00 | 0.00 | 4.33 | 0.00 |
| CTGCCAAAAA | AF112214 | mitochondrial ribosomal protein L13 | 0.18 | 0.39 | 0.78 | 0.36 | 0.20 | 0.53 | 0.00 | 0.00 | 4.33 | 0.00 |
| CTCTCAATAT | Hs.279518 | amyloid precursor protein homolog HSD-2 or amyloid beta (A4) precursor-like protein 2 | 0.18 | 0.39 | 0.78 | 0.36 | 0.20 | 0.53 | 0.33 | 0.00 | 4.33 | 0.45 |
| AAAATAAAGA | D90373 | APEX nuclease (multifunctional DNA repair enzyme) | 0.18 | 0.58 | 0.78 | 0.36 | 0.39 | 0.71 | 0.65 | 0.64 | 4.33 | 1.17 |
| ACCAAATTAA | AF016266 | tumor necrosis factor receptor superfamily, member 10b (TNF receptor) | 0.18 | 0.58 | 0.78 | 0.54 | 0.59 | 0.53 | 0.00 | 0.00 | 4.33 | 0.00 |
| AAATAAAAGC | X51521 | thyroid stimulating hormone receptor or villin 2 (ezrin) | 0.18 | 0.39 | 0.78 | 1.08 | 0.59 | 0.18 | 0.33 | 0.64 | 4.33 | 1.26 |
| CTGTTTGTTC | Hs.288965 | Homo sapiens cDNA: FLJ22300 fis, clone HRC04759 | 0.18 | 0.19 | 0.78 | 0.36 | 0.20 | 0.18 | 0.00 | 0.64 | 4.33 | 1.68 |
| GGCTATGCCA | AF147357 | cisplatin resistance-associated overexpressed protein (EST) or others | 0.37 | 0.77 | 1.57 | 2.17 | 0.00 | 0.00 | 0.00 | 0.00 | 4.24 | 0.00 |
| CTGCTGAGCC | Hs.1742 | IQ motif containing GTPase activating protein 1 | 0.18 | 0.39 | 0.78 | 0.54 | 0.78 | 0.53 | 0.33 | 1.29 | 4.33 | 1.24 |
| AACAACAGTG | AK001472 | anillin (Drosophila Scraps homolog), actin binding protein (EST) or others | 0.18 | 0.58 | 0.78 | 0.36 | 0.00 | 0.00 | 0.00 | 0.00 | 4.33 | 0.00 |
| GTTTGGCTGC | Hs.154443 | minichromosome maintenance deficient (S. cerevisiae) 4 | 0.18 | 0.58 | 0.78 | 0.54 | 0.00 | 0.00 | 0.00 | 0.00 | 4.33 | 0.00 |
| GACTAAAAAA | NM_001019 | ribosomal protein S15a | 0.18 | 0.19 | 0.78 | 0.00 | 0.00 | 0.71 | 0.00 | 0.00 | 4.33 | 0.00 |
| TGTTGGGTTC | AB013385 | MAP4K4; mitogen-activated protein kinase kinase kinase kinase 4 | 0.18 | 0.19 | 0.78 | 0.18 | 0.20 | 0.53 | 0.65 | 0.00 | 4.33 | 0.89 |
| GTGGACCCTG | AF132959 | NOSIP (eNOS interacting ptotein) | 0.18 | 0.39 | 0.78 | 0.36 | 0.78 | 0.71 | 0.65 | 0.32 | 4.33 | 0.65 |
| ACCCCAGCAA | Hs.164036 | glucosamine (N-acetyl)-6-sulfatase (Sanfilippo disease IIID) or putative zinc finger protein | 0.18 | 0.39 | 0.78 | 0.18 | 0.78 | 0.89 | 1.30 | 0.00 | 4.33 | 0.78 |
| GGCGTCCTGG | Hs.44017 | SIR2 (silent mating type information regulation 2, S. cerevisiae, homolog)-like | 0.18 | 0.77 | 0.78 | 0.36 | 1.96 | 1.42 | 0.98 | 0.00 | 4.33 | 0.29 |
| AGGATGGCGG | Hs.182979 | ribosomal protein L12 | 0.55 | 0.58 | 2.35 | 0.90 | 1.37 | 1.42 | 0.98 | 1.29 | 4.27 | 0.81 |
| GAATGCAGTT | Hs.306155 | chorionic somatomammotropin hormone 2 or ESTs | 0.18 | 0.19 | 0.78 | 0.36 | 0.59 | 0.35 | 0.65 | 0.00 | 4.33 | 0.69 |
| GCCAAACTTG | Hs.119598 | ribosomal protein L3 or ALEX3 protein | 0.18 | 0.19 | 0.78 | 0.18 | 0.78 | 0.18 | 0.33 | 0.64 | 4.33 | 1.01 |
| TGGCAAAGTA | Hs.104480 | ESTs | 0.37 | 0.39 | 1.57 | 0.00 | 0.00 | 0.00 | 0.00 | 0.00 | 4.24 | 0.00 |
| AAGCTGTTGT | X63692 | DNA (cytosine-5-)-methyltransferase 1 | 0.18 | 0.39 | 0.78 | 0.18 | 0.00 | 0.00 | 0.00 | 0.00 | 4.33 | 0.00 |
| TGTGGCCAGG | Hs.290357 | ESTs | 0.18 | 0.39 | 0.78 | 0.00 | 0.00 | 0.00 | 0.00 | 0.32 | 4.33 | 0.16 |
| TAACATTGAG | Hs.43756 | ESTs | 0.18 | 0.39 | 0.78 | 0.00 | 0.00 | 0.00 | 0.00 | 0.00 | 4.33 | 0.00 |
| GCCTCCCGCC | BC001211 | kinesin family member C3 | 0.18 | 0.19 | 0.78 | 0.72 | 0.59 | 0.53 | 0.33 | 0.32 | 4.33 | 0.58 |
| AGAATTGTGT | BC004916 | hypothetical protein MGC4767 | 0.18 | 0.19 | 0.78 | 0.18 | 0.39 | 0.89 | 0.33 | 0.00 | 4.33 | 0.26 |
| TGACACCCAC | X17025 | isopentenyl-diphosphate delta isomerase | 0.18 | 0.19 | 0.78 | 0.36 | 0.78 | 0.53 | 1.30 | 0.00 | 4.33 | 0.99 |
| GGTTGAGTGT | Hs.20529 | Homo sapiens cDNA; FLJ21811 fis, clone HEP01037 | 0.18 | 0.39 | 0.78 | 0.36 | 1.76 | 1.06 | 1.95 | 0.32 | 4.33 | 0.80 |
| GCGGCAGTTA | M55268 J02 | CSNK2A2; casein kinase 2, alpha prime polypeptide | 0.18 | 0.19 | 0.78 | 0.36 | 0.59 | 1.06 | 0.33 | 0.64 | 4.33 | 0.59 |
| TGAATACTAC | AB034747 | LPS-induced TNF-alpha factor | 0.18 | 0.19 | 0.78 | 0.18 | 0.00 | 0.00 | 0.33 | 0.00 | 4.33 | 0.17 |
| TGCATTGTTT | AB040915 | KIAA1482 protein | 0.18 | 0.19 | 0.78 | 0.18 | 0.00 | 0.00 | 0.00 | 0.00 | 4.33 | 0.00 |
| GTGTATATTG | Hs.292669 | ESTs | 0.18 | 0.19 | 0.78 | 0.18 | 0.00 | 0.00 | 0.33 | 0.00 | 4.33 | 0.17 |
| GATGCTGATT | Hs.26410 | ESTs | 0.18 | 0.19 | 0.78 | 0.18 | 0.00 | 0.00 | 0.00 | 0.00 | 4.33 | 0.00 |
| CCTTACTTTA | Hs.75415 | beta-2-microglobulin | 0.18 | 0.19 | 0.78 | 0.18 | 0.00 | 0.00 | 0.00 | 0.00 | 4.33 | 0.00 |
| CCACTTCCTC | Hs.77495 | KIAA0242 protein or ESTs | 0.18 | 0.19 | 0.78 | 0.36 | 0.00 | 0.00 | 0.00 | 0.00 | 4.33 | 0.00 |
| CATCCAAAAC | Hs.245710 | heterogeneous nuclear ribonucleoprotein H1 (H) | 0.18 | 0.19 | 0.78 | 0.72 | 0.00 | 0.00 | 0.00 | 0.00 | 4.33 | 0.00 |
| TTGGGGTTCC | Hs.182771 | vitelliform macular dystrophy (Best disease, bestrophin) or retinoblastoma-binding protein 7 or ferritin, | 0.18 | 0.19 | 0.78 | 0.72 | 0.78 | 1.77 | 1.63 | 1.29 | 4.33 | 1.15 |
| | | heavy polypeptide 1 | | | | | | | | | | |
| GTATCTTAAT | AF155568 | NS1-associated protein 1 | 0.18 | 0.00 | 0.78 | 0.00 | 0.20 | 0.00 | 0.65 | 0.00 | 4.33 | 3.25 |
| ATGAATAAAA | X96650 | microtubule-associated protein, RP/EB family, member 2 or Rho GTPase activating protein 6 | 0.18 | 0.00 | 0.78 | 0.00 | 0.20 | 0.00 | 0.00 | 0.00 | 4.33 | 0.00 |
| GCTAAAAACA | Hs.8107 | Homo sapiens mRNA; cDNA DKFZp586B0918 (from clone DKFZp586B0918) | 0.18 | 0.00 | 0.78 | 0.54 | 0.00 | 0.35 | 0.00 | 0.00 | 4.33 | 0.00 |
| AAGTTGCATC | Hs.29131 | nuclear receptor coactivator 2 | 0.18 | 0.00 | 0.78 | 0.18 | 0.20 | 0.18 | 0.00 | 0.00 | 4.33 | 0.00 |
| TAACCAAACA | AJ243671 | KIAA0144 gene product | 0.18 | 0.00 | 0.78 | 0.18 | 0.39 | 0.00 | 0.00 | 0.00 | 4.33 | 0.00 |
| GGCAGCCTGG | U94836 | protein with polyglutamine repeat; calcium (ca2+) homeostasis endoplasmic reticulum protein | 0.18 | 0.00 | 0.78 | 0.18 | 0.39 | 0.00 | 0.65 | 0.64 | 4.33 | 3.31 |
| TATCACTTTT | Z11898 | POU domain, class 5, transcription factor 1 | 0.18 | 0.00 | 0.78 | 0.36 | 0.00 | 0.00 | 0.00 | 0.32 | 4.33 | 0.16 |
| CTACTGTCTA | Hs.108124 | ribosomal protein L41 | 0.18 | 0.00 | 0.78 | 0.00 | 0.00 | 0.00 | 0.65 | 0.64 | 4.33 | 0.65 |
| GTGCTATTCT | AL360136 | B7 homolog 3 | 0.18 | 0.00 | 0.78 | 0.90 | 1.17 | 1.06 | 0.98 | 0.00 | 4.33 | 0.44 |
| GAGCTGGTGA | NM_005830 | imogen 38 | 0.18 | 0.00 | 0.78 | 0.54 | 1.17 | 1.42 | 0.98 | 0.64 | 4.33 | 0.63 |
| GTCTGACCCC | Hs.173902 | protein phosphatase 2 (formerly 2A), regulatory subunit A (PR 65), alpha isoform | 0.37 | 0.00 | 1.57 | 0.54 | 3.13 | 2.48 | 0.98 | 1.29 | 4.24 | 0.40 |
| GTGGCGGGCG | D90313 | carcinoembryonic antigen-related cell adhesion molecule 1 (biliary glycoprotein) or others | 0.92 | 1.74 | 3.73 | 2.89 | 1.37 | 0.89 | 1.63 | 0.97 | 4.05 | 1.15 |

7 genes in FIG. 3(g) (6 genes; 1 novel tag not shown)
SAGE data for genes up-regulated ≧ 4-fold by hypoxia in both 786-0 VHL⁺ cells and RPTECs. Normalized SAGE data from 786-0
VHL⁺ Hyp. (V3) was compared to normalized data from 786-0 VHL⁺ Nor. (V1). The average tag abundance for the two
independent cultures of RPTECs grown under hypoxia (V7, V8) and normoxia (V5, V6) was used. The table is presented in descending order of V3/V1.

| | | | | | | | | | | | | |
|---|---|---|---|---|---|---|---|---|---|---|---|---|
| GACCCACTAC | J03569 | SLC3A2; solute carrier family 3 (activators of dibasic and neutral amino acid transport), member 2 | 0.18 | 0.19 | 1.76 | 0.18 | 0.20 | 0.18 | 0.33 | 1.29 | 9.78 | 4.26 |
| CTCACTTCTT | Hs.165998 | DKFZP564M2423 protein | 0.18 | 0.97 | 0.98 | 0.90 | 0.20 | 0.00 | 0.98 | 0.00 | 5.44 | 4.90 |
| AACTCCCAGT | AL050044 | GADD45B; growth arrest and DNA-damage-inducible, beta | 0.18 | 0.39 | 0.98 | 0.72 | 0.39 | 0.00 | 0.65 | 0.97 | 5.44 | 4.15 |
| CCACGTTCCA | Y00691 M27 | ENO2; enolase 2, (gamma, neuronal) | 0.18 | 1.55 | 0.98 | 1.63 | 0.78 | 1.06 | 5.85 | 16.74 | 5.44 | 12.28 |
| CGGGATGCAG | Hs.155560 | calnexin | 0.18 | 0.97 | 0.78 | 1.08 | 0.59 | 0.18 | 1.95 | 1.29 | 4.33 | 4.21 |
| ATGTGAGGGA | AK000510 | tRNA selenocysteine associated protein | 0.18 | 0.39 | 0.78 | 0.36 | 0.00 | 0.35 | 0.65 | 0.97 | 4.33 | 4.63 |

407 genes In FIG. 3 (347 genes; 60 tags with no matches not shown)
SAGE data for genes up-regulated ≧ 4-fold by hypoxia in RPTECs. The average tag abundance for the two independent cultures of RPTECs grown
under hypoxia (V7, V8) and normoxia (V5, V6) was used. Also shown, for comparison, is the SAGE data for each gene in 786-0 VHL⁺ Hyp (V3) compared
to 786-0 VHL⁻ Nor. (V1). SAGE tag abundances were normalized before calculating ratios, as shown. The table is presented in descending order of V7, V8/V5, V6.

| | | | | | | | | | | | | |
|---|---|---|---|---|---|---|---|---|---|---|---|---|
| AACACAGAGA | D88667 | CST; cerebroside (3′-phosphoadenylylsulfate:galactosylceramide 3′) sulfotransferase | 1.84 | 1.74 | 3.14 | 3.07 | 0.00 | 0.18 | 0.98 | 5.79 | 37.61 | 1.71 |
| GCAGCAGAGG | AF169312 | ANGPTL2; PPAR(gamma) anglopoletin related protein, hepatic | 0.00 | 0.00 | 0.00 | 0.18 | 0.00 | 0.18 | 1.30 | 4.83 | 34.06 | 0.00 |
| CGTATGCCTC | S45875 | LOX; lysyl oxldase | 0.00 | 0.00 | 0.00 | 0.00 | 0.39 | 0.18 | 3.25 | 14.16 | 30.54 | 0.00 |
| TCACAGCTGT | Hs.77054 | B-cell translocation gene 1, anti-proliferative | 1.48 | 0.97 | 1.18 | 1.08 | 0.20 | 0.00 | 0.33 | 4.83 | 25.80 | 0.80 |
| GGCCACGTAG | Hs.155597 | D component of complement (adipsin) | 0.37 | 0.00 | 0.20 | 0.18 | 0.00 | 0.18 | 0.00 | 3.86 | 21.44 | 0.54 |
| CAGGAGGAAA | AK000595 | KIAA0964 protein (EST) | 0.18 | 0.39 | 0.20 | 0.90 | 0.00 | 0.18 | 1.30 | 2.25 | 19.72 | 1.11 |
| GACACTACAC | AF069073 | P8; p8 protein (candidate of metastasis 1) | 0.18 | 0.39 | 0.39 | 0.18 | 0.20 | 0.00 | 3.58 | 0.32 | 19.50 | 2.17 |
| TGGGCTGGGG | Hs.3416 | adipose differentiation-related protein | 4.61 | 5.41 | 2.94 | 2.17 | 0.59 | 1.95 | 7.15 | 42.81 | 19.67 | 0.64 |
| TGGCTTAAAT | AF144755 | HIG2; hypoxia-inducible protein 2 | 0.37 | 1.74 | 0.78 | 2.53 | 0.20 | 0.18 | 1.63 | 5.47 | 18.68 | 2.11 |
| ACACTTGGAG | Hs.301333 | ESTs | 1.29 | 0.39 | 0.98 | 1.44 | 0.00 | 0.18 | 1.30 | 1.93 | 17.94 | 0.76 |
| GCTGGGAACC | X60708 S40 | dipeptidylpeptidase IV (CD26, adenosine deaminase complexing protein 2) | 0.00 | 0.39 | 0.39 | 0.54 | 0.00 | 0.18 | 0.98 | 2.25 | 17.94 | 0.39 |
| CAACACTGTG | Hs.19086 | ESTs | 0.00 | 0.19 | 0.39 | 0.00 | 0.00 | 0.18 | 0.65 | 2.58 | 17.94 | 0.39 |
| GGACTTTCCT | D87953 | NDRG1; N-myc downstream regulated | 0.00 | 0.97 | 0.39 | 1.99 | 0.20 | 0.53 | 3.58 | 9.66 | 18.14 | 0.39 |
| TCAACTTCTG | Hs.7594 | solute carrier family 2 (facilitated glucose transporter), member 3 | 0.00 | 0.00 | 0.00 | 0.00 | 0.00 | 0.18 | 0.33 | 2.90 | 17.94 | 0.00 |
| ACCCGCCTGA | AF147402 | NDRG1; N-myc downstream regulated (EST) | 0.00 | 0.00 | 0.00 | 0.00 | 0.20 | 0.00 | 0.33 | 3.22 | 17.75 | 0.00 |
| GTGGTGGCAG | AK001761 | RAI3; retinoic acid induced 3 | 0.37 | 0.39 | 0.39 | 0.36 | 0.20 | 0.00 | 0.00 | 3.54 | 17.70 | 1.05 |
| GTTTCAGGAG | AB023430 | protein tyrosine phosphatase, non-receptor type substrate 1 | 0.37 | 0.39 | 0.59 | 1.26 | 0.00 | 0.18 | 1.63 | 1.29 | 16.22 | 1.59 |
| GGAGGAATTC | X12451 | cathepsin L | 0.18 | 0.00 | 0.20 | 0.18 | 0.00 | 0.18 | 1.63 | 1.29 | 16.22 | 1.11 |
| GAGCAAGGGG | AF064599 | NOL3; nucleolar protein 3 (apoptosis repressor with CARD domain) | 0.37 | 0.19 | 0.39 | 0.36 | 0.00 | 0.35 | 2.93 | 2.90 | 16.66 | 1.05 |
| CCTGTAAAGC | Hs.9691 | Homo sapiens cDNA: FLJ23249 fis, clone COL04196 | 0.18 | 1.16 | 0.59 | 0.72 | 0.20 | 0.18 | 1.63 | 3.86 | 14.45 | 3.28 |
| CTCAACCCCC | X13916 | low density lipoprotein-related protein 1 (alpha-2-macroglobulin receptor) | 0.18 | 0.97 | 0.00 | 0.54 | 0.00 | 0.18 | 1.63 | 0.97 | 14.44 | 0.00 |
| GCCACGTTGT | AL137588 | HOG3; hypothetical protein DKFZp43K1210 | 0.00 | 1.16 | 0.59 | 1.44 | 0.00 | 0.18 | 0.98 | 1.61 | 14.39 | 0.59 |
| TTCAGGAGGG | M17661 J02 | T-cell receptor alpha delta locus from bases 501613 to 752736 (section 3 of 5) | 0.55 | 0.58 | 0.59 | 0.18 | 0.00 | 0.18 | 0.98 | 1.61 | 14.39 | 1.07 |
| TGGGAACATA | Hs.300171 | ESTs | 0.18 | 0.19 | 0.20 | 0.18 | 0.00 | 0.18 | 0.98 | 1.61 | 14.39 | 1.11 |
| CTCGCGCTGG | Hs.25640 | claudin 3 or ESTs | 0.00 | 0.00 | 0.00 | 0.00 | 0.00 | 0.18 | 0.33 | 2.25 | 14.33 | 0.00 |
| GTAAGTGTAC | S64650 | EST | 5.16 | 3.09 | 0.98 | 1.08 | 3.33 | 4.61 | 59.17 | 53.11 | 14.14 | 0.19 |
| GAGACTCCTG | AF070544 | GLUT1; solute carrier family 2 (facilitated glucose transporter), member 1, glucose transporter | 2.21 | 4.83 | 7.06 | 7.04 | 0.78 | 1.06 | 4.23 | 21.57 | 14.02 | 3.19 |
| AACAGCAAAA | Hs.121576 | aspartate beta-hydroxylase | 0.00 | 0.00 | 0.20 | 0.18 | 0.20 | 0.18 | 3.25 | 1.93 | 13.63 | 0.20 |
| TAACAGTTGT | Hs.155560 | calnexin | 0.37 | 0.58 | 0.78 | 1.81 | 0.00 | 0.35 | 0.98 | 3.86 | 13.63 | 2.11 |
| CCCCCGCGGA | AF130366 | liver-specific bHLH-Zip transcription factor | 0.00 | 0.00 | 0.00 | 0.00 | 0.20 | 0.00 | 1.30 | 1.29 | 12.95 | 0.00 |
| TGTTAGAAAA | U84573 | PLOD2; procollagen-lysine, 2-oxoglutarate 5-dioxygenase (lysine hydroxylase) 2 | 2.03 | 2.90 | 2.94 | 2.71 | 0.00 | 0.18 | 1.30 | 0.97 | 12.61 | 1.45 |
| GGTGAGGGAG | Hs.9071 | progesterone membrane binding protein | 0.37 | 0.19 | 0.39 | 0.18 | 0.00 | 0.18 | 0.98 | 1.29 | 12.61 | 1.05 |
| GCTGGTCTGA | AF119664 | transcriptional regulator protein | 0.00 | 0.19 | 0.20 | 0.54 | 0.00 | 0.18 | 0.98 | 1.29 | 12.61 | 0.20 |
| AGACATTGTA | AB037848 | synaptotagmin XIII | 0.00 | 0.00 | 0.20 | 0.18 | 0.00 | 0.18 | 0.65 | 1.61 | 12.56 | 0.20 |
| ACATTTCATC | Hs.5890 | hypothetical protein FLJ23306 (EST) | 0.37 | 0.19 | 0.00 | 0.00 | 0.00 | 0.18 | 0.33 | 1.93 | 12.56 | 0.00 |
| AGCCTGCTCA | X55740 | 5′ nucleotidase (CD73) | 0.18 | 0.00 | 0.00 | 0.00 | 0.00 | 0.18 | 0.33 | 1.93 | 12.56 | 0.00 |
| TCAGCAAGGG | Hs.298668 | ESTs | 0.00 | 0.00 | 0.00 | 0.18 | 0.00 | 0.18 | 0.33 | 1.93 | 12.56 | 0.00 |
| ATGGGGCAGG | U60060 | fasciculation and elongation protein zeta 1 (zygin I) | 0.00 | 0.00 | 0.00 | 0.00 | 0.20 | 0.00 | 1.30 | 0.97 | 11.35 | 0.00 |
| GGTACTCGAT | AF289489 | aspartate beta-hydroxylase | 2.40 | 2.13 | 2.16 | 1.99 | 0.20 | 0.00 | 0.98 | 1.29 | 11.35 | 0.90 |
| GAGGTCTCCT | AF159092 | N-myc downstream regulated (EST) | 0.00 | 0.19 | 0.00 | 0.00 | 0.20 | 0.00 | 0.98 | 1.29 | 11.35 | 0.00 |
| CTTCTGGTGC | Hs.75431 | fibrinogen, gamma polypeptide | 0.18 | 0.77 | 0.59 | 0.36 | 0.20 | 0.00 | 0.65 | 1.61 | 11.30 | 3.28 |
| GGCCCGCTGC | Hs.3745 | milk fat globule-EGF factor 8 protein | 0.00 | 0.39 | 0.20 | 0.18 | 0.00 | 0.18 | 1.30 | 0.64 | 10.78 | 0.20 |
| AAGGAGGTGG | Hs.85852 | hypothetical protein MGC3169 or ESTs | 0.00 | 0.00 | 0.00 | 0.00 | 0.00 | 0.18 | 1.30 | 0.64 | 10.78 | 0.00 |
| TATAACTTGT | AL132665 | BNIP3L; BCL2/adenovirus E1B 19 kD-interacting protein 3-like | 0.37 | 0.58 | 0.59 | 0.36 | 0.00 | 0.35 | 2.28 | 1.61 | 11.11 | 1.59 |
| CGTTTAATGT | Hs.11637 | ESTs | 0.37 | 0.77 | 0.20 | 0.00 | 0.00 | 0.35 | 1.63 | 2.25 | 11.09 | 0.54 |
| TGTGGCGGGT | Hs.82422 | capping protein (actin filament), gelsolin-like | 0.00 | 0.00 | 0.00 | 0.00 | 0.00 | 0.18 | 0.00 | 1.93 | 10.72 | 0.00 |
| AAGAAAGGGG | U15174 | BNIP3; BCL2/adenovirus E1B 19 kD-interacting protein 3 | 0.37 | 0.19 | 0.39 | 0.00 | 0.39 | 0.18 | 1.95 | 4.18 | 10.75 | 1.05 |
| AGGGGGTTCG | Hs.198281 | pyruvate kinsase, muscle | 0.00 | 0.00 | 0.00 | 0.00 | 0.20 | 0.18 | 1.30 | 2.58 | 10.21 | 0.00 |
| CAAGACGGGG | AB037729 | RALGDS; ral guanine nucleotide dissociation stimulator | 1.11 | 0.19 | 0.78 | 0.90 | 0.00 | 0.53 | 1.63 | 3.86 | 10.36 | 0.70 |
| GTTATTGAGG | AF191020 | hypothetical protein, estradiol-induced | 0.00 | 0.00 | 0.00 | 0.36 | 0.39 | 0.18 | 2.28 | 3.54 | 10.21 | 0.00 |
| TGCAGCGCCT | X90858 | uridine phosphorylase | 0.18 | 0.19 | 0.20 | 0.18 | 0.20 | 0.53 | 1.63 | 5.79 | 10.16 | 1.11 |
| TGTCATCACA | U92015 | LOXL2; lysyl oxidase-like 2 | 4.98 | 9.47 | 10.00 | 9.93 | 1.37 | 0.35 | 12.35 | 4.83 | 9.99 | 2.01 |
| GCTTTGCTTT | Hs.298668 | ESTs | 0.00 | 0.00 | 0.00 | 0.36 | 0.20 | 0.35 | 0.65 | 4.83 | 9.96 | 0.00 |
| GGAACAGGGG | AK001306 | Rho GTPase activating protein 8 | 0.00 | 0.19 | 0.20 | 0.18 | 0.20 | 0.00 | 1.30 | 0.64 | 9.70 | 0.20 |
| GTGAGACCCC | AK001001 | hypothetical protein FLJ10139 | 1.29 | 0.77 | 0.39 | 1.26 | 0.20 | 0.00 | 0.98 | 0.97 | 9.75 | 0.30 |
| ACTGCCCCAA | AL137367 | hypothetical protein FLJ21347 | 0.00 | 0.00 | 0.00 | 0.18 | 0.20 | 0.00 | 0.98 | 0.97 | 9.75 | 0.00 |
| TTAGGCAAGT | Hs.75103 | tyrosine 3-monooxygenase/tryptophan 5-monooxygenase activation protein, zeta polypeptide | 1.11 | 1.35 | 1.37 | 1.63 | 0.20 | 0.00 | 0.65 | 1.29 | 9.70 | 1.23 |
| GGCCAAGTGA | X12701 | PAI1; Ser (or Cys) proteinase inhibitor, clade E (nexin, plasminogen activator inhibitor type 1) | 0.00 | 0.00 | 0.00 | 0.00 | 0.20 | 0.00 | 0.65 | 1.29 | 9.70 | 0.00 |
| GTCTTAAAGT | BC001980 | hypothetical protein MGC5618 | 0.92 | 0.58 | 0.98 | 0.36 | 0.20 | 1.06 | 8.45 | 3.86 | 9.77 | 1.07 |
| TGGAGGTGGG | AF086628 | VAMP (vesicle-associated membrane protein)-associated protein B and C | 0.18 | 0.97 | 0.39 | 0.36 | 0.00 | 0.18 | 1.30 | 0.32 | 9.00 | 2.17 |
| GGGCTGGGGG | Hs.183698 | ribosomal protein L29 or profilin 1 | 0.37 | 0.58 | 1.37 | 0.00 | 0.00 | 0.18 | 1.30 | 0.32 | 9.00 | 3.70 |
| GAGTGGAGAG | Hs.77578 | ubiquitin specific protease 9, X chromosome (Drosophila fat facets related) or ESTs | 0.37 | 0.00 | 0.39 | 0.18 | 0.00 | 0.18 | 1.30 | 0.32 | 9.00 | 1.05 |
| TTGGCAGTAT | AJ000534 | (11nt = T) SGCE; sarcoglycan, epsilon | 2.03 | 2.90 | 1.37 | 0.54 | 0.00 | 0.18 | 0.98 | 0.64 | 9.00 | 0.67 |
| GGCAACAGAG | AF009284 | EST | 0.00 | 0.77 | 0.20 | 0.36 | 0.00 | 0.18 | 0.98 | 0.64 | 9.00 | 0.20 |
| CCAACTCTCA | Hs.182447 | heterogeneous nuclear ribonucleoprotein C (C1/C2) | 0.18 | 0.58 | 0.20 | 0.00 | 0.00 | 0.18 | 0.98 | 0.64 | 9.00 | 1.11 |
| ATTGCTTTTG | AF091071 | similar to S. cerevisiae RER1 | 1.11 | 0.39 | 0.20 | 1.08 | 0.00 | 0.18 | 0.98 | 0.64 | 9.00 | 0.18 |
| AAATAAGTCA | AB003476 | A kinase (PRKA) anchor protein (gravin) 12 | 0.00 | 0.00 | 0.00 | 0.00 | 0.00 | 0.18 | 0.98 | 0.64 | 9.00 | 0.00 |
| CACTGAACTC | Hs.172028 | a disintegrin and metalloproteinase domain 10 | 0.37 | 0.97 | 0.59 | 0.90 | 0.00 | 0.18 | 0.65 | 0.97 | 9.00 | 1.59 |
| GCAAAGATTG | Hs.28631 | Homo sapiens cDNA: FLJ22141 fis, clone HEP21327 | 0.00 | 0.39 | 0.20 | 0.54 | 0.00 | 0.18 | 0.65 | 0.97 | 9.00 | 0.20 |
| GCAGAAATGA | AK000476 | hypothetical protein | 0.00 | 0.00 | 0.20 | 0.00 | 0.00 | 0.18 | 0.65 | 0.97 | 9.00 | 0.20 |
| GCCTTCCGTG | Hs.169476 | glyceraldehyde-3-phosphate dehydrogenase | 0.00 | 0.58 | 0.39 | 0.00 | 0.00 | 0.18 | 0.33 | 1.29 | 9.00 | 0.39 |
| AAGGGGGCAA | X53587 | ITGB4; Integrin, beta 4 | 0.00 | 0.00 | 0.00 | 0.00 | 0.00 | 0.18 | 0.33 | 1.29 | 9.00 | 0.00 |
| CGGGGGTGGG | Hs.121849 | Microtubule-associated proteins 1A and 1B, light chain 3 or ESTs | 0.00 | 0.00 | 0.00 | 0.00 | 0.00 | 0.18 | 0.33 | 1.29 | 9.00 | 0.00 |
| GCAGACATTG | Hs.179662 | nucleosome assembly protein 1-like 1 or threonyl-tRNA synthetase | 0.00 | 0.39 | 0.20 | 0.00 | 0.00 | 0.18 | 0.00 | 1.61 | 8.94 | 0.20 |
| TCCTTGGACC | AK001359 | proline dehydrogenase (protein oxidase) | 0.00 | 0.00 | 0.00 | 0.18 | 0.00 | 0.18 | 0.00 | 1.61 | 8.94 | 0.00 |
| ATTTGAGAGT | Hs.146550 | myosin, heavy polypeptide 9, non-muscle | 0.74 | 0.97 | 1.18 | 0.90 | 0.20 | 0.18 | 2.60 | 0.64 | 8.53 | 1.59 |
| TTCGCTGAGG | AF104222 | CD2 antigen (cytoplasmic tail)-binding protein 2 | 0.00 | 0.00 | 0.20 | 0.00 | 0.20 | 0.00 | 1.30 | 0.32 | 8.10 | 0.20 |
| GCCTTGGTAA | Hs.146170 | hypothetical protein FLJ22969 | 0.00 | 0.00 | 0.00 | 0.36 | 0.20 | 0.00 | 1.30 | 0.32 | 8.10 | 0.00 |
| TATGTTTCAG | M93425 | protein tyrosine phosphatase, non-receptor type 12 (PTP-PEST) | 1.66 | 1.93 | 0.59 | 1.08 | 0.20 | 0.00 | 0.98 | 0.64 | 8.10 | 0.36 |
| GAGGAAGAAG | S74942 | (11nt = A) TRA1; tumor rejection antigen/endoplasmic reticular heat (EST) | 0.92 | 2.90 | 2.35 | 2.89 | 0.20 | 0.00 | 0.65 | 0.97 | 8.10 | 2.55 |
| TCTGGGGACG | AA588847 | eukaryotic translation initiation factor 2, subunit 2 (beta 38 kD) (EST) | 3.87 | 2.13 | 1.76 | 1.63 | 0.20 | 0.00 | 0.65 | 0.97 | 8.10 | 0.45 |
| ACCTAGCCAC | U11058 | potassium large conductance calcium-activated channel, subfamily M, alpha member 1 | 0.37 | 1.16 | 0.39 | 0.00 | 0.20 | 0.00 | 0.65 | 0.97 | 8.10 | 1.05 |
| CCGCCGAAGT | Hs.182979 | ribosomal protein L12 | 0.37 | 0.19 | 0.00 | 0.18 | 0.20 | 0.00 | 0.65 | 0.97 | 8.10 | 0.00 |
| CACTCGTGTG | BC002711 | cell division cycle 42 (GTP-binding protein, 25 kD) | 1.29 | 2.32 | 0.98 | 1.26 | 0.20 | 0.00 | 0.33 | 1.29 | 8.10 | 0.76 |
| GTGAGACCCT | X17650 | myogenic factor 3 or polymerase (DNA directed) kappa | 0.74 | 1.16 | 1.57 | 0.54 | 0.20 | 0.00 | 0.33 | 1.29 | 8.10 | 2.12 |
| GTCTCCTAAT | Hs.194691 | retinoic acid induced 3 | 0.18 | 0.19 | 0.20 | 0.00 | 0.20 | 0.00 | 0.33 | 1.29 | 8.10 | 1.11 |
| CCACAATCCT | Hs.171811 | adenylate kinase 2 | 0.00 | 0.19 | 0.39 | 0.54 | 0.00 | 0.35 | 0.98 | 1.93 | 8.31 | 0.39 |
| TGATTGCCCT | M24486 | P4HA1; procollagen-proline, 2-oxoglutarate 4-dioxygenase alpha polypeptide I (prolyl 4-hydroxylase) | 0.00 | 0.19 | 0.20 | 0.54 | 0.20 | 0.18 | 0.33 | 2.58 | 7.66 | 0.20 |
| CACCCCTGAT | M16364 | creatine kinase, brain | 0.00 | 0.00 | 0.00 | 0.00 | 1.57 | 5.85 | 13.00 | 42.81 | 7.52 | 0.00 |
| GGTTATTTTG | X04744 | PAI1; serine (or cysteine) proteinase inhibitor, clade E (nexin, plasminogen activator inhibitor type 1) | 0.00 | 0.39 | 0.59 | 0.54 | 1.76 | 0.35 | 8.78 | 7.08 | 7.52 | 0.59 |
| GAAATGAGCA | D50917 | KIAA0127 gene product | 0.55 | 1.16 | 1.18 | 1.08 | 0.39 | 0.00 | 0.00 | 2.90 | 7.44 | 2.15 |
| CAAATAAAAT | D78130 | squalene epoxidase | 0.55 | 0.58 | 0.39 | 0.54 | 0.00 | 0.18 | 1.30 | 0.00 | 7.22 | 0.71 |
| ATTAGTGTTG | Hs.177781 | superoxide dismutase 2, mitochondrial | 0.18 | 0.00 | 0.20 | 0.00 | 0.00 | 0.18 | 1.30 | 0.00 | 7.22 | 1.11 |
| GTAACAAGCT | AB007898 | KIAA0438 gene product | 1.11 | 0.19 | 0.59 | 0.18 | 0.00 | 0.18 | 0.98 | 0.32 | 7.22 | 0.53 |
| TCTTAATGGG | AF359563 | inner mitochondrial membrane peptidase 2 like | 0.55 | 0.00 | 0.20 | 0.00 | 0.00 | 0.18 | 0.98 | 0.32 | 7.22 | 0.53 |
| AGAGGTTGAT | AF055066 | HLA-A; major histocompatibility complex, class I, A | 0.18 | 0.00 | 0.00 | 0.00 | 0.00 | 0.18 | 0.98 | 0.32 | 7.22 | 0.36 |
| GAACACCGTC | AL353952 | phosphatidylinositol 4-kinase type II | 0.18 | 0.00 | 0.00 | 0.00 | 0.00 | 0.18 | 0.98 | 0.32 | 7.22 | 0.00 |
| AATGACACAA | L23311 | cyclin-dependent kinase 8 (EST) | 0.18 | 0.00 | 0.20 | 0.18 | 0.00 | 0.18 | 0.98 | 0.32 | 7.22 | 1.11 |
| AGCTTGCAGG | Hs.130740 | hypothetical protein FLJ20308 or nicastrin | 0.18 | 0.00 | 0.00 | 0.00 | 0.00 | 0.18 | 0.98 | 0.32 | 7.22 | 0.00 |
| GCACTTGCAT | D50840 | UDP-glucose ceramide glucosyltransferase | 2.21 | 4.25 | 3.53 | 2.89 | 0.00 | 0.35 | 1.30 | 1.29 | 7.40 | 1.60 |
| TGAAGCAGTA | Hs.176626 | hypothetical protein EDAG-1 or ubiquitin-conjugating enzyme E2N (homologous to yeast U8C13) | 1.29 | 0.97 | 0.98 | 0.90 | 0.00 | 0.18 | 0.65 | 0.64 | 7.17 | 0.76 |
| GCATATGAGC | Hs.7149 | Homo sapiens cDNA: FLJ21950 fis, clone HEP04949 | 1.48 | 0.77 | 0.78 | 0.90 | 0.00 | 0.18 | 0.65 | 0.64 | 7.17 | 0.53 |
| GTGAAGCCCC | AK000702 | WW Domain-Containing Gene or hypothetical protein FLJ20695 | 0.37 | 0.58 | 0.39 | 0.18 | 0.00 | 0.18 | 0.65 | 0.64 | 7.17 | 1.05 |
| TCCATAGATT | Hs.75607 | myristoylated alanine-rich protein kinase C substrate (MARCKS, 80K-L) | 0.37 | 0.39 | 0.78 | 0.00 | 0.00 | 0.18 | 0.65 | 0.64 | 7.17 | 2.11 |
| TATCCTGGCT | X85116 | erythrocyte membrane protein band 7.2 (stomatin) | 0.55 | 0.19 | 0.20 | 0.54 | 0.00 | 0.18 | 0.65 | 0.64 | 7.17 | 0.36 |
| TGATGGTGAT | X60673 S41 | ADK3; adenylate kinase 3 | 0.18 | 0.19 | 0.00 | 0.00 | 0.00 | 0.18 | 0.65 | 0.64 | 7.17 | 0.00 |
| CTCAACAGAT | Hs.11916 | ribokinase | 0.18 | 0.19 | 0.39 | 0.00 | 0.00 | 0.18 | 0.65 | 0.64 | 7.17 | 2.17 |
| AACAGTGTGC | Hs.12329 | KIAA0697 protein or serine (or cysteine) proteinase inhibitor, clade E (naxin, plasminogen activator | 0.00 | 0.19 | 0.20 | 0.00 | 0.00 | 0.18 | 0.65 | 0.64 | 7.17 | 0.20 |
| | | inhibitor type 1) | | | | | | | | | | |
| TGCTGAGGAA | Hs.283446 | ESTs | 0.00 | 0.19 | 0.00 | 0.00 | 0.00 | 0.18 | 0.65 | 0.64 | 7.17 | 0.00 |
| GGAGCATCTG | AK002155 | GENX-3414; genethonin 1 | 0.37 | 0.00 | 0.00 | 0.00 | 0.00 | 0.18 | 0.65 | 0.64 | 7.17 | 0.00 |
| AGGCTGGGAG | BC012853 | GNPI; glucosamine-6-phosphate isomerase or hypothetical protein FLJ23360 | 0.37 | 0.00 | 0.20 | 0.00 | 0.00 | 0.18 | 0.65 | 0.64 | 7.17 | 0.54 |
| CGAGTGAGAG | AB033016 | mRNA for KIAA1190 protein, partial cds | 0.18 | 0.00 | 0.00 | 0.00 | 0.00 | 0.18 | 0.65 | 0.64 | 7.17 | 0.00 |
| CCCAAAGACA | NM_005324 | H3 histone, family 3B (H3.3B) | 0.18 | 0.00 | 0.00 | 0.18 | 0.00 | 0.18 | 0.65 | 0.64 | 7.17 | 0.00 |
| TGCACAATAT | AB038783 | MUC3A; mucin 3A, intestinal | 0.00 | 0.00 | 0.00 | 0.00 | 0.00 | 0.18 | 0.65 | 0.64 | 7.17 | 0.00 |
| GAAACTGAAG | AF069987 | nitrilase 1 | 0.00 | 0.00 | 0.20 | 0.00 | 0.00 | 0.18 | 0.65 | 0.64 | 7.17 | 0.20 |
| GACAAAGAGA | AJ243950 | deafness locus associated putative guanine nucleotide exchange factor | 0.00 | 0.00 | 0.20 | 0.00 | 0.00 | 0.18 | 0.65 | 0.64 | 7.17 | 0.20 |
| CAGCCTGTCG | AK001034 | cat eye syndrome chromosome region, candidate 5 (EST) | 0.00 | 0.58 | 0.39 | 0.18 | 0.00 | 0.35 | 0.98 | 1.61 | 7.40 | 0.39 |
| CAGATAACAT | D13641 | translocase of outer mitochondrial membrane 20 (yeast) homolog | 0.55 | 0.77 | 0.78 | 0.54 | 0.00 | 0.18 | 0.33 | 0.97 | 7.22 | 1.42 |
| CCCCCAATGC | AC005263 | splicing factor 3a, subunit 2, 66 kD | 0.18 | 0.58 | 0.00 | 0.36 | 0.00 | 0.18 | 0.33 | 0.97 | 7.22 | 0.00 |
| GCACCATAAT | Hs.43910 | CD164 antigen, sialomucin | 0.18 | 0.58 | 0.00 | 0.72 | 0.00 | 0.18 | 0.33 | 0.97 | 7.22 | 0.00 |
| AACAAGGTGA | AJ223473 | EST | 0.00 | 0.19 | 0.39 | 0.36 | 0.00 | 0.18 | 0.33 | 0.97 | 7.22 | 0.39 |
| TGAGATCTTG | BC001012 | hypothetical protein FLJ20151 | 0.18 | 0.00 | 0.39 | 0.36 | 0.00 | 0.18 | 0.33 | 0.97 | 7.22 | 2.17 |
| TGCGTTGAGA | Hs.181165 | eukaryotic translation elongation factor 1 alpha 1 | 0.18 | 0.00 | 0.00 | 0.00 | 0.00 | 0.18 | 0.33 | 0.97 | 7.22 | 0.00 |
| TAGACCCCTT | M74509 | glyceraldehyde-3-phosphate dehydrogenase (EST) | 0.00 | 0.00 | 0.78 | 0.18 | 0.00 | 0.35 | 0.65 | 1.93 | 7.37 | 0.78 |
| TTTCTTAAAG | AB002322 | RNA binding protein; AT-rich element binding factor | 0.55 | 0.19 | 0.59 | 0.00 | 0.00 | 0.18 | 0.00 | 1.29 | 7.17 | 1.07 |
| CAGTGGAGGG | AF190901 | cell death-inducing DFFA-like effector b | 0.00 | 0.00 | 0.00 | 0.00 | 0.00 | 0.18 | 0.00 | 1.29 | 7.17 | 0.00 |
| GGACTGGGGT | Hs.90436 | sperm associated antigen 7 | 0.00 | 0.00 | 0.39 | 0.00 | 0.00 | 0.18 | 0.00 | 1.29 | 7.17 | 0.39 |
| TCGGAGCTGT | Hs.4055 | G protein-coupled receptor kinase-interactor 1 | 0.00 | 0.00 | 0.00 | 0.00 | 0.20 | 0.18 | 1.30 | 1.29 | 6.82 | 0.00 |
| GTGGAAAAGT | BC012625 | PPP1R3C; protein phosphatase 1, regulatory (inhibitor) subunit 3C | 0.00 | 0.00 | 0.00 | 0.00 | 0.20 | 0.00 | 1.30 | 0.00 | 6.50 | 0.00 |
| CTGCCCTAGT | AL050346 | Novel human gene mapping to chomosome 22. | 0.37 | 0.77 | 0.20 | 0.36 | 0.20 | 0.00 | 0.98 | 0.32 | 6.50 | 0.54 |
| AACTGTCCTT | L37385 | PEA15; phosphoprotein enriched in astrocytes 15 | 0.18 | 0.77 | 0.00 | 0.18 | 0.20 | 0.00 | 0.98 | 0.32 | 6.50 | 0.00 |
| TTGGATCATT | Hs.24719 | MAP-1 protein | 0.18 | 0.39 | 0.39 | 0.36 | 0.20 | 0.00 | 0.98 | 0.32 | 6.50 | 2.17 |
| ACTACAAATA | Hs.157850 | ribosomal protein L9 | 0.37 | 0.97 | 0.20 | 0.36 | 0.20 | 0.00 | 0.65 | 0.64 | 6.45 | 0.54 |
| CTCATATGTT | Hs.84520 | ESTs | 0.18 | 0.39 | 0.00 | 0.36 | 0.20 | 0.00 | 0.65 | 0.64 | 6.45 | 0.00 |
| GCCAAAGTGT | AF237952 | PAN2 protein | 0.55 | 0.19 | 0.39 | 0.36 | 0.20 | 0.00 | 0.65 | 0.64 | 6.45 | 0.71 |
| ACCCCAGGCA | Hs.241558 | ariadne (Drosophila) homolog 2 or ESTs | 0.18 | 0.00 | 0.00 | 0.00 | 0.20 | 0.00 | 0.65 | 0.64 | 6.45 | 0.00 |
| AAAGCGTAAA | J03143 | interferon gamma receptor 1 | 0.00 | 0.00 | 0.20 | 0.00 | 0.20 | 0.00 | 0.65 | 0.64 | 6.45 | 0.20 |
| GTGGTGCACC | Hs.254442 | ESTs | 0.00 | 0.00 | 0.00 | 0.00 | 0.20 | 0.00 | 0.65 | 0.64 | 6.45 | 0.00 |
| CCAGTGTCTG | AF074993 | lysophospholipase I (EST) | 0.37 | 0.97 | 0.78 | 1.99 | 0.20 | 0.00 | 0.33 | 0.97 | 6.50 | 2.11 |
| CCTTCTCACT | Hs.20013 | GCIP-interacting protein p29 | 0.18 | 0.58 | 0.59 | 0.00 | 0.20 | 0.00 | 0.33 | 0.97 | 6.50 | 3.28 |
| ACAGAATGGC | X92963 | ubiquilin-conjugating enzyme E2E 1 (homologous to yeast UBC4/5) | 0.37 | 0.39 | 0.39 | 0.00 | 0.20 | 0.00 | 0.33 | 0.97 | 6.50 | 1.05 |
| CGTCTGTAAG | Hs.111244 | hypothetical protein | 0.18 | 0.19 | 0.00 | 0.36 | 0.20 | 0.00 | 0.33 | 0.97 | 6.50 | 0.00 |
| GCATTTCAGG | AK001835 | sorting nexin 4 | 0.18 | 0.00 | 0.20 | 0.18 | 0.20 | 0.00 | 0.33 | 0.97 | 6.50 | 1.11 |
| GCGGTCATTG | Hs.74561 | alpha-2-macroglobulin | 0.18 | 0.00 | 0.39 | 0.00 | 0.20 | 0.00 | 0.33 | 0.97 | 6.50 | 2.17 |
| GTTGCCCAGA | Hs.225198 | ESTs | 0.00 | 0.00 | 0.00 | 0.00 | 0.20 | 0.00 | 0.33 | 0.97 | 6.50 | 0.00 |
| CCTTTGTAAG | Hs.78465 | v-jun avian sarcoma virus 17 oncogene homolog | 0.74 | 0.77 | 0.39 | 0.18 | 0.00 | 0.35 | 1.63 | 0.64 | 6.49 | 0.53 |
| TTGAAGGGCC | AL110191 | delta sleep inducing peptide, immunoreactor | 0.00 | 0.19 | 0.00 | 0.00 | 0.00 | 0.35 | 1.63 | 0.64 | 6.49 | 0.00 |
| TAGACTGCTG | AF207601 | hypoxia-inducible factor 1, alpha subunit (basic helix-loop-helix transcription factor), HIF1 alpha | 0.00 | 0.00 | 0.00 | 0.00 | 0.00 | 0.35 | 0.98 | 1.29 | 6.49 | 0.00 |
| TGGACCCAAC | AF006621 | chromosome 4 open reading frame 1 | 0.74 | 1.74 | 0.59 | 0.00 | 0.00 | 0.35 | 0.33 | 1.93 | 6.46 | 0.80 |
| ATGCTTGCTT | BC005127 | adipose differentiation-related protein | 3.14 | 5.02 | 5.88 | 2.71 | 0.98 | 3.19 | 6.50 | 19.63 | 6.27 | 1.87 |
| CTTGACATAC | AJ227912 | dual specificity phosphatase 1 | 0.18 | 0.58 | 0.20 | 0.54 | 0.78 | 0.53 | 1.30 | 6.76 | 6.15 | 1.11 |
| TAATAAAGCA | X76092 | regulatory factor X, 3 (influences HLA class II expression) or seryl-tRNA synthetase | 1.11 | 0.97 | 0.59 | 0.72 | 0.20 | 0.18 | 1.30 | 0.97 | 5.97 | 0.53 |
| CCTGTGGTCC | AK000470 | KIAA1609 protein | 0.55 | 0.77 | 0.59 | 0.54 | 0.20 | 0.18 | 0.65 | 1.61 | 5.95 | 1.07 |
| GCTAGTGATG | AF201949 | 60S ribosomal protein L30 isolog | 0.37 | 0.58 | 0.39 | 0.90 | 0.20 | 0.18 | 0.98 | 1.29 | 5.97 | 1.05 |
| AATAAATGGA | Hs.109052 | chromosome 14 open reading frame 2 | 0.55 | 0.39 | 0.78 | 0.72 | 0.20 | 0.18 | 0.98 | 1.29 | 5.97 | 1.42 |
| TGCACGACTA | AB029551 | RING1 and YY1 binding protein | 0.18 | 0.39 | 0.20 | 0.18 | 0.20 | 0.18 | 0.65 | 1.61 | 5.95 | 1.11 |
| TTGGAACAAT | Hs.80305 | Human clone 23719 mRNA sequence | 0.18 | 0.39 | 0.20 | 1.26 | 1.37 | 3.01 | 14.63 | 11.59 | 5.99 | 1.11 |
| ATGTGTGTTG | AF002697 | BNIP3; BCL2/adenovirus E18 19 kD-interacting protein 3, Nip3 | 0.37 | 0.77 | 0.39 | 0.36 | 0.39 | 0.71 | 2.60 | 3.86 | 5.87 | 1.05 |
| TGCTGCATTG | AL050025 | adaptor-related protein complex 1, gamma 1 subunit | 0.37 | 0.39 | 0.98 | 1.44 | 0.59 | 1.42 | 3.90 | 7.72 | 5.78 | 2.65 |
| TCCTGTAAAG | AF070648 | clone 24651 mRNA sequence. | 4.79 | 7.53 | 6.27 | 7.04 | 0.20 | 0.53 | 1.63 | 2.58 | 5.77 | 1.31 |
| AGGAATGTTA | Hs.82689 | tumor rejection antigen (gp96) 1 | 0.74 | 1.16 | 0.78 | 0.90 | 0.00 | 0.18 | 0.98 | 0.00 | 5.44 | 1.05 |
| TATTCTCAAT | U79254 | asparaginyl-tRNA synthetase | 0.37 | 0.58 | 0.39 | 0.36 | 0.00 | 0.18 | 0.98 | 0.00 | 5.44 | 1.05 |
| ATGGTCAGTA | Hs.287955 | Homo sapiens cDNA FLJ13090 fis, clone NT2RP3002142 | 0.18 | 0.58 | 0.39 | 0.54 | 0.00 | 0.18 | 0.98 | 0.00 | 5.44 | 2.17 |
| TGTGATCACA | Y13640 | proteasome (prosome, macropain) subunit, beta type, 10 | 0.55 | 0.39 | 0.39 | 0.18 | 0.00 | 0.18 | 0.98 | 0.00 | 5.44 | 0.71 |
| GAACACCACT | Hs.155595 | neural precursor cell expressed, developmentally down-regulated 5 | 0.55 | 0.39 | 0.98 | 0.36 | 0.00 | 0.18 | 0.98 | 0.00 | 5.44 | 1.78 |
| AGACTATATT | AF195530 | XPNPEP1; X-prolyl aminopeptidase (aminopeptidase P) 1, soluble | 0.37 | 0.39 | 0.39 | 0.18 | 0.00 | 0.18 | 0.98 | 0.00 | 5.44 | 1.05 |
| TGGAGAAGAG | Hs.179526 | upregulated by 1,25-dihydroxyvitamin D-3 | 0.37 | 0.19 | 0.00 | 0.00 | 0.00 | 0.18 | 0.98 | 0.00 | 5.44 | 0.00 |
| TTTGGTGTTT | BC007305 | FASN; fatty acid synthase | 0.18 | 0.00 | 0.00 | 0.36 | 0.00 | 0.18 | 0.98 | 0.00 | 5.44 | 0.00 |
| CCTCCCAAGG | X89602 | rTS beta protein | 0.00 | 0.00 | 0.00 | 0.00 | 0.00 | 0.18 | 0.98 | 0.00 | 5.44 | 0.00 |
| TGTTACTGGA | Hs.288106 | Homo sapiens cDNA: FLJ21168 fis, clone CAS10874 or ESTs | 0.00 | 0.00 | 0.00 | 0.00 | 0.00 | 0.18 | 0.98 | 0.00 | 5.44 | 0.00 |
| CTTAAGGATT | AY032853 | PAI-RBP1; PAI-1 mRNA-binding protein | 1.11 | 1.74 | 1.96 | 1.08 | 0.00 | 0.18 | 0.65 | 0.32 | 5.39 | 1.77 |
| CTGTGCCCAG | Hs.111222 | ribosomal protein L30 | 0.92 | 1.55 | 0.98 | 0.72 | 0.00 | 0.18 | 0.65 | 0.32 | 5.39 | 1.07 |
| GAATGTAAGT | AF061940 | staufen (Drosophila, RNA-binding protein) | 0.74 | 1.35 | 0.39 | 0.54 | 0.00 | 0.18 | 0.65 | 0.32 | 5.39 | 0.53 |
| TAAACCGGAA | AF151036 | hypothetical protein | 1.11 | 0.77 | 1.76 | 0.72 | 0.00 | 0.18 | 0.65 | 0.32 | 5.39 | 1.59 |
| AATACACAGA | AF353720 | DEAD/H (Asp-Glu-Ala-Asp/His) box polypeptide 19 (Dbp5, yeast, homolog) | 0.37 | 0.77 | 0.00 | 0.18 | 0.00 | 0.18 | 0.65 | 0.32 | 5.39 | 0.00 |
| GGTTATTTAG | D67031 | ADD3; adducin 3 (gamma) | 0.37 | 0.77 | 0.59 | 0.36 | 0.00 | 0.18 | 0.65 | 0.32 | 5.39 | 1.59 |
| TCCTTATTAA | Hs.148589 | ESTs | 0.00 | 0.77 | 0.39 | 0.54 | 0.00 | 0.18 | 0.65 | 0.32 | 5.39 | 0.39 |
| GAAGGAGATA | AF047431 | cholinephosphotransferase 1 | 0.74 | 1.35 | 0.39 | 0.00 | 0.00 | 0.35 | 1.30 | 0.64 | 5.54 | 0.53 |
| TTAACCCTCT | Z48950 | H3 histone, family 3B (H3.3B) | 1.29 | 0.58 | 0.20 | 0.72 | 0.00 | 0.18 | 0.65 | 0.32 | 5.39 | 0.16 |
| CCAGAGTCTC | Hs.259347 | Homo sapiens cDNA FLJ11690 fis, clone HEMBA1004978 or vascular endothelial growth factor | 0.00 | 0.39 | 0.39 | 0.18 | 0.00 | 0.18 | 0.65 | 0.32 | 5.39 | 0.39 |
| AATCTGGTTG | Hs.103804 | heterogeneous nuclear ribonucleoprotein U (scaffold attachment factor A) | 0.55 | 0.19 | 0.39 | 0.72 | 0.00 | 0.18 | 0.65 | 0.32 | 5.39 | 0.71 |
| TACTGGTTTA | Hs.30299 | IGF-II mRNA-binding protein 2 | 0.37 | 0.19 | 0.20 | 0.18 | 0.00 | 0.18 | 0.65 | 0.32 | 5.39 | 0.54 |
| GTGGCCTTTG | AB015336 | RNA binding motif protein 12 | 0.00 | 0.19 | 0.39 | 0.00 | 0.00 | 0.18 | 0.65 | 0.32 | 5.39 | 0.39 |
| TGATGACTGT | AL136551 | sestrin 2 | 0.00 | 0.19 | 0.00 | 0.00 | 0.00 | 0.18 | 0.65 | 0.32 | 5.39 | 0.00 |
| GCGGCTGCGC | Hs.74631 | basigin | 0.00 | 0.19 | 0.00 | 0.00 | 0.00 | 0.18 | 0.65 | 0.32 | 5.39 | 0.00 |
| GCAGGATCGG | U25278 | mitogen-activated protein kinase 7 | 0.74 | 0.19 | 0.20 | 0.72 | 0.00 | 0.53 | 1.63 | 1.29 | 5.51 | 0.27 |
| GCAGGAAATA | AL137448 | ankyrin repeat domain 3 | 0.18 | 0.00 | 0.00 | 0.00 | 0.00 | 0.18 | 0.65 | 0.32 | 5.39 | 0.00 |
| ACCTGAAACC | AB014551 | rho/rac guanine nucleotide exchange factor (GEF) 2 or heparan sulfate 6-O-sulfotransferase | 0.00 | 0.00 | 0.00 | 0.00 | 0.00 | 0.18 | 0.65 | 0.32 | 5.39 | 0.00 |
| CCCGAGGCAG | AF055460 | stanniocalcin 2 (STC-2) | 0.00 | 0.00 | 0.00 | 0.18 | 0.00 | 0.18 | 0.65 | 0.32 | 5.39 | 0.00 |
| GGGGCTGGCA | AF062481 | 7-dehydrocholesterol reductase | 0.00 | 0.00 | 0.00 | 0.00 | 0.00 | 0.18 | 0.65 | 0.32 | 5.39 | 0.00 |
| TCCTTTGGAT | Hs.128659 | ESTs | 0.00 | 0.00 | 0.39 | 0.00 | 0.00 | 0.18 | 0.65 | 0.32 | 5.39 | 0.39 |
| AAGATCCTCA | Hs.118127 | actin, alpha, cardiac muscle or others | 0.00 | 0.00 | 0.20 | 0.18 | 0.00 | 0.18 | 0.65 | 0.32 | 5.39 | 0.20 |
| GTGTTCCCAT | Hs.267120 | Human DNA sequence from clone RP3-351K20 on chromosome 6q22.1-22.33. Contains the | 0.00 | 0.00 | 0.00 | 0.36 | 0.00 | 0.18 | 0.65 | 0.32 | 5.39 | 0.00 |
| | | gene for a novel C3HO [symbol] | | | | | | | | | | |
| GCACGTGAGC | Hs.155560 | calnexin | 0.00 | 0.00 | 0.00 | 0.00 | 0.00 | 0.18 | 0.65 | 0.32 | 5.39 | 0.00 |
| AAATGCAGTA | Hs.75262 | cathepsin O or ESTs | 0.00 | 0.00 | 0.00 | 0.00 | 0.00 | 0.18 | 0.65 | 0.32 | 5.39 | 0.00 |
| TAAGCATTAA | AF000652 | syndecan binding protein (syntenin) | 0.55 | 0.00 | 0.00 | 0.54 | 0.00 | 0.35 | 0.98 | 0.97 | 5.57 | 0.00 |
| GCGAGACTCA | AL080085 | Weakly similar to I38022 hypothetical protein [H. sapiens] (EST) | 0.00 | 0.00 | 0.00 | 0.00 | 0.00 | 0.35 | 0.98 | 0.97 | 5.57 | 0.00 |
| GTGTGGGAGA | Hs.16331 | Homo sapiens cDNA: FLJ21482 fis, clone COL05135 | 0.00 | 0.00 | 0.00 | 0.00 | 0.00 | 0.35 | 0.98 | 0.97 | 5.57 | 0.00 |
| CCTTTGGCTA | Hs.108957 | 40S ribosomal protein S27 isoform or KIAA1432 protein | 3.32 | 4.25 | 2.55 | 1.63 | 0.00 | 0.18 | 0.33 | 0.64 | 5.39 | 0.77 |
| CACCATTCAG | AL162046 | hypothetical protein FLJ10856 | 1.29 | 1.74 | 1.37 | 1.26 | 0.00 | 0.18 | 0.33 | 0.64 | 5.39 | 1.06 |
| TAAATAAATA | AB011125 | amyloid beta (A4) precursor protein (protease nexin-II, Alzheimer disease) or others | 0.92 | 0.77 | 0.20 | 1.08 | 0.00 | 0.18 | 0.33 | 0.64 | 5.39 | 0.22 |
| GAGCAGCTGG | U83246 | copine I or SWAP-70 protein | 0.37 | 0.58 | 0.00 | 0.72 | 0.00 | 0.18 | 0.33 | 0.64 | 5.39 | 0.00 |
| CAAATAAATG | AF035292 | KIAA0657 protein | 0.00 | 0.58 | 0.59 | 0.18 | 0.00 | 0.18 | 0.33 | 0.64 | 5.39 | 0.59 |
| ATGTACTAAA | Y07968 | TRK-fused gene | 1.29 | 0.97 | 1.18 | 1.99 | 0.00 | 0.35 | 0.65 | 1.29 | 5.54 | 0.91 |
| TAACTAACAA | D14663 | kynureninase (L-kynurenine hydrolase) | 0.92 | 0.39 | 0.20 | 0.00 | 0.00 | 0.18 | 0.33 | 0.64 | 5.39 | 0.22 |
| GCCGCTTCTA | U24497 U24 | PKD1; polycystic kidney disease 1 (autosomal dominant) protein | 0.55 | 0.39 | 1.18 | 0.18 | 0.00 | 0.18 | 0.33 | 0.64 | 5.39 | 2.15 |
| AACATTGACA | AF130104 | hypothetical protein DKFZp564K142 | 0.00 | 0.39 | 0.20 | 0.36 | 0.00 | 0.18 | 0.33 | 0.64 | 5.39 | 0.20 |
| TCAGAACAGT | U07231 | G-rich RNA sequence binding factor 1 (GRSF-1) | 0.37 | 0.19 | 0.00 | 0.36 | 0.00 | 0.18 | 0.33 | 0.64 | 5.39 | 0.00 |
| AGGGCTCCCA | Hs.29797 | ribosomal protein L10 | 0.18 | 0.19 | 0.20 | 0.36 | 0.00 | 0.18 | 0.33 | 0.64 | 5.39 | 1.11 |
| TGGTCAAGGT | Hs.13996 | ESTs | 0.18 | 0.19 | 0.20 | 0.00 | 0.00 | 0.18 | 0.33 | 0.64 | 5.39 | 1.11 |
| CCAGCCAGGT | AK000849 | KIAA0261 protein (EST) | 0.00 | 0.19 | 0.00 | 0.36 | 0.00 | 0.18 | 0.33 | 0.64 | 5.39 | 0.00 |
| ATCATCGCTG | Hs.242038 | Homo sapiens clone L48 polyadenylated HERV LTR sequence end flanking cellular sequence, mRNA | 0.00 | 0.19 | 0.20 | 0.18 | 0.00 | 0.18 | 0.33 | 0.64 | 5.39 | 0.20 |
| ATACAGATTG | Hs.88799 | anaphase-promoting complex 10 | 0.00 | 0.19 | 0.20 | 0.18 | 0.00 | 0.18 | 0.33 | 0.64 | 5.39 | 0.20 |
| TGTGTGTGTG | AF035305 | (11nt + T) Williams-Beuren syndrome chromosome region 14 and others | 0.55 | 0.19 | 0.20 | 0.18 | 0.00 | 0.35 | 0.65 | 1.29 | 5.54 | 0.36 |
| GAGGCCAAGA | M21758 | glutathione S-transferase A2 | 0.18 | 0.00 | 0.00 | 0.00 | 0.00 | 0.18 | 0.33 | 0.64 | 5.39 | 0.00 |
| ATGGCAGGCG | Hs.186613 | sphingosine-1-phosphate lyase 1 or diaphorase (NADH/NADPH) (cytochrome b-5 reductase) | 0.18 | 0.00 | 0.00 | 0.18 | 0.00 | 0.18 | 0.33 | 0.64 | 5.39 | 0.00 |
| TCCTGGGCCT | AB033831 | PDGFC; platelet derived growth factor C | 0.00 | 0.00 | 0.00 | 0.18 | 0.00 | 0.18 | 0.33 | 0.64 | 5.39 | 0.00 |
| GCTATTTCCT | AL133572 | protein containing CXXC domain 2 | 0.00 | 0.00 | 0.00 | 0.18 | 0.00 | 0.18 | 0.33 | 0.64 | 5.39 | 0.00 |
| GTCTGCCCTC | M88468 | mevalonate kinase (mevalonic aciduria) | 0.00 | 0.00 | 0.20 | 0.00 | 0.00 | 0.18 | 0.33 | 0.64 | 5.39 | 0.20 |
| GCCTGAGCTG | S81737 | alpha 1 syntrophin [human, mRNA Partial, 1771 nt] | 0.00 | 0.00 | 0.00 | 0.00 | 0.00 | 0.18 | 0.33 | 0.64 | 5.39 | 0.00 |
| TGGGGACAGG | U09117 | phospholipase C, delta 1 | 0.00 | 0.00 | 0.00 | 0.00 | 0.00 | 0.18 | 0.33 | 0.64 | 5.39 | 0.00 |
| TTGGCCAGGG | Hs.269592 | ESTs | 0.00 | 0.00 | 0.20 | 0.18 | 0.00 | 0.18 | 0.33 | 0.64 | 5.39 | 0.20 |
| CAGTTGCCAG | Hs.93379 | eukaryotic translation initiation factor 4B | 0.00 | 0.00 | 0.20 | 0.00 | 0.00 | 0.18 | 0.33 | 0.64 | 5.39 | 0.20 |
| GGCCTGGCTA | Hs.62119 | Homo sapiens clone 25020 mRNA sequence | 0.00 | 0.00 | 0.00 | 0.36 | 0.00 | 0.18 | 0.33 | 0.64 | 5.39 | 0.00 |
| GTTGTATGTA | Hs.175043 | ancient conserved domain protein 4 | 0.00 | 0.00 | 0.00 | 0.18 | 0.00 | 0.18 | 0.33 | 0.64 | 5.39 | 0.00 |
| CCCACCTCAA | Hs.154104 | pleiomorphic adenoma gene-like 2 | 0.00 | 0.00 | 0.00 | 0.00 | 0.00 | 0.18 | 0.33 | 0.64 | 5.39 | 0.00 |
| GGACCGAGGG | BC003353 | hypothetical protein MGC5309 | 0.37 | 0.39 | 0.20 | 0.18 | 0.00 | 0.18 | 0.00 | 0.97 | 5.39 | 0.54 |
| GTGGGACCAT | Hs.11774 | protein (peptidyl-prolyl cis/trans isomerase) NIMA-Interacting, 4 (parvulin) | 0.00 | 0.39 | 0.59 | 0.36 | 0.00 | 0.18 | 0.00 | 0.97 | 5.39 | 0.59 |
| ATTGCACCAC | S81734 | transglutaminase 2 or 5-hydroxytryptamine (serotonin) receptor 3B or others | 0.18 | 0.19 | 0.59 | 1.08 | 0.00 | 0.18 | 0.00 | 0.97 | 5.39 | 3.28 |
| GCACACGTTT | Hs.241913 | ESTs | 0.18 | 0.19 | 0.20 | 0.36 | 0.00 | 0.18 | 0.00 | 0.97 | 5.39 | 1.11 |
| CGTAAAAAGG | AF051321 | Sam68-like phosphotyrosine protein, T-STAR | 0.00 | 0.19 | 0.00 | 0.00 | 0.00 | 0.18 | 0.00 | 0.97 | 5.39 | 0.00 |
| ATGGTGGGGG | M92843 | zinc finger protein homologous to Zfp-36 in mouse | 0.00 | 0.19 | 0.00 | 0.18 | 0.00 | 0.18 | 0.00 | 0.97 | 5.39 | 0.00 |
| ATCAAGAATC | Hs.14623 | interferon, gamma-inducible protein 30 | 0.55 | 0.00 | 0.39 | 0.00 | 0.00 | 0.18 | 0.00 | 0.97 | 5.39 | 0.71 |
| CAGTAGACAG | Y17392 | PFDN1; prefoldin 1 | 0.37 | 0.00 | 0.20 | 0.54 | 0.00 | 0.18 | 0.00 | 0.97 | 5.39 | 0.54 |
| CCTTGAGTAC | AF054987 | ALDOC; aldolase C, fructose-bisphosphate | 0.00 | 0.00 | 0.00 | 0.00 | 0.00 | 0.35 | 0.00 | 1.93 | 5.51 | 0.00 |
| TACTTGTATT | U90914 | carboxypeptidase D | 0.00 | 0.00 | 0.00 | 0.00 | 0.00 | 0.18 | 0.00 | 0.97 | 5.39 | 0.00 |
| GCTGCCATCC | X61094 | GM2 ganglloside activator protein | 0.00 | 0.00 | 0.00 | 0.18 | 0.00 | 0.18 | 0.00 | 0.97 | 5.39 | 0.00 |
| GAGCAAAGGA | Hs.181156 | ESTs | 0.00 | 0.00 | 0.20 | 0.00 | 0.00 | 0.18 | 0.00 | 0.97 | 5.39 | 0.20 |
| ACATCAAAGT | Hs.118630 | MAX-interacting protein 1 or four and a half LIM domains 1 | 0.00 | 0.00 | 0.00 | 0.18 | 0.00 | 0.18 | 0.00 | 0.97 | 5.39 | 0.00 |
| CCATCCAGGC | AF121860 | sorting nexin 10 | 0.00 | 0.58 | 0.39 | 0.18 | 0.20 | 0.53 | 0.33 | 3.54 | 5.30 | 0.39 |
| GTGAGTGTGT | AL118520 | MAP1 light chain 3-like protein 1 | 0.00 | 0.58 | 0.00 | 0.00 | 0.98 | 0.00 | 2.93 | 2.25 | 5.29 | 0.00 |
| TTTGTGTGTG | Hs.129915 | phosphotriesterase related or EH-domain containing 3 or ESTs | 0.00 | 0.00 | 0.00 | 0.36 | 0.20 | 0.18 | 1.95 | 0.00 | 5.13 | 0.00 |
| ACTGAGGAAA | X64875 | IGFBP3; insulin-like growth factor binding protein 3 | 3.50 | 5.80 | 9.02 | 9.21 | 1.57 | 0.18 | 4.55 | 4.51 | 5.18 | 2.58 |
| TGGCTGGGTG | Hs.252189 | syndecan 4 (amphiglycan, ryudocan) | 0.18 | 0.19 | 0.00 | 0.00 | 0.20 | 0.18 | 0.65 | 1.29 | 5.11 | 0.00 |
| TGTGGAAACC | U77735 | pim-2 oncogene | 0.00 | 0.00 | 0.20 | 0.72 | 0.39 | 0.35 | 0.33 | 3.54 | 5.23 | 0.20 |
| AAGATTGGTG | X60111 | CD9 antigen (p24) | 0.00 | 0.00 | 0.00 | 0.00 | 0.00 | 0.89 | 0.65 | 3.86 | 5.07 | 0.00 |
| GGCATCAGGG | AL110276 | hypothetical protein, estradiol-induced | 0.00 | 0.19 | 0.00 | 0.54 | 0.59 | 0.18 | 1.95 | 1.93 | 5.04 | 0.00 |
| TGCATCTGGT | X87949 | heat shock 70 kD protein 5 (glucose-regulated protein, 78 kD) (BiP protein) | 7.75 | 10.24 | 7.84 | 10.11 | 7.83 | 10.99 | 77.70 | 17.38 | 5.05 | 1.01 |
| AGGAGCGGGG | Hs.252189 | syndecan 4 (amphiglycan, ryudocan) | 0.92 | 0.58 | 1.37 | 1.63 | 0.20 | 1.42 | 1.63 | 6.44 | 4.98 | 1.49 |
| TACTGGAGTA | AY007810 | mesoderm development candidate 1 | 0.74 | 1.16 | 0.00 | 0.18 | 0.20 | 0.00 | 0.98 | 0.00 | 4.90 | 0.00 |
| GGCAACAAAA | BC003572 | karyopherin (importin) beta 1 | 0.55 | 0.39 | 0.78 | 0.54 | 0.20 | 0.00 | 0.98 | 0.00 | 4.90 | 1.42 |
| AACTATGCCA | AF121859 | sorting nexin 9 (SNX9) | 0.00 | 0.19 | 0.20 | 0.00 | 0.20 | 0.00 | 0.98 | 0.00 | 4.90 | 0.20 |
| TGCAGAAGTA | D38293 | adaptor-related protein complex 3, mu 2 subunit | 0.00 | 0.00 | 0.00 | 0.54 | 0.20 | 0.00 | 0.98 | 0.00 | 4.90 | 0.00 |
| AAAATGAAGA | Y12393 | karyopherin alpha 4 (importin alpha 3) | 0.00 | 0.00 | 0.20 | 0.00 | 0.20 | 0.00 | 0.98 | 0.00 | 4.90 | 0.20 |
| TGTGTACCTA | Hs.208321 | ESTs | 0.00 | 0.00 | 0.00 | 0.00 | 0.20 | 0.00 | 0.98 | 0.00 | 4.90 | 0.00 |
| GAAGATGTGG | Hs.118064 | similar to rat nuclear ubiquitous casein kinase 2 | 2.40 | 1.74 | 1.57 | 1.99 | 0.20 | 0.00 | 0.65 | 0.32 | 4.85 | 0.65 |
| TTTGTTGCTT | AF150734 | PC326 protein | 0.92 | 0.97 | 1.96 | 1.44 | 0.20 | 0.00 | 0.65 | 0.32 | 4.85 | 2.13 |
| AAGGTCCTAG | L49054 | myeloid leukemia factor 1 | 0.55 | 0.39 | 0.59 | 0.54 | 0.20 | 0.00 | 0.65 | 0.32 | 4.85 | 1.07 |
| ATCCTGCTGG | Hs.83583 | actin related protein 2/3 complex, subunit 2 (34 kD) | 0.00 | 0.39 | 0.00 | 0.00 | 0.20 | 0.00 | 0.65 | 0.32 | 4.85 | 0.00 |
| AGGCTTTATG | U68494 | hbc647 mRNA sequence | 0.55 | 0.19 | 0.39 | 0.36 | 0.20 | 0.00 | 0.65 | 0.32 | 4.85 | 0.71 |
| TGCTGCCTCA | AF044924 | hook2 protein (HOOK2) | 0.37 | 0.19 | 0.39 | 0.36 | 0.20 | 0.00 | 0.65 | 0.32 | 4.85 | 1.05 |
| CTGGACATTG | Hs.274317 | DEAD-box protein abstrakt | 0.37 | 0.19 | 0.00 | 0.00 | 0.20 | 0.00 | 0.65 | 0.32 | 4.85 | 0.00 |
| GAGGGAGGTT | AL121886 | ribosomal protein L27a | 0.18 | 0.19 | 0.00 | 0.00 | 0.20 | 0.00 | 0.65 | 0.32 | 4.85 | 0.00 |
| GGCACCTGGC | S71404 | interleukin-9 receptor {5′ region} [human, megakar | 0.18 | 0.19 | 0.00 | 0.00 | 0.20 | 0.00 | 0.65 | 0.32 | 4.85 | 0.00 |
| TGCTAAGAAT | M74297 | homeo box A4 | 0.00 | 0.19 | 0.00 | 0.36 | 0.20 | 0.00 | 0.65 | 0.32 | 4.85 | 0.00 |
| AGTAGCGAAC | X81003 | protein phosphatase 1, regulatory (inhibitor) subunit 11 | 0.00 | 0.19 | 0.00 | 0.00 | 0.20 | 0.00 | 0.65 | 0.32 | 4.85 | 0.00 |
| CATAGAGCCA | AK001830 | mitochondrial ribosomal protein S4 | 0.18 | 0.00 | 0.00 | 0.00 | 0.20 | 0.00 | 0.65 | 0.32 | 4.85 | 0.00 |
| AAGTTTGTGG | Hs.252259 | ribosomal protein S3 or Homo sapiens mRNA; cDNA DKFZp434A128 (from clone | 0.18 | 0.00 | 0.39 | 0.00 | 0.20 | 0.00 | 0.65 | 0.32 | 4.85 | 2.17 |
| | | DKFZp434A128); partial cds | | | | | | | | | | |
| GGTGCCCAGT | Hs.75607 | myristoylated alanine-rich protein kinase C substrate (MARCKS, 80K-L) | 0.18 | 0.00 | 0.59 | 0.00 | 0.20 | 0.00 | 0.65 | 0.32 | 4.85 | 3.28 |
| GTCCGGAGAT | Hs.19769 | ESTs | 0.18 | 0.00 | 0.00 | 0.00 | 0.20 | 0.00 | 0.65 | 0.32 | 4.85 | 0.00 |
| TGCTTTGCTT | D86962 | growth factor receptor-bound protein 10 | 0.00 | 0.00 | 0.20 | 0.00 | 0.20 | 0.00 | 0.65 | 0.32 | 4.85 | 0.20 |
| TCTGTTCTGG | L22005 | cell division cycle 34 | 0.00 | 0.00 | 0.00 | 0.00 | 0.20 | 0.00 | 0.65 | 0.32 | 4.85 | 0.00 |
| TTTACTTTGG | L27479 | Friedreich ataxia region gene X123 | 0.00 | 0.00 | 0.00 | 0.00 | 0.20 | 0.00 | 0.65 | 0.32 | 4.85 | 0.00 |
| CCCCTCTGAG | U18121 | adenosine deaminase, RNA-specific | 0.00 | 0.00 | 0.00 | 0.36 | 0.20 | 0.00 | 0.65 | 0.32 | 4.85 | 0.00 |
| AAGGACATTC | X13939 S93 | LAMC1; laminin, gamma 1 (formerty LAMB2) | 0.00 | 0.00 | 0.20 | 0.00 | 0.20 | 0.00 | 0.65 | 0.32 | 4.85 | 0.20 |
| GACTTGATAT | Hs.75586 | cyclin D2 | 0.00 | 0.00 | 0.00 | 0.00 | 0.39 | 0.00 | 1.30 | 0.64 | 4.97 | 0.00 |
| GCAGCTGCCT | Hs.270264 | ESTs | 0.00 | 0.00 | 0.20 | 0.18 | 0.20 | 0.00 | 0.65 | 0.32 | 4.85 | 0.20 |
| TTTGAGGGAA | Hs.6727 | Ras-GTPase activating protein SH3 domain-binding protein 2 or ESTs | 0.00 | 0.00 | 0.00 | 0.00 | 0.20 | 0.00 | 0.65 | 0.32 | 4.85 | 0.00 |
| CTGACAGTTC | Hs.15423 | hypothetical protein HDCMC04P or calmodulin 2 (phosphorylase kinase, delta) | 0.74 | 0.97 | 1.37 | 0.90 | 0.20 | 0.00 | 0.33 | 0.64 | 4.85 | 1.85 |
| CCTCCTCTGA | Hs.28505 | ubiquitin-conjugating enzyme E2H (homologous to yeast UBC8) | 2.03 | 1.74 | 1.37 | 1.63 | 0.39 | 0.00 | 0.65 | 1.29 | 4.97 | 0.67 |
| GGAAACTGAT | Hs.83916 | NADH dehydrogenase (ubiquinone) 1 alpha subcomplex, 5 (13 kD, B13) | 0.74 | 0.58 | 0.59 | 0.18 | 0.20 | 0.00 | 0.33 | 0.64 | 4.85 | 0.80 |
| ATGATGCCTC | AB018345 | KIAA0802 protein | 0.18 | 0.58 | 0.59 | 1.26 | 0.20 | 0.00 | 0.33 | 0.64 | 4.85 | 3.28 |
| TGGAGATGTG | AK000926 | likely ortholog of mouse ubiquitin-conjugating enzyme E2-230K | 0.37 | 0.39 | 0.39 | 0.36 | 0.20 | 0.00 | 0.33 | 0.64 | 4.85 | 1.05 |
| AATCCAAAGG | U39656 | mitogen-activated protein kinase kinase 6 or phasphorylase, glycogen: liver (Hers disease, | 0.37 | 0.19 | 0.39 | 0.18 | 0.20 | 0.00 | 0.33 | 0.64 | 4.85 | 1.05 |
| | | glycogen storage diseas [symbol] | | | | | | | | | | |
| GTCACCCAAA | U73522 | associated molecule with the SH3 domain of STAM | 0.18 | 0.19 | 0.39 | 0.18 | 0.20 | 0.00 | 0.33 | 0.64 | 4.85 | 2.17 |
| TTAATTGATA | M68857 J03 | monoamine oxidase A | 0.00 | 0.19 | 0.00 | 0.00 | 0.20 | 0.00 | 0.33 | 0.64 | 4.85 | 0.00 |
| TTACGATGAA | Hs.6335 | phosphatidylinositol-4-phosphate 5-kinase, type II, beta | 0.55 | 0.00 | 0.20 | 0.54 | 0.20 | 0.00 | 0.33 | 0.64 | 4.85 | 0.36 |
| AAAAGATACT | AF109161 | Cbp/p300-interacting transactivator, with Glu/Asp-rich C-terminal domain, 2, p35srj (MRG1) | 0.37 | 0.00 | 0.20 | 0.18 | 0.20 | 0.00 | 0.33 | 0.64 | 4.85 | 0.54 |
| GCAAATCTGA | AK001489 | ADP-ribosylation factor-like 1 (EST) | 0.37 | 0.00 | 0.00 | 0.54 | 0.20 | 0.00 | 0.33 | 0.64 | 4.85 | 0.00 |
| TATTATTAAA | AK000436 | hypothetical protein FLJ20429 | 0.18 | 0.00 | 0.00 | 0.00 | 0.20 | 0.00 | 0.33 | 0.64 | 4.85 | 0.00 |
| CCCTGCCTTG | X55110 | midkine (neurite growth-promoting factor 2) | 0.18 | 0.00 | 0.00 | 0.00 | 0.20 | 0.00 | 0.33 | 0.64 | 4.85 | 0.00 |
| ATCCGGACCC | U83981 | growth arrest and DNA-damage-inducible 34 (GADD34) | 0.00 | 0.00 | 0.00 | 0.00 | 0.20 | 0.00 | 0.33 | 0.64 | 4.85 | 0.00 |
| GCGGGGGTGT | Hs.5831 | tissue Inhibitor of metalloproteinase 1 (erythroid potentiating activity, collagenase inhibitor) | 0.00 | 0.00 | 0.00 | 0.00 | 0.20 | 0.00 | 0.33 | 0.64 | 4.85 | 0.00 |
| TTACAGAGCT | AF265215 | zinc finger protein 313 | 0.18 | 0.58 | 0.59 | 0.00 | 0.20 | 0.00 | 0.00 | 0.97 | 4.85 | 3.28 |
| TGGCTGCATA | AF174596 | hypothetical protein FLJ21939 similar to 5-azacytidine induced gene 2 (EST) | 0.18 | 0.39 | 0.20 | 0.00 | 0.20 | 0.00 | 0.00 | 0.97 | 4.85 | 1.11 |
| TGCCCAGGGG | Hs.231616 | ESTs | 0.00 | 0.19 | 0.00 | 0.18 | 0.20 | 0.00 | 0.00 | 0.97 | 4.85 | 0.00 |
| GGAGAGAAAA | U34690 | coronin, actin-binding protein, 1A or claudin 12 | 0.37 | 0.00 | 0.20 | 0.36 | 0.20 | 0.00 | 0.00 | 0.97 | 4.85 | 0.54 |
| TTACTTGGCC | BC001310 | hypothetical protein MGC5509 | 0.00 | 0.00 | 0.00 | 0.18 | 0.20 | 0.00 | 0.00 | 0.97 | 4.85 | 0.00 |
| AACTCTTCAC | M34175 J05 | adaptor-related protein complex 2, beta 1 subunit | 0.00 | 0.00 | 0.20 | 0.18 | 0.20 | 0.00 | 0.00 | 0.97 | 4.85 | 0.20 |
| GCACCATTCC | Hs.260855 | Homo sapiens mRNA; cDNA DKFZp761G2311 (from clone DKFZp 761G2311) | 0.00 | 0.00 | 0.00 | 0.00 | 0.20 | 0.00 | 0.00 | 0.97 | 4.85 | 0.00 |
| AGAACACAGT | Hs.85339 | G protein-coupled receptor 39 | 0.00 | 0.00 | 0.00 | 0.00 | 0.20 | 0.00 | 0.00 | 0.97 | 4.85 | 0.00 |
| TTCCCCAGAA | Hs.155212 | methylmalonyl Coenzyme A mutase | 0.00 | 0.00 | 0.00 | 0.00 | 0.20 | 0.00 | 0.00 | 0.97 | 4.85 | 0.00 |
| GATCAATGGA | AF029914 | glucosamine-6-phosphate isomerase | 0.74 | 0.58 | 0.20 | 0.90 | 0.00 | 0.53 | 1.63 | 0.97 | 4.91 | 0.27 |
| CAAATGAGGA | AB020692 | NRAS-related gene | 2.21 | 2.90 | 1.96 | 4.70 | 0.78 | 1.42 | 5.53 | 5.15 | 4.85 | 0.89 |
| AGAAGACTGG | Hs.71523 | ESTs | 0.55 | 0.39 | 0.20 | 0.00 | 0.00 | 0.53 | 0.65 | 1.93 | 4.87 | 0.36 |
| AGTCTGTCCA | X05130 | P4HB: procoliagen-proline, 2-oxoglutarate 4-dioxygenase (proline 4-hydroxylase), beta polypeptide | 0.18 | 0.00 | 0.20 | 0.00 | 0.39 | 0.35 | 2.93 | 0.64 | 4.82 | 1.11 |
| | | (protein disulfide [symbol] | | | | | | | | | | |
| GACTCAGGGA | AK000430 | GTP binding protein 2 | 0.37 | 0.58 | 0.39 | 1.44 | 0.20 | 0.35 | 2.28 | 0.32 | 4.73 | 1.05 |
| CTGAGCACAA | Hs.14070 | Homo sapiens cDNA FLJ14166 fis, clone NT2RP1000796 or cathepsin B | 0.00 | 0.00 | 0.20 | 0.00 | 0.20 | 0.35 | 0.65 | 1.93 | 4.69 | 0.20 |
| TCACTGATGG | AB026289 | NIMA (never in mitosis gene a)-related kinase 6 | 0.37 | 0.77 | 0.20 | 0.54 | 0.20 | 0.35 | 0.33 | 2.25 | 4.69 | 0.54 |
| TACATTCTGT | L08246 | MCL1; myeloid cell leukemia sequence 1 (BCL2-related) | 0.37 | 0.77 | 0.39 | 0.90 | 0.59 | 0.18 | 1.63 | 1.93 | 4.62 | 1.05 |
| TTACTAAATG | BC003552 | CANX; calnexin | 1.66 | 1.35 | 1.37 | 2.89 | 0.59 | 0.53 | 2.93 | 2.25 | 4.63 | 0.83 |
| TAACTTAAGC | AF135161 | CGI-127 protein | 0.74 | 0.19 | 0.59 | 0.18 | 0.00 | 0.35 | 1.63 | 0.00 | 4.66 | 0.80 |
| TACAAAACCA | AK000221 | nucleolin | 1.84 | 1.93 | 0.98 | 2.53 | 0.00 | 0.35 | 1.30 | 0.32 | 4.63 | 0.53 |
| TCAGAGATGA | AB002559 | syntaxin binding protein 2 | 0.55 | 0.19 | 0.20 | 0.18 | 0.39 | 0.18 | 2.28 | 0.32 | 4.56 | 0.36 |
| ATGAAAGGTT | U85044 | hypoxia-inducible factor 1, alpha subunit (basic HLH transcription factor), HIF1 alpha | 0.00 | 0.00 | 0.00 | 0.00 | 0.00 | 0.35 | 0.98 | 0.64 | 4.63 | 0.00 |
| CAGGGGTGAC | Hs.79748 | solute carrier family 3 (activators of dibasic end neutral amino acid transport), member 2 | 1.11 | 2.13 | 1.57 | 1.08 | 0.00 | 0.35 | 0.65 | 0.97 | 4.63 | 1.41 |
| TAGGATGGGG | U51478 | ATPase, Na+/K+ transporting, beta 3 polypeptide | 1.29 | 0.58 | 0.78 | 1.08 | 0.00 | 0.35 | 0.65 | 0.97 | 4.63 | 0.60 |
| GTGTCTTGTA | AK002058 | hypothetical protein FLJ11196 | 0.55 | 0.19 | 0.00 | 0.00 | 0.78 | 0.35 | 3.58 | 1.61 | 4.59 | 0.00 |
| TAACAGCCAG | M69043 | nuclear factor of kappa light polypeptide gene enhancer In B-cells inhibitor, alpha | 0.55 | 0.58 | 0.78 | 0.90 | 0.39 | 0.18 | 1.63 | 0.97 | 4.56 | 1.42 |
| TGTTCCCTTT | L07648 | MAX-interacting protein 1 | 0.00 | 0.58 | 0.20 | 0.18 | 0.39 | 0.18 | 1.63 | 0.97 | 4.56 | 0.20 |
| GCTGGGAGGG | Hs.20733 | EH-domain containing 2 | 0.55 | 1.55 | 0.39 | 1.08 | 4.11 | 2.13 | 8.45 | 19.96 | 4.55 | 0.71 |
| AACTTGATAC | AB032261 | SCD; stearoyl-CoA desaturase (delta-9-desaturase) | 0.00 | 0.00 | 0.00 | 0.00 | 0.20 | 0.53 | 3.25 | 0.00 | 4.45 | 0.00 |
| GAAAAATAGT | Hs.211584 | neurofilament, light polypeptide (68 kD) or ESTs | 0.00 | 0.00 | 0.00 | 0.18 | 0.20 | 0.53 | 2.28 | 0.97 | 4.45 | 0.00 |
| TTTGAAATGA | M55580 | spermidine/spermine N1-acetyltransferase | 0.74 | 0.39 | 0.20 | 0.36 | 0.39 | 1.60 | 5.20 | 3.54 | 4.39 | 0.27 |
| GCAGCTGACG | Hs.227751 | lectin, galactoside-binding, soluble, 1 (galectin 1) | 0.55 | 0.19 | 0.98 | 0.54 | 0.78 | 0.18 | 1.95 | 2.25 | 4.38 | 1.78 |
| CACTGCCTTG | Hs.62954 | ferritin, heavy polypeptide 1 | 0.18 | 0.58 | 0.59 | 0.54 | 0.00 | 0.89 | 1.63 | 2.25 | 4.36 | 3.28 |
| GGGGGCTCCT | X97198 | protein tyrosine phosphatase, receptor type, U | 0.00 | 0.00 | 0.00 | 0.18 | 0.20 | 0.18 | 1.30 | 0.32 | 4.26 | 0.00 |
| ACAAACTGTG | Hs.90370 | actin related protein 2/3 complex, subunit 1A (41 kD) | 1.29 | 0.97 | 0.78 | 0.36 | 0.20 | 0.18 | 0.98 | 0.64 | 4.26 | 0.60 |
| GGAGAGACAG | Hs.170980 | Homo sapiens cDNA: FLJ21849 fis, clone HEP01928 | 0.37 | 0.77 | 0.39 | 1.44 | 0.20 | 0.18 | 0.98 | 0.64 | 4.26 | 1.05 |
| TACCACCAAT | Hs.169476 | glyceraldehyde-3-phosphate dehydrogenase or ESTs | 0.00 | 0.58 | 0.39 | 0.00 | 0.20 | 0.18 | 0.98 | 0.64 | 4.26 | 0.39 |
| CAGCCGTGAT | M86707 | N-myristoyltransferase 1 | 0.74 | 1.35 | 1.37 | 0.54 | 0.20 | 0.18 | 0.65 | 0.97 | 4.26 | 1.85 |
| TTCTCATAGG | Hs.82911 | protein tyrosine phosphatase type IVA, member 2 | 0.18 | 0.19 | 0.59 | 0.54 | 0.20 | 0.18 | 0.65 | 0.97 | 4.26 | 3.28 |
| GTGGTGATGT | Hs.96593 | hypothetical protein | 0.18 | 0.19 | 0.00 | 0.18 | 0.20 | 0.18 | 0.65 | 0.97 | 4.26 | 0.00 |
| TACCATCGAT | Hs.169476 | glyceraldehyde-3-phosphate dehydrogenase | 0.00 | 0.19 | 0.20 | 0.18 | 0.20 | 0.18 | 0.65 | 0.97 | 4.26 | 0.20 |
| CTGCAGAATA | Hs.4310 | eukaryotic translation initiation factor 1A | 0.18 | 0.19 | 0.00 | 0.18 | 0.20 | 0.18 | 0.00 | 1.61 | 4.24 | 0.00 |
| CCACTGTAAG | Hs.211380 | ESTs | 0.00 | 0.00 | 0.00 | 0.00 | 0.00 | 0.53 | 1.63 | 0.64 | 4.28 | 0.00 |
| GACTTCTGAG | AK001255 | DEAD-box protein abstrakt | 0.00 | 0.00 | 0.39 | 0.00 | 0.00 | 0.53 | 0.65 | 1.61 | 4.26 | 0.39 |
| CAAAAGGCTC | AL122115 | potassium channel modulatory factor | 0.92 | 0.97 | 0.59 | 0.36 | 0.00 | 0.53 | 0.33 | 1.93 | 4.26 | 0.64 |
| GCGTGAGTGC | Hs.85155 | butyrate response factor 1 (EGF-response factor 1) | 0.00 | 0.58 | 0.39 | 0.18 | 0.39 | 0.53 | 1.30 | 2.58 | 4.22 | 0.39 |
| CGTGGGTGGG | X06985 | HMOX1; heme oxygenase (decycling) 1 | 0.74 | 0.39 | 0.39 | 0.54 | 17.03 | 14.19 | 61.77 | 67.92 | 4.15 | 0.53 |
| GATTGAACCT | U60205 | sterol-C4-methyl oxidase-like (ERG25) | 0.18 | 0.19 | 0.00 | 0.18 | 0.39 | 0.00 | 1.30 | 0.32 | 4.15 | 0.00 |
| ACCTGGGGAG | AF011374 | 1-acylglycerol-3-phosphate O-acyltransferase 2 (lysophosphatidic acid acyltransferase, beta) | 0.55 | 0.39 | 0.39 | 0.00 | 0.39 | 0.00 | 0.33 | 1.29 | 4.15 | 0.71 |
| GACTTGGCGG | BC008993 | Similar to RIKEN cDNA 5730528L13 | 0.37 | 0.39 | 0.39 | 0.36 | 0.20 | 0.35 | 1.30 | 0.97 | 4.13 | 1.05 |
| GGAGACTTCC | M82809 | annexin A4 | 1.48 | 1.93 | 2.35 | 2.53 | 0.59 | 1.77 | 5.53 | 4.18 | 4.11 | 1.59 |
| GTGACAGAAT | U27460 | UDP-glucose pyrophosphorylase 2 | 0.18 | 0.00 | 0.20 | 0.36 | 0.20 | 0.35 | 0.98 | 1.29 | 4.13 | 1.11 |
| AGTATGTATG | AL034548 | protein kinase domains containing protein similar to phosphoprotein C8FW | 0.37 | 0.77 | 0.20 | 0.90 | 0.00 | 0.71 | 1.30 | 1.61 | 4.10 | 0.54 |
| AGGAGCAAAG | D32143 | biliverdin reductase B (flavin reductase (NADPH)) | 3.14 | 4.64 | 2.94 | 4.52 | 0.98 | 2.66 | 2.60 | 12.23 | 4.07 | 0.94 |

| | | | | | | | | | | | | |
|---|---|---|---|---|---|---|---|---|---|---|---|---|
| | Accession/ | | | | | | | | | | | |
| SAGE Tag | UniGene ID | Description | V1 | V2 | V3 | V4 | V5 | V6 | V7 | V8 | V4/V2 | V3/V1 |

87 genes in FIG. 4(h) (83 genes; 4 tags with no matches not shown) SAGE data for genes up-regulated ≧ 4-fold by hypoxia in 786-0 VHL⁻ Hyp. (V4) when compared to those in 786-0 VHL⁻ Nor. (V2). Also shown,
for comparison, is the SAGE data for each gene in 786-0 VHL⁺ Hyp. (V3) compared to 786-0 VHL⁺ Nor. (V1). SAGE tag abundances were
normalized before calculating ratios, as shown. The table is presented in descending order of V4/V2.

| | | | | | | | | | | | | |
|---|---|---|---|---|---|---|---|---|---|---|---|---|
| TCTTTACTTG | AF004561 | (11nt = A) ARPC3; actin related protein 2/3 complex, subunit 3 (21 kD) | 1.66 | 0.19 | 1.57 | 1.81 | 1.37 | 0.89 | 0.33 | 0.32 | 9.53 | 0.95 |
| CAATGGAGCT | BC000202 | (11nt = T) hypothetical protein FLJ10199 | 1.29 | 0.19 | 1.76 | 1.81 | 1.37 | 1.24 | 1.30 | 0.64 | 9.53 | 1.36 |
| TAACCCAACA | M83088 | PGM1; phosphoglucomutase 1 | 1.11 | 0.19 | 1.18 | 1.63 | 0.39 | 0.71 | 0.00 | 0.00 | 8.58 | 1.06 |
| TCAGTTTGGA | U44772 | palmitoyl protein thioesterase | 0.74 | 0.19 | 0.39 | 1.63 | 0.59 | 0.89 | 0.98 | 0.00 | 8.58 | 0.53 |
| TTGTTGAAGC | AK000638 | kinesin family member 4A | 0.92 | 0.19 | 0.59 | 1.63 | 0.00 | 0.00 | 0.00 | 0.00 | 8.58 | 0.64 |
| AATAAAGCAA | AB022427 | SERP1; stress-associated endoplasmic reticulum protein 1; ribosome associated membrane protein 4 | 0.92 | 0.19 | 0.39 | 1.44 | 0.20 | 0.35 | 0.00 | 0.00 | 7.58 | 0.42 |
| CTAATAAATG | Hs.43621 | ESTs | 1.48 | 0.19 | 0.78 | 1.44 | 1.57 | 1.77 | 0.33 | 0.00 | 7.58 | 0.53 |
| TCTCCAACAA | Hs.288966 | ceruloplasmin (ferroxidase) | 0.74 | 0.19 | 1.37 | 1.26 | 0.00 | 0.18 | 0.00 | 0.64 | 6.63 | 1.85 |
| GTGGTGCCGC | NM_024907 | hypothetical protein FLJ11798 | 0.37 | 0.19 | 0.39 | 1.26 | 0.00 | 0.18 | 0.00 | 0.32 | 6.63 | 1.05 |
| TATTAAATAG | Hs.155543 | proteasome (prosome, macropain) 26S subunit, non-ATPase, 7 (Mov34 homolog) | 0.74 | 0.19 | 0.59 | 1.26 | 0.20 | 0.00 | 0.00 | 0.32 | 6.63 | 0.80 |
| ATGTCCAATT | AL050226 | succinate-CoA ligase, GDP-forming, beta subunit | 0.18 | 0.19 | 0.20 | 1.26 | 0.20 | 0.00 | 0.00 | 0.00 | 6.63 | 1.11 |
| AGCGAGAGAG | Hs.232146 | hypothetical protein FLJ13441 or KIAA1224 protein | 0.18 | 0.19 | 0.59 | 1.26 | 0.20 | 0.00 | 0.00 | 0.00 | 6.63 | 3.28 |
| GTCCGGAGTC | AK001381 | protein phosphatase methylesterase-1 | 1.66 | 0.19 | 0.78 | 1.26 | 0.39 | 0.35 | 0.65 | 0.97 | 6.63 | 0.47 |
| ACAGCCTGCA | AL10980 | (11nt + G) CDC25B; cell division cycle 25B | 0.74 | 0.19 | 0.39 | 1.26 | 0.20 | 0.71 | 0.00 | 0.32 | 6.63 | 0.53 |
| AGAACCTGCA | NM_004424 | E4F transcription factor 1 | 1.11 | 0.19 | 0.20 | 1.26 | 0.20 | 1.06 | 0.00 | 0.00 | 6.63 | 0.18 |
| CCTGTGACAG | Hs.120 | anti-oxidant protein 2 (non-selenium glutathione peroxidase, acidic calcium-independent phospholipase A2) | 0.55 | 0.19 | 1.18 | 1.26 | 2.15 | 2.48 | 0.00 | 1.29 | 6.63 | 2.15 |
| ATCTTGTTAC | X02761 K00 | (11nt = T) FN1; fibronectin (FN precursor). | 6.82 | 0.19 | 1.76 | 1.26 | 6.27 | 8.69 | 22.76 | 10.94 | 6.63 | 0.26 |
| ATTGCACCAC | S81734 | transglutaminase 2 or 5-hydroxytryptamine (serotonin) receptor 3B or others | 0.18 | 0.19 | 0.59 | 1.08 | 0.00 | 0.18 | 0.00 | 0.97 | 5.68 | 3.28 |
| AGGCCAACAG | Hs.77492 | heterogeneous nuclear ribonucleoprotein A0 | 0.55 | 0.19 | 0.39 | 1.08 | 0.39 | 0.00 | 0.65 | 0.00 | 5.68 | 0.71 |
| TTGTTTGTTT | Hs.105695 | ESTs | 1.11 | 0.19 | 0.78 | 1.08 | 0.00 | 0.00 | 0.00 | 0.00 | 5.68 | 0.70 |
| TGTAGGTATT | U19769 | centromere protein F (350/400 kD, mitosin), CENP-F kinetochore protein | 0.74 | 0.19 | 0.20 | 1.08 | 0.00 | 0.00 | 0.00 | 0.00 | 5.68 | 0.27 |
| TTCCGCGTGC | AL049952 | PLOD3; procollagen-lysine, 2-oxoglutarate 5-dioxygenase 3 (lysyl hydroxylase isoform 3) | 0.92 | 0.19 | 1.57 | 1.08 | 0.78 | 1.42 | 0.65 | 0.32 | 5.68 | 1.71 |
| AAAAAAAAAC | Hs.103391 | Human insulin-like growth factor binding protein 5 (IGFBP5) mRNA or enolase 1, (alpha) or solute carrier | 0.18 | 0.19 | 0.00 | 0.90 | 0.00 | 0.18 | 0.00 | 0.00 | 4.74 | 0.00 |
| | | family 25 (mitochondrial carri [symbol] | | | | | | | | | | |
| CACCACCACG | Hs.5862 | hypothetical protein | 0.00 | 0.19 | 0.78 | 0.90 | 0.00 | 0.18 | 0.00 | 0.00 | 4.74 | 0.78 |
| TGAGGACACA | Hs.139709 | hypothetical protein FLJ12572 | 0.00 | 0.19 | 0.00 | 0.90 | 0.00 | 0.18 | 0.00 | 0.00 | 4.74 | 0.00 |
| TGGCTTGCTC | AB034205 | acid-inducible phosphoprotein or cisplatin resistance-associated overexpressed protein | 0.74 | 0.39 | 0.39 | 1.81 | 0.20 | 0.18 | 0.33 | 0.00 | 4.64 | 0.53 |
| CTAGAACATC | Hs.81424 | ubiquitin-like 1 (sentrin) | 0.55 | 0.19 | 0.20 | 0.90 | 0.20 | 0.00 | 0.00 | 0.00 | 4.74 | 0.36 |
| AACATCAAAC | AF006088 | actin related protein 2/3 complex, subunit 5 (16 kD), Arp2/3 protein complex subunit | 0.37 | 0.19 | 0.20 | 0.90 | 0.20 | 0.00 | 0.33 | 0.00 | 4.74 | 0.54 |
| TACCTGTGCC | Hs.75968 | thymosin, beta 4, X chromosome | 0.37 | 0.19 | 0.78 | 0.90 | 0.20 | 0.00 | 0.33 | 0.00 | 4.74 | 2.11 |
| AAGAACTAAA | AY007148 | hypothetical protein | 0.55 | 0.19 | 0.00 | 0.90 | 0.00 | 0.35 | 0.65 | 0.00 | 4.74 | 0.00 |
| TGGCGTGGCC | Hs.8854 | Human transcription unit PVT gene, exons I-III | 0.55 | 0.19 | 0.39 | 0.90 | 0.00 | 0.35 | 0.00 | 0.00 | 4.74 | 0.71 |
| TAACTTGTGA | Hs.28777 | H2A histone family, member L | 4.06 | 1.16 | 3.53 | 5.42 | 1.17 | 1.24 | 2.60 | 0.97 | 4.67 | 0.87 |
| CATTTTTCCC | Hs.90336 | ATPase, H+ transporting, lysosomal (vacuolar proton pump), member J | 0.92 | 0.19 | 0.20 | 0.90 | 0.39 | 0.00 | 0.65 | 0.00 | 4.74 | 0.22 |
| ATCAGCAAGT | Hs.178485 | Homo sapiens cDNA FLJ13919 fis, clone Y79AA1000410 | 0.00 | 0.19 | 0.39 | 0.90 | 0.39 | 0.00 | 0.33 | 0.00 | 4.74 | 0.39 |
| CAAGACGGGG | AB037729 | RALGDS; ral guanine nucleotide dissociation stimulator | 1.11 | 0.19 | 0.78 | 0.90 | 0.00 | 0.53 | 1.63 | 3.86 | 4.74 | 0.70 |
| TTGTTATATT | AF208844 | hypothetical protein | 1.48 | 0.19 | 1.37 | 0.90 | 0.20 | 0.35 | 0.33 | 0.32 | 4.74 | 0.93 |
| GAGCTGTTGG | L25851 | ITGAE; integrin alpha E (antigen CD103, human mucosal lymphocyte antigen 1; alpha polypeptide) | 1.11 | 0.19 | 0.98 | 0.90 | 0.39 | 0.18 | 0.00 | 0.32 | 4.74 | 0.88 |
| TAATATTTTT | D17110 | actinin, alpha 4 | 0.92 | 0.19 | 0.98 | 0.90 | 0.20 | 0.35 | 0.00 | 0.00 | 4.74 | 1.07 |
| CTGGGGAGTG | AF160212 | VAMP (vesicle-associated membrane protein)-associated protein B and C | 0.37 | 0.19 | 0.39 | 0.90 | 0.20 | 0.35 | 0.00 | 0.00 | 4.74 | 1.05 |
| AACCAGAATG | AF243433 | emopamil binding related protein, delta8-delta7 sterol isomerase related protein | 0.18 | 0.19 | 0.39 | 0.90 | 0.20 | 0.35 | 0.00 | 0.32 | 4.74 | 2.17 |
| GCCTTAAAAA | Hs.104222 | hypothetical protein FLJ10702 or Homo sapiens mRNA; cDNA DKFZp566L034 (from clone | 0.00 | 0.19 | 0.59 | 0.90 | 0.39 | 0.18 | 0.33 | 0.00 | 4.74 | 0.59 |
| | | DKFZp566L034) | | | | | | | | | | |
| AGCCACCACA | AK001160 | hypothetical protein FLJ10298 | 0.74 | 0.19 | 0.98 | 0.90 | 0.00 | 0.71 | 0.33 | 0.00 | 4.74 | 1.32 |
| GAACCCTGGG | BC001182 | hypothetical protein MGC2616 | 1.29 | 0.39 | 1.37 | 1.81 | 0.59 | 0.89 | 1.30 | 0.00 | 4.64 | 1.06 |
| AGGTGCCTCG | AL031714 | ubiquitin-conjugating enzyme E2I (homologous to yeast UBC9) | 0.00 | 0.19 | 0.39 | 0.90 | 0.39 | 0.35 | 0.65 | 0.32 | 4.74 | 0.39 |
| AATCTGCGCC | M13755 | interferon-stimulated protein, 15 kDa | 0.55 | 0.39 | 0.39 | 1.81 | 0.98 | 0.53 | 1.30 | 0.32 | 4.64 | 0.71 |
| AGGTCAAAAA | Hs.158196 | actin related protein 2/3 complex; subunit 4 (20 kD) | 0.18 | 0.19 | 0.59 | 0.90 | 0.39 | 0.53 | 0.00 | 0.00 | 4.74 | 3.28 |
| GTTCAAAGAC | Z24725 | mitogen inducible 2, mig-2 | 0.55 | 0.19 | 0.20 | 0.90 | 0.59 | 0.35 | 0.33 | 0.64 | 4.74 | 0.36 |
| AAGGTGCCTC | BC007913 | hypothetical protein MGC14421 | 0.37 | 0.19 | 0.98 | 0.90 | 0.20 | 0.89 | 0.33 | 0.97 | 4.74 | 2.65 |
| ATGGGCACTG | AF173378 | 60S acidic ribosomal protein PO | 0.18 | 0.19 | 0.39 | 0.90 | 0.20 | 0.89 | 0.00 | 0.00 | 4.74 | 2.17 |
| CAGCTGTAGT | D79996 | KIAA0174 gene product | 0.55 | 0.19 | 0.20 | 0.90 | 0.98 | 0.35 | 0.33 | 0.00 | 4.74 | 0.36 |
| GGGTTTGAAC | AF167572 | skb1 (S. pombe) homolog, methyltransferase (JBP1) | 0.55 | 0.19 | 0.98 | 0.90 | 0.59 | 0.89 | 0.00 | 0.64 | 4.74 | 1.78 |
| TATTTATTCC | D89077 | (11nt = T) Src-like-adapter or diaphorase | 1.11 | 0.19 | 0.78 | 0.90 | 0.78 | 0.71 | 0.00 | 0.00 | 4.74 | 0.70 |
| AGGGTGAAAC | U30825 | splicing factor, arginine/serine-rich 9 | 0.55 | 0.19 | 0.78 | 0.90 | 0.39 | 1.24 | 0.33 | 1.61 | 4.74 | 1.42 |
| TGGAACAGGA | X89750 | TG-interacting factor (TALE family homeobox) | 0.18 | 0.19 | 0.59 | 0.90 | 0.98 | 0.89 | 0.98 | 1.29 | 4.74 | 3.28 |
| TGATCTTTTC | AF012281 | PDZ domain containing 1 | 1.84 | 0.19 | 0.59 | 0.90 | 0.00 | 0.00 | 0.33 | 0.00 | 4.74 | 0.32 |
| GACACTGAAA | BC000006 | ATPase, Na+/K+ transporting, beta 1 polypeptide | 0.74 | 0.19 | 0.20 | 0.90 | 0.00 | 0.00 | 0.00 | 0.00 | 4.74 | 0.27 |
| ATTCCAAGAA | BC005878 | hypothetical protein FLJ20311 | 0.37 | 0.19 | 0.59 | 0.90 | 0.00 | 0.00 | 0.00 | 0.00 | 4.74 | 1.59 |
| CCTCAGCCAG | X55177 | EDN2; endothelin 2 | 0.37 | 0.19 | 0.59 | 0.90 | 0.00 | 0.00 | 0.33 | 0.00 | 4.74 | 1.59 |
| GCTAATAGTA | Hs.182937 | peptidylprolyl isomerase A (cyclophilin A) | 0.37 | 0.19 | 0.39 | 0.90 | 0.00 | 0.00 | 0.33 | 0.00 | 4.74 | 1.05 |
| TAAATAAAGG | AF161503 | HSPC154 protein | 0.18 | 0.19 | 0.59 | 0.90 | 0.00 | 0.00 | 0.00 | 0.32 | 4.74 | 3.28 |
| GCTCATTAAA | Hs.112237 | ESTs | 0.18 | 0.19 | 0.00 | 0.90 | 0.00 | 0.00 | 0.33 | 0.64 | 4.74 | 0.00 |
| TTTTATAGCT | Hs.125346 | ESTs | 0.18 | 0.19 | 0.00 | 0.90 | 0.00 | 0.00 | 0.00 | 0.00 | 4.74 | 0.00 |
| TAAAACGTGA | AL157954 | Novel human mRNA from chromosome 1, clone Z98884. | 0.00 | 0.19 | 0.20 | 0.90 | 0.00 | 0.00 | 0.00 | 0.00 | 4.74 | 0.20 |
| AAGGTAACTA | NM_000791 | dihydrofolate reductase | 0.00 | 0.19 | 0.20 | 0.90 | 0.00 | 0.00 | 0.00 | 0.00 | 4.74 | 0.20 |
| AAAGATTGGA | U58130 | solute carrier family 12 (sodium/potassium/chloride transporters), member 1 | 0.00 | 0.19 | 0.20 | 0.90 | 0.00 | 0.00 | 0.00 | 0.00 | 4.74 | 0.20 |
| CCTGTACCCC | AF072836 | high-mobility group 20B | 0.55 | 0.19 | 0.59 | 0.90 | 0.78 | 1.60 | 0.65 | 0.00 | 4.74 | 1.07 |
| TAGACTTATT | M22632 | glutamic-oxaloacetic transaminase 2, mitochondrial (aspartate aminotransferase 2) | 1.29 | 0.19 | 0.39 | 0.90 | 1.17 | 1.24 | 0.33 | 0.00 | 4.74 | 0.30 |
| GGGGTCTGGG | AK000852 | hypothetical protein PP2447 | 0.37 | 0.19 | 0.00 | 0.90 | 1.17 | 1.77 | 0.33 | 0.00 | 4.74 | 0.00 |
| AGCTGGAGTC | U78518 | cAMP-binding guanine nucleotide exchange fac | 0.37 | 0.19 | 0.39 | 0.90 | 2.74 | 1.60 | 2.93 | 1.61 | 4.74 | 1.05 |
| CGACCGTGGC | AF070665 | HSPC009 protein | 0.92 | 0.19 | 0.39 | 0.90 | 1.76 | 2.84 | 0.65 | 0.64 | 4.74 | 0.42 |
| AAGGACCTTT | AF304163 | SH3 domain binding glutamic acid-rich protein like 3 | 1.29 | 0.19 | 0.78 | 0.90 | 3.72 | 1.24 | 3.25 | 0.97 | 4.74 | 0.60 |
| TCTGCCTGGG | Hs.24379 | MUM2 protein | 0.74 | 0.19 | 1.18 | 0.90 | 4.11 | 2.31 | 4.23 | 3.86 | 4.74 | 1.59 |
| TGTTTGGGGG | NM_024656 | hypothetical protein FLJ22329 | 1.11 | 0.58 | 0.39 | 2.53 | 0.39 | 0.53 | 0.33 | 0.32 | 4.36 | 0.35 |
| TTTGAGGATT | Hs.147916 | DEAD/H (Asp-Glu-Ala-Asp/His) box polypeptide 3 or thioredoxin-like, 32 kD | 0.92 | 0.39 | 0.39 | 1.63 | 0.20 | 0.00 | 0.33 | 0.32 | 4.18 | 0.42 |
| CAGGCTACTG | AF102265 | N-acetylglucosamine-phosphate mutase | 0.92 | 0.39 | 0.59 | 1.63 | 0.20 | 0.18 | 0.65 | 0.32 | 4.18 | 0.64 |
| CAAACCTTTA | Hs.75133 | transcription factor 6-like 1 (mitochondrial transcription factor 1-like) or others | 1.48 | 0.39 | 0.98 | 1.63 | 0.20 | 0.35 | 0.33 | 0.00 | 4.18 | 0.66 |
| AGTTGAAATT | AK000752 | KIAA1181 protein | 0.37 | 0.39 | 0.59 | 1.63 | 0.39 | 0.18 | 0.00 | 0.00 | 4.18 | 1.59 |
| TTCCAGACCT | Y00282 | glyceraldehyde-3-phosphate dehydrogenase or ribophorin II | 2.40 | 0.77 | 1.37 | 3.25 | 0.39 | 1.06 | 0.33 | 0.32 | 4.22 | 0.57 |
| CACACCATTG | Hs.136644 | CS box-containing WD protein | 0.74 | 0.39 | 1.37 | 1.63 | 0.39 | 0.53 | 0.33 | 0.00 | 4.18 | 1.85 |
| GAAAGGTCTG | Hs.118778 | KDEL (Lys-Asp-Glu-Leu) endoplasmic reticulum protein retention receptor 2 | 1.11 | 0.39 | 2.55 | 1.63 | 0.20 | 0.89 | 1.30 | 0.00 | 4.18 | 2.30 |
| GTTTTCATTC | AF100746 | ancient ubiquitous protein 1 | 1.66 | 0.39 | 1.18 | 1.63 | 0.98 | 0.18 | 0.00 | 0.32 | 4.18 | 0.71 |
| ATGGGCTTGA | AF042284 | CGI-44 protein; sulfide dehydrogenase like (yeast) | 0.55 | 0.39 | 0.59 | 1.63 | 2.74 | 2.31 | 1.95 | 2.25 | 4.18 | 1.07 |
| GTGCCTAGGG | Hs.12854 | ATRAP protein | 1.11 | 0.58 | 0.98 | 2.35 | 1.17 | 2.13 | 0.00 | 1.93 | 4.05 | 0.88 |

4 genes in FIG. 4(i)
SAGE data for genes up-regulated ≧ 4-fold by hypoxia in 786-0 cells, regardless of VHL status (VHL⁺, V3 and VHL⁻, V4) with respect
to 786-0 cells grown under nomoxia (VHL⁺, V1 and VHL⁻, V2). SAGE tag abundances were normalized before calculating ratio, as shown. The table is presented in descending order of V4/V2.

| | | | | | | | | | | | | |
|---|---|---|---|---|---|---|---|---|---|---|---|---|
| CCCAGCTAAT | AF339827 | ATPase, H+ transporting, lysosomal (vacuolar proton pump), alpha polypeptide, 70 kD, isoform 1 (11nt) or | 0.18 | 0.19 | 1.18 | 1.26 | 0.20 | 0.18 | 0.33 | 0.32 | 6.63 | 6.56 |
| | | FOXJ2 forkhead factor (11r [symbol] | | | | | | | | | | |
| TAGTAAGTCA | AL110153 | Homo sapiens, clone IMAGE: 3834726, mRNA | 0.18 | 0.19 | 0.98 | 0.90 | 0.00 | 0.18 | 0.00 | 0.00 | 4.74 | 5.44 |
| CAGAGTTGTA | AB014733 | hypothetical protein AF140225 | 0.18 | 0.19 | 0.98 | 0.90 | 0.59 | 0.18 | 0.33 | 0.32 | 4.74 | 5.44 |
| TCAGTTTGTC | U68566 | HAX1; HS1 binding protein | 0.37 | 0.39 | 1.96 | 1.63 | 2.74 | 2.13 | 0.98 | 1.29 | 4.18 | 5.30 |

156 genes in FIG. 4 (137 genes; 19 tags with no matches not shown)
SAGE data for genes up-regulated ≧ 4-fold by hypoxia in 786-0 VHL⁺ Hyp. (V3) when compared to 786-0 VHL⁺ Nor. (V1).
Also shown, for comparison, is the SAGE data for each gene in 786-0 VHL⁻ Hyp. (V4) when compared to 786-0 VHL⁻ Nor. (V2).
SAGE tag abundances were normalized before calculating ratios, as shown. The table is presented in descending order of V3/V1.

| | | | | | | | | | | | | |
|---|---|---|---|---|---|---|---|---|---|---|---|---|
| GTGGTGTGTG | AB033023 | nuclear factor of activated T-cells, cytoplasmic, calcineurin-dependent 3 or others | 0.18 | 0.58 | 1.76 | 0.54 | 0.39 | 0.18 | 0.00 | 0.97 | 9.78 | 0.93 |
| GACCCACTAC | J03569 | SLC3A2; solute carrier family 3 (activators of dibasic and neutral amino acid transport), member 2 | 0.18 | 0.19 | 1.76 | 0.18 | 0.20 | 0.18 | 0.33 | 1.29 | 9.78 | 0.95 |
| GTAAAAGTTC | D21262 | nucleolar and coiled-body phosphprotein 1 | 0.18 | 0.39 | 1.57 | 0.54 | 0.20 | 0.18 | 0.00 | 0.00 | 8.72 | 1.38 |
| AGAATGTACG | AL049851 | hypothetical protein | 0.37 | 1.35 | 2.94 | 1.81 | 0.20 | 0.35 | 0.33 | 0.64 | 7.95 | 1.34 |
| GAGCCGCCTC | AK001809 | HSPC182 protein | 0.18 | 0.39 | 1.37 | 0.90 | 0.59 | 1.06 | 0.00 | 0.32 | 7.61 | 2.31 |
| CCACAGGAGA | AJ251830 | p53-induced protein PIGPC1; THW tumor suppressor | 0.18 | 0.39 | 1.37 | 0.90 | 1.17 | 1.77 | 1.95 | 2.58 | 7.61 | 2.31 |
| GTGAACTTAC | AB013803 | contactin 5; NB-2; hNB-2 | 0.18 | 0.00 | 1.37 | 0.36 | 0.59 | 0.18 | 0.98 | 0.00 | 7.61 | 0.36 |
| AGGAAGAGCC | AF125535 | pp21 homolog | 0.18 | 1.16 | 1.18 | 0.00 | 0.39 | 0.00 | 0.65 | 0.64 | 6.56 | 0.00 |
| GTTGTGATGT | X66899 | Ewing sarcoma breakpoint region 1 | 0.18 | 0.97 | 1.18 | 1.08 | 0.00 | 0.35 | 0.00 | 0.00 | 6.56 | 1.11 |
| GTGCTTGTAC | AB001106 | GMFB; glia maturation factor, beta | 0.18 | 1.35 | 1.18 | 1.08 | 0.20 | 0.35 | 0.33 | 0.97 | 6.56 | 0.80 |
| ACCTGCTGGT | AK000447 | GTPase Rab14 | 0.18 | 1.16 | 1.18 | 0.00 | 0.20 | 0.35 | 0.00 | 0.97 | 6.56 | 0.00 |
| AGGGAGGCAG | Hs.74368 | transmembrane protein (63 kD), endoplasmic reticulum/Golgi intermediate compartment | 0.18 | 0.77 | 1.18 | 0.90 | 0.39 | 0.00 | 0.33 | 0.00 | 6.56 | 1.17 |
| AGCAACAGTG | U10117 | SCYE1; small inducible cytokine subfamily E, member 1 (endothelial monocyte-activating) | 0.18 | 0.77 | 1.18 | 0.18 | 0.39 | 0.00 | 0.33 | 0.97 | 6.56 | 0.23 |
| ATGAAACCCC | U67806 | small inducible cytokine A5 (RANTES) or others | 0.37 | 1.16 | 2.35 | 0.72 | 0.59 | 0.35 | 0.00 | 0.64 | 6.35 | 0.62 |
| CAGCTGGGGC | X66975 S41 | polypyrimidine tract binding protein (heterogeneous nuclear ribonucleoprotein I) | 0.18 | 0.77 | 1.18 | 0.18 | 1.37 | 0.71 | 0.00 | 0.00 | 6.56 | 0.23 |
| TAGTCATCTT | U68758 | pyrroline-5-carboxylate synthetase (glutamate gamma-semialdehyde synthetase) | 0.18 | 0.58 | 1.18 | 0.18 | 0.00 | 0.00 | 0.00 | 0.00 | 6.56 | 0.31 |
| ACGGCTCCGA | Hs.48563 | ESTs | 0.18 | 0.77 | 1.18 | 0.90 | 2.15 | 0.71 | 0.98 | 0.97 | 6.56 | 1.17 |
| TGGGCTCTGA | D12676 | CD36 antigen (collagen type I receptor, thrombospondin receptor)-like 2 (lysosomal integral membrane | 0.18 | 0.77 | 1.18 | 0.90 | 0.98 | 2.13 | 0.98 | 1.61 | 6.56 | 1.17 |
| | | protein II) | | | | | | | | | | |
| GGAAGTCCTG | Hs.118787 | transforming growth factor, beta-induced, 68 kD | 0.18 | 0.39 | 1.18 | 1.08 | 0.00 | 0.00 | 0.33 | 0.00 | 6.56 | 2.77 |
| GCCAAGATGC | BC004944 | Similar to RIKEN cDNA 2310040G17 gene | 0.18 | 0.58 | 1.18 | 1.44 | 4.50 | 2.48 | 1.63 | 1.29 | 6.56 | 2.48 |
| ATGCAAGAGA | AB051504 | KIAA1717 protein | 0.18 | 0.00 | 1.18 | 0.00 | 0.00 | 0.00 | 0.00 | 0.00 | 6.56 | 0.00 |
| CCAGTGTGCA | Hs.249247 | heterogeneous nuclear protein similar to rat helix destabilizing protein | 0.18 | 0.00 | 1.18 | 0.36 | 0.00 | 0.00 | 0.00 | 0.00 | 6.56 | 0.36 |
| CTCACTTCTT | Hs.165998 | DKFZP564M2423 protein | 0.18 | 0.97 | 0.98 | 0.90 | 0.20 | 0.00 | 0.98 | 0.00 | 5.44 | 0.93 |
| GCTAGGTATT | AJ250865 | testin | 0.18 | 0.77 | 0.98 | 1.08 | 0.00 | 0.18 | 0.33 | 0.00 | 5.44 | 1.40 |
| CTAAGGTGGG | L76702 | protein phosphatase 2, regulatory subunit B (B56), delta isoform | 0.18 | 0.97 | 0.98 | 0.18 | 0.00 | 0.35 | 0.33 | 0.64 | 5.44 | 0.19 |
| TGGAAATAAA | BC000547 | mitochondrial ribosomal protein S6 | 0.18 | 0.77 | 0.98 | 0.36 | 0.00 | 0.35 | 0.00 | 0.64 | 5.44 | 0.47 |
| GGTGCTCCCT | AB015633 | transmembrane protein 5 | 0.18 | 0.39 | 0.98 | 0.00 | 0.20 | 0.00 | 0.33 | 0.32 | 5.44 | 0.00 |
| GGGCGGGGGC | M81735 | polymerase (DNA directed), delta 1, catalytic subunit (125 kD) | 0.18 | 0.97 | 0.98 | 1.99 | 0.39 | 0.18 | 0.00 | 0.00 | 5.44 | 2.05 |
| AACTCCCAGT | AL050044 | GADD45B; growth arrest and DNA-damage-inducible, beta | 0.18 | 0.39 | 0.98 | 0.72 | 0.39 | 0.00 | 0.65 | 0.97 | 5.44 | 1.85 |
| GAGGAGTGGG | Hs.206770 | zinc finger protein 297 | 0.18 | 0.19 | 0.98 | 0.36 | 0.20 | 0.00 | 0.00 | 0.00 | 5.44 | 1.89 |
| AATAGGGTCA | AF086183 | amyloid beta (A4) precursor-like protein 2 (EST) | 0.37 | 1.16 | 1.96 | 0.90 | 0.59 | 0.71 | 0.00 | 0.00 | 5.30 | 0.78 |
| CCACGTTCCA | Y00691 M27 | ENO2; enolase 2, (gamma, neuronal) | 0.18 | 1.55 | 0.98 | 1.63 | 0.78 | 1.06 | 5.85 | 16.74 | 5.44 | 1.05 |
| GGCTGCAGTC | AL110253 | double ring-finger protein, Dorfin | 0.18 | 0.39 | 0.98 | 0.00 | 0.59 | 0.00 | 0.65 | 0.97 | 5.44 | 0.00 |
| ATGCGCAAGG | Z36785 | ribosomal protein, large, P0 | 0.18 | 1.55 | 0.98 | 0.90 | 1.96 | 1.77 | 0.98 | 1.61 | 5.44 | 0.58 |
| GGAACGGATG | AJ131186 | nuclear matrix protein NMP200 related to splicing factor PRP19 | 0.18 | 0.39 | 0.98 | 0.00 | 0.78 | 0.18 | 0.65 | 0.00 | 5.44 | 0.00 |
| CTTTCTTCCC | X79067 X71 | ZFP36L1; Zinc finger protein 36, C3H type-like 1; butyrate response factor 1 (EGF-response factor 1) | 0.18 | 0.39 | 0.98 | 0.36 | 0.98 | 0.00 | 1.30 | 1.61 | 5.44 | 0.92 |
| GTGCCTAGGA | Hs.25999 | hypothetical protein FLJ22195 | 0.18 | 1.16 | 0.98 | 1.63 | 0.78 | 2.13 | 1.95 | 1.29 | 5.44 | 1.41 |
| GTCTTTCTGG | AB046781 | uveal autoantigen with coiled coil domains and ankyrin repeats | 0.18 | 0.77 | 0.98 | 0.54 | 0.00 | 0.00 | 0.00 | 0.64 | 5.44 | 0.70 |
| GCTACTATTA | Hs.154443 | minichromosome maintenance deficient (S. cerevisiae) 4 | 0.18 | 0.77 | 0.98 | 0.54 | 0.00 | 0.00 | 0.00 | 0.00 | 5.44 | 0.70 |
| TTCTCTCAAC | AF089106 | unknown | 0.18 | 0.77 | 0.98 | 0.72 | 0.78 | 1.42 | 2.28 | 0.97 | 5.44 | 0.94 |
| TAAACTTCAA | Hs.87627 | disrupter of silencing 10 | 0.18 | 0.19 | 0.98 | 0.54 | 0.20 | 0.35 | 0.65 | 0.00 | 5.44 | 2.84 |
| GTGCAAAATG | Hs.247280 | HBV associated factor | 0.18 | 0.39 | 0.98 | 1.08 | 0.39 | 1.06 | 1.95 | 2.90 | 5.44 | 2.77 |
| GCTATGCTCC | BC003653 | presenilins associated rhomboid-like protein | 0.18 | 0.39 | 0.98 | 0.72 | 0.78 | 0.71 | 0.33 | 0.97 | 5.44 | 1.85 |
| CAGAAGAGGC | AK001528 | DIGeorge syndrome critical region gene 6 | 0.18 | 0.97 | 0.98 | 0.36 | 1.96 | 2.13 | 0.98 | 0.97 | 5.44 | 0.37 |
| GAGGTGCTCT | AF220050 | uncharacterized hemalopoietic stem/progenitor cells protein MDS030 | 0.18 | 0.39 | 0.98 | 0.00 | 1.17 | 0.53 | 0.65 | 0.32 | 5.44 | 0.00 |
| ATGGTCTACG | AK000254 | hypothetical protein | 0.18 | 0.39 | 0.98 | 0.18 | 1.17 | 0.71 | 2.60 | 0.97 | 5.44 | 0.46 |
| TAATGACAAT | U60115 | four and a half LIM domains 1 | 0.18 | 0.39 | 0.98 | 0.36 | 0.00 | 0.00 | 0.00 | 0.00 | 5.44 | 0.92 |
| AAGGAGTTTG | M33374 | NADH dehydrogenase (ubiquinone) 1 beta subcomplex, 7 (18 kD, B18) | 0.37 | 0.39 | 1.96 | 0.90 | 2.15 | 1.06 | 0.33 | 0.97 | 5.30 | 2.31 |
| GTGGTATGGC | AF151076 | hypothetical protein | 0.18 | 0.19 | 0.98 | 0.36 | 0.98 | 1.06 | 0.33 | 0.97 | 5.44 | 1.89 |
| TGGGGAAACT | X52333 | APMCF1 protein (EST) | 0.18 | 0.00 | 0.98 | 0.18 | 0.20 | 0.00 | 0.00 | 0.00 | 5.44 | 0.18 |
| GCACAAGTAA | AF339775 | unknown | 0.18 | 0.00 | 0.98 | 0.90 | 0.20 | 0.35 | 0.65 | 0.00 | 5.44 | 0.90 |
| GCAAACAAAA | Hs.429 | ATP synthase, H+ transporting, mitochondrial F0 complex, subunit c (subunit 9) isoform 3 | 0.18 | 0.00 | 0.98 | 0.18 | 0.59 | 0.71 | 0.00 | 0.00 | 5.44 | 0.18 |
| ACAAATCCTT | M34539 | FK506-binding protein 1A (12 kD) | 0.18 | 0.00 | 0.98 | 0.72 | 1.96 | 0.00 | 2.28 | 0.64 | 5.44 | 0.72 |
| GAGTGCAGGT | M25077 | Sjogren syndrome antigen A2 (60 kD, ribonucleoprotein autoantigen SS-A/Ro | 0.18 | 0.00 | 0.98 | 0.18 | 0.00 | 0.00 | 0.00 | 0.32 | 5.44 | 0.18 |
| CGCTTTTGTA | AL110124 | DKFZP564A2416 protein | 0.55 | 2.90 | 2.75 | 1.81 | 0.98 | 1.77 | 1.95 | 1.61 | 5.00 | 0.62 |
| GAGTAGAGAA | Hs.145279 | SET translocation (myeloid leukemia-associated) | 0.37 | 1.55 | 1.76 | 1.44 | 0.39 | 0.35 | 0.33 | 0.32 | 4.76 | 0.93 |
| TACTGTGGAT | Hs.21537 | protein phosphatase 1, catalytic subunit, beta isoform | 0.37 | 1.93 | 1.76 | 0.90 | 0.78 | 0.53 | 0.65 | 0.97 | 4.76 | 0.47 |
| TATGTGCCAC | AK001323 | hypothetical protein FLJ10461 | 0.55 | 1.93 | 2.35 | 0.36 | 0.20 | 0.00 | 0.00 | 0.00 | 4.27 | 0.19 |
| GTAAAACCCT | AF232009 | HSA250303; peroxisomal trans 2-enoyl CoA reductase; putative short chain alcohol dehydrogenase | 0.18 | 0.77 | 0.78 | 0.00 | 0.20 | 0.00 | 0.00 | 0.32 | 4.33 | 0.00 |
| TGTGACCTCT | AF061729 | dolichyl-phosphate mannosyltransferase polypeptide 2, regulatory subunit | 0.18 | 0.58 | 0.78 | 0.18 | 0.00 | 0.18 | 0.00 | 0.00 | 4.33 | 0.31 |
| AAGACAAGTG | Hs.184109 | ribosomal protein L37a | 0.18 | 0.58 | 0.78 | 0.18 | 0.00 | 0.18 | 0.00 | 0.00 | 4.33 | 0.31 |
| CTGTTGGCAT | Hs.184108 | ribosomal protein L21 (gene or pseudogene) | 0.18 | 1.74 | 0.78 | 1.08 | 0.20 | 0.35 | 0.33 | 1.61 | 4.33 | 0.62 |
| AACGGGCCGG | Hs.26484 | HIRA-Interacting protein 3 | 0.18 | 1.55 | 0.78 | 0.54 | 0.39 | 0.18 | 0.33 | 0.32 | 4.33 | 0.35 |
| TTCTGGCACT | AF161402 | DKFZP566C134 protein | 0.18 | 0.97 | 0.78 | 1.08 | 0.20 | 0.18 | 0.00 | 0.00 | 4.33 | 1.11 |
| GTGAAGTCTT | AF055031 | solute carrier family 3 (activators of dibasic and neutral amino acid transport), member 2 | 0.37 | 1.55 | 1.57 | 0.72 | 0.39 | 0.35 | 0.98 | 0.97 | 4.24 | 0.46 |
| GCGAGCAGCG | Hs.300141 | ribosomal protein L39 | 0.18 | 0.39 | 0.78 | 0.72 | 0.20 | 0.00 | 0.00 | 0.64 | 4.33 | 1.85 |
| CACTGAGCCA | AK000034 | DnaJ (Hsp40) homolog, subfamily B, member 12 | 0.37 | 1.35 | 1.57 | 1.08 | 0.00 | 0.71 | 0.33 | 0.32 | 4.24 | 0.80 |
| GCCAGGGGCC | BC002542 | KIAA1273 protein | 0.18 | 0.58 | 0.78 | 0.18 | 0.20 | 0.18 | 0.00 | 0.00 | 4.33 | 0.31 |
| CAGAGTGACT | L07758 | nuclear phosphoprotein similar to S. cerevisiae PWP1 | 0.18 | 0.58 | 0.78 | 1.26 | 0.39 | 0.00 | 0.33 | 0.32 | 4.33 | 2.17 |
| CTATATTTTT | X87212 | cathepsin C | 0.18 | 0.58 | 0.78 | 0.18 | 0.39 | 0.00 | 0.00 | 0.00 | 4.33 | 0.31 |
| GAGAATCTGC | AF075110 | chromosome 14 open reading frame 4 | 0.18 | 0.77 | 0.78 | 0.72 | 0.39 | 0.18 | 0.00 | 0.00 | 4.33 | 0.94 |
| AAAAACCATA | U21242 | general transcription factor IIA, 2 (12 kD subunit) | 0.18 | 0.77 | 0.78 | 0.54 | 0.59 | 0.00 | 0.00 | 0.00 | 4.33 | 0.70 |
| CGGGATGCAG | Hs.155560 | calnexin | 0.18 | 0.97 | 0.78 | 1.08 | 0.59 | 0.18 | 1.95 | 1.29 | 4.33 | 1.11 |
| ATGTGAGGGA | AK000510 | tRNA selenocysteine associated protein | 0.18 | 0.39 | 0.78 | 0.36 | 0.00 | 0.35 | 0.65 | 0.97 | 4.33 | 0.92 |
| CCTGAATCTG | Hs.179779 | ribosomal protein L37 | 0.18 | 0.58 | 0.78 | 0.36 | 0.20 | 0.35 | 0.00 | 0.00 | 4.33 | 0.62 |
| CAGAAGTGTC | D13640 | KIAA0015 gene product | 0.18 | 0.58 | 0.78 | 0.54 | 0.39 | 0.18 | 0.65 | 0.00 | 4.33 | 0.93 |
| GCTCTGTAAG | AK000222 | putative methyltransferase | 0.18 | 0.39 | 0.78 | 0.72 | 0.20 | 0.18 | 0.00 | 0.32 | 4.33 | 1.85 |
| TCTCTAGAAT | X75342 | SHB adaptor protein (a Src homology 2 protein) | 0.18 | 0.39 | 0.78 | 0.36 | 0.20 | 0.18 | 0.00 | 0.00 | 4.33 | 0.92 |
| CTTATGATCA | AJ238095 | Lsm3 protein | 0.18 | 0.97 | 0.78 | 0.54 | 0.78 | 0.18 | 0.33 | 0.00 | 4.33 | 0.56 |
| TGCAAAAAAA | BC000610 | hypothetical protein FLJ10986 | 0.18 | 0.39 | 0.78 | 0.18 | 0.39 | 0.00 | 0.33 | 0.00 | 4.33 | 0.46 |
| GTTGTGGCCA | Hs.165563 | Homo sapiens cDNA FLJ13070 fis, clone NT2RP3001777 | 0.18 | 0.19 | 0.78 | 0.36 | 0.20 | 0.00 | 0.00 | 0.32 | 4.33 | 1.89 |
| TTTAATTGTG | Hs.145279 | SET translocation (myeloid teukemia-associated) or etoposide-induced mRNA | 0.18 | 0.19 | 0.78 | 0.00 | 0.20 | 0.00 | 0.33 | 0.00 | 4.33 | 0.00 |
| GTGGTGGGTG | X58236 | solute carrier family 14 (urea transporter), member 1 (Kidd blood group) | 0.18 | 1.16 | 0.78 | 0.90 | 0.39 | 0.89 | 0.65 | 0.32 | 4.33 | 0.78 |
| CTGAGCTGTA | AK001080 | WD repeat domain 6 | 0.18 | 0.58 | 0.78 | 0.18 | 0.39 | 0.35 | 0.33 | 0.32 | 4.33 | 0.31 |
| TGTGTGGGGC | AK024450 | unknown | 0.18 | 0.58 | 0.78 | 0.00 | 0.39 | 0.35 | 0.00 | 0.00 | 4.33 | 0.00 |
| TGTGAGCCCC | AF265209 | enigma (LIM domain protein) | 0.37 | 0.58 | 1.57 | 0.36 | 0.59 | 0.18 | 0.00 | 0.00 | 4.24 | 0.62 |
| CCGGACCTGT | Hs.277777 | paired immunoglobulin-like receptor beta | 0.18 | 1.16 | 0.78 | 1.08 | 1.17 | 0.35 | 0.98 | 0.97 | 4.33 | 0.93 |
| CGTGAACAAT | AF226049 | GL004 protein | 0.18 | 0.39 | 0.78 | 0.18 | 0.20 | 0.35 | 0.00 | 0.32 | 4.33 | 0.46 |
| AGCCTCGGGC | Hs.75922 | brain protein I3 | 0.18 | 0.39 | 0.78 | 0.72 | 0.39 | 0.18 | 0.33 | 0.32 | 4.33 | 1.85 |
| TTCTCTCTGT | M57567 | ADP-ribosylation factor 5 | 0.18 | 0.58 | 0.78 | 0.72 | 0.20 | 0.71 | 0.00 | 0.00 | 4.33 | 1.24 |
| CTGCCAAAAA | AF112214 | mitochondrial ribosomal protein L13 | 0.18 | 0.39 | 0.78 | 0.36 | 0.20 | 0.53 | 0.00 | 0.00 | 4.33 | 0.92 |
| CTCTCAATAT | Hs.279518 | amyloid precursor protein homolog HSD-2 or amyloid beta (A4) precursor-like protein 2 | 0.18 | 0.39 | 0.78 | 0.36 | 0.20 | 0.53 | 0.33 | 0.00 | 4.33 | 0.92 |
| AAAATAAAGA | D90373 | APEX nuclease (multifunctional DNA repair enzyme) | 0.18 | 0.58 | 0.78 | 0.36 | 0.39 | 0.71 | 0.65 | 0.64 | 4.33 | 0.62 |
| ACCAAATTAA | AF016266 | tumor necrosis factor raceptor superfamily, member 10b (TNF receptor) | 0.18 | 0.58 | 0.78 | 0.54 | 0.59 | 0.53 | 0.00 | 0.00 | 4.33 | 0.93 |
| AAATAAAAGC | X51521 | thyroid stimulating hormone receptor or villin 2 (ezrin) | 0.18 | 0.39 | 0.78 | 1.08 | 0.59 | 0.18 | 0.33 | 0.64 | 4.33 | 2.77 |
| CTGTTTGTTC | Hs.288965 | Homo sapiens cDNA: FLJ22300 fis, clone HRC04759 | 0.18 | 0.19 | 0.78 | 0.36 | 0.20 | 0.18 | 0.00 | 0.64 | 4.33 | 1.89 |
| GGCTATGCCA | AF147357 | cisplatin resistance-associated overexpressed protein (EST) or others | 0.37 | 0.77 | 1.57 | 2.17 | 0.00 | 0.00 | 0.00 | 0.00 | 4.24 | 2.82 |
| CTGCTGAGCC | Hs.1742 | IQ motif containing GTPase activating protein 1 | 0.18 | 0.39 | 0.78 | 0.54 | 0.78 | 0.53 | 0.33 | 1.29 | 4.33 | 1.38 |
| AACAACAGTG | AK001472 | anillin (Drosophila Scraps homolog), actin binding protein (EST) or others | 0.18 | 0.58 | 0.78 | 0.36 | 0.00 | 0.00 | 0.00 | 0.00 | 4.33 | 0.62 |
| GTTTGGCTGC | Hs.154443 | minichromosome maintenance deficient (S. cerevisiae) 4 | 0.18 | 0.58 | 0.78 | 0.54 | 0.00 | 0.00 | 0.00 | 0.00 | 4.33 | 0.93 |
| GACTAAAAAA | NM_001019 | ribosomal protein S15a | 0.18 | 0.19 | 0.78 | 0.00 | 0.00 | 0.71 | 0.00 | 0.00 | 4.33 | 0.00 |
| TGTTGGGTTC | AB013385 | MAP4K4; mitogen-activated protein kinase kinase kinase kinase 4 | 0.18 | 0.19 | 0.78 | 0.18 | 0.20 | 0.53 | 0.65 | 0.00 | 4.33 | 0.95 |
| GTGGACCCTG | AF132959 | NOSIP (eNOS interacting protein) | 0.18 | 0.39 | 0.78 | 0.36 | 0.78 | 0.71 | 0.65 | 0.32 | 4.33 | 0.92 |
| ACCCCAGCAA | Hs.164036 | glucosamine (N-acetyl)-6-sulfatase (Sanfilippo disease IIID) or putative zinc finger protein | 0.18 | 0.39 | 0.78 | 0.18 | 0.78 | 0.89 | 1.30 | 0.00 | 4.33 | 0.46 |
| GGCGTCCTGG | Hs.44017 | SIR2 (silent mating type information regulation 2, S. cerevisiae, homolog)-like | 0.18 | 0.77 | 0.78 | 0.36 | 1.96 | 1.42 | 0.98 | 0.00 | 4.33 | 0.47 |
| AGGATGGCGG | Hs.182979 | ribosomal protein L12 | 0.55 | 0.58 | 2.35 | 0.90 | 1.37 | 1.42 | 0.98 | 1.29 | 4.27 | 1.55 |
| GAATGCAGTT | Hs.306155 | chorionic somatomammotropin hormone 2 or ESTs | 0.18 | 0.19 | 0.78 | 0.36 | 0.59 | 0.35 | 0.65 | 0.00 | 4.33 | 1.89 |
| GCCAAACTTG | Hs.119598 | ribosomal protein L3 or ALEX3 protein | 0.18 | 0.19 | 0.78 | 0.18 | 0.78 | 0.18 | 0.33 | 0.64 | 4.33 | 0.95 |
| TGGCAAAGTA | Hs.104480 | ESTs | 0.37 | 0.39 | 1.57 | 0.00 | 0.00 | 0.00 | 0.00 | 0.00 | 4.24 | 0.00 |
| AAGCTGTTGT | X63692 | DNA (cytosine-5-)-methyltransferase 1 | 0.18 | 0.39 | 0.78 | 0.18 | 0.00 | 0.00 | 0.00 | 0.00 | 4.33 | 0.46 |
| TGTGGCCAGG | Hs.290357 | ESTs | 0.18 | 0.39 | 0.78 | 0.00 | 0.00 | 0.00 | 0.00 | 0.32 | 4.33 | 0.00 |
| TAACATTGAG | Hs.43756 | ESTs | 0.18 | 0.39 | 0.78 | 0.00 | 0.00 | 0.00 | 0.00 | 0.00 | 4.33 | 0.00 |
| GCCTCCCGCC | BC001211 | kinesin family member C3 | 0.18 | 0.19 | 0.78 | 0.72 | 0.59 | 0.53 | 0.33 | 0.32 | 4.33 | 3.79 |
| AGAATTGTGT | BC004916 | hypothetical protein MGC4767 | 0.18 | 0.19 | 0.78 | 0.18 | 0.39 | 0.89 | 0.33 | 0.00 | 4.33 | 0.95 |
| TGACACCCAC | X17025 | isopentenyl-diphosphate delta isomerase | 0.18 | 0.19 | 0.78 | 0.36 | 0.78 | 0.53 | 1.30 | 0.00 | 4.33 | 1.89 |
| GGTTGAGTGT | Hs.20529 | Homo sapiens cDNA: FLJ21811 fis, clone HEP01037 | 0.18 | 0.39 | 0.78 | 0.36 | 1.76 | 1.06 | 1.95 | 0.32 | 4.33 | 0.92 |
| GCGGCAGTTA | M55268 J02 | CSNK2A2; casein kinase 2, alpha prime polypeptide | 0.18 | 0.19 | 0.78 | 0.36 | 0.59 | 1.06 | 0.33 | 0.64 | 4.33 | 1.89 |
| TGAATACTAC | AB034747 | LPS-Induced TNF-alpha factor | 0.18 | 0.19 | 0.78 | 0.18 | 0.00 | 0.00 | 0.33 | 0.00 | 4.33 | 0.95 |
| TGCATTGTTT | AB040915 | KIAA1482 protein | 0.18 | 0.19 | 0.78 | 0.18 | 0.00 | 0.00 | 0.00 | 0.00 | 4.33 | 0.95 |
| GTGTATATTG | Hs.292669 | ESTs | 0.18 | 0.19 | 0.78 | 0.18 | 0.00 | 0.00 | 0.33 | 0.00 | 4.33 | 0.95 |
| GATGCTGATT | Hs.26410 | ESTs | 0.18 | 0.19 | 0.78 | 0.18 | 0.00 | 0.00 | 0.00 | 0.00 | 4.33 | 0.95 |
| CCTTACTTTA | Hs.75415 | beta-2-microglobulin | 0.18 | 0.19 | 0.78 | 0.18 | 0.00 | 0.00 | 0.00 | 0.00 | 4.33 | 0.95 |
| CCACTTCCTC | Hs.77495 | KIAA0242 protein or ESTs | 0.18 | 0.19 | 0.78 | 0.36 | 0.00 | 0.00 | 0.00 | 0.00 | 4.33 | 1.89 |
| CATCCAAAAC | Hs.245710 | heterogeneous nuclear ribonucleoprotein H1 (H) | 0.18 | 0.19 | 0.78 | 0.72 | 0.00 | 0.00 | 0.00 | 0.00 | 4.33 | 3.79 |
| TTGGGGTTCC | Hs.182771 | vitelliform macular dystrophy (Best disease, bestrophin) or retinoblastoma-binding protein 7 or ferritin, | 0.18 | 0.19 | 0.78 | 0.72 | 0.78 | 1.77 | 1.63 | 1.29 | 4.33 | 3.79 |
| | | heavy polypeptide 1 | | | | | | | | | | |
| GTATCTTAAT | AF155568 | NS1-associated protein 1 | 0.18 | 0.00 | 0.78 | 0.00 | 0.20 | 0.00 | 0.65 | 0.00 | 4.33 | 0.00 |
| ATGAATAAAA | X96650 | microtubule-associated protein, RP/EB family, member 2 or Rho GTPase activating protein 6 | 0.18 | 0.00 | 0.78 | 0.00 | 0.20 | 0.00 | 0.00 | 0.00 | 4.33 | 0.00 |
| GCTAAAAACA | Hs.8107 | Homo sapiens mRNA; cDNA DKFZp586B0918 (from clone DKFZp586B0918) | 0.18 | 0.00 | 0.78 | 0.54 | 0.00 | 0.35 | 0.00 | 0.00 | 4.33 | 0.54 |
| AAGTTGCATC | Hs.29131 | nuclear receptor coactivator 2 | 0.18 | 0.00 | 0.78 | 0.18 | 0.20 | 0.18 | 0.00 | 0.00 | 4.33 | 0.18 |
| TAACCAAACA | AJ243671 | KIAA0144 gene product | 0.18 | 0.00 | 0.78 | 0.18 | 0.39 | 0.00 | 0.00 | 0.00 | 4.33 | 0.18 |
| GGCAGCCTGG | U94836 | protein with polyglutamine repeat; calcium (ca2+) homeostasis endoplasmic reticulum protein | 0.18 | 0.00 | 0.78 | 0.18 | 0.39 | 0.00 | 0.65 | 0.64 | 4.33 | 0.18 |
| TATCACTTTT | Z11898 | POU domain, class 5, transcription factor 1 | 0.18 | 0.00 | 0.78 | 0.36 | 0.00 | 0.00 | 0.00 | 0.32 | 4.33 | 0.36 |
| CTACTGTCTA | Hs.108124 | ribosomal protein L41 | 0.18 | 0.00 | 0.78 | 0.00 | 0.00 | 0.00 | 0.65 | 0.64 | 4.33 | 0.00 |
| GTGCTATTCT | AL360136 | B7 homolog 3 | 0.18 | 0.00 | 0.78 | 0.90 | 1.17 | 1.06 | 0.98 | 0.00 | 4.33 | 0.90 |
| GAGCTGGTGA | NM_005830 | imogen 38 | 0.18 | 0.00 | 0.78 | 0.54 | 1.17 | 1.42 | 0.98 | 0.64 | 4.33 | 0.54 |
| GTCTGACCCC | Hs.173902 | protein phosphatase 2 (formerly 2A), regulatory subunit A (PR 65), alpha isoform | 0.37 | 0.00 | 1.57 | 0.54 | 3.13 | 2.48 | 0.98 | 1.29 | 4.24 | 0.54 |
| GTGGCGGGCG | D90313 | carcinoembryonic antigen-related cell adhesion molecule 1 (biliary glycoprotein) or others | 0.92 | 1.74 | 3.73 | 2.89 | 1.37 | 0.89 | 1.63 | 0.97 | 4.05 | 1.66 |

What is claimed is:

1. A method of screening for candidate agents capable of altering the biological activity of a polypeptide encoded by a polynucleotide involved in hypoxia-related tumorigenesis comprising contacting a test agent with a target cell expressing the polynucleotide, and monitoring activity of the expressed polypeptide product, wherein the test agent which modifies the activity of the polypeptide is a candidate agent.

2. The method of claim 1, wherein the biological activity is the induction of hypoxia-related gene enolase 2 (GenBank No. Y00691M27) or a biological equivalent thereof.

3. The method of claim 1, wherein the biological activity is the induction of a hypoxia-related gene, inducible in the absence of VHL, wherein the gene is selected from the group consisting of integrin alpha E (GenBank No. L25851), endothelin 2 (GenBank No. X55177), stress-induced endoplastic reticulum protein 1 (GenBank No. AB022427), phosphoglutamase 1 (GenBank No. M83088), cell-division cycle 25B (AL10980), and biological equivalents thereof.

4. The method of claim 1, wherein the biological activity is differential expression in a neoplastic cell as compared to a normal counterpart cell and the gene is selected from the group consisting of enolase 2 (GenBank No. Y00691M27) and glia maturation factor B (GenBank No. ABOO1106) and biological equivalents thereof.

5. The method of claim 1, wherein the biological activity is induction of a gene, induced in the absence of VHL but not hypoxia, wherein the gene is selected from the group consisting of metalloprotease 1 (GenBank No. AK001183), insulin-like growth factor binding protein 3 (GenBank No. Hs. 77326), and biological equivalents thereof.

6. The method of claim 1, wherein the target cell is a renal carcinoma cell.

7. The method of any one of claims 1 through 4, further comprising contacting a normal, healthy counterpart cell to the test cell and monitoring the activity of the polypeptide.

8. A method of analyzing an expressed gene in a cell comprising comparing a transcript isolated from the cell with a database comprising a polynucleotide or transcript of a gene identified in Table 2.

9. A method of analyzing the effect of an agent on the expression of at least one gene in a cell, comprising comparing at least one transcript(s) of the gene(s) isolated from the cell with a database comprising a polynucleotide or transcript of a gene identified in Table 2.
